(19) United States
(12) Patent Application Publication
(10) Pub. No.: US 2010/0104555 A1
(43) Pub. Date: Apr. 29, 2010

(54) HCV neutralizing epitopes

(75) Inventors: Mansun Law, San Diego, CA (US); Toshiaki Maruyama, La Jolla, CA (US); Dennis R. Burton, La Jolla, CA (US); Jonathan K. Ball, Newark (GB); Norman M. Kneteman, Edmonton (CA)

Correspondence Address: K&L Gates LLP, 3580 Carmel Mountain Road, Suite 200, San Diego, CA, 92130, US

(73) Assignee: The Scripps Research Institute, La Jolla, CA (US)

(21) Appl. No.: 12/290,017

(22) Filed: Oct. 24, 2008

(51) Int. Cl. A61K 38/17; A61K 38/16; C07H 21/04; C12N 15/63; C12N 1/21; C12N 5/10; C12N 5/06; C07K 16/00; A61K 31/7088; A61K 39/395; C12Q 1/70; A61P 31/12

(52) U.S. Cl. 424/130.1; 530/326; 530/324; 536/23.1; 435/320.1; 435/252.3; 435/325; 435/358; 514/12; 530/387.1; 530/388.1; 514/44 R; 435/5

(57) ABSTRACT

The invention relates to modified hepatitis C virus E2 polypeptides that are effective in eliciting the production of cross-neutralizing antibodies against hepatitis C virus. The invention provides modified hepatitis C virus E2 polypeptides, preparations and pharmaceutical compositions containing them, as well as methods for using these modified E2 polypeptides.

### CROSS REFERENCE TO RELATED APPLICATIONS

This application is related to PCT/US02/02303 filed Jan. 25, 2002 (published as WO 02/059340 on Aug. 1, 2002), and to U.S. Provisional Application Ser. No. 60/264,451, filed Jan. 26, 2001, the disclosures of which are specifically incorporated by reference herein in their entireties.

### BACKGROUND OF THE INVENTION

It is estimated that hepatitis C virus (HCV) infects about 2-3% of the world population, i.e. 120 to 170 million people worldwide. HCV infection predisposes the patient to chronic liver cirrhosis, cancer and liver failure. About 85% of individuals initially infected with HCV become chronically infected. Once established, chronic HCV infection causes an inflammation of the liver, and this can progress to scarring and eventually, liver cirrhosis. Some patients with cirrhosis will go on to develop liver failure or liver cancer. In the United States and Western Europe, the complications of chronic hepatitis and cirrhosis are the most common reasons for liver transplantation. In addition, liver disease caused by HCV is the leading cause of death in patients co-infected with human immunodeficiency virus. Given the large number of infected people worldwide, HCV infection can be a burden on health care systems worldwide.

Accordingly, there is a need for therapeutic agents and methods for the treatment of hepatitis C viral infections.

### SUMMARY OF THE INVENTION

The invention relates to modified hepatitis C virus E2 polypeptides containing conserved neutralizing epitopes, preparations and pharmaceutical compositions containing the polypeptides, as well as methods for using these modified E2 polypeptides. The invention is based on discovery of conformation-dependent cross-neutralizing antibodies against hepatitis C virus (HCV), the identification of discontinuous epitopes involved in binding to cross-neutralizing antibodies, and the discovery of immunodominant epitopes that can be altered to focus the immune response to conserved neutralizing epitopes. The invention provides modified HCV E2 polypeptides, nucleic acids encoding the modified HCV E2 polypeptides, and expression vectors for producing HCV E2 polypeptides, which can be incorporated into a vaccine for HCV. The invention also provides a cell comprising such nucleic acid or expression vector, a preparation or pharmaceutical composition comprising a modified HCV E2 polypeptide, as well as a method of eliciting an immune response in a mammal comprising administering a modified HCV E2 polypeptide, a method for determining whether a mammal has been infected with an HCV, and a method for identifying an anti-HCV agent.

In one aspect, the invention provides a modified hepatitis C viral (HCV) E2 polypeptide (i.e. polypeptide of the invention) having a discontinuous epitopes that includes, from the amino to the carboxy termini: (1) an amino acid segment, the sequence of which corresponds to amino acid residues 396 to 424 of a select HCV, (2) an amino acid segment, the sequence of which corresponds to amino acid residues 436 to 447 of the select HCV, and (3) an amino acid segment, the sequence of which corresponds to amino acid 523 to 540 of the select HCV. The polypeptide also has two or more amino acid substitutions at positions 416, 417, 483, 484, 485, 538, 540, 544, 545, 547, 549 or any combinations thereof, and a deletion of amino acid residues 384 to 395 relative to the full-length E2 polypeptide of the select HCV.

In some embodiments, the first amino acid segment has the sequence of any one of SEQ ID NOs: 791-815; the second amino acid segment has the sequence of any one of SEQ ID NOs: 815-840 and the third amino acid segment has the sequence of any one of SEQ ID NOs: 841-865. In one embodiment, the first amino acid segment is TAGLVGLLTPGAKQNIQLINTNGSWHINS (SEQ ID NO: 694), the second amino acid segment is GWLAGLFYQHKF (SEQ ID NO: 695) and the third amino acid segment is GAPTYSWGANDTDVFVLN (SEQ ID NO: 696).

In some embodiments, the first and second segments are separated by about 10 amino acid residues. In some embodiments, the second and third segments are separated by about 50 amino acid residues. In some embodiments, the first and second segments are separated by about nine amino acid residues, and the second and third segments are separated by about 50 amino acid residues. In some embodiments, the polypeptide has the sequence of SEQ ID NO: 866, 867, 868, 869 or 870. In another embodiment, the polypeptide sequence consists of SEQ ID NO: 866, 867, 868, 869 or 870.

In some embodiments, the sequence of the polypeptide includes: (1) a segment defined by amino acids 396 to 746 of an HCV; (2) a segment defined by amino acids 396 to 717 of an HCV; (3) a segment defined by amino acids 396 to 661 of an HCV; (4) a segment defined by amino acids 396 to 647 of an HCV or (5) a segment amino acids 396 to 645 of an HCV.

In some embodiments, the polypeptide has an amino or carboxy terminal tag. In some embodiments, the tag is a poly-histidine sequence, a FLAG sequence, an HA sequence, a myc sequence, a V5 sequence, a chitin binding protein sequence, a maltose binding protein sequence, a glutathione-S-transferase sequence or an N-terminal ubiquitin signal.

In another aspect, the invention provides an isolated nucleic acid that encodes a polypeptide of the invention. In some embodiments, the isolated nucleic acid has a sequence encoding a polypeptide of SEQ ID NO: 866, 867, 868, 869 or 870. In some embodiments, the isolated nucleic acid has the sequence of SEQ ID NO: 874, 875, 876, 877, 878, 879, 880 or 881. In one embodiment, the isolated nucleic acid consists of the sequence of SEQ ID NO: 874, 875, 876, 877, 878, 879, 880 or 881. In some embodiments, the isolated nucleic acid that encodes a polypeptide of the invention is operably linked to an expression control sequence. In some embodiments, the expression control sequence is a viral, phage, bacterial, or mammalian promoter.

In another aspect, the invention provides an expression vector that has a nucleic acid sequence encoding a polypeptide of the invention. In some embodiments, the nucleic acid encoding the polypeptide is operably linked to an expression control sequence. In some embodiments, the expression control sequence is a promoter. In one embodiment, the promoter is a viral promoter, a bacterial promoter or a mammalian promoter.

In another aspect, the invention provides a cell that has the expression vector having a nucleic acid sequence encoding a polypeptide of the invention. The cell can be a bacterial cell, mammalian cell or a Chinese hamster ovary cell.

In another aspect, the invention provides a method of eliciting an immune response in a mammal that involves administering to the mammal a polypeptide of the invention. In some embodiments, the mammal is a mouse, sheep, goat, horse, rabbit, hamster, rat or human.

In some embodiments, the method also involves obtaining a blood sample from the mammal. In one embodiment, the method involves further isolating an antibody or antibody-producing cell from the mammal. In some embodiments, the antibody is a cross-neutralizing antibody. In some embodiments, the antibody is a murine antibody.

In some embodiments, the polypeptide is in a pharmaceutical composition with a pharmaceutically acceptable carrier. In some embodiments, the polypeptide is in an amount effective to prevent or treat HCV infection in the mammal. In some embodiments, the method also involves administering to the mammal a second dose of the polypeptide at a selected time after the first administration. In some embodiments, the method involves eliciting an immune response in a mammal that has been exposed to HCV. In some embodiments, the mammal is a human.

In another aspect, the invention provides an antibody isolated using the method described above. In some embodiments, the antibody is a single chain variable fragment (scFv) or an antigen binding fragment, e.g. Fab or F(ab′)2. In some embodiments, the antibody is Fab C1, J2, H3 or L4. In some embodiments, the antibody is a monoclonal antibody, e.g. an IgG antibody. In some embodiments, the IgG antibody is AR3A, AR3B, AR3C or AR3D.

In another aspect, the invention provides a method of eliciting an immune response in a mammal that involves administering to the mammal a nucleic acid that encodes a polypeptide of the invention. In another aspect, the invention provides a method of eliciting an immune response in a mammal that involves administering to the mammal an expression vector that includes a nucleic acid sequence encoding a polypeptide of the invention. In some embodiments, the nucleic acid has a sequence encoding a polypeptide of SEQ ID NO: 866, 867, 868, 869 or 870. In some embodiments, the nucleic acid has a sequence that includes the sequence of SEQ ID NO: 874, 875, 876, 877, 878, 879, 880 or 881. In some embodiments, the nucleic acid is operably linked to an expression control sequence. In some embodiments, the expression control sequence is a viral, phage, bacterial, or mammalian promoter. In some embodiments, the promoter is a SV40 promoter, a Rous Sarcoma Virus promoter, or a cytomegalovirus immediate early promoter.

In another aspect, the invention provides a pharmaceutical composition that includes (1) a polypeptide of the invention, (2) an isolated nucleic acid that encodes a polypeptide of the invention, (3) an expression vector that includes a nucleic acid sequence encoding a polypeptide of the invention, or (4) an antibody of the invention, and a pharmaceutically acceptable carrier.

In another aspect, the invention provides a purified preparation of a polypeptide of the invention in which at least 80% of the polypeptides are in a conformation capable of binding to a conformation-dependent cross-neutralizing antibody.

In another aspect, the invention provides a purified preparation of an antibody of the invention in which the antibody is at least 5% of the antibodies in the preparation.

In another aspect, the invention provides a method for determining whether a mammal has been infected with an HCV that involves contacting a blood sample from the mammal with a polypeptide of the invention and determining whether the polypeptide binds specifically with an antibody from the blood of the mammal to form a polypeptide-antibody complex, wherein the presence of the complex indicates that the mammal has been infected with an HCV and the absence of the complex indicates that the mammal has not been infected with the virus.

The invention involves the discovery of conserved neutralizing epitopes and immunodominant epitopes useful for generating modified HCV E2 polypeptides that are effective in eliciting an immuno response directed against conserved neutralizing epitopes. Thus, the invention provides for modified HCV E2 polypeptides correctly presenting a conserved HCV E2 conformational epitope that are useful as immunogens, e.g. in an HCV vaccine, for raising cross-neutralizing antibodies against HCV. The corresponding coding nucleic acids can be used as DNA-based vaccines.

Any feature or combination of features described herein are included within the scope of the present invention provided that the features included in any such combination are not mutually inconsistent, as will be apparent from the context, this specification and the knowledge of one of ordinary skill in the art.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents and other references mentioned herein are incorporated by reference in their entirety. Amino acid designations may include full name, three-letter, or single-letter designations as commonly understood by one of ordinary skill in the art to which this invention belongs. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF THE DRAWINGS

FIG. 1A-C illustrate properties of anti-HCV E2 Fabs isolated as described herein. FIG. 1A illustrates the specificity of anti-E2 Fabs. The binding of Fabs to GST-E1E2 complex and E2 is compared. The GST-E1E2 fusion protein was captured by a goat anti-GST antibody while soluble E2 and ovalbumin were coated directly onto ELISA plates. Fabs were added to the antigens and subsequently detected with phosphatase-conjugated goat anti-human F(ab)′2 IgG. Recombinant Fabs were produced in cleared lysate of E. coli transformed with the corresponding phagemids. FIG. 1B illustrates competition between MAb AR3A and anti-E2 Fabs. Vaccinia-expressed E1E2 was captured onto ELISA wells by lectin and preincubated with saturating concentration of soluble Fabs before the addition of MAb AR3A. Binding of MAb AR3A was detected with a goat anti-human IgG Fc antibody and the % reduction of binding compared to that in the absence of a Fab is shown. Lightly-shaded bars indicate that Fabs bind E2 better than E1E2; while bars of medium shading indicate that Fabs bind E1E2 better than E2. FIG. 1C illustrates the inhibition of anti-E2 Fab binding to E1E2 by mouse MAb H53. E1E2 was captured onto ELISA wells in the same manner as shown for FIG. 1B and was pre-incubated with a saturating concentration of MAb H53 before the addition of soluble Fabs. Binding of Fabs was detected with a goat anti-human IgG F(ab)′2 antibody and the % reduction of binding compared to that without MAb H53 is shown. Lightly-shaded bars indicate that Fabs bind E2 better than E1E2; while bars of medium shading indicate that Fabs bind E1E2 better than E2.

FIG. 2 shows neutralization of HCVpp by human Fabs. Infectivity in Relative Light Units (RLU) is shown for infection of pseudotype virus particles generated with viral Env gene from murine leukemia virus (MLV), H77 (GT 1a), OH8 (GT 1b), CON1 (GT 1b) or J6 (GT 2a) in the presence of 10 μg/mL Fabs. AR1-Fabs: B2, D1 & E; AR2-Fabs: F & G; AR3-Fabs: C1, J2, J3 & L4. Control, anti-HIV gp120 Fab b12; Empty, background infectivity from pseudotype virus generated without Env gene. Dotted lines indicate HCVpp infectivity in the absence of any antibody. Error bars represent SEM calculated from three experiments performed in the same manner.

FIG. 3 is a schematic diagram of E2 regions important for binding of AR3-specific antibodies. E2 (residues 384-746) is a transmembrane glycoprotein, and a truncated form of E2 (residues 384-661) can be expressed as a soluble protein that retains its ability to bind cell lines expressing HCV receptors and CD81-LEL (Michalak, J. P. et al. J. Gen. Virol. 78, 2299-2306 (1997)). The regions that were investigated by antibody competition and alanine mutagenesis are indicated by dotted and solid boxes, respectively. The AR3 discontinuous epitopes include: (1) amino acids 396-424 having the sequence TAGLVGLLTPGAKQNIQLINTNGSWHINS (SEQ ID NO: 694); (2) amino acids 436-447 having the sequence GWLAGLFYQHKF (SEQ ID NO: 695), and (3) amino acids 523-540 having the sequence GAPTYSWGANDTDVFVLN (SEQ ID NO: 696). The crucial residues in these regions are S424, G523, P525, G530, D535, V538 and N540. The locations of the N-linked glycans are indicted by branched forks. The hypervariable regions (see Troesch, M. et al. Virology 352, 357-367 (2006) and the transmembrane regions are indicated by the designation HVRs and TM.

FIG. 4A-D illustrate the kinetics and toxicity of human MAbs in Alb-uPA/SCID mice. Previous passive antibody studies in animal models have reported relatively high antibody concentrations are needed for protection. For instance, to achieve sterilizing immunity by single mAB treatment against HIV in hu-PBL/SCID mice [Gauduin et al. Nat. Med. 3, 1389-1393 (1997)] and against chimeric simian/human immunodeficiency virus (SHIV) in macaques [Parren et al. J. Virol. 75, 8340-8347 (2001)], serum concentrations in the animals of the order of 100-fold in vitro 90% neutralization titers (IC₉₀) have been required. The IC₉₀ titers (against HCVpp-H77) for MAb AR3A and AR3B are 11 and 20 g/mL, respectively, suggesting that relatively large doses of antibody may be required for protection. The kinetics of the human MAbs AR3A (FIG. 4A), AR3B (FIG. 4B) and b6 (FIG. 4C) in control Alb-uPA/SCID mice were studied. Transplanted Alb-uPA/SCID mice with a low level of human liver chimerism were injected intraperitoneally with 100, 150 or 200 mg/kg MAb, and blood samples were collected by tail bleed. Human antibody in the murine sera was measured by a quantitative sandwich ELISA using conjugated and unconjugated goat anti-human F(ab)′₂ antibody. The mouse identity (ID) (e.g. N329), antibody dose used (bracket) and the mean±s.d. of each treatment are indicated. FIG. 4D shows the health status of the animals as monitored by their general wellness and weight change. No specific weight loss or signs of illness associated with the administration of the MAbs were noted in the mice during the experiment. One mouse (N457) was euthanized due to unrelated morbidity at Day 7.

FIG. 5A-B illustrate the specific virus neutralizing activity of human MAbs in Alb-uPA/SCID mice. The neutralizing activity in mouse sera collected ten days after injection was determined by HCVpp-H77 neutralization assay. FIG. 5A illustrates neutralization with serially diluted mouse sera. Mouse sera containing anti-HCV MAbs AR3A and AR3B (filled symbols) neutralized 50% of HCVpp infectivity (IC₅₀) in the range of 1:200 to 1:1000. Control mouse sera from mice injected with an isotype MAb DEN3 (dark lines/open symbols) or PBS (light lines/open symbols) neutralized non-specifically 50% virus infectivity at 10-fold dilution. Non-specific neutralization was not observed when the control sera were diluted 100-fold. FIG. 5B illustrates the conservation of virus neutralizing activity of anti-HCV MAbs. The data shown in FIG. 5A were normalized to the level of human IgG in the mouse sera quantified in FIG. 4. In this experiment, MAbs AR3A (open diamond) and AR3B (open square) were titrated alongside with the mouse sera to construct the standard curves and the IC₅₀ titers of MAbs AR3A (open diamond) and AR3B (open square) are 0.4 and 1 μg/mL, respectively. The IC₅₀ titers of mouse sera containing anti-HCV MAbs AR3A and AR3B are in the range of 0.4-1.1 (mean 0.8±s.d. 0.3) and 0.5-3 (mean 1.2±s.d. 0.9) μg/mL, respectively. Isotype control MAbs b6 & DEN3 (open symbols) do not neutralize HCVpp.

FIG. 6 illustrates the levels of human MAb in human liver-chimeric mice 24 hours post-administration. Human liver-chimeric mice (n=6) were injected intraperitoneally with a dose of 200 mg/kg of the isotype control mAb b6, AR3A or AR3B and blood samples were collected at 24 hours before challenging with a genotype la HCV-infected human serum KP (100 μL) by intrajugular venous injection. Intravenous administration of human serum is the most reliable way to assure delivery of human serum but a stressful procedure: 5 of 18 treated mice did not recover after the procedure. Human IgG in mouse sera were quantified as in FIG. 4. Filled bars, mice that died after intrajugular injection of KP serum; Open bars, mice survived the procedure and used in the protection experiments. The mean serum human IgG levels±s.d. in the surviving mice of group b6, AR3A and AR3B are 2.5±0.3, 3.1±0.5 and 2.6±0.3 mg/mL, respectively. Note that decay of the human MAbs following virus challenge, which may be an explanation for the infection of several antibody-treated mice at later time points, could not be determined as in FIG. 4 because the infected human serum contains normal human antibodies interfering with IgG quantification. In a control experiment using transplanted mice with low level of human chimerism, human IgG antibodies were readily detected in the mice challenged with 100 μL of the infected serum (n=5, Day 1 mean±s.d. mouse serum human IgG concentration: 2.6±0.5 mg/mL) (data not shown).

FIG. 7A-D demonstrate passive antibody protection against an HCV population. Human liver-chimeric mice (n=6) injected intraperitoneally with 200 mg/kg of the isotype control mAb b6 (A), mAb AR3A (B) or AR3B (C), were challenged 24 hours later by intrajugular venous injection of genotype 1 a HCV-infected human serum (˜2×10⁵ HCV RNA copies). One or two mice per group did not recover from anesthesia after intrajugular injection. Data shown are serum viral load in mice quantified by real-time TaqMan PCR. Owing to morbidity, mice N680 and N672 were killed on days 41 and 45, respectively. IU, international units; ID, identification number; i.p., intraperitoneally; i.v., intravenously. FIG. 7A-C are results showing the absence of serum HCV RNA 6 days after viral challenge in mice injected with mAb AR3A and mAb AR3B. FIG. 7D is a sequence comparison of a viral quasispecies population in the HCV genotype 1a-infected human serum. Partial E2 amino acid sequences (residues 384-622) of a total of 40 clones (represented by KP S9 (SEQ ID NO: 701), KP R14 (SEQ ID NO: 702), KP S6 (SEQ ID NO: 703), KP S18 (SEQ ID NO: 704), KP S16 (SEQ ID NO: 705), KP R8 (SEQ ID NO: 706), KP S20 (SEQ ID NO: 707), KP S4 (SEQ ID NO: 708), KP R3 (SEQ ID NO: 709), KP S3 (SEQ ID NO: 710), KP S12 (SEQ ID NO: 711), KP S15 (SEQ ID NO: 712), KP S5 (SEQ ID NO: 713), KP R7 (SEQ ID NO: 714), KP R11 (SEQ ID NO: 715), KP R1 (SEQ ID NO: 716), KP R12 (SEQ ID NO: 717), KP S7 (SEQ ID NO: 718), KP R15 (SEQ ID NO: 719), KP R18 (SEQ ID NO: 720), KP S11 (SEQ ID NO: 721) and KP R20 (SEQ ID NO: 722)) randomly selected from two independent RT-PCR cloning are shown. See also Table E-9. The top sequence, clone KP S9, represents the consensus and dominant sequence in this infectious serum. The periods indicate regions of amino acid sequence identity. The frequency of each clone is bracketed. Hypervariable regions (HVRs) are within the dashed-line boxes. Regions important for binding of AR3-antibodies are within the solid-line boxes. The corresponding sequences of isolates H77 (SEQ ID NO: 723) and UKN1b12.16 (SEQ ID NO: 724), sharing 87% and 75% amino acid identity with KP S9, respectively, are shown for comparison.

FIG. 8 is a schematic illustration of a panel of recombinant E2 fragments. Full length E2 (residues 384-746) is shown at the top and the relative locations of N-glycans and cysteines are marked by light and darker vertical lines, respectively. The hypervariable region 1 (HVR1) at the N-terminus and transmembrane region at the C-terminus of E2 are shaded. The positions of N— or C-terminal truncation in the mutants are indicated, and the Flag tags are indicated by boxes at the C-termini. Fragments are named according to the primer sets used in gene amplification. According to the E2 model proposed by Yagnik et al., Proteins 40, 355-66 (2000), disulfide bridges are predicted to form between C1-C16 (i.e. residues C429-C644), C2-C4 (C452-C486), C8-C9 (C552-C564), C13-C14 (C597-C607), and C7-C11 (C508-581) or C7-C12 (C581-585).

FIG. 9A-H illustrate the binding properties of E2 fragments. 293T cells were transfected with DNA plasmids encoding the E2 fragments depicted in FIG. 8 and the expression of the corresponding proteins was assayed by sandwich ELISA. ELISA wells were pre-coated with MAbs specific to the 3 different E2 antigenic regions (AR1, AR2 and AR3), or CD81-LEL, to capture the recombinant proteins in serially diluted cell supernatants. The reagents used in the capture are indicated on the left of the bar charts. Bound E2 fragments were detected using the mouse anti-Flag tag M2 MAb (Sigma). Data shown are means of duplicate measurements.

FIG. 10 is an SDS-PAGE analysis of the purification of E2f1r2a using a MAb AR3A-conjugated affinity column. Three batches of E2f1r2a were produced by transient transfection of 293T cells (˜5×10⁸ cells per batch) with the corresponding expression plasmid. Batch 3 was produced in the presence of 10 μM kifunesine (BIOMOL), a potent inhibitor of the glycoprotein processing α-mannosidase I and is used to improve glycan homogeneity in the glycoproteins. Cell supernatants were loaded onto an antibody-affinity column (MAb AR3A, 5 mL) by gravity flow and bound proteins were eluted with a low pH buffer (0.2 M glycine, pH 2.7). Batches 2 and 3 was purified twice to monitor purification efficiency. Note that the majority of E2f1r2a was isolated in the first round for these batches. The eluents were collected into tubes with 0.1 volume of neutralizing buffer (2 M Tris-HCl, pH 9). The eluants were analyzed by 4-15% gradient SDS-PAGE (BIO-RAD). The pre-stained protein standard SeeBlue Plus2 (Invitrogen) is shown on the left. For Batches 1 and 2, monomeric E2f1r2a was purified to greater than 90% purity and has a similar apparent size (5565 kDa) under both reducing and non-reducing conditions. For Batch 3, more higher molecular impurities were found, which can probably be removed by a second chromatographic step. In the presence of kifunensine, the E2f1r2a protein bands appear less diffused and more distinct, indicating that N-glycans on the Batch 3 recombinant proteins (glycoforms) are more homogeneous than that of Batches 1 and 2. The yields of E2f1r2a in the three batches were approximately 1 mg.

FIG. 11A-B are analytical results from the size-exclusion purification of E2f1r2a. E2f1r2a purified by MAb AR3A affinity column was concentrated to 0.5 mL using an ultra-centrifugal filter device with a 30 kDa nominal molecular weight limit (Millipore). The concentrated proteins were loaded onto a Sephedex 75 size-exclusion column (GE Healthcare) using a ÄKTA Fast Protein Liquid Chromatography (FPLC) system (GE Healthcare). The proteins were separated in Tris buffer (0.1 M Tris-HCl pH 7.4 and 150 mM NaCl) and elution fractions of 0.5 mL were collected by an automatic fractionator. The chromatogram of E2f1r2a was shown as an overlay with the chromatogram of protein standards (peaks labeled A, B, C and D) (FIG. 11A). The protein standards are: (A) blue dextran 2000, (B) bovine serum albumin 67 kDa, (C) ovalbumin 43 kDa, and (D) chymotrypsinogen 25 kDa (GE Healthcare). Fractions 14-22 were analyzed by non-reducing SDS-PAGE (4-15% gradient, BIO-RAD) (FIG. 11B). The pre-stained protein standard SeeBlue Plus2 (Invitrogen) is shown on the left. The results showed that the high molecular weight impurities eluted from the MAb AR3A-affinity column were separated from the glycoforms of E2f1r2a, which appear to be monomers of size between 43-67 kDa in gel filtration.

FIG. 12 is an SDS-PAGE analysis of the purification of E2f1r2a using neutral pH elution buffer. E2f1r2a was produced in the presence of kifunensine, loaded onto a MAb AR3A-affinity column and eluted with an increasing step-gradient of the chaotropic salt sodium thiocyanate (NaSCN). Lane: (1) E2f1r2a eluted with low pH buffer as a control; (2) E2f1r2a eluted with 0.5 M NaSCN; (3) 1 M NaSCN; and (4) 2 M NaSCN. The purified proteins were analyzed by non-reducing SDS-PAGE (4-15% gradient, BIO-RAD). The prestained protein standard SeeBlue Plus2 (Invitrogen) is shown on the left. Note that most of the high molecular weight impurities were eluted at 0.5 M NaSCN.

FIG. 13 is an SDS-PAGE analysis of the purification of E2f1r2a using high pH elution buffer. E2f1r2a was produced in the presence of kifunensine, loaded onto a MAb AR3A-affinity column and eluted with a step-gradient of buffers with increasing pH. The eluents were collected into tubes with 0.1 volume of neutralizing buffer (2 M Tris-HCl, pH 7.4). Lane: (1) E2f1r2a eluted with 2 M NaSCN as a control; (2) E2f1r2a eluted with 0.2 M glycine pH 9.5; (3) pH 10.5; (4) pH 11.5; (5) pH 12.5; and (6) lane 4 sample filtered through an ultra-centrifugal filter device with a 100 kDa nominal molecular weight limit (Millipore). The purified proteins were analyzed by non-reducing SDS-PAGE (4-15% gradient, BIO-RAD). The pre-stained protein standard SeeBlue Plus2 (Invitrogen) is shown on the right.

FIG. 14 is an SDS-PAGE analysis of the purification of E2ΔTM using a MAb AR3A-conjugated affinity column. E2f1r2a (lanes 1 & 2) and E2ΔTM (lanes 3-6) were purified and eluted at pH 7.4 (2M sodium thiocyanate). The high molecular weight impurities were removed by filtering through an ultracentrifugal filter device with a 100 kDa nominal molecular weight limit (Millipore). The purified proteins were analyzed by 4-15% gradient non-reducing SDS-PAGE (BIO-RAD). The pre-stained protein standard SeeBlue Plus2 (Invitrogen) is shown on the left. Lane: (1) E2f1r2a produced in the presence of kifunensine; (2) filtered lane 1 sample; (3) E2ΔTM; (4) filtered lane 3 sample; (5) E2ΔTM produced in the presence of kifunensine; and (6) filtered lane 5 sample.

FIG. 15A-E are graphs illustrating the antigenic properties of E2f1r2a. E2f1r2a produced in the presence of kifunensine was purified using a MAb AR3A-affinity column and was eluted with low pH (0.2 M glycine pH 2.2), 2 M NaSCN (pH 7.4) or high pH (0.2 M glycine pH 11.5) buffer. The different purified E2f1r2a monomers were titrated from 4 μg/mL (˜145 nM, 5-fold serial dilution) to investigate their antigenicities. To study their binding to anti-E2 antibodies, the purified proteins were captured onto microwells precoated with Galanthus nivalis lectins (5 μg/mL) and the captured proteins detected with the indicated human anti-E2 monoclonal antibodies (MAbs). To study their binding to CD81-LEL, microwells coated with maltose binding protein (MBP)-fused CD81-large extracellular loop (LEL) (10 μg/mL) were used to captured the purified proteins and bound proteins were detected with the mouse anti-FLAG tag MAb M2. Bound human or mouse MAbs were detected with peroxidase-conjugated anti-human or anti-mouse secondary antibodies and TMB substrate. The results show that E2f1r2a monomers eluted by buffers with different pH are similar antigenically.

FIG. 16A-E are graphs illustrating the antigenic properties of E2ΔTM. E2ΔTM produced in the presence of kifunensine was purified using a MAb AR3A-affinity column and was eluted with 2 M NaSCN buffer (pH 7.4). The effect of pH on the antigenicity of the protein was investigated. Purified E2ΔTM monomers were exposed briefly to low or high pH by adding 10-fold excess volume of 0.2M glycine pH 2.2 or pH 11.5, respectively. After 10 minutes, the solutions were neutralized by adding equal volume of 2M Tris-HCl pH 7.4 and treated and untreated E2ΔTM monomers were titrated from 5.5 μg/mL (˜145 nM, 5-fold serial dilution). To study their binding to anti-E2 antibodies, the purified proteins were captured onto microwells precoated with Galanthus nivalis lectins (5 μg/mL) and the captured proteins detected with the indicated human anti-E2 monoclonal antibodies (MAbs). To study their binding to CD81-LEL, microwells coated with maltose binding protein (MBP)-fused CD81-large extracellular loop (LEL) (10 μg/mL) were used to captured the purified proteins and bound proteins were detected with the mouse anti-FLAG tag MAb M2. Bound human or mouse MAbs were detected with peroxidase-conjugated anti-human or anti-mouse secondary antibodies and TMB substrate. The results show that pH does not have a significant effect on the antigenicity of E2ΔTM.

### DETAILED DESCRIPTION OF THE INVENTION

The invention relates to modified hepatitis C virus E2 polypeptides, preparations and pharmaceutical compositions containing them, and methods for using them. The invention is based on discovery of conformation-dependent cross-neutralizing antibodies against hepatitis C virus (HCV), the identification of discontinuous epitopes involved in binding to such cross-neutralizing antibodies, and discovery of immunodominant residues that can be altered to focus the immune response to conserved neutralizing epitopes. The invention provides modified HCV E2 polypeptides, nucleic acids encoding the modified HCV E2 polypeptides and expression vectors for producing HCV E2 polypeptides. The invention also provides a cell comprising such nucleic acid or expression vector, a preparation or pharmaceutical composition comprising a modified HCV E2 polypeptide, as well as a method of eliciting an immune response in a mammal comprising administering a modified HCV E2 polypeptide, a method for determining whether a mammal has been infected with an HCV, and a method for identifying an anti-HCV agent.

Hepatitis C Virus

Hepatitis C virus (HCV) is a noncytopathic, positive-stranded RNA virus that causes acute and chronic hepatitis and hepatocellular carcinoma. Hoofnagle, J. H. (2002) Hepatology 36, S21-29. The hepatocyte is the primary target cell, although various lymphoid populations, especially B cells and dendritic cells may also be infected at lower levels. Kanto et al. (1999) J. Immunol. 162, 5584-5591; Auffermann-Gretzinger et al. (2001) Blood 97, 3171-3176; Hiasa et al. (1998) Biochem. Biophys. Res. Commun. 249, 90-95. A striking feature of HCV infection is its tendency towards chronicity with at least 70% of acute infections progressing to persistence (Hoofnagle, J. H. (2002) Hepatology 36, S21-29). HCV chronicity is often associated with significant liver disease, including chronic active hepatitis, cirrhosis and hepatocellular carcinoma (Alter, H. J. & Seeff, L. B. (2000) Semin. Liver Dis. 20, 17-35). With over 170 million people currently infected (id.), HCV represents a growing public health concern.

As used herein, the term “hepatitis C virus,” “HCV,” or “HCVs” includes different viral genotypes, subtypes and quasispecies. It includes any noncytopathic RNA virus that has a single and positive-stranded RNA genome belonging to the Hepacivirus genus of the Flaviviridae family. The term includes different isolates of HCV such as, without limitation, those having polyprotein sequences and accession numbers shown above, as well as any others in the NCBI database. Examples of different genotypes encompassed by this term include, without limitation, genotype 1, 2, 3, 4, 5 and 6. Examples of different subtypes include, without limitation, 1a, 1b, 1c, 2a, 2b, 2c, 2i, 2k, 3a, 3b, 3k, 4a, 4d, 4f, 5a, 6a, 6b, 6c, 6d, 6e, 6f, 6g, 6h, 6i, 6j, 6k, 6l, 6m, 6n, 6o, 6q, 6p and 6t. See http://www.hcvdb.org/. The term also includes cell culture HCVs (HCVcc) and pseudotype HCVs (HCVpp), as well as HCV quaqsispecies. Various HCVs are described by Simmonds P. in Genetic diversity and evolution of hepatitis C virus—15 years on, J Gen Virol 85:3173-3188 (2004) and Simmonds et al. in Consensus proposals for a unified system of nomenclature of hepatitis C virus genotypes, Hepatology 42:962-973 (2005). HCV nucleotide sequences are known in the art and can be found at http://www.hcvdb.org/ and http://hcv.lanl.gov/content/sequence/LOCATE/locate.html.

The single stranded HCV RNA genome has a single open reading frame (ORF) encoding a large polyprotein. The polyprotein has about 3010-3033 amino acids (Q.-L. Choo, et al. Proc. Natl. Acad. Sci. USA 88, 2451-2455 (1991); N. Kato et al., Proc. Natl. Acad. Sci. USA 87, 9524-9528 (1990); A. Takamizawa et al., J. Virol. 65, 1105-1113 (1991)). Nucleic acid and amino acid sequences for different isolates of HCV can be found in the art, for example, in the National Center for Biotechnology Information (NCBI) database. See ncbi.nlm.nih.gov.

An example of an HCV subtype 1a is strain H77, which can be found in the NCBI database as accession number AF009606. Its polyprotein sequence (AAB66324) is as follows:

(SEQ ID NO: 763)

| | |
|---|---|
| 1 | MSTNPKPQRK TKRNTNRRPQ DVKFPGGGQI VGGYLLPRR GPRLGVRATR KTSERSQPRG |
| 61 | RRQPIPKARR PEGRTWAQPG YPWPLYGNEG CGWAGWLLSP RGSRPSWGPT DPRRRSRNLG |
| 121 | KVIDTLTCFG ADLMGYIPLV GAPLGGAARA LAHGVRVLED GVNYATGNLP GCSFSIFLLA |
| 181 | LLSCLTVPAS AYQVRNSSGL YHVTNDCPNS SIVYEAADAI LHTPGCVPCV REGNASRCWV |
| 241 | AVTPTVATRD GKLPTTQLRR HIDLLVGSAT LCSALYVGDL CGSVFLVGQL FTFSPRRHWT |
| 301 | TQDCNCSIYP GHITGHRMAW DMMMNWSPTA ALVVAQLLRI PQAIMDMIAG AHWGVLAGIA |
| 361 | YFSMVGNWAK VLVVLLLFAG VDAETHVTGG SAGRTTAGLV GLLTPGAKQN IQLINTNGSW |
| 421 | HINSTALNCN ESLNTGWLAG LFYQHKFNSS GCPERLASCR RLTDFAQGWG PISYANGSGL |
| 481 | DERPYCWHYP PRPCGIVPAK SVCGPVYCFT PSPVVVGTTD RSGAPTYSWG ANDTDVFVLN |
| 541 | NTRPPLGNWF GCTWMNSTGF TKVCGAPPCV IGGVGNNTLL CPTDCFRKHP EATYSRCGSG |
| 601 | PWITPRCMVD YPYRLWHYPC TINYTIFKVR MYVGGVEHRL EAACNWTRGE RCDLEDRDRS |
| 661 | ELSPLLLSTT QWQVLPCSFT TLPALSTGLI HLHQNIVDVQ YLYGVGSSIA SWAIKWEYVV |
| 721 | LLFLLLADAR VCSCLWMMLL ISQAEAALEN LVILNAASLA GTHGLVSFLV FFCFAWYLKG |
| 781 | RWVPGAVYAF YGMWPLLLLL LALPQRAYAL DTEVAASCGG VVLVGLMALT LSPYYKRYIS |
| 841 | WCMWWLQYFL TRVEAQLHVW VPPLNVRGGR DAVILLMCVV HPTLVFDITK LLLAIFGPLW |
| 901 | ILQASLLKVP YFVRVQGLLR ICALARKIAG GHYVQMAIIK LGALTGTYVY NHLTPLRDWA |
| 961 | HNGLRDLAVA VEPVVFSRME TKLITWGADT AACGDIINGL PVSARRGQEI LLGPADGMVS |
| 1021 | KGWRLLAPIT AYAQQTRGLL GCIITSLTGR DKNQVEGEVQ IVSTATQTFL ATCINGVCWT |
| 1081 | VYHGAGTRTI ASPKGPVIQM YTNVDQDLVG WPAPQGSRSL TPCTCGSSDL YLVTRHADVI |
| 1141 | PVRRRGDSRG SLLSPRPISY LKGSSGGPLL CPAGHAVGLF RAAVCTRGVA KAVDFIPVEN |
| 1201 | LETTMRSPVF TDNSSPPAVP QSFQVAHLHA PTGSGKSTKV PAAYAAQGYK VLVLNPSVAA |
| 1261 | TLGFGAYMSK AHGVDPNIRT QVRTITTGSP ITYSTYGKFL ADGGCSGGAY DIIICDECHS |
| 1321 | TDATSILGIG TVLDQAETAG ARLVVLATAT PPGSVTVSHP NIEEVALSTT GEIPFYGKAI |
| 1381 | PLEVIKGGRH LIFCHSKKKC DELAAKLVAL GINAVAYYRG LDVSVIPTSG DVVVVSTDAL |
| 1441 | MTGFTGDFDS VIDCNTCVTQ TVDFSLDPTF TIETTTLPQD AVSRTQRRGR TGRGKPGIYR |
| 1501 | FVAPGERPSG MFDSSVLCEC YDAGCAWYEL TPAETTVRLR AYMNTPGLPV CQDHLEFWEG |
| 1561 | VFTGLTHIDA HFLSQTKQSG ENFPYLVAYQ ATVCARAQAP PPSWDQMWKC LIRLKPTLHG |
| 1621 | PTPLLYRLGA VQNEVTLTHP ITKYIMTCMS ADLEVVTSTW VLVGGVLAAL AAYCLSTGCV |
| 1681 | VIVGRIVLSG KPAIIPDREV LYQEFDEMEE CSQHLPYIEQ GMMLAEQFKQ KALGLLQTAS |
| 1741 | RQAEVITPAV QTNWQKLEVF WAKHMWNFIS GIQYLAGLST LPGNPAIASL MAFTAAVTSP |
| 1801 | LTTGQTLLFN ILGGWVAAQL AAPGAATAFV GAGLAGAAIG SVGLGKVLVD ILAGYGAGVA |
| 1861 | GALVAFKIMS GEVPSTEDLV NLLPAILSPG ALVVGVVCAA ILRRHVGPGE GAVQWMNRLI |
| 1921 | AFASRGNHVS PTHYVPESDA AARVTAILSS LTVTQLLRRL HWQISSECTT PCSGSWLRDI |
| 1981 | WDWICEVLSD FKTWLKAKLM PQLPGIPFVS CQRGYRGVWR GDGIMHTRCH CGAEITGHVK |
| 2041 | NGTMRIVGPR TCRNMWSGTF PINAYTTGPC TPLPAPNYKF ALWRVSAEEY WEIRRVGDFH |
| 2101 | YVSGMTTDNL KCPCQIPSPE FFTELDGVRL HRFAPPCKPL LREEVSFRVG LHEYPVGSQL |
| 2161 | PCEPEPDVAV LTSMLTDPSH ITAEAAGRRL ARGSPPSMAS SSASQLSAPS LKATCTANHD |
| 2221 | SPDAELIEAN LLWRQEMGGN ITRVESENKV VILDSFDPLV AEEDEREVSV PAEILRKSRR |
| 2281 | FARALPVWAR PDYNPPLVET WKKPDYEPPV VHGCPLPPPR SPPVPPPRKK RTVVLTESTL |
| 2341 | STALAELATK SFGSSSTSGI TGDNTTTSSE PAPSGCPPDS DVESYSSMPP LEGELGDPDL |
| 2401 | SDGSWSTVSS GADTEDVVCC SMSYSWTGAL VTPCAAEEQK LPINALSNSL LRHHNLVYST |
| 2461 | TSRSACQRQK KVTFDRLQVL DSHYQDVLKE VKAAASKVKA NLLSVEEACS LTPPHSAKSK |
| 2521 | FGYGAKDVRC HARKAVAHIN SVWKDLLEDS VTPIDTTIMA KNEVFCVQPE KGGRKPARLI |
| 2581 | VFPDLGVRVC EKMALYDVVS KLPLAVMGSS YGFQYSPGQR VEFLVQAWKS KKTPMGFSYD |
| 2641 | TRCFDSTVTE SDIRTEEAIY QCCDLDPQAR VAIKSLTERL YVGGPLTNSR GENCGYRRCR |
| 2701 | ASGVLTTSCG NTLTCYIKAR AACRAAGLQD CTMLVCGDDL VVICESAGVQ EDAASLRAFT |
| 2761 | EAMTRYSAPP GEPPQPEYDL ELITSCSSNV SVAHDGAGKR VYYLTRDPTT PLARAAWETA |
| 2821 | RHTPVNSWLG NIIMFAPTLW ARMILMTHFF SVLIARDQLE QALNCEIYGA CYSIEPLDLP |
| 2881 | PIIQRLHGLS AFSLHSYSPG EINRVAACLR KLGVPPLRAW RHRARSVRAR LLSRGGRAAI |
| 2941 | CGKYLFNWAV RTKLKLTPIA AAGRLDLSGW FTAGYSGGDI YHSVSHARPR WFWFCLLLLA |
| 3001 | AGVGIYLLPN R |

An example of an HCV subtype 1b is strain HCV-L2, which can be found in the NCBI database as accession number U01214 (gi 437107). Its polyprotein sequence (AAA75355 ) is as follows:

| (SEQ ID NO: 764) | |
|---|---|
| 1 | MSTNPKPQRK TKRNTNRRPQ DVKFPGGGQI VGGVYLLPRR GPRLGVRATR KTSERSQPRG |
| 61 | RRQPIPKARQ PEGRAWAQPG YPWPLYANEG LGWAGWLLSP RGSRPSWGPT DPRRRSRNLG |
| 121 | KVIDTPTCGF ADLMGYPLLV GAPLGGVARA LAHGVRVLED SVNYATGNLP GCSGSIFLLA |
| 181 | LLSVLTVPAS AYEVRNVSGI YHVTNDCSNS SIVYEAADLI MHTPGCVPCV REANSSRCWV |
| 241 | ALTPTLAARD SSIPTATIRR HVDLLVGAAA FCSAMYVGDL CGSVFLVSQL FTFSPRLHQT |
| 301 | VQDCNCSIYP GHLTGHRMAW DMMMNWSPTA ALVVSQLLRI PQAIVDMVAG AHWGVLAGLA |
| 361 | YYPMVGNWAK VLIVMLLFAG VDGTTVTMGG TVARTTYGFT GLFRPGASQK IQLINTNGSW |
| 421 | HINRTALNCN DSLNTGFLAA LFYTHRFNAS GCPERMASCQ SIDKFVQGWG PITYAENGSS |
| 481 | DQRPYCWHYA PRRCGIVPAS QVCGPVYCFT PSPVVVGTTD RSGAPTYSWG ENETDVLLLN |
| 541 | NTRPPQGNWF GCTWMSSTGF TKTCGGPPCN IGGAGNNTLT CPTDCFRKHP EATYTKCGSG |
| 601 | PWLTPRCLVD YPYRLWHYPC TVNFTTFKVR MYVGGVEHRL IAACNWTRGE RCNLEDRDRS |
| 661 | ELSPLLLSTT EWQILPCSYT TLPALSTGLI HLHQNIVDVQ YLYGIGSAVV SFVIKWEYVL |
| 721 | LFFLLLADAR VCACLWMILL IAWAEAALEN LVVLNAASVA GAHGILSFLV FFCAAWYIKG |
| 781 | RLVPGAAYAS YGVWPLLLLL LALPPRAYAM DQGMAASSGG TVLVGLMLLT LSPYYKVVLA |
| 841 | RLIWWLQYFI TRAEAHLQVW VPPLNVRGGR DAVILLTCAV YPELVFDITK LLLAIFGPLM |
| 901 | VLQAGIIKMP YFVRAQGLIR ACMLVRKVAG GHYVQMAFMK LAALTGTYVY DHLTPLRDWA |
| 961 | HTGLRDLAVA VEPVVFSDME TKIITWGADT AECGDIILGY RSSARRGREI LLGPADSLEG |
| 1021 | QGWRLLAPIT AYAQQTRGLL GCIITLSTGR DKNQVEGEVQ VVSTATQSFL ATCVNGVCWT |
| 1081 | VFHGAGSKTL AGPKGPTIQM YTNVDQDLVG WQAAPGMRSL TPCTCGSSDL YLVTRHADVI |
| 1141 | PVRRRGDGRG SLLSPRPVSY LKGSSGGPLL WPSGHAVGIF RAAVCTRGVA KAVDFVPVES |
| 1201 | METTMRSPVF TDNSSPPAVP QTFQVAHLHA PTGSGKSTKV PAAYAAQGYK VLVLNPSVAA |
| 1261 | TLGFGAYMSK AHGTDPNIRT GARTITTGAP ITYSTYGKFF ADGGCSGGAY DIIICDECHS |
| 1321 | TDSTTILGIG TVLDRAETAG ARLVVLATAT PPGSTTVPHP NIEEVALPNT GEIPFYGRAI |
| 1381 | PEIFIKGGRH LIFCPSKKKC DELAAKLSAL GINAVAYYRG LDVSVIPTSG DVVVVATDAL |
| 1441 | MTGYTGDFDS VIDCNTCVTQ TVDFSLDPTF TIETTTVPQD AVSRTQRRGR TGRGRGGIYR |
| 1501 | FVTPGERPSG MFDSSVLCEC YDAGCAWYEL TPAETTVRLR AYLNTPGLPV CQDHLEFWES |
| 1561 | VFTGLNHIDA HFLSQTKQAG DNFPYLVAYQ ATVCARAQAP PPSWDQMWKC LIWLKPVLHG |
| 1621 | PTPLLYRLGA VQNEITLTHP ITKLIMASMS ADLEVVTSTW VLVGGVLAAL AAYCLTTGSV |
| 1681 | VIVGRIILSG RPAVIPDREV LYREFDEMEE CASHLPYIEQ GVQLAEQFKQ KALGLLQTAT |
| 1741 | KQAEAAAPVV ESKWRALETF WAKHMWNFIS GIQYLAALST LPGNPAIASL MAFTASITSP |
| 1801 | LTTQNTLLFN ILGGWVAAQL APASAASAFV GAGSAGAAIG TIGLGKVLVD ILAGYGAGVA |
| 1861 | GALVAFKVMS GEMPSTEDLV NLLPAILSPG ALVVGVVCAA ILRRHVGPGE GAVQWMNRLI |
| 1921 | AFASRGNHDS PTHYVPESDA AARVTQILSS LTITQLLKRL HQWINEDCST PCSGSWLRDV |
| 1981 | WDWICTVLTD FKTWLQSKLL PRLPGVPFFS CQRGYKGVWR GDGIMQTTCP CGAQITGHVK |
| 2041 | NGSMRIVGPK TCSNTWHGTF PINAYTTGPC TPAPTPNYSR ALWRVAAEEY VEVTRVGDFH |
| 2101 | YVTGMTTDNV KCPCQVPAPE FFTEVDGVRL HRYAPACKTL LREEVTFQVG LNQYLGVSQL |
| 2161 | PCEPEPDVAV LTSMLTDPSH ITAETAKRRL ARGSPPSLAS SSASQLSAPS LKATCTTHHD |
| 2221 | SPDADLIEAN LLWRQEMGGN ITRVESESKV VILDSFDPLR AEEGEGEVSV AAEILRKSKK |
| 2281 | FPPALPEEAR PDYNPPLLES WKDPDYVPPV VHGCPLPPAK APPIPPPRRK RTVVLTESTV |
| 2341 | SSALAELAVK TFGSSESSAV DSGTATAPPD QVSDNGDKGS DAESYSSMPP LEGEPGDPDL |
| 2401 | SDGSWSTVSE EASEDVVCCS MSYSWTGALI TPCAAEESKL PINALSNSLL RHHNMVYATT |
| 2461 | SRSAGLRQKK VTFDRLQVLD DHYRDVLKEM KAKASTVKAK LLSVEEACKL TPPHSAKSKF |
| 2521 | GYGAKDVRNL SSRAVNHIRS VWKDLLEDTE TPIDTTIMAK SEVFCVQPEK GGRKPARLIV |
| 2581 | FPDLGVRVCE KMALYDVVST LPQAVMGPSY GFQYSPGQRV EFLVNAWKSK KCPMGFSYDT |
| 2641 | FCFDSTVTES DIRTEESIYQ CCDLAPEAKQ AIKSLTERLY IGGPLTNSKG QNCGYRRCRA |
| 2701 | SVVLTTSCGN TLTCYLKASA ACRAAKLQDC TMLVNGDDLV VICESAGTQE DAANLRAFTE |
| 2761 | AMTRYSAPPG DPPQPEYDLE LITSCSSNVS VAHDASGKRV YYLTRDPTTP LARAAWETAR |
| 2821 | HTPVNSWLGN IIMYAPTLWA RMILMTHFFS ILLAQEQLEK ALECQIYGAC YSIEPLDLPQ |
| 2881 | IIERLHGLSA FSLHSYSPGE INRVASCLRK LGVPPLRVWR HRARRVRAKL LSQGGRAATC |
| 2941 | GKYLFNWAVR TKLKLTPIPA ASRLDLSSWF VAGYSGGDIY HSVSHARPRW FMLCLLLLSV |
| 3001 | GVGIYLLPNR |

An example of an HCV subtype 1c strain HC-G9 can be found in the NCBI database as accession number D14853 (gi 464177). The polyprotein sequence (BAA03581.1) is as follows:

| (SEQ ID NO: 765) | |
|---|---|
| 1 | MSTNPKPQRK TKRNTNRRPQ DVKFPGGGQI VGGVYLLPRR GPRVGVRATR KTSERSQPRG |
| 61 | RRQPIPKARR PEGRSWAQPG YPWPLYGNEG CGWAGWLLSP RGSRPSWGPS DPRRRSRNLG |
| 121 | KVIDTLTCGF ADLMGYIPLV GAPLGGAARA LAHGVRVLED GVNYATGNLP GCSGSIFLLA |
| 181 | LLSCLTVPAS AVGVRNSSGV YHVTNDCPNA SVVYETENLI MHLPGCVPYV REGNASRCWV |
| 241 | SLSPTVAARD SRVPVSEVRR RVDSIVGAAA FCSAMYVGDL CGSIFLVGQI FTFSPRHHWT |
| 301 | TQDCNCSIYP GHVTGHRMAW DMMMNWSPTG ALVVAQLLRI PQAIVDMIAG AHWGVLAGLA |
| 361 | YYSMVGNWAK VVVVLLLFAG VDAETRVTGG AAGHTAFGFA SFLAPGAKQK IQLINTNGSW |
| 421 | HINRTALNCN ESLDTGWLAG LLYYHKFNSS GCPERMASCQ PLTAFDQGWG PITHEGNASD |
| 481 | DQRPYCWHYA LRPCGIVPAK KVCGPVYCFT PSPVVVGTTD RAGVPTYRWG ANETDVLLLN |
| 541 | NSRPPMGNWF GCTWMNSSGF TKTCGAPACN IGGSGNNTLL CPTDCFRKHP DATYSRCGSG |
| 601 | PWLTPRCLVD YPYRLWHYPC TVNYTIFKIR MFVGGVEHRL DAACNWTRGE RCDLDDRDRA |
| 661 | ELSPLLLSTT QWQVLPCSFT TLPALSTGLI HLHQNIVDVQ YLYGLSSAVT SWVIKWEYVV |
| 721 | LLFLLLADAR ICACLWMMLL ISQVEAALEN LIVLNAASLV GTHGIVPFFI FFCAAWYLKG |
| 781 | KWAPGLAYSV YGMWPLLLLL LALPQRAYAL DQELAASCGA TVFICLAVLT LSPYYKQYMA |
| 841 | RGIWWLQYML TRAEALLQVW VPPLNARGGR DGVVLLTCVL HPHLLFEITK IMLAILGPLW |
| 901 | ILQASLLKVP YFVRAHGLIR LCMLVRKTAG GQYVQMALLK LGAFAGTYIY NHLSPLQDAW |
| 961 | HSGLRDLAVA TEPVIFSRME IKTITWGADT AACGDIINGL PVASRRGREV LLGPADALTD |
| 1021 | KGWRLLAPIT AYAQQTRGLL GCIITSLTGR DKNQVEGEVQ IVSTATQTFL ATCVNGVCWT |
| 1081 | VYHGAGSRTI ASASGPVIQM YTNVDQDLVG WPAPQGARSL TPCTCGASDL YLVTRHADVI |
| 1141 | PVRRRGDNRG SLLSPRPISY LKGSSGGPLL CPMGHAVGIF RAAVCTRGVA KAVDFVPVES |
| 1201 | LETTMRSPVF TDNSSPPTVP QSYQVAHLHA PTGSGKSTKV PAAYAAQGYK VLVLNPSVAA |
| 1261 | TLGFGAYMSK AHGIDPNVRT GVRTITTGSP ITHSTYGKFL ADGGCSGGAY DIIICDECHS |
| 1321 | VDATSILGIG TVLDQAETAG VRLTILATAT PPGSVTVPHS NIEEVALSTE GEIPFYGKAI |
| 1381 | PLNYIKGGRH LIFCHSKKKC DELAAKLVGL GVNAVAFYRG LDVSVIPTTG DVVVVATDAL |
| 1441 | MTGYTGDFDS VIDCNTCVVQ TVDFSLDPTF SIETSTVPQD AVSRSQRRGR TGRGKHGIYR |
| 1501 | YVSPGERPSG MFDSVVLCEC YDAGCAWYEL TPAETTVRLR AYLNTPGLPV CQDHLEFWES |
| 1561 | VFTGLTHIDA HFLSQTKQSG ENFPYLVAYQ ATVSARAKAP PPSWDQMWKC LIRLKPTLTG |
| 1621 | ATPLLYRLGG VQNEITLTHP ITKYIMACMS ADLEVVTSTW VLVGGVLAAL AAYCLSTGSV |
| 1681 | VIVGRIILSG KPAVIPDREV LYREFDEMEE CAAHIPYLEQ GMHLAEQFKQ KALGLLQTAS |
| 1741 | KQAETITPAV HTNWQKLESF WAKHMWNFVS GIQYLAGLST LPGNPAIASL MSFTAAVTSP |
| 1801 | LTTQQTLLFN ILGGWVAAQL AAPAAATAFV GAGITGAVIG SVGLGKVLVD ILAGYGAGVA |
| 1861 | GALVAFKIMS GEAPTAEDLV NLLPAILSPG ALVVGVVCAA ILRRHVGPGE GAVQWMNRLI |
| 1921 | AFASRGNHVS PTHYVPESDA SVRVTHILTS LTVTQLLKRL HVWISSDCTA PCAGSWLKDV |
| 1981 | WDWICEVLSD FKSWLKAKLM PQLPGIPFVS CQRGYRGVWR GEGIMHARCP CGADITGHVK |
| 2041 | NGSMRIVGPK TCSNTWRGSF PINAHTTGPC TPSPAPNYTF ALWRVSAEEY VEVRRLGDFH |
| 2101 | YITGVTTDKI KCPCQVPSPE FFTEVDGVRL HRYAPPCKPL LRDEVTFSIG LNEYLVGSQL |
| 2161 | PCEPEPDVAV LTSMLTDPSH ITAETAARRL NRGLPPSLAS SSASQLSAPS LKATCTTHHD |
| 2221 | SPDADLITAN LLWRQEMGGN ITRVESENKI VILDSFDPLV AEEDDREISV PAEILLKSKK |
| 2281 | FPPAMPIWAR PDYNPPLVEP WKRPDYEPPL VHGCPLPPPK PTPVPPPRRK RTVVLDESTV |
| 2341 | SSALAELATK TFGSSTTSGV TSGEAAESSP APSCDGELDS EAESYSSMPP LEGEPGDPDL |
| 2401 | SDGSWSTVSS DGGTEDVVCC SMSYSWTGAL ITPCAAEETK LPINALSNSL LRHHNLVYST |
| 2461 | TSRSAGQRQK KVTFDRLQVL DDHYRDVLKE AKAKASTVKA KLLSVEEACS LTPPHSARSK |
| 2521 | FGYGAKDVRS HSSKAIRHIN SVWQDLLEDN TTPIDTTIMA KNEVFCVKPE KGGRKPARLI |
| 2581 | VYPDLGVRVC EKRALYDVVK QLPIAVMGTS YGFQYSPAQR VDFLLNAWKS KKNPMGFSYD |
| 2641 | TRCFDSTVTE ADIRTEEDLY QSCDLVPEAR AAIRSLTERL YIGGPLTNSK GQNCGYRRCR |
| 2701 | ASGVLTTSCG NTITCYLKAS AACRAAKLRD CTMLVCGDDL VVICESAGVQ EDAANLRAFT |
| 2761 | EAMTRYSAPP GDPPQPEYDL ELITSCSSNV SVAHDGAGKR VYYLTRDPET PLARAAWETA |
| 2821 | RHTPVNSWLG NIIMFAPTLW VRMVLMTHFF SILIAQEHLE KALDCEIYGA VHSVQPLDLP |
| 2881 | EIIQRLHGLS AFSLHSYSPG EINRVAACLR KLGVPPLRAW RHRARSVRAT LLSQGGRAAI |
| 2941 | CGKYLFNWAV KTKLKLTPLP SASQLDLSNW FTGGYSGGDI YHSVSHVRPR WFFWCLLLLS |
| 3001 | VGVGIYLLPN R |

Other HCV polyprotein sequences are known in the art, see for example, the web sites http://www.hcvdb.org/viruses.asp; http://www.ncbi.nlm.nih.gov/ and http://hcv.lanl.gov/content/sequence/LOCATE/locate.html. Additional examples include a Taiwan isolate of hepatitis C virus available in the NCBI database at accession number P29846 (gi: 266821). Other examples of HCV polyprotein sequences include those at the NCBI accession number AF009606, AY734971, AJ238799, AY545953, AY734974, AB047639, AF177036, AY734977, AY734982, AY734984, AY734987, EF427672, and AY736194.

### Modified E2 Polypeptide of the Invention

In one aspect, the invention provides a modified HCV E2 polypeptide.

As used herein, the term “polypeptide” refers to a polymer of three or more amino acids, regardless of post-translational modifications such as methylation, glycosylation or phosphorylation.

An “E2” polypeptide is the HCV viral envelope protein that forms a heterodimer with the E1 glycoprotein through non-covalent interactions. HCV E1 and E2 envelop glycoproteins are exposed on the viral surface where they function in viral attachment and fusion to target cells. In the prototype HCV strain H77 (shown above as SEQ ID NO: 763), the E2 glycoprotein is residues 384 to 746.

The term “modified” as used in reference to an E2 polypeptide of the invention means that the polypeptide is free of sequences in the hypervariable region of the E2 polypeptide, in particular, sequences that correspond to the segment defined by amino acid residues 384 to 395. A modified E2 polypeptide of the invention also has at least one amino acid substitutions at positions 416, 417, 483, 484, 485, 538, 540, 544, 545, 547, 549 or any combinations thereof relative to the E2 polypeptide sequence of HCV stain H77. Accordingly, an “modified E2 polypeptide” of the invention has a structure that is different from that of any naturally-occurring HCV E2 polypeptides.

As used herein, numeric terms identifying amino acid residues or positions in a polypeptide, i.e. the protein or polypeptide “coordinates,” for example, the term “residues 396 to 424,” “residue 416,” or “amino acid 416,” are based on the absolute amino acid numbering system for HCV described by Kuiken et al. in Hepatology 44: 1355-1361 (2006), which is incorporated herein by reference in its entirety. Briefly, the polyprotein sequence of HCV strain H77 is used as a reference in the numbering system, and the first amino acid of the core protein is amino acid residue number 1. Other HCV polyprotein sequences are compared with the H77 polyprotein sequences by sequence alignment. Insertions in other non-H77 sequences are identified using a residue number/alphabet designation relative to the H77 reference. For example, three inserted amino acids in a non-H77 polyprotein sequence inserted between amino acid residues 396 and 397 of the reference H77 sequence would be identified as follows: residue 396a, 396b and 396c. Insertions longer than the length of the alphabet would be identified as follows: . . . 396x, 396y, 396z, 396aa, 196ab, 396ac, . . . 396ax, 396ay, 396az, 396ba, 396bb . . . Deletions in a non-H77 sequence relative to the H77 reference sequence can be indicated by identifying the residue deleted. For example, a missing residue, i.e. a “deletion”, in a non-H77 sequence relative to the H-77 reference sequence identified in a sequence alignment such as a deletion of amino acid residue 396 is indicated by the term “del 396”. Thus, according to the numbering system used herein, a polypeptide coordinate or coordinates, such as “amino acid 396,” “residue 396,” or “amino acids 396 to 424,” refer to analogous residues or segments in HCV polyproteins from different isolates, strains, subtypes or genotypes. Analogous residues or segments can be identified by sequence alignment as described below. A similar system is used for identifying HCV nucleotide sequence.

Generally, the amino acid sequences of two or more HCV E2 polypeptides can be compared by alignment using methods known in the art including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo et al., Applied Math., 48:1073 (1988), the teachings of which are incorporated herein by reference. Two HCV polyprotein sequences can be compared by sequence alignment in a manner to produce the highest degree of sequence similarity or identity. Upon such alignment, sequence identity is determined on a position-by-position basis, e.g., the sequences are “identical” at a particular position if at that position, the amino acid residues are identical. Preferred methods to determine sequence identity between two sequences are designed to give the largest match between the sequences tested. Methods to determine sequence identity are codified in publicly available computer programs. Examples of such programs include, but are not limited to, the GCG program package (Devereux, et al., Nucleic Acids Research, 12:387 (1984)), BLASTP, BLASTN and FASTA (Altschul et al., J. Molec. Biol., 215:403 (1990)). The BLASTX program is publicly available from NCBI and other sources (BLAST Manual, Altschul et al., NCVI NLM NIH Bethesda, Md. 20894, Altschul et al., J. Molec. Biol., 215:403 (1990), the teachings of which are incorporated herein by reference). These programs optimally align sequences using default gap weights in order to produce the highest level of sequence identity between sequences. For HCV sequence analysis tools, see also http://hcv.lanl.gov/content/sequence/HCV/ToolsOutline.html. See also the Sequence Locator tool available at the American HCV database website (http://hcv.lanl.gov/content/hcv-db/LOCATE/locate.html) or the Number tool on the European HCV database website (http://euhcvdb.ibcp.fr/euHCVdb/).

Once an HCV amino acid sequence is optimally aligned with that of the HCV strain H77, the E2 amino acid sequence can be identified based on its correspondence with the HCV strain H77 amino acid residues 384 to 746. Accordingly, the term “amino acid 396” or “amino acids 396 to 424” refers to analogous residues in different HCVs including, for example, HCVs of different isolates, strains, species, quasispecies, subtypes or genotypes.

Select examples of naturally-occurring HCV E2 sequences are shown below.

| HCV origin | |
|---|---|
| (accession number) | Amino acid sequence |
| 1a.US.H77 | ETHVTGGSAGRTTAGLVGLLTPGAKQNIQLINTNGSWHINSTALNCNESLNTGWLAGLFYQHKFNSSGCPERLASCRRLTDFAQG |
| (AF009606) | WGPISYANGSGLDERPYCWHYPPRPCGIVPAKSVCGPVYCFTPSPVVVGTTDRSGAPTYSWGANDTDVFVLNNTRPPLGNWFGCT |
| | WMNSTGFTKVCGAPPCVIGGVGNNTLLCPTDCFRKHPEATYSRCGSGPWITPRCMVDYPYRLWHYPCTINYTFIKVRMYVGGVEH |
| | RLEAACNWTRGERCDLEDRDRSELSPLLLSTTQWQVLPCSFTTLPALSTGLIHLHQNIVDVQYLYGVGSSIASWAIKWEYVVLLL |
| | FLLLADARVCSCLWMMLLISQAEA |
| | (SEQ ID NO: 766) |
| 1a.JP.HC-J1 | ETIVSGGQAARAMSGLVSLFTPGAKQNIQLINTNGSWHINSTALNCNESLNTGWLAGLIYQHKFNSSGCPERLASCRRLTDFDQG |
| (D10749) | WGPISHANGSGPDQRPYCWHYPPKPCGIVPAKSVCGPVYCFTPSPVVVGTTDRSGAPTYNWGANDTDVFVLNNTRPPLGNWFGCT |
| | WMNSTGFTKVCGAPPCVIGGGGNNTLHCPTDCFRKHPEATYSRCGSGPWITPRCLVDYPYRLWHYPCTINYTIFKVRMYVGGVEH |
| | RLDAACNWTRGERCDLEDRDRSELSPLLLSTTQWQVLPCSFTTLPALSTGLIHLHQNIVDVQYLYGVGSSIASWAIKWEYVVLLF |
| | LLLADARVCSCLWMMLLISQAEA |
| | (SEQ ID NO: 767) |
| 1b.KR.HCV-L2 | TTVTMGGTVARTTYGFTGLFRPGASQKIQLINTNGSWHINRTALNCNDSLNTGFLAALFYTHRFNASGCPERMASCQSIDKFVQG |
| (U01214) | WGPITYAENGSSDQRPYCWHYAPRRCGIVPASQVCGPVYCFTPSPVVVGTTDRSGAPTYSWGENETDVLLLNNTRPPQGNWFGCT |
| | WMSSTGFTKTCGGPPCNIGGAGNNTLTCPTDCFRKHPEATYTKCGSGPWLTPRCLVDYPYRLWHYPCTVNFTTFKVRMYVGGVEH |
| | RLIAACNWTRGERCNLEDRDRSELSPLLLSTTEWQILPCSYTTLPALSTGLIHLHQNIVDVQYLYGIGSAVVSFVIKWEYVLLFF |
| | LLLADARVCACLWMILLIAQAEA |
| | (SEQ ID NO: 768) |
| 1c.IN | TTQVTGGTAGRNAYRLASLFSTGPSQNIQLINSNGSWHINRTALNCNDSLHTGWVAALFYSHKFNSSGRPERMASCRPLTAFDQGW |
| (AY051292) | GPITYGGKASNDQRPYCWHYAPRPCGIVPAKEVCGPVYCFTPSPVVVGTTDKYGVPTYTWGENETDVLLLNNSRPPIGNWFGCTWM |
| | NSTGFTKTCPAPACNVGGSETNTLSCPTDCFRRHPDATYAKCGSGPWLNPRCMVDYPYRLWHYPCTVNYTIFKIRMFVGGIEHRLT |
| | AACNWTRGERCDLDDRDRAELSPLLLSTTQWQVLPCSFTTLPALSTGLIHLHQNIVDVQYLYGLSSVVTSWAIRWEYVVLLFLLLA |
| | DARICACLWMMLLISQVEA |
| | (SEQ ID NO: 769) |
| 2a.JP | YTHTVGGAAASTANSIAGLLSRGPRQNLQLINSNGSWHINRTALNCHDSLQTGFITALFYARHFNSSGCPERLAACRNIEAFRVG |
| (AY746460) | WGALQYEDNVTNPEDMRPYCWHYPPKQCGIVPARSVCGPVYCFTPSPVVVGTTDKLGVPTYTWGENETDVFLLNSTRPPQGPWFG |
| | CTWMNSTGFTKTCGAPPCRTRADFNASTDLLCPTDCFRKHPDATYNKCGSGPWLTPRCLIDYPYRLWHYPCTVNYTTFKIRMYVG |
| | GVEHRLMAACNFTRGDSCDLSQRDRGQLSPLLHSTTEWAILPCFSFDLPALSTGLLHLHQNIVDVQYMYGLSPALTKYIVRWEWV |
| | VLLFLLLADARVCACIWMLILLGQAEA |
| | (SEQ ID NO: 770) |
| 2b.JP.MD2b1-2 | RHHTTGLQVGKTLARVTSLFSIGPKQNIGLINTNGSWHINRTALNCNDSLQTGFIASLFYVNNINSSGCPERMSSCRELDDFRIG |
| (AY232731) | WGTLEYETNVTNDEDMRPYCWHYPPKPCGIVPARTVCGPVYCFTPSPIVVGTTDKQGVPTYSWGENETDVFLLNSTRPPRGSWFG |
| | CTWMNGTGFTKTCGAPPCRIRRDYNSTLDLLCPTDCFRKHPDTTYLKCGSGPWLTPKCLVEYPYRLWHYPCTVNFTIFKVRMYVG |
| | GVEHRFSAACNFTRGDRCRLEDRDRGQQSPLLHSTTEWAVLPCSFSDLPALSTGLLHLHQNIVDVQYLYGLSPAITRYIVKWEWV |
| | VLLFLLLADARVCACLWMLIILGQAEA |
| | (SEQ ID NO: 771) |
| 2c.BEBE1 | STYTTGAVVGRSTHLFTSMFSLGSQQRVQLIHTNGSWHINRTALNCNDSLETGFLAALFYTSSFNSSGCPERLAACRSIESFRIG |
| (D50409) | WGSLEYEESVTNDADMRPYCWHYPPRPCGIVPARTVCGPVYCFTPSPVVVGTTDRAGAPTYNWGENETDVFLLNSTRPPKGAWFG |
| | CTWMNGTGFTKTCGAPPCRIRKDFNASEDLLCPTDCGRKHPGATYIKCGAGPWLTPRCLVDYPYRLWHYPCTVNYTIYKVRMFVG |
| | GIEHRLQAACNFTRGDRCNLEDRDRSQLSPLLHSTTEWAILPCSYTDLPALSTGLLHLHQNIVDVQYLYGLSPAITKYVVKWEWV |
| | VLLFLLLADARVCACLWMLLLLGQAEA |
| | (SEQ ID NO: 772) |
| 2i.VN.D54 | STYSTGAQAGRAASGFAGLFTRGARQNIQLINTNGSWHINRTALNCNDSLQTGFIASLFYANSFNSSGCPERMAHCRSIEHFRIG |
| (DQ155561) | WGALEYEENVINEEDMRPYCWHYPPKPCGVVPAKSVCGPVYCFTPSPVVVGTTDKRGVPTYNWGDNETDVFLLNSTRPPKGAWFG |
| | CTWMNGTGFTKTCGAPPCRIRRDFNASEDLLCPTDCFRKHPEATYSKCGAGPWLTPRCLIDYPYRLWHYPCTFNYTIFKIRMFVG |
| | GIEHRLQAACNFTRGDRCNLDDRDRSQLSPLLHSTTEWAILPCSFTDLPALSTGLIHLHQNIVDVQYLYGLTPAITKYVVKWEWV |
| | VLLFLLLADARVCACLWMLILLGQAEA |
| | (SEQ ID NO: 773) |
| 2k.MD.VAT96 | QTHTISGHAARTTHGLVSLFTPGSQQNIQLVNTNGSWHINRTALNCNDSLKTFGIAALFYSHKFNSSGCPQRMSSCRSIEEFRIG |
| (AB031663) | WGNLEYEENVTNDDNMRPYCWHYPPRPCGIVPAQTVCGPVYCFTPSPVVVGTTDRRGVPTYTWGENDTDVFLLNSTRPPRGAWFG |
| | CTWMNSTGFTKTCGAPPCRIRPDFNSSEDLLCPTDCFRKHSEATYTRCGAGPWLTPKCLFHYPYRLWHYPCTINFTIHKIRMFIG |
| | GVEHRLEAACNFTRGDRCNLEDRDRSQLSPLLHSTTEWAILPCTFSDMPALSTGLLHLHQNIVDVQYLYGLSPAITKYIVKWEWV |
| | VLLFLLLADARVCACLWMLLLLGQAEA |
| | (SEQ ID NO: 774) |
| 3a.CH.452 | TTYTTGGNAARGASGIVSLFTPGAKQNLQLVNTNGSWHINRTALNCNDSINTGFIAGLIYYHKFNSTGCPQRLSSCKPITFFRQG |
| (DQ437509) | WGSLTDANITGPSDDKPYCWHYPPRPCDTIRASSVCGPVYCFTPSPVVVGTTDAKGAPTYNWGANETDMFLLQSLRPPSGRWFGC |
| | TWMNSTGFTKTCGAPPCNIYGGGGNLNNESDLFCPTDCFRKHPEATYSRCGAGPWLTPRCLVDYPYRLWHYPCTVNFTLFRMRTF |
| | VGGFEHRFTAACNWTRGERCNIEDRDRSEQHPLLHSTTELAILPCSFTPMPALSTGLIHLHQNIVDVQYLYGIGSGVVGWALKWE |
| | FVILVFLLLADARVCVALWLMLMISQAEA |
| | (SEQ ID NO: 775) |
| 3b.JP.HCV-Tr | TTYTTGGNAARGASGIVSLFTPGAKQNLQLVNTNGSWHINRTALNCNDSINTGFIAGLIYYHKFNSTGCPQRLSSCKPITFFRQG |
| (D49374) | WGPLTDANINGPSEDRPYCWHYPPRPCNITKPLNVCGPVYCFTPSPVVVGTTDIKGLPTYRFGVNESDVFLLTSLRPPQGRWFGC |
| | VWMNSTGFVKTCGAPPCNIYGGMKDIEANQTHLKCPTDCFRKHHDATFTRCGSGPWLTPRCLVDYPYRLWHYPCTVNFSIFKVRM |
| | FVGGHEHRFSAACNWTRGERCDLEDRDRSEQQPLLHSTTDSLILPCSFTPMRRLSTGLIHLHQNIVDVQYLYGVGSAVVGWALKW |
| | EFVVLVFLLLADARVCVALWMMLLISQAEA |
| | (SEQ ID NO: 776) |
| 3k.ID.JK049 | STTITGGVAASGAFTITSLFSTGAKQPLHLVNTNGSWHINRTALNCNDSLNTGFIAGLLYYHKFNSSGCVERMSACSPLDRFAQG |
| (D63821) | WGPLGPANISGPSSEKPYSWHYAPRPCDTVPAQSVCGPVYCFTPSPVVVGATDKRGAPTYTWGENESDVFLLESARPPTEPWFGC |
| | TWMNGSGYVKTCGAPPCHIYGGREGKSNNSLVCPTDCFRKHPDATYNRCGAGPWLTPRCLVDYPYRLWHYPCTVNYTIFKVRMFV |
| | GGLEHRFNAACNWTRGERCNLEDRDRSEMYPLLHSTTEQAILPCSFVPIPALSTGLIHLHQNIVDVQYLYGISSGLVGWAIKWEF |
| | VILIFLLLADARVCVVLWMMMLISQAEA |
| | (SEQ ID NO: 777) |
| 4a.EG.ED43 | ETHVSGAAVGRSTAGLANLFSSGSKQNLQLINSNGSWHINRTALNCNDSLNTGFLASLFYTHKFNSSGCSERLACCKSLDSYGQG |
| (Y11604) | WGPLGVANISGSSDDRPYCWHYAPRPCGIVPASSVCGPVYCFTPSPVVVGTTDHVGVPTYTWGENETDVFLLNSTRPPHWAWFGC |
| | VWMNSTGFTKTCGAPPCEVNTNNGTWHCPTDCFRKHPETTYAKCGSGPWITPRCLIDYPYRLWHFPCTANFSVFNIRTFVGGIEH |
| | RMQAACNWTRGEVCGLEHRDRVELSPLLLTTTAWQILPCSFTTLPALSTGLIHLHQNIVDVQYLYGVGSAVVSWALKWEYVVLAF |
| | LLLADARVSAYLWMMFMVSQVEA |
| | (SEQ ID NO: 778) |
| 4d.24 | QTHITGGKAGRDALTFAGLFTMGGQQHIQLINTNGSWHINRTALNCNDSLNTGFLASLFYYRRFNSSGCPERLASCSSLDSLPQG |
| (DQ516083) | WGPLGIYQPNVPDTRPYCWNYTPRPCGTVSALTVCGPVYCFTPSPVVVGTTDRRGAPTYTWGENETDVFLLNTTRPPRGAWFGCT |
| | WMNSTGFTKSCGGPPCSITANGSTWGCPTDCFRKHPEATYTKCGSGPWLTPRCLVDYPYRLWHYPCTVNYTVFKVRMYIGGIEHR |
| | LDAACNWTRGEPCDLEHRDRTEISPLLLSTTQWQVLPCSFTTLPALSTGLIHLHQNIVDVQYLYGVGSAVVSWALXWEYVVLAFL |
| | LLAGARICACLWMMLLVAQVEA |
| | (SEQ ID NO: 779) |
| 4f.FR.IFBT84 | VTYTTGSSAGSTIHGIANLFTPGSKQNLQLINTNGSWHINRTALNCNDSLQTGFIAGLIYRNKFNSSGCPERLSRCKRLDDLAQG |
| (EF589160) | WGKLGAANITGSSDDRPYCWHYAPRPCGVVPASEVCGPVYCFTPSPVAVGTTDRLGVPTYSWGANETDVFILNSTRPPRGAWFGC |
| | TWMNGTGFTKTCGAPPCQVQASVANQSWSCPDCFRKHPETTYTKCGSGPWLTPRCLIDYPYRLWHYPCTVNFSIFKVRMFVVAGV |
| | EHRLEAACNWTRGEPCGLEHRDRAELSPLLLSTTQWQVLPCSFTPLPASLSTGLIHLHQNIVDVQYLYGIGSVVVSWALKEYVVL |
| | AFLLLADARVCACLWMMLLVSQVEA |
| | (SEQ ID NO: 780) |
| 5a.ZA.SA13 | NTRTVGGSAAQGARGLASLFTPGPQQNLQLINTNGSWHINRTALNCNDSLQTGFVAGLLYYHKFNSTGCPQRMASCRPLAAFDQG |
| (AF064490) | WGTISYAAVSGPSDDKPYSWHYPPRPCGIVPARGVCGPVYCFTPSPVVVGTTDRKGNPTYSWGENETDIFLLNNTRPPTGNWFGC |
| | TWMNSTGFVKTCGAPPCNLGPTGNNSLKCPTDCFRKHPDATYTKCGSGPWLTPRCLVHYPYRLWHYPCTLNYTIFKVRMYIGGLE |
| | HRLEVACNWTRGERCDLEDRDRAELSPLLHTTTQWAILPCSFTPTPASLTGLIHLHQNIVDTQYLYGLSSSIVSWAVKWEYIVLA |
| | FLLLADARICTCLWIMLLVCQAEA |
| | (SEQ ID NO: 781) |
| 6a.HK.6a33 | TTTVGHGVARTTAGITGLFSPGASQNLQLIKNGSSWHINRTALNCNDSLQTGFLASLFYVRKFNSSGCPERMAVCKSLADFRQGW |
| (AY859526) | GQITYKVNISGPSDDRPYCWHYAPRPCDVVPASTVCGPVYCFTPSPVVIGTTDRRGNPTYTWGENETDVFMLESLRPPTGGWFGC |
| | TWMNSTGFTKTCGAPPCQIIPGDYNSSANELLCPTDCFRKHPEATYQRCGSGPWVTPRCLVDYPYRLWHYPCTVNFTVHKVRMFV |
| | GGIEHRFDAACNWTRGERCELHDRDRIEMSPLLFSTTQLAILPCSFSTMPALSTGLIHLHQNIVDVQYLYGVSSSVTSWVVKWEY |
| | IVLMFLVLADARICTCLWLMLLISNVEA |
| | (SEQ ID NO: 782) |
| 6b.Th580 | TTTVGRAAGRSAYLFTSIFSSGPNQKIQLINTNGSWHINRTALNCIDSLQTGFLSALFYRSNFNSTGCSERLGACKPLEHFQQGW |
| (NC009827) | GPITHKSNITGPSEDRPYCWHYAPRECSVVPASSVCGPVYCFTPSPVVVGTTDRLGNPTYNWGENETDVFMLESLRPPQGGWFGC |
| | TWMNSTGFTKTCGAPPCQLIPGDYNSSSNQLLCPTDCFRKHPEATYQKCGSGPWLTPRCLVDYPYRLWHYPCTVNYTIHKVRMFI |
| | GGVEHRFDAACNWTRGDRCDLYDRDRIEMSPLLFSTTQLAILPCSFTTMPALSTGLIHLHQNIVDVQYLYGVSSSIVSWAVKWEY |
| | VVLMFLVLADARICTCLWLMLLVGKVEA |
| | (SEQ ID NO: 783) |
| 6d.VN.D88 | ETYVTGSVTGQTITGFSGLFSSGSQQKLQLVNTNGSWHINRTALNCNDSLQTGFIAALFYTYRFNASGCPARVSSCKPLTYFDQG |
| (EF420130) | WGPISYANVSGSSEDKPYCWHYPPRPCGVVPASQVCGPVYCFTPSPVVVGTTDRKGLPTYSEGENESDVFLLESLRPPKGGWYGC |
| | TWMNSTGFVKTCGAPPCNIRPDSTGANTTLICPTDCFRKHPEATYAKCGSGPWLTPRCVVDYPYRLWHYPCTQNYTLHKVRMFIG |
| | GLEHRFQAACNWTRGDPCNLEDRDRVEMSPLLFSTTELAILPCSFTTMPALSTGLIHLHQNIVDVQYLYGISPSVTSWVIKWEYV |
| | VLAFLVLADARICACLWLMLLIGQAEA |
| | (SEQ ID NO: 784) |
| 6e.CN.GX004 | HTHVTGAVAGRTVGNIASLFSPGSRQNLQLINSNGSWHINRTALNCNDSLQTGFIASLFYFNKFNASGCPDRMSSCKPLTYFDQG |
| (DQ314805) | WGPISYANVSGSSEDKPYCWHYPPRPCGVVPASQVCGPVYCFTPSPVVVGTTDKKGLPTYTWGENESDVFLLESLRPPKGGWYGC |
| | TWMNSTGYVKTCGAPPCNIKPDASSSNTTLTCPTDCFRKHPEATYTRCGSGPWLTPRCLVDYPYRLWHYPCTQNYTIHKVRMFVG |
| | GLEHRFQAACNWTRGAPCNLDDRDRVEMSPLLFSTTELAILPCSFTTMPALSTGLIHLHQNIVDVQYLYGISPSITSWVIKWEYI |
| | VLAFLLLADARICACLWLMLLIGQAEA |
| | (SEQ ID NO: 785) |
| 6f.TH.C-0046 | TTDVAHSAARTTHGIASLFSPGAHQRLQLINSNGSWHINRTALNCNDSLHTGFLANLFYVHKINDSGCPDRMSSCKPLTSFNKGW |
| (DQ835764) | GPITYATIEGPSSDRPYCWHYAPRPCGVEPAKNVCGPVYCFTPSPVVVGTTDRVGLPTYTWGENETDVFILESVRPPQGGWFGCT |
| | WMNSTGFVKTCGAPPCKLGPGTNNSLVCPTDCFRKHPGATYAKCGSGPWLTPRCLVDYPYRLWHYPCTVNFTLHKIRMYVGGVEH |
| | RLTAACNWTRGDPCSLGRRDRAELSPLLFSTTELAILPCTFTPMPALSTGLIHLHQNIVDVQYLYGLTPSVVSWSIKWEYLVLAF |
| | LVLADARICACLWLMLMIAQVEA |
| | (SEQ ID NO: 786) |
| 6g.ID.JK046 | STYVASSVSQATSGLVSLFSAGARQNLQLINTNGSWHINRTALNCNDSLQTGFIASLFYRNKFNATGCPERLSACKTLDSFDQGW |
| (D63822) | GPITYANISGPAVEKPYCWHYPPRPCEVVSALNVCGPVYCFTPSPVVLGTTDRRGNPTYTWGANETDVFMMSSLRPPAGGWYGCT |
| | WMNTSGGVKTCGAPPCNIRPNPEENRTETLRCPTDCFRKHPGATYAKCGSGPWLTPRCLVDYPYRLWHYPCTVNYTLKKVRMYIA |
| | GSEHRFTAACNWTRGERCDLADRDRIEMSPLLFSTTELAILPCSFTTMPALSTGLIHLHQNVVDVQYLYGLSTSIVNWAIKWEYV |
| | VLLFLVLADSRICLALWLMLLIGQAEA |
| | (SEQ ID NO: 787) |
| 6k.VN.VN405 | TTHIGSSASATTNRLTSFFSPGSKQNVQLIKTNGSWHINRTALNCNDSLHTGFIAGLLYAHRFNSSGCPERLSSCRPLHAFEQGW |
| (D84264) | GPLTYANISGPSNDKPYSWHYPPRPCDIVPARSVCGPVYCFTPSPVVVGTTDRKGLPTYTWGANESDVFLLRSTRPPRGSWFGCT |
| | WMNSTGFVKTCGAPPCNTRPVGSGNDTLVCPTDCFRKHPEATYARCGSGPWLTPRCLVNYPYRLWHYPCTVNYTHIKVRMFVGGI |
| | EHRFEAACNWTRGERCELDDRDRVEMSPLLFSTTQLSILPCSFTTMPALSTGLIHLHQNIVDVQYLYGVSSAVVSWAVKWEUIVL |
| | AFLVLAVARVCACLWLMFLVGQAEA |
| | (SEQ ID NO: 788) |
| 6m.TH.C-0208 | TTGIGYAVSRATSGLTGLFTPGARQNIQLINTNGSWHINRTALNSNDSLQTGFIAGLIYAHKFNSTGCPDRLSWCRSLRSFDQGW |
| (DQ835763) | GPITYANVSGSSDDRPYCWHYAPRPCTVVPASSVCGPVYCFTPSPVVIGTTDKKGFPTYSWGGNETDVFLLQSARPPRGAWFGCT |
| | WMNSTGFVKTVGAPPCNISPPSSSNNSLKCPTDCFRKHPGATYAKCGSGPWLTPRCLVDYPYRLWXYPCTVNYTIHKVRLYLWGI |
| | EHRFNAACNWTRGERCELDXRDRIEMSPLLFSTTELSILPCSFTTTPALSTGLIHLHQNVVDVQYLYGLSTAVVSWAVKWEYVVL |
| | AFLVLADARICACLWLMFLVGQAEA |
| | (SEQ ID NO: 789) |
| 6n.CN.KM42 | TTYTTGGTAAHSVYGLSTLFTRGSQQNIQLVNSNGSWHVNRTALNCNDSLQTGFIAGLFYYHKFNSSGCLERMSSCKPITLFDQG |
| (DQ278894) | WGPITYANTSGPSEDRPYCWHYPPRPCGIVPAREVCGPVYCFTPSPVVIGTTDKKGLPTYNWGENMSDVFLLQSARPPRGAWFGC |
| | TWMNSTGYVKTXGAPPCNXGPNTNTSLXCPTDCFRKHPDATYSRCGSGPWLTPRCLVDYPYRLWHYPCTINFTIHKVRMFLGGVE |
| | HRFSAACNWTRGERCELDDRDRVEMSPLLFSTTELAILPCSFTTMPALSTGLIHLHQNIVDIQYLYGVSTVLVSWAIKWEYVVLA |
| | FLVLADARICACMWLMFLVGQAEA |
| | (SEQ ID NO: 790) |

A modified E2 polypeptide of the invention differs from the naturally-occurring E2 polypeptide of HCV in that one or more immunodominant epitopes in the naturally-occurring E2 polypeptide are eliminated or its immunogenicity is attenuated, while the immunogenicity of conserved or cross-neutralizing epitopes are augmented. For example, when the naturally-occurring E2 polypeptide is used an an immunogen, greater than half of antibodies generated are directed against immunodominant epitopes such as, for example, the hypervariable region 1 (amino acid residues 384 to 410) or the epitopes recognized by the AR1A and AR1B antibodies that include the residues T416, T416, N417, R483, P484, Y485, V538, N540, P544, P545, G547 and W549. The modified E2 polypeptide of the invention differs from the corresponding naturally-occurring E2 amino acid sequence in that the modified E2 polypeptide of the invention (1) does not include the segment defined by amino acid residues 384 to 395 of the hypervariable region 1 of the naturally-occurring E2 polypeptide and (2) has at least one amino acid substitution at position 416, 417, 483, 484, 485, 538, 540, 544, 545, 547, 549 or any combinations thereof. In some embodiments, a modified E2 polypeptide of the invention has at least two amino acid substitutions at these positions, e.g. 2, 3, 4, 5, 6, 7, 8, 9, 10 or 11 substitutions.

The amino acid that can be substituted at these positions can be one that has a different chemical or physical property from the naturally-occurring residue. For example, the proline residues at position 484, 544 or 545 can be substituted with an amino residue that enable the polypeptide to be more flexible such as for example an alanine, valine or other non-cyclic residues. The glycine residue at position 547 can be substituted with an amino acid that has a bulkier side chain such as, for example, valine, leucine, methionine, phenylalanine, tyrosine, tryptophan, histidine, lysine, arginine, aspartic acid, glutamic acid, asparagine or glutamine, while the tryptophan residue at position 549 can be substituted with an amino acid residue that has a less bulky side chain, for example, glycine, alanine, valine, serine, systeine, or threonine. The threonine residue at position 416 can be substituted with a residue that does not have a hydroxyl or sulfur-containing side chain. The acidic asparagine residue at position 417 or 540 can be substituted with, for example, a basic amino acid residue such as histidine, lysine or arginine, while the basic arginine residue at position 483, for example, can be substituted with, for example, an acidic residue such as aspartic acid, glutamic acid, asparagine or glutamine. The aromatic amino acid tyrosine at position 485 can be substituted with, for example, a non-aromatic residue, while the valine at position 538 can be substituted with a residue having a bulkier side chain, a basic or acidic residue, or one with an aromatic, hydroxyl or sulfur-containing side chain. A preferred substitution or combination of substitutions is one that decreases the immunogenicity or function of epitopes recognized by the AR1 antibodies such as AR1A and AR1B.

The modified E2 polypeptide of the invention can also have one or more other substitutions, insertions or deletions relative to a naturally-occurring E2 polypeptide as long as the modified E2 polypeptide sequence includes the discontinuous epitopes described herein that come together to form a conformational epitope recognized by a conformation-dependent cross-neutralizing antibody such as the AR3A, AR3B, AR3C or AR3D antibody.

As used herein, the term “conformation-dependent,” in reference to an antibody, means that the antibody recognizes and binds specifically with discontinuous epitopes composed of amino acid residues that are located at some distance from each other, i.e. the residues are discontinuous in the polypeptide sequence. The discontinous epitopes come together through proper folding of the polypeptide to form a binding site, i.e. a conformational epitope that is recognized by a conformation-dependent antibody.

As used herein, the term cross-neutralizing means the ability to neutralize at least two HCV strains, isolates, species, quasispecies, subtypes or genotypes. The term “neutralize,” as used herein in reference to an antibody, means that the antibody can prevent or reduce HCV infection or replication in a cell culture or in a mammal, as well as alleviate one or more symptoms associated with HCV infection in a mammal. The term “reduce,” as used herein, means a decrease in any amount such as a 5%, 10%, 15%, 20%, 25%, 30%, 35%, 40%, 45%, 50%, 55%, 60%, 65% or more than 65%. HCV infection or replication can be detected by examining HCV RNA levels, virus particles count or clinical symptoms associated with HCV infection. Whether an antibody will prevent or reduce HCV infection or replication or alleviate associated symptions can be determined using methods known in the art, as well as the methods described herein, including determining the level of HCV RNA in a sample from a mammal that has been infected with HCV or detecting reduction of signals from a reporter gene encoded by the virus such as, for example, the relative light unit (RLU) for luciferase or the mean fluorescence intensity (MFI) of green fluorescent protein (GFP).

As used herein, the term “binds specifically” or “specifically binds,” in reference to an antibody/antigen interaction, means that the antibody binds with a particular antigen without substantially binding to other unrelated antigens. For example, the antibody has at least 50% or greater affinity, preferably about 75% or greater affinity, and more preferably, about 90% or greater affinity, to a particular polypeptide than to other unrelated polypeptides. Examples of cross-neutralizing antibodies that bind specifically with the discontinuous epitopes of the invention include AR3A, AR3B, AR3C and AR3D.

The conformational epitope of an E2 polypeptide of the invention comprises, from the amino to carboxy termini, the following amino acid segments: (1) a segment defined by amino acid residues 396 to 424 of the naturally-occurring E2 polypeptide; (2) a segment defined by amino acid residues 436 to 447 of the naturally-occurring E2 polypeptide; and (3) a segment defined by amino acid 523 to 540 of the naturally-occurring E2 polypeptide. The first segment, defined by amino acid residues 396 to 424 of the naturally-occurring E2 polypeptide, can be separated from the second segment, defined by amino acid residues 436 to 447 of the naturally-occurring E2 polypeptide, by at least 6, 7, 8, 9, 10, 11, 12, 13, 14, 15 or more than 15 amino acid residues. The second segment can be separated from the third segment, defined by amino acid 523 to 540 of the naturally-occurring E2 polypeptide, by at least 20, 22, 24, 26, 28, 30,32, 34, 36, 38, 40, 42, 44, 46, 48, 50, 52, 54, 56, 58, 60 or more than 60 amino acid residues. Preferable the first and second segments are separated by about 10 residues and the second and third segments are separated by about 50 residues.

These segments are the discontinuous epitopes of a modified E2 polypeptide of the invention, and they can have sequences of segments defined by amino acids 396 to 424, 436 to 447 and 396 to 424 of HCV strain H77 or the sequences of the regions defined by amino acids 396 to 424, 436 to 447, and 396 to 424 of other HCV strains, isolates, species, quasispecies, subtypes or genotypes. Sequences of the discontinuous epitopes can be determined based on sequence alignment of the HCV E2 or HCV polyprotein sequence with the sequence of strain H77 using the method described above.

Examples of the amino acid sequences of discontinuous epitopes of select E2 polypeptides of the invention and their HCV origin are shown below.

| HCV origin | Discontinuous Epitopes: Residues | | |
|---|---|---|---|
| (accession number) | 396-424 | 436-447 | 523-540 |

| | | | |
|---|---|---|---|
| H77 | TAGLVGLLTPGAKQNIQLINTNGSWHINS | GWLAGLFYQHKF | GAPTYSWGANDTDVFVLN |
| (AF009606) | (SEQ ID NO: 791) | (SEQ ID NO: 816) | (SEQ ID NO: 841) |
| HC-J1 | MSGLVSLFTPGAKQNIQLINTNGSWHINS | GWLAGLIYQHKF | GAPTYNWGANDTDVFVLN |
| (D10749) | (SEQ ID NO: 792) | (SEQ ID NO: 817) | (SEQ ID NO: 842) |
| HCV-L2 | TYGFTGLFRPGASQKIQLINTNGSWHINR | GFLAALFYTHRF | GAPTYSWGENETDVLLLN |
| (U01214) | (SEQ ID NO: 793) | (SEQ ID NO: 818) | (SEQ ID NO: 843) |
| India | AYRLASLFSTGPSQNIQLINSNGSWHINR | GWVAALFYSHKF | GVPTYTWGENETDVLLLN |
| (AY051292) | (SEQ ID NO: 794) | (SEQ ID NO: 819) | (SEQ ID NO: 844) |
| subtype 2a | ANSIAGLLSRGPRQNLQLINSNGSWHINR | GFITALFYARHF | GVPTYTWGENETDVFLLN |
| (AY746460) | (SEQ ID NO: 795) | (SEQ ID NO: 820) | (SEQ ID NO: 845) |
| MD2b1-2 | LARVTSLFSIGPKQNIQLINTNGSWHINR | GFIASLFYVNNI | GVPTYSWGENETDVFLLN |
| (AY232731) | (SEQ ID NO: 796) | (SEQ ID NO: 821) | (SEQ ID NO: 846) |
| BEBE1 | THLFTSMFSLGSQQRVQLIHTNGSWHINR | GFLAALFYTSSF | GAPTYNWGE3NETDVFLLN |
| (D50409) | (SEQ ID NO: 797) | (SEQ ID NO: 822) | (SEQ ID NO: 847) |
| D54 | ASGFAGLFTRGARQNIQLINTNGSWHINR | GFIASLFYANSF | GVPTYNWGDNETDVFLLN |
| (DQ155561) | (SEQ ID NO: 798) | (SEQ ID NO: 823) | (SEQ ID NO: 848) |
| VAT96 | THGLVSLFTPGSQQNIQLVNTNGSWHINR | GFIAALFYSHKF | GVPTYWGENDTDVFLLN |
| (AB031663) | (SEQ ID NO: 799) | (SEQ ID NO: 824) | (SEQ ID NO: 849) |
| 452 | ASGIVSLFTPGAKQNLQLVNTNGSWHINR | GFIAGLIYYHKF | GAPTYNWGANETDMFLLQ |
| (DQ437509) | (SEQ ID NO: 800) | (SEQ ID NO: 825) | (SEQ ID NO: 850) |
| HCV-Tr | TAGFTSFFTRGPSQNLQLVNSNGSWHINS | GFIAGLFYYHKF | GLPTYRFGVNESDVFLLT |
| (D49374) | (SEQ ID NO: 801) | (SEQ ID NO: 826) | (SEQ ID NO: 851) |
| JK049 | AFTITSLFSTGAKQPLHLVNTNGSWHINR | GFIAGLLYYHKF | GAPTYWGENESDVFLLE |
| (D63821) | (SEQ ID NO: 802) | (SEQ ID NO: 827) | (SEQ ID NO: 852) |
| ED43 | TAGLANLFSSGSKQNLQLINSNGSWHINR | GFLASLFYTHKF | GVPTYTWGENETDVFLLN |
| (Y11604) | (SEQ ID NO: 803) | (SEQ ID NO: 828) | (SEQ ID NO: 853) |
| 24 | ALTFAGLFTMGGQQHIQLINTNGSWHINR | GFLASLFYYRRF | GAPTYTWGENETDVFLLN |
| (DQ516083) | (SEQ ID NO: 804) | (SEQ ID NO: 829) | (SEQ ID NO: 854) |
| IFBT84 | IHGIANLFTPGSKQNLQLINTNGSWHINR | GFIAGLIYRNKF | GVPTYSWGANETDVFILN |
| (EF589160) | (SEQ ID NO: 805) | (SEQ ID NO: 830) | (SEQ ID NO: 855) |
| SA13 | ARGLASLFTPGPQQNLQLINTNGSWHINR | GVFAGLLYYHKF | GNPTYSWGENETDIFLLN |
| (AF064490) | (SEQ ID NO: 806) | (SEQ ID NO: 831) | (SEQ ID NO: 856) |
| 6a33 | TAGITGLFSPGASQNLQLIKNGSSWHINR | GFLASLFYVRKF | GNPTYTWGENETDVFMLE |
| (AY859526) | (SEQ ID NO: 807) | (SEQ ID NO: 832) | (SEQ ID NO: 857) |
| Th580 | AYLFTSIFSSGPNQKIQLINTNGSWHINR | GFLSALFYRSNF | GNPTYNWGENETDVFMLE |
| (NC009827) | (SEQ ID NO: 808) | (SEQ ID NO: 833) | (SEQ ID NO: 858) |
| D88 | ITGFSGLFSSGSQQKLQLVNTNGSWHINR | GFIAALFYTYRF | GLPTYSWGENESDVFLLE |
| (EF420130) | (SEQ ID NO: 809) | (SEQ ID NO: 834) | (SEQ ID NO: 859) |
| GX004 | VGNIASLFSPGSRQNLQLINSNGSWHINR | GFIASLFYFNKF | GLPTYTWGENESDVFLLE |
| (DQ314805) | (SEQ ID NO: 810) | (SEQ ID NO: 835) | (SEQ ID NO: 860) |
| C-0046 | THGIASLFSPGAHQRLQLINSNGSWHINR | GFLANLFYVHKI | GLPTYTWGENETDVFILE |
| (DQ835764) | (SEQ ID NO: 811) | (SEQ ID NO: 836) | (SEQ ID NO: 861) |
| JK046 | TSGLVSLFSAGARQNLQLINTNGSWHINR | GFIASLFYRNKF | GNPTYTWGANETDVFMMS |
| (D63822) | (SEQ ID NO: 812) | (SEQ ID NO: 837) | (SEQ ID NO: 862) |
| VN405 | TNRLTSFFSPGSKQNVQLIKTNGSWHINR | GFIAGLLYAHRF | GLPTYTWGANESDVFLLR |
| (D84264) | (SEQ ID NO: 813) | (SEQ ID NO: 838) | (SEQ ID NO: 863) |
| C-0208 | TSGLTGLFTPGARQNIQLINTNGSWHINR | GFIAGLIYAHKF | GFPTYSWGGNETDVFLLQ |
| (DQ835763) | (SEQ ID NO: 814) | (SEQ ID NO: 839) | (SEQ ID NO: 864) |
| KM42 | VYGLSTLFTRGSQQNIQLVNSNGSWHVNR | GFIAGLFYYHKF | GLPTYNWGENMSDVFLLQ |
| (DQ278894) | (SEQ ID NO: 815) | (SEQ ID NO: 840) | (SEQ ID NO: 865) |

Non-limiting examples of modified E2 polypeptides of the invention include the following.

| Modified E2 | |
|---|---|
| Polypeptides | Polypeptide Sequences |
| E2₍₃₉₆₋₇₄₆₎ | TAGLVGLLTPGAKQNIQLINTNGSWHINSTAL |
| | NCNESLNTGWLAGLFYQHKFNSSGCPERLASC |
| | RRLTDFAQGWGPISYANGSGLDERPYCWHYPP |
| | RPCGIVPAKSVCGPVYCFTPSPVVVGTTDRSG |
| | APTYSWGANDTDVFVLNNTRPPLGNWFGCTWM |
| | NSTGFTKVCGAPPCVIGGVGNNTLLCPTDCFR |
| | HKPEATYSRCGSGPWITPRCMVDYPYRLWHYP |
| | CTINYTIFKVRMYVGGVEHRLEAACNWTRGER |
| | CDLEDRDRSELSPLLLSTTQWQVLPCSFTTLP |
| | ALSTGLIHLHQNIVDVQYLYGVGSSIASWAIK |
| | WEYVVLLFLLLADARVCSCLWMMLLISQAEA |
| | (SEQ ID NO: 866) |
| E2₍₃₉₆₋₇₁₇₎ | TAGLVGLLTPGAKQNIQLINTNGSWHINSTAL |
| (Δ₃₈₄₋₃₉₅₎ΔTM | NCNESLNTGWLAGLFYQHKFNSSGCPERLASC |
| | RRLTDFAQGWGPISYANGSGLDERPYCWHYPP |
| | RPCGIVPAKSVCGPVYCFTPSPVVVGTTDRSG |
| | APTYSWGANDTDVFVLNNTRPPLGNWFGCTWM |
| | NSTGFTKVCGAPPCVIGGVGNNTLLCPTDCFR |
| | KHPEATYSRCGSGPWITPRCMVDYPYRLWHYP |
| | CTINYTIFKVRMYVGGVEHRLEAACNWTRGER |
| | CDLEDRDRSELSPLLLSTTQWQVLPCSFTTLP |
| | ALSTGLIHLHQNIVDVQYLYGVGSSIASWAIK |
| | WE |
| | (SEQ ID NO: 867) |
| E2₃₉₆₋₆₆₁ | TAGLVGLLTPGAKQNIQLINTNGSWHINSTAL |
| | NCNESLNTGWLAGLFYQHKFNSSGCPERLASC |
| | RRLTDFAQGWGPISYANGSGLDERPYCWHYPP |
| | RPCGIVPAKSVCGPVYCFTPSPVVVGTTDRSG |
| | APTYSWGANDTDVFVLNNTRPPLGNWFGCTWM |
| | NSTGFTKVCGAPPCVIGGVGNNTLLVTPDCFR |
| | KHPEATYSRCGSGPWITPRCMVDYPYRLWHYP |
| | CTINYTIFKVRMYVGGVEHRLEAACNWTRGER |
| | CDLEDRDRSE |
| | (SEQ ID NO: 868) |
| E2₃₉₆₋₆₄₇ | TAGLVGLLTPGAKQNIQLINTNGSWHINSTAL |
| | NCNESLNTGWLAGLFYQHKFNSSGCPERLASC |
| | RRLTDFAQGWGPISYANGSGLDERPYCWHYPP |
| | RPCGIVPAKSVCGPVYCFTPSPVVVGTTDRSG |
| | APTYSWGANDTDVFVLNNTRPPLGNWFGCTWM |
| | NSTGFTKVCGAPPCVIGGVGNNTLLCPTDCFR |
| | KHPEATYSRCGSGPWITPRCMVDYPRYLWHYP |
| | CTINYTIFKVRMYVGGVEHRLEAACNWT |
| | (SEQ ID NO: 869) |
| E2₃₉₆₋₆₄₅ | TAGLVGLLTPGAKQNIQLINTNGSWHINSTAL |
| | NCNESLNTGWLAGLFYQHKFNSSGCPERLASC |
| | RRLTDFAQGWGPISYANGSGLDERPYCWHYPP |
| | RPCGIVPAKSVCGPVYCFTPSPVVVGTTDRSG |
| | APTYSWGANDTDVFVLNNTRPPLGNWFGCTWM |
| | NSTGFTKVCGAPPCVIGGVGNNTLLCPTDCFR |
| | KHPEATYSRCGSGPWITPRCMVDYPYRLWHYP |
| | CTINYTIFKVRMYVGGVEHRLEAACN |
| | (SEQ ID NO: 870) |
| E2(Δ₃₈₄₋₃₉₅)ΔN5 | TAGLVGLLTPGAKQNIQLINTNGSWHINSTAL |
| | NCNESLNTGWLAGLFYQHKFNSSGCPERLASC |
| | GSSGCWHYPPRPCGIVPAKSVCGPVYCFTPSP |
| | VVVGTTDRSGAPTYSWGANDTDVFVLNNTRPP |
| | LGNWFGCTWMNSTGFTKVCGAPPCVIGGVGNN |
| | TLLCPTDCFRKHPEATYSRCGSGPWITPRCMV |
| | DYPYRLWHYPCTINYTIFKVRMYVGGVEHRLE |
| | AACN |
| | (SEQ ID NO: 871) |
| E2(₃₈₄₋₃₉₅)ΔN9 | TAGLVGLLTPGAKQNIQLINTNGSWHINSTAL |
| | NCNESLNTGWLAGLFYQHKFNSSGCPERLASC |
| | RRLTDFAQGWGPISYANGSGLDERPYCWHYPP |
| | RPCGIVPAKSVCGPVYCFTPSPVVVGTTDRSG |
| | APTYSWGANDTDVFVLNNTRPPLGNWFGCTWM |
| | NSTGFTKVCGAPPCGSSGCPTDCFRKHPEATY |
| | SRCGSGPWITPRCMVDYPYRLWHYPCTINYTI |
| | FKVRMYVGGVEHRLEAACN |
| | (SEQ ID NO: 872) |
| E2(Δ₃₈₄₋₃₉₅)ΔN5N9 | TAGLVGLLTPGAKQNIQLINTNGSWHINSTAL |
| | NCNESLNTGWLAGLFYQHKFNSSGCPERLASC |
| | GSSGCWHYPPRPCGIVPAKSVCGPVYCFTPSP |
| | VVVGTTDRSGAPTYSWGANDTDVFVLNNTRPP |
| | LGNWFGCTWMNSTGFTKVCGAPPCGSSGCPTD |
| | CFRKHPEATYSRCGSGPWITPRCMVDYPYRLW |
| | HYPCTINYTIFKVRMYVGGVEHRLEAACN |
| | (SEQ ID NO: 873) |

A polypeptide of the invention can also include non-E2 sequences at the N or C terminus. Non-E2 sequences can be, for example, a tag such as an N-terminal ubiquitin signal, a poly-histidine sequence, a FLAG (DYKDDDDK) sequence, an HA sequence, a myc sequence, a V5 sequence, a chitin binding protein sequence, a maltose binding protein sequence or a glutathione-S-transferase sequence.

Nucleic Acids Encoding Modified E2 Polypeptides

The invention also provides isolated nucleic acids encoding modified E2 polypeptides. As used herein, the term “nucleic acid” refers to a polymer of deoxynucleic ribose nucleic acids (DNA), as well as ribose nucleic acids (RNA). The term includes linear molecules, as well as covalently closed circular molecules. It includes single stranded molecules, as well as double stranded molecules.

The term “isolated,” as used herein with reference to a nucleic acid molecule, means that the nucleic acid molecule is free of unrelated nucleic acid sequences, i.e. nucleic acid sequences encoding other genes or non-E2 polypeptide sequences, or those involved in the expression of such other genes, that flank it's 5′ and 3′ ends in the naturally-occurring genome of the organism from which the nucleic acid of the invention is derived. Accordingly, an “isolated nucleic acid” of the invention has a structure that is different from that of any naturally occurring nucleic acid or to that of any fragment of a naturally occurring genomic nucleic acid spanning more than three separate genes. Thus, the term “isolated nucleic acid molecule” includes, for example, (1) a DNA molecule that has the sequence of part of a naturally occurring genomic DNA molecule, but is not flanked by both of the coding sequences that flank that part of the molecule in the genome of the organism in which it naturally occurs; (2) a nucleic acid incorporated into a vector or into the genomic DNA of a prokaryote or eukaryote in a manner such that the resulting molecule is not identical to any naturally-occurring vector or genomic DNA; (3) a separate molecule such as a cDNA, a genomic fragment, a fragment produced by polymerase chain reaction (PCR), or a restriction fragment; and (4) a recombinant nucleotide sequence that is part of a hybrid gene, i.e. a gene encoding a fusion protein. Specifically excluded from this definition are nucleic acids present in mixtures of (1) DNA molecules, (2) transfected cells, and (3) cell clones, e.g., as these occur in a DNA library such as a cDNA or genomic DNA library.

Examples of nucleic acid sequences encoding modified E2 polypeptides of the invention are shown below.

| Modified E2 | |
|---|---|
| Polypeptides | Nucleic Acid Sequences |
| E2(₃₉₆₋₇₄₆) | ACGGCTGGGCTTGTTGGTCTCCTTACACCAGG |
| (Δ₃₈₄₋₃₉₅) | CGCCAAGCAGAACATCCAACTGATCAACACCA |
| | ACGGCAGTTGGCACATCAATAGCACGGCCTTG |
| | AACTGCAATGAAAGCCTTAACACCGGCTGGTT |
| | AGCAGGGCTCTTCTATCAGCACAAATTCAACT |
| | CTTCAGGCTGTCCTGAGAGGTTGGCCAGCTGC |
| | CGACGCCTTACCGATTTTGCCCAGGGCTGGGG |
| | TCCTATCAGTTATGCCAACGGAAGCGGCCTCG |
| | ACGAACGCCCCTACTGCTGGCACTACCCTCCA |
| | AGACCTTGTGGCATTGTGCCCGCAAAGAGCGT |
| | GTGTGGCCCGGTATATTGCTTCACTCCCAGCC |
| | CCGTGGTGGTGGGAACGACCGACAGGTCGGGC |
| | GCGCCTACCTACAGCTGGGGTGCAAATGATAC |
| | GGATGTCTTCGTCCTTAACAACACCAGGCCAC |
| | CGCTGGGCAATTGGTTCGGTTGTACCTGGATG |
| | AACTCAACTGGATTCACCAAAGTGTGCGGAGC |
| | GCCCCCTTGTGTCATCGGAGGGGTGGGCAACA |
| | ACACCTTGCTCTGCCCCACTGATTGTTTCCGC |
| | AAGCATCCGGAAGCCACATACTCTCGGTGCGG |
| | CTCCGGTCCCTGGATTACACCCAGGTGCATGG |
| | TCGACTACCCGTATAGGCTTTGGCACTATCCT |
| | TGTACCATCAATTACACCATATTCAAAGTCAG |
| | GATGTACGTGGGAGGGGTCGAGCACAGGCTGG |
| | AAGCGGCCTGCAACTGGACGCGGGGCGAACGC |
| | TGTGATCTGGAAGACAGGGACAGGTCCGAGCT |
| | CAGCCCATTGCTGCTGTCCACCACACAGTGGC |
| | AGGTCCTTCCGTGTTCTTTCACGACCCTGCCA |
| | GCCTTGTCCACCGGCCTCATCCACCTCCACCA |
| | GAACATTGTGGACGTGCAGTACTTGTACGGGG |
| | TAGGGTCAAGCATCGCGTCCTGGGCCATTAAG |
| | TGGGAGTACGTCGTTCTCCTGTTCCTCCTGCT |
| | TGCAGACGCGCGCGTCTGCTCCTGCTTGTGGA |
| | TGATGTTACTCATATCCCAAGCGGAGGCG |
| | (SEQ ID NO: 874) |
| E2(₃₉₆₋₇₁₇) | ACGGCTGGGCTTGTTGGTCTCCTTACACCAGG |
| (Δ₃₈₄₋₃₉₅) ΔTM | CGCCAAGCAGAACATCCAACTGATCAACACCA |
| | ACGGCAGTTGGCACATCAATAGCACGGCCTTG |
| | AACTGCAATGAAAGCCTTAACACCGGCTGGTT |
| | AGCAGGGCTCTTCTATCAGCACAAATTCAACT |
| | CTTCAGGCTGTCCTGAGAGGTTGGCCAGCTGC |
| | CGACGCCTTACCGATTTTGCCCAGGGCTGGGG |
| | TCCTATCAGTTATGCCAACGGAAGCGGCCTCG |
| | ACGAACGCCCCTACTGCTGGCACTACCCTCCA |
| | AGACCTTGTGGCATTGTGCCCGCAAAGAGCGT |
| | GTGTGGCCCGGTATATTGCTTCACTCCCAGCC |
| | CCGTGGTGGTGGGAACGACCGACAGGTCGGGC |
| | GCGCCTACCTACAGCTGGGGTGCAAATGATAC |
| | GGATGTCTTCGTCCTTAACAACACCAGGCCAC |
| | CGCTGGGCAATTGGTTCGGTTGTACCTGGATG |
| | AACTCAACTGGATTCACCAAAGTGTGCGGAGC |
| | GCCCCCTTGTGTCATCGGAGGGGTGGGCAACA |
| | ACACCTTGCTCTGCCCCACTGATTGTTTCCGC |
| | AAGCATCCGGAAGCCACATACTCTCGGTGCGG |
| | CTCCGGTCCCTGGATTACACCCAGGTGCATGG |
| | TCGACTACCCGTATAGGCTTTGGCACTATCCT |
| | TGTACCATCAATTACACCATATTCAAAGTCAG |
| | GATGTACGTGGGAGGGGTCGAGCACAGGCTGG |
| | AAGCGGCCTGCAACTGGACGCGGGGCGAACGC |
| | TGTGATCTGGAAGACAGGGACAGGTCCGAGCT |
| | CAGCCCATTGCTGCTGTCCACCACACAGTGGC |
| | AGGTCCTTCCGTGTTCTTTCACGACCCTGCCA |
| | GCCTTGTCCACCGGCCTCATCCACCTCCACCA |
| | GAACATTGTGGACGTGCAGTACTTGTACGGGG |
| | TAGGGTCAAGCATCGCGTCCTGGGCCATTAAG |
| | TGGGAG |
| | (SEQ ID NO: 875) |
| E2₃₉₆₋₆₆₁ | ACGGCTGGGCTTGTTGGTCTCCTTACACCAGG |
| | CGCCAAGCAGAACATCCAACTGATCAACACCA |
| | ACGGCAGTTGGCACATCAATAGCACGGCCTTG |
| | AACTGCAATGAAAGCCTTAACACCGGCTGGTT |
| | AGCAGGGCTCTTCTATCAGCACAAATTCAACT |
| | CTTCAGGCTGTCCTGAGAGGTTGGCCAGCTGC |
| | CGACGCCTTACCGATTTTGCCCAGGGCTGGGG |
| | TCCTATCAGTTATGCCAACGGAAGCGGCCTCG |
| | ACGAACGCCCCTACTGCTGGCACTACCCTCCA |
| | AGACCTTGTGGCATTGTGCCCGCAAAGAGCGT |
| | GTGTGGCCCGGTATATTGCTTCACTCCCAGCC |
| | CCGTGGTGGTGGGAACGACCGACAGGTCGGGC |
| | GCGCCTACCTACAGCTGGGGTGCAAATGATAC |
| | GGATGTCTTCGTCCTTAACAACACCAGGCCAC |
| | CGCTGGGCAATTGGTTCGGTTGTACCTGGATG |
| | AACTCAACTGGATTCACCAAAGTGTGCGGAGC |
| | GCCCCCTTGTGTCATCGGAGGGGTGGGCAACA |
| | ACACCTTGCTCTGCCCCACTGATTGTTTCCGC |
| | AAGCATCCGGAAGCCACATACTCTCGGTGCGG |
| | CTCCGGTCCCTGGATTACACCCAGGTGCATGG |
| | TCGACTACCCGTATAGGCTTTGGCACTATCCT |
| | TGTACCATCAATTACACCATATTCAAAGTCAG |
| | GATGTACGTGGGAGGGGTCGAGCACAGGCTGG |
| | AAGCGGCCTGCAACTGGACGCGGGGCGAACGC |
| | TGTGATCTGGAAGACAGGGACAGGTCCGAG |
| | (SEQ ID NO: 876) |
| E2₃₉₆₋₆₄₇ | ACGGCTGGGCTTGTTGGTCTCCTTACACCAGG |
| | CGCCAAGCAGAACATCCAACTGATCAACACCA |
| | ACGGCAGTTGGCACATCAATAGCACGGCCTTG |
| | AACTGCAATGAAAGCCTTAACACCGGCTGGTT |
| | AGCAGGGCTCTTCTATCAGCACAAATTCAACT |
| | CTTCAGGCTGTCCTGAGAGGTTGGCCAGCTGC |
| | CGACGCCTTACCGATTTTGCCCAGGGCTGGGG |
| | TCCTATCAGTTATGCCAACGGAAGCGGCCTCG |
| | ACGAACGCCCCTACTGCTGGCACTACCCTCCA |
| | AGACCTTGTGGCATTGTGCCCGCAAAGAGCGT |
| | GTGTGGCCCGGTATATTGCTTCACTCCCAGCC |
| | CCGTGGTGGTGGGAACGACCGACAGGTCGGGC |
| | GCGCCTACCTACAGCTGGGGTGCAAATGATAC |
| | GGATGTCTTCGTCCTTAACAACACCAGGCCAC |
| | CGCTGGGCAATTGGTTCGGTTGTACCTGGATG |
| | AACTCAACTGGATTCACCAAAGTGTGCGGAGC |
| | GCCCCCTTGTGTCATCGGAGGGGTGGGCAACA |
| | ACACCTTGCTCTGCCCCACTGATTGTTTCCGC |
| | AAGCATCCGGAAGCCACATACTCTCGGTGCGG |
| | CTCCGGTCCCTGGATTACACCCAGGTGCATGG |
| | TCGACTACCCGTATAGGCTTTGGCACTATCCT |
| | TGTACCATCAATTACACCATATTCAAAGTCAG |
| | GATGTACGTGGGAGGGGTCGAGCACAGGCTGG |
| | AAGCGGCCTGCAACTGGACG |
| | (SEQ ID NO: 877) |
| E2₃₉₆₋₆₄₅ | ACGGCTGGGCTTGTTGGTCTCCTTACACCAGG |
| | CGCCAAGCAGAACATCCAACTGATCAACACCA |
| | ACGGCAGTTGGCACATCAATAGCACGGCCTTG |
| | AACTGCAATGAAAGCCTTAACACCGGCTGGTT |
| | AGCAGGGCTCTTCTATCAGCACAAATTCAACT |
| | CTTCAGGCTGTCCTGAGAGGTTGGCCAGCTGC |
| | CGACGCCTTACCGATTTTGCCCAGGGCTGGGG |
| | TCCTATCAGTTATGCCAACGGAAGCGGCCTCG |
| | ACGAACGCCCCTACTGCTGGCACTACCCTCCA |
| | AGACCTTGTGGCATTGTGCCCGCAAAGAGCGT |
| | GTGTGGCCCGGTATATTGCTTCACTCCCAGCC |
| | CCGTGGTGGTGGGAACGACCGACAGGTCGGGC |
| | GCGCCTACCTACAGCTGGGGTGCAAATGATAC |
| | GGATGTCTTCGTCCTTAACAACACCAGGCCAC |
| | CGCTGGGCAATTGGTTCGGTTGTACCTGGATG |
| | AACTCAACTGGATTCACCAAAGTGTGCGGAGC |
| | GCCCCCTTGTGTCATCGGAGGGGTGGGCAACA |
| | ACACCTTGCTCTGCCCCACTGATTGTTTCCGC |
| | AAGCATCCGGAAGCCACATACTCTCGGTGCGG |
| | CTCCGGTCCCTGGATTACACCCAGGTGCATGG |
| | TCGACTACCCGTATAGGCTTTGGCACTATCCT |
| | TGTACCATCAATTACACCATATTCAAAGTCAG |
| | GATGTACGTGGGAGGGGTCGAGCACAGGCTGG |
| | AAGCGGCCTGCAAC |
| | (SEQ ID NO: 878) |
| E2 (Δ₃₈₄₋₃₉₅) ΔN5 | ACGGCTGGGCTTGTTGGTCTCCTTACACCAGG |
| | CGCCAAGCAGAACATCCAACTGATCAACACCA |
| | ACGGCAGTTGGCACATCAATAGCACGGCCTTG |
| | AACTGCAATGAAAGCCTTAACACCGGCTGGTT |
| | AGCAGGGCTCTTCTATCAGCACAAATTCAACT |
| | CTTCAGGCTGTCCTGAGAGGTTGGCCAGCTGC |
| | GGCTCTAGCGGATGCTGGCACTACCCTCCAAG |
| | ACCTTGTGGCATTGTGCCCGCAAAGAGCGTGT |
| | GTGGCCCGGTATATTGCTTCACTCCCAGCCCC |
| | GTGGTGGTGGGAACGACCGACAGGTCGGGCGC |
| | GCCTACCTACAGCTGGGGTGCAAATGATACGG |
| | ATGTCTTCGTCCTTAACAACACCAGGCCACCG |
| | CTGGGCAATTGGTTCGGTTGTACCTGGATGAA |
| | CTCAACTGGATTCACCAAAGTGTGCGGAGCGC |
| | CCCCTTGTGTCATCGGAGGGGTGGGCAACAAC |
| | ACCTTGCTCTGCCCCACTGATTGTTTCCGCAA |
| | GCATCCGGAAGCCACATACTCTCGGTGCGGCT |
| | CCGGTCCCTGGATTACACCCAGGTGCATGGTC |
| | GACTACCCGTATAGGCTTTGGCACTATCCTTG |
| | TACCATCAATTACACCATATTCAAAGTCAGGA |
| | TGTACGTGGGAGGGGTCGAGCACAGGCTGGAA |
| | GCGGCCTGCAAC |
| | (SEQ ID NO: 879) |
| E2 (Δ₃₈₄₋₃₉₅) ΔN9 | ACGGCTGGGCTTGTTGGTCTCCTTACACCAGG |
| | CGCCAAGCAGAACATCCAACTGATCAACACCA |
| | ACGGCAGTTGGCACATCAATAGCACGGCCTTG |
| | AACTGCAATGAAAGCCTTAACACCGGCTGGTT |
| | AGCAGGGCTCTTCTATCAGCACAAATTCAACT |
| | CTTCAGGCTGTCCTGAGAGGTTGGCCAGCTGC |
| | CGACGCCTTACCGATTTTGCCCAGGGCTGGGG |
| | TCCTATCAGTTATGCCAACGGAAGCGGCCTCG |
| | ACGAACGCCCCTACTGCTGGCACTACCCTCCA |
| | AGACCTTGTGGCATTGTGCCCGCAAAGAGCGT |
| | GTGTGGCCCGGTATATTGCTTCACTCCCAGCC |
| | CCGTGGTGGTGGGAACGACCGACAGGTCGGGC |
| | GCGCCTACCTACAGCTGGGGTGCAAATGATAC |
| | GGATGTCTTCGTCCTTAACAACACCAGGCCAC |
| | CGCTGGGCAATTGGTTCGGTTGTACCTGGATG |
| | AACTCAACTGGATTCACCAAAGTGTGCGGAGC |
| | GCCCCCTTGTGGAAGCTCTGGCTGCCCCACTG |
| | ATTGTTTCCGCAAGCATCCGGAAGCCACATAC |
| | TCTCGGTGCGGCTCCGGTCCCTGGATTACACC |
| | CAGGTGCATGGTCGACTACCCGTATAGGCTTT |
| | GGCACTATCCTTGTACCATCAATTACACCATA |
| | TTCAAAGTCAGGATGTACGTGGGAGGGGTCGA |
| | GCACAGGCTGGAAGCGGCCTGCAAC |
| | (SEQ ID NO: 880) |
| E2 (Δ₃₈₄₋₃₉₅) | ACGGCTGGGCTTGTTGGTCTCCTTACACCAGG |
| ΔN5N9 | CGCCAAGCAGAACATCCAACTGATCAACACCA |
| | ACGGCAGTTGGCACATCAATAGCACGGCCTTG |
| | AACTGCAATGAAAGCCTTAACACCGGCTGGTT |
| | AGCAGGGCTCTTCTATCAGCACAAATTCAACT |
| | CTTCAGGCTGTCCTGAGAGGTTGGCCAGCTGC |
| | GGCTCTAGCGGATGCTGGCACTACCCTCCAAG |
| | ACCTTGTGGCATTGTGCCCGCAAAGAGCGTGT |
| | GTGGCCCGGTATATTGCTTCACTCCCAGCCCC |
| | GTGGTGGTGGGAACGACCGACAGGTCGGGCGC |
| | GCCTACCTACAGCTGGGGTGCAAATGATACGG |
| | ATGTCTTCGTCCTTAACAACACCAGGCCACCG |
| | CTGGGCAATTGGTTCGGTTGTACCTGGATGAA |
| | CTCAACTGGATTCACCAAAGTGTGCGGAGCGC |
| | CCCCTTGTGGAAGCTCTGGCTGCCCCACTGAT |
| | TGTTTCCGCAAGCATCCGGAAGCCACATACTC |
| | TCGGTGCGGCTCCGGTCCCTGGATTACACCCA |
| | GGTGCATGGTCGACTACCCGTATAGGCTTTGG |
| | CACTATCCTTGTACCATCAATTACACCATATT |
| | CAAAGTCAGGATGTACGTGGGAGGGGTCGAGC |
| | ACAGGCTGGAAGCGGCCTGCAAC |
| | (SEQ ID NO: 881) |

Nucleic acids encoding modified E2 polypeptides of the invention can be generated from nucleic acids encoding the naturally-occurring HCV polyprotein using methods known to those of skilled in the art. For example, nucleic acids encoding modified E2 polypeptides containing various amino acid substitutions can be produced by site-specific mutagenesis and polymerase chain reaction (PCR) amplification from the nucleic acids encoding the naturally-occurring HCV polyprotein. Nucleic acids encoding modified E2 polypeptides, i.e. polypeptides that do not include amino acid residues 384 to 410 of the hypervariable region of the naturally occurring E2 protein, can be produced by PCR using primers that do not encompass the nucleotides coding for amino acid residues 384 to 410. Nucleic acid sequences encoding the naturally-occurring HCV polyproteins are disclosed at the NCBI website (www.ncbi.nlm.nih.gov). Selected accession numbers for nucleic acids encoding the naturally-occurring HCV polyproteins are as follows: AF009606; D10749; U01214; AY051292; AY746460; AY232731; D50409; DQ155561; AB031663; DQ437509; D49374; D63821; Y11604; DQ516083; EF589160; AF064490; AY859526; NC009827; EF420130; DQ314805 ; DQ835764; D63822; D84264; DQ835763; and DQ278894.

Methods for isolating nucleic acids encoding the naturally-occurring HCV polyprotein, as well as technologies for generation of nucleic acids encoding E2 polypeptides of the invention are known of skill in the art. See for example, CURRENT PROTOCOLS IN MOLECULAR BIOLOGY, Ausubel et al. edts. (John Wiley & Sons, Inc., 1999) or MOLECULAR CLONING: A LABORATORY MANUAL, Sambrook et al. (Cold Spring Harbor Laboratory Press, New York, 1989).

Nucleic acids encoding a polypeptide of the invention can be used for recombinant expression of the E2 polypeptide of the invention. Nucleic acids encoding a polypeptide of the invention can also be used in a nucleic acid-based vaccine to elicit an immune response against an HCV.

Nucleic acid encoding a polypeptide of the invention can be operably-linked to an expression control sequence in an expression vector, which can be introduced into a host cell for expression of the encoded polypeptide or administered to a mammal to elicit an immune response against the polypeptide.

As used herein, the term “operably linked” means that a nucleic acid and an expression control sequence are positioned in such a way that the expression control sequence directs expression of the nucleic acid when the appropriate molecules such as transcriptional activator proteins are bound to the expression control sequence. Thus, the term “expression control sequence” means a nucleic acid sequence sufficient to direct transcription of another nucleic acid sequence that is operably linked to the expression control sequence to produce an RNA transcript when appropriate molecules such as transcriptional activator proteins are bound the expression control sequence.

An “expression vector” is a nucleic acid molecule capable of transporting and/or allowing for the expression of another nucleic acid to which it has been linked. Expression vectors contain appropriate expression control sequences that direct expression of a nucleic acid that is operably linked to the expression control sequence to produce a transcript. The product of that expression is referred to as a messenger ribose nucleic acid (mRNA) transcript.

The expression vector may also include other sequences such as enhancer sequences, synthetic introns, adenovirus tripartite leader (TPL) sequences and modified polyadenylation and transcriptional termination sequences, e.g. bovine growth hormone or rabbit beta-globulin polyadenylation sequences, to improve expression of the nucleic acid encoding the E2 polypeptide.

Nucleic acids encoding E2 polypeptides of the invention can be incorporated into viral, bacterial, insect, yeast or mammalian expression vectors. As such, nucleic acids encoding E2 polypeptides can be operably-linked to expression control sequences such as viral, bacterial, insect, yeast or mammalian promoters and enhancers. Examples of expression control sequences such as enhancers and promoters include viral promoters such as SV 40 promoter, Rous Sarcoma Virus (RSV) promoter, and cytomegalovirus (CMV) immediate early promoter. Examples of viral vectors include retrovirus-based vectors, e.g. Lentiviruses, Adenoviruses and Adeno-associated viruses. These are particularly useful as DNA-based vaccines.

The nucleic acid encoding an E2 polypeptide of the invention can also be linked to nucleic acid sequences that code for unrelated amino acid sequences such as N-terminal ubiquitin signals to improve antigen targeting, a poly-histidine sequence, a FLAG (DYKDDDDK) sequence, an HA sequence, a myc sequence, a V5 sequence, a chitin binding protein sequence, a maltose binding protein sequence or a glutathione-S-transferase sequence

Expression vectors containing nucleic acids encoding E2 polypeptides can be introduced into bacterial, insect, yeast or mammalian host cells for expression using conventional methods including, without limitation, transformation, transduction and transfection. Expression vectors containing nucleic acids encoding E2 polypeptides, in saline for example, can be introduced into a mammal, e.g. mammalian tissues, using standard methods including, for example, injection using a standard hypodermic need, by a gene gun delivery, jet injection or liposome-mediated delivery. Injection can be intramuscular or intradermal. Electroporation, myotoxins such as buivacaine or hypertonic solutions of saline or sucrose can also aid in delivery.

When expressed in bacterial, yeast, insect or mammalian host cells, E2 polypeptides of the invention can be purified using a method provided by the invention. Specifically, E2 polypeptides of the invention are purified by affinity chromatography using a cross-neutralizing antibody such as, for example, AR3A, AR3B, AR3C or AR3D in combination with size exclusion chromatography. More specifically, an E2 polypeptide of the invention can be separated from unrelated proteins by affinity chromatography using a conformation-dependent antibody of the invention such as AR3A. The E2 polypeptide can be eluted at acidic, neutral or basic pH using: (1) 0.2M glycine pH 2.2, (2) 2M sodium thiocyanate (pH adjusted to pH 7.4 with 50 mM Tris-HCl); or (3) 0.2M glycine pH 11.5, and then further purified by size-exclusion chromatography. The method provided by the invention for purifying E2 polypeptide allows for the purification of E2 polypeptides that properly fold to form the conformational epitope described herein.

When introduced into a mammal or mammalian tissue, nucleic acids encoding E2 polypeptides, incorporated in a viral vector, for example, can be used as a nucleic acid-based vaccine to elicit an immune response against HCV.

### Cross-Neutralizing Antibodies of the Invention

The invention also provides an antibody that binds specifically with a modified E2 polypeptide of the invention. The antibody is a cross-neutralizing antibody, i.e. one that neutralizes at least two HCV strains, isolates, species, quasispecies, subtypes or genotypes.

The term “antibody,” as used herein, refers to a full-length immunoglobulin molecule or an immunologically-active fragment of an immunoglobulin molecule such as the Fab or F(ab′)₂ fragment generated by, for example, cleavage of the antibody with an enzyme such as pepsin or co-expression of an antibody light chain and an antibody heavy chain in bacteria, yeast, insect cell or mammalian cell. An “antibody of the invention” can be a Fab, bivalent F(ab′)₂, IgG, IgD, IgA, IgE or IgM.

As discussed above, the term “bind selectively” or “selectively binds,” in reference to an antibody of the invention, means that the antibody binds with a particular antigen without substantially binding to other unrelated antigens. For example, the antibody has at least 50% or greater affinity, preferably about 75% or greater affinity, and more preferably, about 90% or greater affinity, to a particular polypeptide than to other unrelated polypeptides.

The term “neutralize,” as used herein in reference to an antibody, means that the antibody can prevent or reduce HCV infection or replication in a cell culture or in a mammal, as well as alleviate one or more symptoms associated with HCV infection in a mammal. The term “reduce,” as used herein, means a decrease in any amount such as a 5%, 10%, 15%, 20%, 25%, 30%, 35%, 40%, 45%, 50%, 55%, 60%, 65% or more than 65%. HCV infection or replication can be detected by examining HCV RNA levels, virus particles count or clinical symptoms associated with HCV infection using methods known to those of skill in the art. Whether an antibody will prevent or reduce HCV infection or replication or alleviate associated symptions can be determined using methods known in the art, as well as the methods described herein, including determining the level of HCV RNA in a sample from a mammal that has been infected with HCV or detecting reduction of signals from a reporter gene encoded by the virus such as, for example, the relative light unit (RLU) for luciferase or the mean fluorescence intensity (MFI) of green fluorescent protein (GFP).

Thus, whether an antibody will bind selectively to HCV and neutralize it can be determined using methods known in the art, as well as the methods described herein, including determining the level of HCV RNA or detecting reduction of signals from a reporter gene encoded by the virus such as, for example, the relative light unit (RLU) for luciferase or the mean fluorescence intensity (MFI) of green fluorescent protein (GFP).

An antibody of the invention can be a polyclonal or monoclonal antibody. Polyclonal antibodies can be obtained by immunizing a mammal with a modified polypeptide of the invention, and then isolating antibodies from the blood of the mammal using standard techniques including, for example, enzyme linked immunosorbent assay (ELISA) to determine antibody titer and protein A chromatography to obtain the antibody-containing IgG fraction.

A monoclonal antibody is a population of molecules having a common antigen binding site that binds specifically with a particular antigenic epitope. A monoclonal antibody can be obtained by selecting an antibody-producing cell from a mammal that has been immunized with a modified polypeptide of the invention and fusing the antibody-producing cell, e.g. a B cell, with a myeloma to generate an antibody-producing hybridoma. A monoclonal antibody of the invention can also be obtained by screening a recombinant combinatorial library such as an antibody phage display library using, for example, a modified polypeptide of the invention. See, for example, PHAGE DISPLAY—A LABORATORY MANUAL, Barbas, et al., eds. Cold Spring Harbor Laboratory Press, Cold Spring Harbor, N.Y., 2001; and Kontermann & Dübel, ANTIBODY ENGINEERING, Heidelberg: Springer-Verlag. Berlin, 2001.

An immunologically-active fragment of an antibody is the biologically active fragment of an immunoglobulin molecule, for example, the F(ab) or F(ab′)₂ fragment generated by cleavage of the antibody with an enzyme such as pepsin.

An antibody of the invention can also be a murine, chimeric, humanized or fully human antibody. A murine antibody is an antibody derived entirely from a murine source, for example, an antibody derived from a murine hybridoma generated from the fusion of a mouse myeloma cell and a mouse B-lymphocyte cell. A chimeric antibody is an antibody that has variable regions derived from a non-human source, e.g. murine or primate, and constant regions derived from a human source. A humanized antibody has antigen-binding regions, e.g. complementarity-determining regions, derived from a mouse source, and the remaining variable regions and constant regions derived from a human source. A fully human antibody is antibody from human cells or derived from transgenic mice carrying human antibody genes.

Methods to generate antibodies are well known in the art. For example, a polyclonal antibody of the invention can be prepared by immunizing a suitable mammal with a modified polypeptide of the invention. The mammal can be, for example, a rabbit, goat, sheep, rabbit, hamster, cow, or mouse. At the appropriate time after immunization, antibody molecules can be isolated from the mammal, e.g. from the blood or other fluid of the mammal, and further purified using standard techniques that include, without limitation, precipitation using ammonium sulfate, gel filtration chromatography, ion exchange chromatography or affinity chromatography using protein A. In addition, an antibody-producing cell of the mammal can be isolated and used to prepare a hybridoma cell that secretes a monoclonal antibody of the invention. Techniques for preparing monoclonal antibody-secreting hybridoma cells are known in the art. See, for example, Kohler and Milstein, Nature 256:495-97 (1975) and Kozbor et al. Immunol Today 4: 72 (1983). A monoclonal antibody of the invention can also be prepared using other methods known in the art, such as, for example, expression from a recombinant DNA molecule, or screening of a recombinant combinatorial immunoglobulin library using a modified polypeptide of the invention.

Methods to generate chimeric and humanized monoclonal antibodies are also well known in the art and include, for example, methods involving recombinant DNA technology. A chimeric antibody can be produced by expression from a nucleic acid that encodes a non-human variable region and a human constant region of an antibody molecule. See, for example, Morrison et al., Proc. Nat. Acad. Sci. U.S.A. 86: 6851 (1984). A humanized antibody can be produced by expression from a nucleic acid that encodes non-human antigen-binding regions (complementarity-determining regions) and a human variable region (without antigen-binding regions) and human constant regions. See, for example, Jones et al., Nature 321:522-24 (1986); and Verhoeven et al., Science 239:1534-36 (1988). Completely human antibodies can be produced by immunizing engineered transgenic mice that express only human heavy and light chain genes. In this case, therapeutically useful monoclonal antibodies can then be obtained using conventional hybridoma technology. See, for example, Lonberg & Huszar, Int. Rev. Immunol. 13:65-93 (1995). Nucleic acids and techniques involved in design and production of antibodies are well known in the art. See, for example, Batra et al., Hybridoma 13:87-97 (1994); Berdoz et al., PCR Methods Appl. 4: 256-64 (1995); Boulianne et al. Nature 312:643-46 (1984); Carson et al., Adv. Immunol. 38:274-311 (1986); Chiang et al., Biotechniques 7:360-66 (1989); Cole et al., Mol. Cell Biochem. 62:109-20 (1984); Jones et al., Nature 321:522-25 (1986); Larrick et al., Biochem Biophys. Res. Commun. 160:1250-56 (1989); Morrison, Annu. Rev. Immunol. 10:239-65 (1992); Morrison et al., Proc. Nat'l Acad. Sci. USA 81:6851-55 (1984); Orlandi et al., Pro. Nat'l Acad. Sci. U.S.A. 86:3833-37 (1989); Sandhu, Crit. Rev. Biotechnol. 12:437-62 (1992); Gavilondo & Larrick, Biotechniques 29: 128-32 (2000); Huston & George, Hum. Antibodies. 10:127-42 (2001); Kipriyanov & Le Gall, Mol. Biotechnol. 26: 39-60 (2004).

Diagnostic Uses

A polypeptide or cross-neutralizing antibody of the invention can be used to detect the presence of HCV in a sample from a mammal. Such a diagnostic use is based on the detection of antibodies generated by a mammal that has been infected with HCV. Diagnostic use can also be based on detection of HCV antigens. In either case, detection of an antibody-antigen complex indicates that the mammal has been exposed to or infected with HCV.

Thus, the invention provides a method for determining whether a mammal such as a human has been or is infected with an HCV. To determine whether a mammal has been infected with HCV, a modified polypeptide of the invention can be used to detect the presence of anti-HCV antibodies in a sample from the mammal. Alternatively, a cross-neutralizing antibody of the invention can be used to detect HCV particles or antigens in the sample.

The sample from the mammal can be a biological fluid such as blood or a cell or tissue sample.

Either the modified polypeptide or the antibody of the invention can be labeled with a detectable label. Thus, to facilitate detection, the polypeptide or cross-neutralizing antibody of the invention can be labeled with a detectable molecule, which can be an enzyme such as alkaline phosphatase, acetylcholinesterase, β-galactosidase or horseradish peroxidase; a prosthetic group such as streptavidin, biotin, or avidin; a fluorescent group such as dansyl chloride, dichlorotriazinylamine, dichlorotriazinylamine fluorescein, fluorescein, fluorescein isothiocyanate, phycoerythrin, rhodamine, umbelliferone; a luminescent group such as luminal; a bioluminescent group such as aequorin, luciferase, and luciferin; or a radioisotope such as ³H, ¹²⁵I, ¹³¹I, ³⁵S.

The formation of an antibody-antigen complex indicates that the mammal has been or is infected with HCV. The presence of HCV particles or antigens in the sample indicates that that mammal is infected with HCV. The presence of HCV antibodies in the sample indicates that the mammal has been or is infected with HCV.

Development of Anti-HCV Therapeutic Agents

A polypeptide of the invention can be used to generate cross-neutralizing antibodies against HCV. For example, a polypeptide of the invention can be used to elicit an immune response in a mammal. Antibodies that bind specifically with the modified E2 polypeptide of the invention can be isolated using known methods as described above. A modified polypeptide of the invention is particularly useful to focus the immune response to the conserved AR3 neutralizing epitopes as the immunogenicity of the hypervariable regions and the AR1 residues are dampened by deletion of a large portion of the hypervariable region and substitution of important selected AR1 residues.

Thus, the invention provides a method of eliciting an immune response in a mammal comprising administering to the mammal modified polypeptide of the invention and then isolating antibodies or antibody producing cells from the mammal using methods known to those of skilled in the art. The mammal can be a rabbit, rat, mouse, sheep, cow, monkey, horse, goat or a pig. The method is particularly useful to generate antibodies against conserved HCV epitopes. Thus, the method can be used to develop passive vaccines containing one or more anti-HCV antibodies of the invention.

A polypeptide of the invention can also be used to screen for anti-HCV agents, such as those that block viral entry into target cells. Since the discontinuous epitopes of the E2 polypeptide described herein are involved in binding to cell receptors, an E2 polypeptide of the invention can be used to screen for agents that bind to an E2 polypeptide of the invention and prevent binding of the E2 polypeptide with a cell receptor.

Therapeutic or Prophylactic Uses

A polypeptide, the coding nucleic acid or a cross-neutralizing antibody of the invention can be used to prevent or treat a new or recurring HCV infection, or prevent or reduce HCV replication, as well as treat the associated disease condition or clinical symptoms. HCV infection or replication is indicated by the amount of HCV particles or the amount of HCV RNA in a sample from the mammal determined using methods known in the art and also those described herein. HCV infection is also indicated by clinical symptoms described further below.

The term “prevent,” “preventing” or “prevention” refers to use in a prophylactic manner that includes, for example, preventing a new infection or viral replication, as well as preventing the onset of symptoms and/or their underlying cause. The terms “treat,” “treating” and “treatment,” include reducing viral replication, reducing the severity and/or frequency of symptoms, eliminating the symptoms and/or underlying cause or improving or remediating damage associated with the infection. The term “reduce” or “reduction” means a decrease in any amount, for example, a decrease of 5%, 10%, 20%, 40%, 50%, 60%, 70% or more than70%.

Thus, the E2 polypeptide of the invention, corresponding nucleic acid or cross-neutralizing antibody of the invention can be used to prevent or reduce transmission, to prevent or treat disease progression, and to prevent or reduce HCV replication or reduce viral load. Treatment includes the alleviation or diminishment of at least one symptom typically associated with the infection. Ideally, the treatment cures, e.g., substantially inhibits viral infection and/or eliminates the symptoms associated with the infection. Symptoms of HCV exposure or infection include, without limitation, inflammation of the liver, decreased appetite, fatigue, abdominal pain, jaundice, flu-like symptoms, itching, muscle pain, joint pain, intermittent low-grade fevers, sleep disturbances, nausea, dyspepsia, cognitive changes, depression headaches and mood changes.

Mammals that can benefit from the polypeptide, nucleic acid or antibody of the invention can be identified using the diagnostic and screening techniques discussed above. Thus, HCV infection can be diagnosed by detecting antibodies to the virus using the modified E2 polypeptide of the invention, detecting the HCV itself using a cross-neutralizing antibody of the invention, detecting liver inflammation by biopsy, liver cirrhosis, portal hypertension, thyroiditis, cryoglobulinemia and glomerulonephritis. In addition, diagnosis of exposure or infection or identification of one who is at risk of exposure to HCV could be based on medical history, abnormal liver enzymes or liver function tests during routine blood testing. Generally, infection can be diagnosed using polymerase chain reaction (PCR) for detecting viral nucleic acids, enzyme linked immunosorbent assay (ELISA) for detecting viral antigens or anti-viral antibodies, and immunofluorescence for detecting viral antigens. For example, a polypeptide or antibody of the invention can be combined with an appropriate sample from the patient to produce a complex. The complex in turn can be detected with a marker reagent for binding with such a complex. Typical marker reagents include secondary antibodies selective for the complex, secondary antibodies selective for certain epitopes of the polypeptide or antibody or a label attached to the polypeptide or antibody itself. In particular, radioimmunoassay (RIA), radioallergosorbent test (RAST), radioimmunosorbent test (RIST), immunradiometric assay (IRMA) Farr assay, fluorescence immunoassay (FIA), sandwich assay, enzyme linked immunosorbent assay (ELISA) assay, northern or southern blot analysis, and color activation assay may be used following protocols well known for these assays. See for example Immunology, An Illustrated Outline by David Male, C. V. Mosby Company, St Louis, Mo., 1986 and the Cold Spring Harbor Laboratory Manuals cited above. Labels including radioactive labels, chemical labels, fluorescent labels, luciferase and the like may also be directly attached to the polypeptide according to the techniques described in U.S. Pat. No. (BN patent cite), the disclosure of which is incorporated herein by reference.

A mammal that can benefit from a polypeptide, nucleic acid or cross-neutralizing antibody of the invention includes one who is likely to be or has been exposed to HCV. Such a mammal includes, without limitation, someone present in an area where HCV is prevalent or commonly transmitted, e.g., Africa, Southeast Asia, China, South Asia, Australia, India, the United States, Russia, as well as Central and South American countries. A mammal who is likely to be or has been exposed to HCV also includes a recipient of donated body tissues or fluids including, for example, a recipient of blood or one or more of its components such as plasma, platelets, or stem cells and an organ or cell transplant recipient such as a liver transplantee. A mammal who is likely to be or has been exposed to HCV can also include medical, clinical or dental personnel handling body tissues and fluids. A mammal who has been exposed to HCV includes, without limitation, someone who has had contact with the body tissue or fluid, e.g. blood, of an infected person or otherwise have come in contact with HCV. A mammal that can benefit from a polypeptide or cross-neutralizing antibody of the invention includes one who is susceptible to HCV infection or one who has recurring HCV infection.

Thus, the invention provides a method for preventing a new or recurring HCV infection and its associated symptoms and/or complications such as by preventing or reducing HCV replication in a mammal infected with HCV. A polypeptide, nucleic acid or cross-neutralizing antibody of the invention can be used prophylactically to prevent a susceptible individual from being infected with HCV or to prevent recurring HCV infection, for example, in an individual who has received a liver transplant.

A polypeptide or cross-neutralizing antibody of the invention can be used to prevent or treat infection of a mammalian cell, such as a human cell. A polypeptide, nucleic acid or cross-neutralizing antibody of the invention can be used to prevent or treat a new or recurring HCV infection, or prevent or reduce HCV replication, in a mammal such as a human. Thus, an E2 polypeptide or a nucleic acid encoding an E2 polypeptide of the invention can be used as an active vaccine, a nucleic acid or DNA-based vaccine, or be incorporated into vaccine carriers, to elicit a protective immune response in a mammal.

Methods of preventing or treating HCV infection include contacting a cell with an effective amount of an antibody of the invention; mixing biological fluids, cells or tissues to be administered or transplanted into a mammal with a polypeptide, nucleic acid or antibody of the invention prior to the administration or transplant; or administering to a mammal such as a human a therapeutically effective amount of a polypeptide, nucleic acid or antibody of the present invention. Thus, the invention provides in vitro methods of preventing HCV infection or transmission by contacting biological samples such as fluids, cells or tissues containing the virus with an effective amount of the polypeptide, nucleic acid or antibody of the invention, as well as in vivo methods of treating or preventing HCV infection by administering the polypeptide, nucleic acid or antibody to the mammal.

A polypeptide, nucleic acid or antibody of the invention can be administered in a variety of ways. Routes of administration include, without limitation, oral, parenteral (including subcutaneous, intravenous, intramuscular and intraperitoneal), rectal, vaginal, dermal, transdermal (topical), transmucosal, intrathoracic, intrapulmonary and intranasal (respiratory) routes. The means of administration may be by injection, using a pump or any other appropriate mechanism.

A polypeptide, nucleic acid or antibody of the invention may be administered in a single dose, in multiple doses, in a continuous or intermittent manner, depending, for example, upon the recipient's physiological condition, whether the purpose of the administration is therapeutic or prophylactic, and other factors known to skilled practitioners. The administration of the polypeptide, nucleic acid or antibody of the invention may be essentially continuous over a pre-selected period of time or may be in a series of spaced doses. For example, the invention provides a method of eliciting an immune response in a mammal that involves administering a modified polypeptide, nucleic acid or antibody of the invention at a select time and then administering a second, third, forth or additional doses at select times after the first administration. Both local and systemic administrations are contemplated.

The dosage to be administered to a mammal may be any amount appropriate to reduce or prevent viral infection or to treat at least one symptom associated with the viral infection. Some factors that determine appropriate dosages are well known to those of ordinary skill in the art and may be addressed with routine experimentation. For example, determination of the physicochemical, toxicological and pharmacokinetic properties may be made using standard chemical and biological assays and through the use of mathematical modeling techniques known in the chemical, pharmacological and toxicological arts. The therapeutic utility and dosing regimen may be extrapolated from the results of such techniques and through the use of appropriate pharmacokinetic and/or pharmacodynamic models. Other factors will depend on individual patient parameters including age, physical condition, size, weight, the condition being treated, the severity of the condition, and any concurrent treatment. The dosage will also depend on the polypeptide or antibody chosen and whether prevention or treatment is to be achieved, and if the polypeptide or antibody is chemically modified. Such factors can be readily determined by the clinician employing viral infection models such as in vitro HCV infection system described herein, or other animal models or test systems that are available in the art.

The precise amount to be administered to a mammal such as a human will be the responsibility of the attendant physician. The amount useful to establish treatment of HCV can be determined by diagnostic and therapeutic techniques well known to those of ordinary skill in the art. The dosage may be determined by titrating a sample of the patient's blood sera with the polypeptide or antibody to determine the end point beyond which no further immunocomplex is formed. Such titrations may be accomplished by the diagnostic techniques discussed below. Available dosages include administration of from about 1 to about 1 million effective units of antibody per day, wherein a unit is that amount of polypeptide, which will provide at least 1 microgram of antigen-polypeptide complex. Preferably, from about 10 to about 100,000 units of antibody per day can be administered.

To achieve the desired effect(s), one or more modified polypeptides or antibody of the invention may be administered as single or divided dosages, for example, of at least about 0.01 mg/kg to about 500, 750 or 1000 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. For post-exposure prophylactic use, the one or more polypeptide or antibody of the invention may be administered as soon as possible, e.g. within 24 hours if possible, after exposure to HCV. To prevent recurrent HCV infection, e.g. in a transplant recipient such as a liver transplant recipient, a modified polypeptide or antibody of the invention may be administered prior to and after transplantation. For example, the polypeptide or antibody of the invention can be administered during the anhepatic phase, as well as during the post-operative phase. The polypeptide, nucleic acid or antibody of the invention may be administered daily, biweekly or monthly after the transplant. The polypeptide, nucleic acid or antibody of the invention can be administered daily for the first week after transplant, weekly for two, three or more weeks after the transplant and then monthly.

The absolute weight of a polypeptide or antibody included in a unit dose can vary widely. For example, about 0.01 to about 2 g, or about 0.1 to about 500 mg, of at least one polypeptide, nucleic acid or antibody of the invention, or a plurality of polypeptides, nucleic acids or antibodies can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

The daily dose of a polypeptide, nucleic acid or antibody of the invention can vary as well. Such daily dose can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

A polypeptide, nucleic acid or antibody of the invention may be used alone or in combination with a second medicament. The second medicament can be another polypeptide or antibody of the invention, a known antiviral agent such as, for example, an interferon-based therapeutic or another type of antiviral medicament such as ribavirin.

The second medicament can also be an anticancer, antibacterial, or another antiviral agent. The antiviral agent may act at any step in the life cycle of the virus from initial attachment and entry to egress. Thus, the second antiviral agent may interfere with attachment, fusion, entry, trafficking, translation, viral polyprotein processing, viral genome replication, viral particle assembly, egress or budding. Stated another way, the antiviral agent may be an attachment inhibitor, entry inhibitor, a fusion inhibitor, a trafficking inhibitor, a replication inhibitor, a translation inhibitor, a protein processing inhibitor, an egress inhibitor, in essence an inhibitor of any viral function. The effective amount of the second medicament will follow the recommendations of the manufacturer of the second medicament, as well as the judgment of the attending physician, and will be guided by the protocols and administrative factors for amounts and dosing as indicated in the PHYSICIAN'S DESK REFERENCE.

To determine the effectiveness of a polypeptide, nucleic acid or antibody of the invention for inhibition and treatment of HCV infection, methods available in the art and those described herein can be used. The effectiveness of the method of treatment can be assessed by monitoring the patient for signs or symptoms of the viral infection as discussed above, as well as determining the presence and/or amount of viral particle or viral RNA present in the blood, e.g. the viral load, using methods known in the art. Viral infection or replication in the presence or absence of a polypeptide or antibody of the invention can be evaluated, for example, by determining intracellular viral RNA levels or the number of viral foci by immunoassays using antibody against viral proteins as described herein. A polypeptide or antibody is effective for treatment and inhibition of HCV if can inhibit or reduce viral infection or replication by any amount, for example, by 2 fold or more than 2 fold. For example, a polypeptide or antibody of the invention can inhibit or reduce HCV infection by 2-5 folds, 5-10 folds, or more than 10 folds.

A polypeptide, nucleic acid or antibody of the invention can also be used to increase the safety of blood and blood products, to increase the safety of clinical laboratory samples and to increase the safety of biological tissues as well as articles, devices, or instruments intended for preventative or therapeutic use. For example, a polypeptide, nucleic acid or antibody of the invention can be added to blood or blood products such as plasma, platelets, and blood or marrow cells prior to use. A polypeptide, nucleic acid or antibody of the invention can be combined with cells or tissues prior to use or administration. It can be coated on articles, devices or instruments such as, for example, valves, bags and stents.

### Preparations and Compositions of the Invention

In one aspect, the invention provides a purified preparation containing a modified polypeptide of the invention or a preparation a cross-neutralizing antibody of the invention.

In a purified preparation of a modified polypeptide of the invention, at least 50% of the modified polypeptides in the preparation are folded in a conformation such that the discontinuous epitopes (i.e. amino acid segments corresponding to amino acids 396 to 424, amino acids 436 to 447 and amino acids 523 to 540 of HCV strain H77) come together to form a conformational epitope that can bind with a conformation-dependent cross-neutralizing antibody, for example, AR3A, AR3B, AR3C or AR3D. In such a polypeptide preparation, at least about 50%, 55%, 60%, 65%, 70%, 75%, 80%, 85%, 90% or 95% of the modified polypeptides are folded as described above. For example, in such a polypeptide preparation of the invention, about 85%, 88%, 90%, 92%, 94%, 95%, 96%, 97%, 98%, or 99% of the modified polypeptides are folded as described.

In a preparation of a cross-neutralizing antibody of the invention, a larger proportion of the antibodies are cross-neutralzing antibodies. For example, such an antibody preparation can be a biological sample such as blood or plasma obtained from a mammal that has been immunized with a modified polypeptide of the invention. In this case, the blood sample contains a larger proportion of cross-neutralizing antibodies than a blood sample obtained from a similar animal that has been immunized with a naturally-occurring E2 polypeptide.

Such a cross-neutralizing antibody preparation can be a partially purified or purified polypeptide preparation, i.e. a preparation resulting from one or more protein purification steps known in the art as well as those discussed herein. Such cross-neutralizing antibody preparation of the invention contains at least about 2%, 5%, 10%, 20%, 30%, 40%, 50%, 55%, 60%, 65%, 70%, 75%, or 80% cross-neutralizing anti-HCV antibodies. For example, such cross-neutralizing antibody preparation of the invention can contains about 5%, 6%, 7%, 8%, 9%, 10%, 12%, 13%, 14%, 15%, 16%, 17%, 18%, 19%, 20%, 22%, 23%, 24%, 25%, 26%, 27%, 28%, 29%, 30%, 32%, 33%, 34%, 35%, 36%, 37%, 38%, 39%, 40%, 42%, 43%, 44%, 45%, 46%, 47%, 48%, 49%, 50%, 52%, 53%, 54%, 55%, 56%, 57%, 58%, 59%, 60%, 62%, 63%, 64%, 65%, 66%, 67%, 68%, 69%, 70%, 72%, 73%, 74%, 75%, 76%, 77%, 78%, 79%, 80%, 82%, 83%, 84%, 85%, 86%, 87%, 88%, 89%, 90%, 92%, 93%, 94%, 95%, 96%, 97%, 98%, 99% cross-neutralizing anti-HCV antibodies.

As used herein, the term “purified” with reference to a polypeptide or antibody preparation means that the polypeptide or antibody in the preparation is substantially free of naturally-associated components, i.e. components that accompany it in its natural state. A chemically synthesized polypeptide, one produced using recombinant DNA technology, or one produced in a cellular system different from the cell system from which the polypeptide of the invention naturally originates, is substantially free from its naturally associated components. The term “purified” also encompasses a biological sample such as a blood sample that has been subject to at least one separation step, for example, centrifugation to separate cellular components from non-cellular components. In this case, both fractions of the original blood sample is encompassed by the term “purified.” The term “purified” does not encompass a polypeptide or antibody separated in a lane of a protein gel in which multiple unrelated polypeptides or antibodies have been separated. In general, a polypeptide or antibody of the invention can constitute at least about 25% by weight of a sample containing the polypeptide of the invention, and usually constitutes at least about 50%, at least about 75%, at least about 85%, at least about 90% of a sample, particularly at least about 95% of the sample or 99% or more.

Methods of preparing modified polypeptides and cross-neutralizing antibodies of the invention are described above. Preparations of these can be obtained using protein purifications procedures known to those in the art. See, for example, CURRENT PROTOCOLS IN PROTEIN SCIENCE, Coligan et al., eds., John Wiley & Sons, Inc., 1997.

In another aspect, the invention provides a pharmaceutical composition comprising a modified polypeptide, nucleic acid or antibody of the invention. To prepare such a pharmaceutical composition, a modified polypeptide, nucleic acid or antibody of the invention is obtained, e.g. by expression in a host cell or using polymerase chain reaction, purified as necessary or desired and then lyophilized and stabilized. The polypeptide, nucleic acid or antibody can then be adjusted to the appropriate concentration and then combined with other agent(s) or pharmaceutically acceptable carrier(s). By “pharmaceutically acceptable” it is meant a carrier, diluent, excipient, and/or salt that is compatible with the other ingredients of the formulation, and not deleterious to the recipient thereof, for example, a buffered aqueous, oil or organic medium containing optional stabilizing agents and adjuvants for stimulation of immune binding.

A pharmaceutical formulation containing therapeutic amounts of one or more polypeptides, nucleic acids or antibodies of the invention can be prepared by procedures known in the art using well-known and readily available ingredients. For example, one or more polypeptides, nucleic acids or antibodies can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone.

Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For oral administration, one or more polypeptides, nucleic acids or antibodies may be present as a powder, a granular formulation, a solution, a suspension, an emulsion or in a natural or synthetic polymer or resin for ingestion of the active ingredients from a chewing gum. The active agents may also be presented as a bolus, electuary or paste. The formulations may, where appropriate, be conveniently presented in discrete unit dosage forms and may be prepared by any of the methods well known to the pharmaceutical arts including the step of mixing the therapeutic agent with liquid carriers, solid matrices, semi-solid carriers, finely divided solid carriers or combinations thereof, and then, if necessary, introducing or shaping the product into the desired delivery system. The total active ingredients in such formulations comprise from 0.1 to 99.9% by weight of the formulation.

One or more polypeptides, nucleic acids or antibodies of the invention can also be formulated as elixirs or solutions for convenient oral administration or as solutions appropriate for parenteral administration, for instance by intramuscular, subcutaneous, intraperitoneal or intravenous routes. A pharmaceutical formulation containing one or more therapeutic polypeptides, nucleic acids or antibodies of the invention can also take the form of an aqueous or anhydrous solution or dispersion, or alternatively the form of an emulsion or suspension or salve.

Thus, one or more polypeptides, nucleic acids or antibodies may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The polypeptides, nucleic acids or antibodies and other ingredients may form suspensions, solutions, or emulsions in oily or aqueous vehicles, and may contain formulatory agents such as suspending, stabilizing and/or dispersing agents. Alternatively, the polypeptides, nucleic acids or antibodies and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if necessary, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and a-tocopherol and its derivatives can be added.

In some embodiments the one or more polypeptides, nucleic acids or antibodies are formulated as a microbicide, which is administered topically or to mucosal surfaces such as the vagina, the rectum, eyes, nose and the mouth. For topical administration, the therapeutic agents may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Thus, in one embodiment, an agent of the invention can be formulated as a vaginal cream or a microbicide to be applied topically. Other conventional forms for this purpose include wound dressings, coated bandages or other polymer coverings, ointments, creams, lotions, pastes, jellies, sprays, and aerosols. Thus, the one or more polypeptides, nucleic acids or antibodies of the invention can be delivered via patches or bandages for dermal administration. Alternatively, the polypeptides, nucleic acids or antibodies can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active agents can also be delivered via iontophoresis, e.g., as disclosed in U.S. Pat. Nos. 4,140,122; 4,383,529; or 4,051,842. The percent by weight of one or more polypeptides, nucleic acids or antibodies of the invention present in a topical formulation will depend on various factors, but generally will be from 0.01% to 95% of the total weight of the formulation, and typically 0.1-85% by weight.

Drops, such as eye drops or nose drops, may be formulated with one or more of the polypeptides, nucleic acids or antibodies in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The one or more polypeptides, nucleic acids or antibodies may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The polypeptides, nucleic acids or antibodies of the invention can also be administered to the respiratory tract. Thus, the present invention also provides aerosol pharmaceutical formulations and dosage forms for use in the methods of the invention. In general, such dosage forms comprise an amount of at least one of the polypeptides, nucleic acids or antibodies of the invention effective to treat or prevent the clinical symptoms of the viral infection. Any statistically significant attenuation of one or more symptoms of the infection that has been treated pursuant to the method of the present invention is considered to be a treatment of such infection within the scope of the invention.

Alternatively, for administration by inhalation or insufflation, the composition may take the form of a dry powder, for example, a powder mix of one or more polypeptides, nucleic acids or antibodies and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

The one or more polypeptides, nucleic acids or antibodies of the present invention can also be administered in an aqueous solution when administered in an aerosol or inhaled form. Thus, other aerosol pharmaceutical formulations may comprise, for example, a physiologically acceptable buffered saline solution containing between about 0.1 mg/mL and about 100 mg/mL of one or more of the polypeptides, nucleic acids or antibodies of the present invention specific for the indication or disease to be treated. Dry aerosol in the form of finely divided solid polypeptide, nucleic acid or antibody particles that are not dissolved or suspended in a liquid are also useful in the practice of the present invention. Polypeptides, nucleic acids or antibodies of the present invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular infection, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the one or more polypeptides, nucleic acids or antibodies of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

A preferred formulation involves lyophilized polypeptides, nucleic acids or antibodies and separate pharmaceutical carrier. Immediately prior to administration, the formulation is constituted by combining the lyophilized polypeptides, nucleic acids or antibodies and pharmaceutical carrier. Administration by a parenteral or oral regimen will deliver the polypeptides, nucleic acids or antibodies to the desired site of action. Pharmaceutical formulations of the polypeptides, nucleic acids or antibodies of the invention can prepared as liquids, gels and suspensions. The formulations are preferably suitable for injection, insertion or inhalation. Injection may be accomplished by needle, cannula catheter and the like. Insertion may be accomplished by lavage, trochar, spiking, surgical placement and the like. Inhalation may be accomplished by aerosol, spray or mist formulation. The polypeptides, nucleic acids or antibodies of the invention may also be administered topically such as to the epidermis, the buccal cavity and instillation into the ear, eye and nose. The polypeptides, nucleic acids or antibodies may be present in the pharmaceutical formulation at concentrations ranging from about 1 percent to about 50 percent, preferably about 1 percent to about 20 percent, more preferably about 2 percent to about 10 percent by weight relative to the total weight of the formulation.

A polypeptide, nucleic acid or antibody of the invention may also be used in combination with one or more known therapeutic agents, for example, a pain reliever; an antiviral agent such as an anti-HBV, other anti-HCV (HCV inhibitor, HCV protease inhibitor) or an anti-herpetic agent; an antibacterial agent; an anti-cancer agent; an anti-inflammatory agent; an antihistamine; a bronchodilator and appropriate combinations thereof, whether for the conditions described or some other condition.

Miscellaneous Compositions and Articles of Manufacture

The invention also provides an article of manufacture that includes a pharmaceutical composition containing one or more polypeptides, nucleic acids or antibodies of the invention for controlling microbial infections. Such articles may be a useful device such as a vaginal ring, a condom, a bandage or a similar device. The device holds a therapeutically effective amount of a pharmaceutical composition for controlling viral infections. The device may be packaged in a kit along with instructions for using the pharmaceutical composition for control of the infection. The pharmaceutical composition includes at least one polypeptide, nucleic acid or antibody of the present invention, in a therapeutically effective amount such that viral infection is controlled.

An article of manufacture may also be a vessel or filtration unit that can be used for collection, processing or storage of a biological sample containing a polypeptide or antibody of the invention. The vessel may be evacuated. Vessels include, without limitation, a capillary tube, a vacutainer, a collection bag for blood or other body fluids, a cannula, a catheter. The filtration unit can be part of another device, for example, a catheter for collection of biological fluids. Moreover, the one or more polypeptides or antibodies of the invention can also be adsorbed onto or covalently attached to the article of manufacture, for example, a vessel or filtration unit. Thus, when material in the article is decanted therefrom or passed through, the material will not retain substantial amounts of the polypeptides or antibodies. However, adsorption or covalent attachment of the one or more polypeptides or antibodies to the article kills viruses or prevents their transmission, thereby helping to control viral infection. Thus, for example, the one or more polypeptides or antibodies of the invention can be in filtration units integrated into biological collection catheters and vials, or added to collection vessels to remove or inactivate viral particles that may be present in the biological samples collected, thereby preventing transmission of the disease.

The invention also provides a composition comprising one or more polypeptides, nucleic acids or antibodies of the invention and one or more clinically useful agents such as a biological stabilizer. Biological stabilizer includes, without limitation, an anticoagulant, a preservative and a protease inhibitor. Anticoagulants include, without limitation, oxalate, ethylene diamine tetraacetic acid, citrate and heparin. Preservatives include, without limitation, boric acid, sodium formate and sodium borate. Protease inhibitors include inhibitors of dipeptidyl peptidase IV. Compositions comprising an agent of the invention and a biological stabilizer may be included in a collection vessel such as a capillary tube, a vacutainer, a collection bag for blood or other body fluids, a cannula, a catheter or any other container or vessel used for the collection, processing or storage of a biological samples.

The invention also provides a composition comprising one or more polypeptides, nucleic acids or antibodies of the invention and a biological sample such as blood, semen or other body fluids that is to be analyzed in a laboratory or introduced into a recipient mammal. For example, one or more polypeptides, nucleic acids or antibodies of the invention can be mixed with blood prior to laboratory processing and/or transfusions. The one or more polypeptides, nucleic acids or antibodies is present in at least about 0.15 mg/mL of the sample, e.g. 0.16 mg/mL, 0.17 mg/mL, 0.18 mg/mL, 0.19 mg/mL, 0.2 mg/mL, 0.22 mg/mL, 0.24 mg/mL, 0.25 mg/mL, 0.27 mg/mL, 0.3 mg/mL, 0.35 mg/mL, 0.4 mg/mL or more than 0.4 mg/mL of sample.

In another embodiment, the one or more polypeptides, nucleic acids or antibodies of the invention can be included in physiological media used to store and transport biological tissues, including transplantation tissues. Thus, for example, liver, heart, kidney and other tissues can be bathed in media containing the present agents to inhibit viral transmission to transplant recipients. In this case, the one or more polypeptides, nucleic acids or antibodies is present in at least about 1.5 mg/kg of the sample, e.g. 1.6 mg/kg, 1.7 mg/kg, 1.8 mg/kg, 1.9 mg/kg, 2 mg/kg, 2.2 mg/kg, 2.4 mg/kg, 2.5 mg/kg, 2.7 mg/kg, 2.8 mg/kg, 2.9 mg/kg, 3 mg/kg, 3.5 mg/kg, 4 mg/kg or more than 4 mg/kg.

The invention is further illustrated by the following non-limiting Examples, which do not limit the scope of the invention described in the statements.

### EXAMPLES

### Example 1

### Materials and Methods

This Example describes some of the procedures and materials used in developing the invention.

Cells, Antibodies and Viruses.

Huh-7 (Zhong, J. et al. Proc. Natl. Acad. Sci. U.S.A. 102, 9294-9299 (2005)) and 293T cells were grown in Dulbecco's Modified Eagle Medium (D-MEM) supplemented with 10% fetal calf serum (FCS) (Invitrogen). The human MAbs CBH-2, CBH-5, CBH-4B and CBH-7, Mouse MAbs A4, H53, AP33, AP320 and ALP98, and rat MAbs 7/59, 9/27, 3/11, 1/39, 2/69A, 7/16B, 11/20, 9/75 and 6/53 have been described elsewhere. Keck, Z. Y. et al. J. Virol. 78, 9224-9232 (2004); Keck, Z. Y. et al. J. Virol. 81, 1043-1047 (2007); Keck, Z. Y. et al. J. Virol 79, 13199-13208 (2005); Dubuisson, J. et al. J. Virol. 68, 6147-6160 (1994); Clayton, R. F. et al. J. Virol. 76, 7672-7682 (2002); Deleersnyder, V. et al. J. Virol. 71, 697-704 (1997); Owsianka, A. et al. J. Virol. 79, 11095-11104 (2005); Tarr, A. W. et al. Hepatology 43, 592-601 (2006); Flint, M. et al. J. Virol. 73, 6235-6244 (1999); Hsu, M. et al. Proc. Natl. Acad. Sci. U.S.A. 100, 7271-7276 (2003); Maruyama, T. et al. Am. J. Pathol. 165, 53-61 (2004). The panel of linear epitope-specific MAbs covers known linear regions. The generation of HCVpp has been described below.

Phage Display Antibody Library Construction.

In a study of autoantibodies in patients with Sjögren's syndrome, bone marrow mononuclear cell RNA from a 35-year-old female patient with Sjögren's syndrome and chronic HCV infection was used as source material for an IgG1 Fab phage display library (Maruyama, T. et al. Am. J. Pathol. 165, 53-61 (2004)). The donor was diagnosed with HCV in 1991 and developed mixed cryoglobulinemia, symptoms of Sjögren's syndrome and tested positive for antinuclear antibody in 1994. The donor was treated with interferon-α with initial decrease in viral load but the treatment was stopped due to severe drop in platelet count (idiopathic thrombocytopenic purpura). Bone marrow samples were collected for the evaluation of neutropenia as an outpatient clinical procedure at Scripps Clinic. After meeting the needs of clinical pathology, a fraction of the biopsy was used to construct the antibody library. The human subjects protocol was approved by the Human Subjects Committee for General Clinical Research Center of Scripps Clinic and informed consent was obtained from the donor. Due to subsequent relapse of HCV, the donor underwent a liver transplant in 2000 and has been maintained on anti-rejection medications since. The viral genotype in this donor was not determined at the time of tissue donation but was found to be genotype 1a seven years later. The bone marrow (˜2 ml) was separated by Histopaque-1077 gradient (Sigma-Aldrich) and RNA was extracted from mononuclear cells (7×10⁷ cells) homogenized in 10 mL of TRI reagent (Sigma-Aldrich). First-strand cDNA was synthesized using SuperScript First-Strand Synthesis Kit (Invitrogen), and the light chain and IgG1 heavy chain fragments were amplified by PCR using gene-specific primers and were sequentially cloned into the SacI/XbaI and XhoI/SpeI sites of a phagemid vector, pComb3H, as described previously (Maruyama, T. et al. J. Infect. Dis. 179 Suppl 1, S235-239 (1999)). The Fab heavy chains were expressed as a fusion protein with the phage gene III surface protein for display. The library was amplified in XL-1 Blue cells (Stratagene) using 0.3% SeaPrep agarose (BioWhittaker) in SuperBroth (SB) Medium by a semi-solid phase amplification method.

Library Panning on HCVE2 Glycoprotein

The phagemid library was transformed into E. coli (XL-1 Blue) (Stratagene) by electroporation and the phage was propagated overnight with VCS-M13 helper phage (Stratagene). Recombinant E2 glycoprotein (genotype 1a, amino acids 388-644; Lesniewski, R. et al. J. Med. Virol. 45, 415-422 (1995)) was coated directly onto a microtiter plate overnight at 4° C. (Costar). The wells were washed and then blocked with 4% non-fat dry milk in phosphate-buffered saline (PBS). The phage library was added to the wells and incubated for 1-2 hours at 37° C. and unbound phage washed away with PBS. Bound phage were eluted and used to infect freshly grown E. coli (XL1-Blue) (Stratagene) for titration on LB agar plates with carbenicillin. The phage libraries were panned for four consecutive rounds with increasing washing stringency.

Library Panning by an Epitope Masking Strategy.

In order to broaden the diversity of antibody specificities selected, library panning was repeated using recombinant E1E2 fused to glutathione S transferase (GST-E1E2; Chan-Fook, C. et al. Virology 273, 60-66 (2000)) pre-incubated with Fabs obtained above. GST-E1E2 was first captured with goat anti-GST antibody (Amersham Biosciences) and the wells were washed and blocked with 4% non-fat dry milk in PBS. Fabs obtained from the panning using E2 antigen above were added to the captured antigens to mask corresponding specific epitopes. The epitope-masked GST-E1E2 was used to pan the phage library as described above. It is important to note that, highly isolate-specific antibodies, e.g. those against HVR1, were not selected due to the use of heterologous antigens in panning.

Screening of Fab Displayed Phage.

Single individual colonies were isolated from titration plates after the 2nd, the 3rd, and the 4th round. The colonies were grown in SB medium with carbenicillin and tetracycline and Fab-phage production was induced with the addition of helper phage overnight at 30° C. The specificities of the Fab-phage were assessed by ELISA and the DNA sequences of the Fab-phage that bound with high specificity were determined. To produce soluble Fabs, the phage gene III surface protein in fusion with the Fab heavy chain was excised by restriction enzymes SpeI and NheI. The cut phagemids were self-ligated and transformed into XL1-Blue cells for the production of soluble Fabs by standard protocols. Barbas III, C. F., Burton, D. R., Scott, J. K. & Silverman, G. J. Phage Display: A Laboratory Manual, (Cold Spring Harbor Laboratory Press, New York, 2001).

Conversion of Fab into IgG1

The vectors pDR12 (Burton, D. R. et al. Science 266, 1024-1027 (1994)) and pIgG1 encoding the leader sequence and constant region of human IgG1 gene were used for the cloning and expression of full length IgG1. Vector pIgG1 is a derivative of pDR12 in which heavy and light chain cloning sites were altered to XhoI/BstEII and SacI/XbaI sites to facilitate direct cloning of the antibody gene fragments. For pDR12, the heavy and light chain genes of Fab C1 were amplified by PCR then inserted sequentially into the SacI/XbaI and HindIII/EcoRI sites of the vector (Burton, D. R. et al. Science 266, 1024-1027 (1994)). For pIgG1, the heavy and light chain gene fragments were excised from the phagemids and inserted sequentially into the XhoI/BstEII and SacI/XbaI sites of the vector. The recombinant plasmids were transfected into Chinese hamster ovarian (CHO) cells. Stable cell clones were established by selection with L-methionine sulfoxide (MSX) and by limiting dilution. Cell clones expressing high IgG levels were amplified and the IgGs were purified using a protein A-agarose column (Pharmacia).

ELISA. (i) To study the relative reactivity of Fabs to GST-E1E2 and E2, soluble Fabs were added to ELISA wells coated with soluble E2 (4 μg/mL), with GST-E1E2 (8 μg/mL) captured by pre-coated goat anti-GST-antibody (10 μg/mL), or with ovalbumin (4 μg/mL). Specific binding was detected by alkaline phosphatase (AP)-conjugated goat anti-human IgG F(ab′)₂ antibody (Pierce) (1:500) in 1% BSA/PBS and disodium p-nitrophenyl phosphate (Sigma). (ii) To study the relationship of different ARs to the mouse MAb epitope H53 (Cocquerel et al., J. J. Virol. 72, 2183-2191 (1998)), a saturating concentration of MAb H53 was added to vaccinia-expressed E1E2 (isolate HCV-1, obtained through the NIH AIDS Research and Reference Reagent Program: rVV E12 C/B from Chiron Corporation; Cooper, S. et al. Immunity 10, 439-449 (1999); Selby, M. et al. J. Immunol. 162, 669-676 (1999)) captured by pre-coated Galanthus nivalis lectin (5 μg/mL, Sigma) for 30 min before the addition of soluble Fabs (2 μg/mL). Non-fat milk (4%, BioRad) in PBS was used as a blocker in assays using lectin-captured antigens. The ELISA plates were washed after a 1 hour incubation and binding of human Fabs was detected by peroxidase (HRP)-conjugated goat anti-human IgG F(ab′)₂ antibody (1:2000) (Pierce) and TMB substrate (Pierce). The level of inhibition by MAb H53 was calculated as the % reduction of optical density signals produced by the human Fabs in the presence of H53. (iii) To study whether the MAbs recognized continuous or discontinuous epitopes, vaccinia-expressed E1E2 was either captured directly onto ELISA wells pre-coated with lectin (folded protein), or unfolded with 0.1% SDS, 50 mM DTT and incubated at 100° C. for 5 minutes before capture onto ELISA wells (unfolded protein). Binding of the MAbs to folded and unfolded proteins was detected using the peroxidase system. Mouse MAb A4 (Dubuisson, J. et al. J. Virol. 68, 6147-6160 (1994)), specific for a linear epitope in E1, was used as a positive control. (iv) To study the ability of MAb in inhibiting E1E2 binding to CD81, serially diluted MAbs (4-fold dilution from 10 μg/mL) were incubated with E1E2 (isolate H77) for 30 min before adding to ELISA wells pre-coated with the large extracellular loop of CD81 (CD81-LEL). After 1 hour incubation, the plates were washed and binding of E1E2 to CD81-LEL was detected with an anti-E1 mouse MAb A4 (Dubuisson, J. et al. J. Virol. 68, 6147-6160 (1994)), HRP-conjugated goat anti-mouse Fc antibody (Pierce) (1:2000) and TMB substrate. Two forms of recombinant CD81-LEL, either in fusion with glutathione S-transferase (GST) (Owsianka, A. M. et al. J. Virol. 80, 8695-8704 (2006)) or maltose binding protein (MBP) (Chan-Fook, C. et al. Virology 273, 60-66 (2000)), were used and the results were equivalent. (v) To study the apparent affinity of the MAbs, serially diluted MAbs (2-fold dilution from 20 μg/mL) were added to lectin-captured E1E2 antigens for 1 hour. E1E2 antigens were prepared from cell lysates from vaccinia-expressed HCV-1 E1E2, 293T cells transfected with H77 E1E2-expression plasmid (McKeating, J. A. et al. J. Virol. 78, 8496-8505 (2004)). The binding of human MAbs was detected by HRP-conjugated goat anti-human IgG F(ab′)₂ antibody as above. Non-infected/non-transfected cell lysate were used as negative controls to determine background for each MAb. Apparent affinity was defined by the concentration of MAbs that produced half of the maximal specific binding in the titration curves. (vi) To construct the MAb competition matrix, saturating concentrations of blocking MAbs (typically at 20 μg/mL or undiluted hybridoma supernatants) were added to lectin-captured vaccinia-expressed HCV-1 E1E2 for 30 minutes before the addition of an equal volume of biotinylated human MAbs (2 μg/mL). The E1E2 antigens were titrated to ensure that saturating concentrations of the blocking MAbs were used in the assays. It is important to note that, MAbs recognizing linear epitopes bind to both folded and unfolded proteins but the biotinylated human MAbs bind conformational epitopes on folded E2. Consequently, competition is performed with the MAbs to linear epitopes as blocking MAbs to eliminate potential non-specific signals caused by misfolded proteins in the system. After incubation for 1 h, the ELISA plates were washed and binding of biotinylated MAbs was detected with HRP-conjugated streptavidin (1:2000, Sigma-Aldrich) in PBS with 1% BSA and TMB substrate (Pierce). Competition was determined by the % change in binding signals in the presence of the blocking antibodies. (vii) To study the effect of alanine substitution in E2 on MAb binding, E1E2 mutant antigens were produced by transfection of 293T cells with the corresponding expression plasmids and the antigens in clarified cell lysate were captured by lectin as above. A panel of 38 H77 E1E2 mutants having the conserved residues in the putative CD81 binding pocket substituted with alanine was used in this study (Owsianka, A. M. et al. J. Virol. 80, 8695-8704 (2006)). The binding signals of the human MAbs to the alanine mutants were compared to the wildtype H77 E1E2 to determine the importance of the residues in the antibody-antigen interaction. (viii) To quantify human IgG in mouse serum, diluted mouse sera in triplicate were added to ELISA wells coated with human goat anti-human IgG F(ab)′₂ (10 μg/mL, Pierce) for 1 hour and bound human IgG was detected with AP-conjugated goat anti-human F(ab)′₂ (10 μg/mL) (Pierce). Serially diluted MAb AR3A (2-fold dilution starting from 4 g/mL) was used to construct a standard curve in each ELISA plate. The concentration of human IgG in each serum sample was calculated from the 4-parameter fitted standard curve using SOFTmax Pro Software (Molecular Devices).

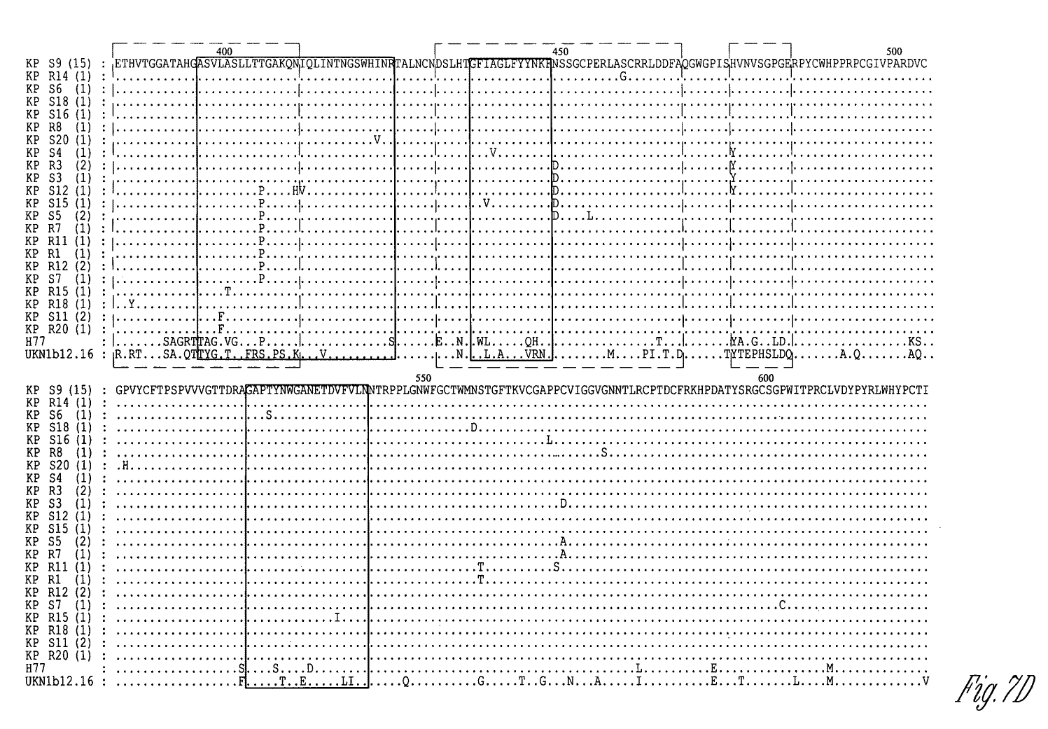

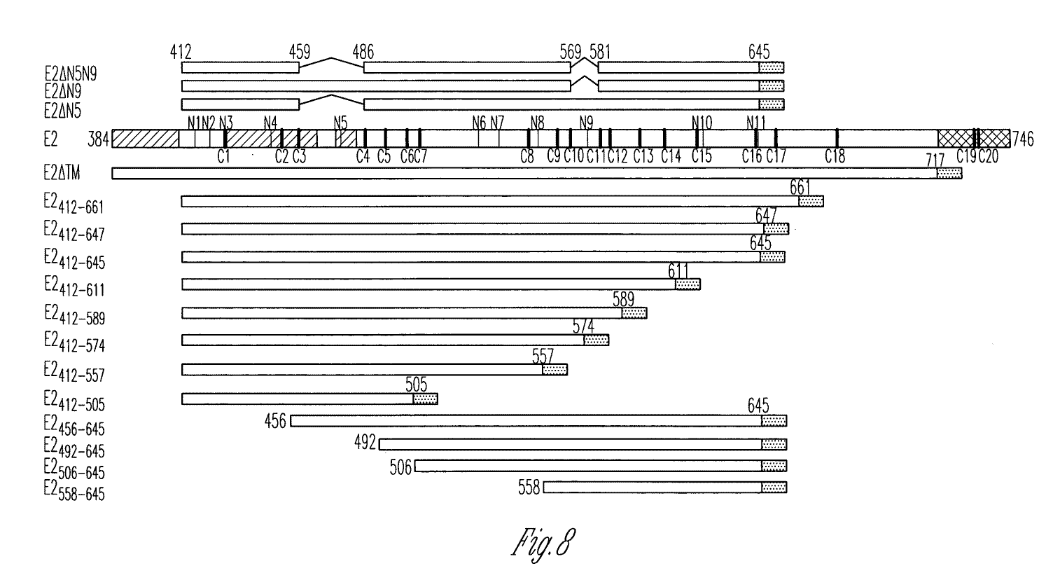

HCV neutralization assays. The neutralization assays were performed in Dulbecco's Modified Eagle Medium (D-MEM) supplemented with 10% fetal calf serum (FCS) (Invitrogen). For HCVpp neutralization, HCVpp was generated by co-transfection of 293T cells with pNL4-3.lucR-E- (Connor et al., Virology 206, 935-944 (1995); He, J. et al. J. Virol. 69, 6705-6711 (1995)) and the corresponding expression plasmids encoding the E1E2 genes at 4:1 ratio by polyethylenimine (Boussif, O. et al. Proc. Natl. Acad. Sci. U.S.A. 92, 7297-7301 (1995)). Virus infectivity was detected using the firefly luciferase assay system (Promega). Background infectivity of the pseudotype virus was determined using cells transfected with pNL4-3.lucR-E- only. The E1E2 expression plasmids for the isolates H77, H, CH35, OH8, UKN1B12.16, J6, UKN2A1.2, UKN2B1.1, UKN3A13.6, UKN3A1.28c, UKN4.21.16, UKN5.15.7 and UKN6.5.8 have been described previously (Owsianka, A. et al. J. Virol. 79, 11095-11104 (2005); McKeating, J. A. et al. J. Virol. 78, 8496-8505 (2004); Lavillette, D. et al. Hepatology 41, 265-274 (2005)). The expression plasmids encoding E1E2 of the virus in an infected human serum (KP) used in the protection experiment are described below (see also, FIG. 7B). The majority of HCV Envs, except H77, H and OH8, produce only low levels of HCVpp (<5,000 Relative Light Unit, RLU). To ensure the quality of data for determining virus neutralization, 1 HCVpp of low infectivity was concentrated 10-20 fold by centrifugation at 38,000×g for 1 hour. Serially diluted antibodies were first incubated with the virus for 1 hour at 37° C. before adding to Huh-7 cells in triplicate and the cells were incubated for 3 days. After background subtraction, virus neutralization was determined by the % change of RLU in the presence of antibodies. Virus concentration did not affect the neutralization of the prototype HCVpp-H77 by the MAbs in comparison to unconcentrated virus (data not shown). Although virus concentration improved the signals of several HCV Envs (Table E-4), consistent signals were not obtained with HCVpp displaying CH35, UKN3A1.28c, UKN6.5.8 or KP E1E2 in repeated experiments and these Envs were excluded in the analysis.

Cloning of E1E2 from an Infected Human Serum.

Total RNA in the HCV GT1a-infected human serum KP (140 μl) was purified using a QIAamp Viral RNA Mini Kit (Qiagen). First strand cDNA was generated using either a reverse primer specific to HCV1a (HCV1aOuterR, GGGATGCTGCATTGAGTA, (SEQ ID NO: 697); Lavillette, D. et al. Hepatology 41, 265-274 (2005)) or random hexamer using the SuperScript III reverse transcriptase (Invitrogen). The GT1a E1E2 genes were amplified by a nested PCR as described previously (Lavillette, D. et al. Hepatology 41, 265-274 (2005)) and the PCR products were cloned using the pcDNA3.1/V5-His TOPO TA Expression Kit (Invitrogen). An HIV-positive human serum was used as a negative control throughout the experiments and no non-specific product was generated. The sequences of 40 clones were determined by DNA sequencing and analyzed using VectorNTI software (Invitrogen). Expression of E1E2 proteins was confirmed by the presence of folded E2 proteins in cell lysates, prepared from 293T cells transfected with the corresponding DNA plasmids, by ELISA using MAb AR3A.

Antibody Protection Studies.

Human liver-chimeric mice were prepared as described previously. Mercer, D. F. et al. Nat. Med. 7, 927-933 (2001); Kneteman, N. M. et al. Hepatology 43, 1346-1353 (2006). The animal experiments were approved by the University of Alberta Animal Care and Use Committee for Health Sciences. All human liver biopsies and sera were collected under informed consent and the human subjects protocols were approved by the University of Alberta Health Research Ethics Board (Biomedical Panel). Colonization of human hepatocytes in the livers of Alb-uPA/SCID mice was confirmed by the presence of human alpha-1-anti-trypsin (hAAT) in the mice. Only mice with serum levels of hAAT greater than 60 μg/mL at 6 weeks and 100 μg/mL at 8 weeks, an indication for successful transplantation, were used in the protection study (˜50% of transplanted mice). Mice with low level of human liver chimerism were used in preliminary experiments to measure the toxicity and kinetics of MAbs in Alb-uPA/SCID mice, and the level of human IgG present in mice injected with a genotype 1a HCV-infected human serum KP. This serum, serially diluted from 1:50 to 1:4050, did not neutralize HCVpp-H77 (data not shown). Different doses of MAbs, at 10 mg/mL, were injected into the mice via the intraperitoneal route. For virus challenge, the experiments were conducted in blinded fashion; the identity of the MAbs was not provided to the technicians performing the animal procedures and HCV RNA tests. Human liver-chimeric mice were given MAbs by intraperitoneal injection (200 mg/kg) 24 hours before virus challenge. Mice were anesthetized and injected intrajugularly with 100 μL of infected serum KP (2.3×10⁶ IU/mL). Blood was sampled by tail bleed and sera were prepared by centrifugation of clotted blood for ELISA and viral load measurement.

HCV RNA Quantification.

HCV RNA in mouse serum was quantified by a real-time TaqMan PCR assay. The two primers in the real-time PCR system were designed to produce a 194 bp PCR fragment corresponding to the 5′ non-coding region with maximum specificity to all HCV genotypes. The forward primer (T-149-F, 5′-TGCGGAACCGGTGAGTACA, (SEQ ID NO: 698) and reverse primer (T-342-R, 5′-AGGTTTAGGATTCGTGCTCAT, (SEQ ID NO: 699) were designed with the aid of software Primer Express (PE biosystems) and were purchased from PE Applied Biosystems. To quantify HCV RNA, total RNA in serum was isolated by the guanidinium thiocyanate (GuSCN) and silico method (Boom, R. et al. J. Clin. Microbiol. 28, 495-503 (1990)). Briefly, 30 μL of serum was mixed with 500 μl GuSCN lysis buffer and 20 μL size-fractionated silica particles for 15 minutes. The silica particles were pelleted and washed twice with 500 μL washing buffer, twice with 70% ethanol and once with acetone. The pellet was dried for 10 min on heat block, and RNA was eluted in 20 μL distilled water and quantified by optical density. SuperScript II First-Strand Synthesis Kit (Invitrogen) was used to synthesize first-strand cDNA for PCR. Five μL of the serum RNA was mixed with 100 μM of SuperScript II reverse transcriptase, 20 μM of RNAseOut and 14 μL reaction cocktail (which includes 1×first-strand buffer, 5 μM DTT, 375 nM dNTP, 1.25 μM T-342-R primer) and incubated at 42° C. for 60 min then 70° C. for 15 minutes. For quantitative PCR, a 50 μL1 mixture contained 9 μL of template HCV cDNA, 1×TaqMan Universal PCR Master Mixture (Applied Biosystems Inc.), 375 nM dNTP, 400 nM of T-149-F and T-342-R primers and 200 nM TaqMan probe (6-FAM18 CACCCTATCAGGCAGTACCACAAGGCC-TAMRA, (SEQ ID NO: 700). Thermocycling was performed on a Taqman 7300 (Applied Biosystems Inc.) using the default setting program recommended by the manufacturer: 50° C. for 2 min, 95° C. for 10 min, and 45 cycles of 95° C. for 15 s and 60° C. for 60 s. A serial dilution of HCV cDNA, including 1.5×10⁶, 1.5×10⁵, 1.5×10⁴, 1.5×10³, 1.5×10², 1.5×10¹, 1.5×10⁰ UI, was used to generate a standard curve for calculation of HCV RNA copy number. The dynamic range of HCV RNA detection for the two step RT-PCR procedure is 6.0×10² IU/ml to 3.0×10⁸ IU/mL. Each assay run incorporates in duplicate a negative control and an HCV RNA positive control. The positive control is the OptiQual HCV RNA 1 Control purchased from AcroMetrix which has been calibrated to the WHO first International Standard for HCV RNA.

Statistical Analysis.

GraphPad Prism 4 software was used for statistical analysis of the antibody protection experiment. Animals seropositive for HCV RNA by the quantitative PCR assay at or after day 7 post-infection were scored as “infected” subjects and animals seronegative up to week 6 were scored as “censored” subjects. The scores were used to construct the Kaplan-Meier survival (infection in this case) curves to calculate statistical significance between the neutralizing antibody-treated and isotype antibody control groups by a two-tailed log rank test within the experimental period. Motulsky, H. Survival curves. in GraphPad Prism4 Statistics Guide: Statistical analyses for laboratory and clinical researchers 107-117 (GraphPad Software, San Diego, 2005).

### Example 2

### Anti-HCV Neutralizing Antibodies

The Example describes the identification of human monoclonal antibodies (mAbs) that neutralize genetically diverse HCV isolates and protect against heterologous HCV quasispecies challenge in a human liver-chimeric mouse model. The results provide evidence that broadly neutralizing antibodies to HCV protect against heterologous viral infection and suggest that a prophylactic vaccine against HCV may be achievable.

A total of 115 clones that exhibit specific binding to HCV E2 glycoprotein were isolated from an antibody antigen-binding fragment (Fab) phage display library generated from a donor chronically infected with HCV (see Example 1). DNA sequence analysis identified 36 distinct Fabs with 13 unique heavy chain sequences. The sequences of the 36 distinct Fabs belonging to 13 groups based on the heavy chain sequences are also shown in Table E-1 below. Fabs with the same designation and * or ** have the same heavy chain but distinct light chains, e.g. H1, H1* and H1** have the same heavy chain, but 3 different light chains.

TABLE E-1
Fab HCDR3 Sequences

| | | |
|---|---|---|
| | Isolated by | |
| Fab | masking with Fab | HCDR3 sequence |

| | | | |
|---|---|---|---|
| 1 | A | | ENKFRYCRGGSCYSGAFDM |
| | | | (SEQ ID NO: 140) |
| 2 | B1 | | DPYVYAGDDVWSLSR |
| | | | (SEQ ID NO: 141) |
| 3 | B2 | | DPYVYAGDDVRSLSR |
| | | | (SEQ ID NO: 142) |
| 4 | B3 | | DPYVYAGDDVWSLSR |
| | | | (SEQ ID NO: 143) |
| 5 | C1 | | PETPRYVSGGFCYGEFDN |
| | | | (SEQ ID NO: 144) |
| 6 | C1* | B1 | PETPRYCSGGFCYGEFDN |
| | | | (SEQ ID NO: 144) |
| 7 | C2 | B1 | PETPRYCRGGFCYGEFDN |
| | | | (SEQ ID NO: 145) |
| 8 | C2* | B1 | PETPRYCRGGFCYGEFDN |
| | | | (SEQ ID NO: 145) |
| 9 | C3 | B1 | PETPRYCSGGVCYGEFDN |
| | | | (SEQ ID NO: 146) |
| 10 | C4 | B1 | PETPRYCSGGFCYGEFDN |
| | | | (SEQ ID NO: 147) |
| 11 | C5 | B1 | PETPRYCSGGFCYGEFDN |
| | | | (SEQ ID NO: 148) |
| 12 | C6 | B1 | PETPRYCSGGFCYGEFDN |
| | | | (SEQ ID NO: 149) |
| 13 | D1 | C1 | DPLLFAGGPNWFDH |
| | | | (SEQ ID NO: 150) |
| 14 | D2 | C1 | DPLLFAGGPNWFDH |
| | | | (SEQ ID NO: 151) |
| 15 | D3 | C1, B1 & C1 | DPLLFAGGPNWFDH |
| | | | (SEQ ID NO: 152) |
| 16 | D4 | B1 & C1 | DPLLFAGGPNWFDH |
| | | | (SEQ ID NO: 153) |
| 17 | E | C1 | GPYVGLGEGFSE |
| | | | (SEQ ID NO: 154) |
| 18 | F | B1 & C1 | GGGTE |
| | | | (SEQ ID NO: 155) |
| 19 | G | B1 & C1 | DRGLAINGVVFPYFGLDV |
| | | | (SEQ ID NO: 156) |
| 20 | H1 | B1 | SVTPRHCGGGFCYGEFDY |
| | | | (SEQ ID NO: 157) |
| 21 | H1* | B1 | SVTPRHCGGGFCYGEFDY |
| | | | (SEQ ID NO: 157) |
| 22 | H1** | B1 | SVTPRHCGGGFCYGEFDY |
| | | | (SEQ ID NO: 157) |
| 23 | H2 | B1 | SVTPRHCGGGFCYGEFDY |
| | | | (SEQ ID NO: 158) |
| 24 | H3 | B1 | SVTPRYCGGGFCYGEFDY |
| | | | (SEQ ID NO: 159) |
| 25 | I | B1 | PHGPGLSLGIYSAEYFDE |
| | | | (SEQ ID NO: 160) |
| 26 | J1 | B1 | VGVRGIILVGGLAMNWLDP |
| | | | (SEQ ID NO: 161) |
| 27 | J2 | B1 | VGLRGIVMVGGLAMNWLDP |
| | | | (SEQ ID NO: 162) |
| 28 | J3 | B1 | VGLRGITLVGGLAMNWLDP |
| | | | (SEQ ID NO: 163) |
| 29 | J3* | B1 | VGLRGITLVGGLAMNWLDP |
| | | | (SEQ ID NO: 163) |
| 30 | J4 | B1 | VGLRGINMVGGLAMNWFDP |
| | | | (SEQ ID NO: 164) |
| 31 | K | B1 & C1 | DFYIGPTRDVYYGMDV |
| | | | (SEQ ID NO: 165) |
| 32 | L1 | B1 | AGDLSVGGVLAGGVPHLRHFDP |
| | | | (SEQ ID NO: 166) |
| 33 | L2 | B1 | AGDLSVGGVLAGGVPHLRHFDP |
| | | | (SEQ ID NO: 167) |
| 34 | L3 | B1 | AGDLSVGGVLAGGVPHLRHFDP |
| | | | (SEQ ID NO: 168) |
| 35 | L4 | B1 | AGDLSVGGVLAGGVPHLRHFDP |
| | | | (SEQ ID NO: 169) |
| 36 | M | B1 | ESLYMIAFGRVIWPPLDY |
| | | | (SEQ ID NO: 170) |

TABLE E-2
Anti-HCV E2 Fabs (IgGκ, heavy chain)

| | | |
|---|---|---|
| Fab | FRAMEWORK 1 | CDR1 |

| | | | | | |
|---|---|---|---|---|---|
| 1 | A | LEQSGAEVKKPGSSVKVSCKASGG | (SEQ ID NO: 309) | TFSSFVIN | (SEQ ID NO: 78) |
| 2 | B1 | LEQSGAEVKKPGSSVKVSCRASGS | (SEQ ID NO: 310) | PFSSYTIT | (SEQ ID NO: 79) |
| 3 | B2 | LEQSGAEVKKPGSSVKVSCRASGS | (SEQ ID NO: 311) | PYSSYTIT | (SEQ ID NO: 80) |
| 4 | B3 | LEQSGAEVKKPGSSVKVSCRASGS | (SEQ ID NO: 312) | PYSSYTIT | (SEQ ID NO: 81) |
| 5 | C1 | LEQSGAEVKTPGSSVRVSCRPPGG | (SEQ ID NO: 313) | NFNSYSIN | (SEQ ID NO: 82) |
| 6 | C1* | LEQSGAEVKTPGSSVRVSCRPPGG | (SEQ ID NO: 313) | NFNSYSIN | (SEQ ID NO: 82) |
| 7 | C2 | LEQSGAEVKKPGSSVRVSCRAPGG | (SEQ ID NO: 314) | TFNSYSVN | (SEQ ID NO: 83) |
| 8 | C2* | LEQSGAEVKKPGSSVRVSCRAPGG | (SEQ ID NO: 314) | TFNSYSVN | (SEQ ID NO: 83) |
| 9 | C3 | LEQSGAEVKEPGSSVRVSCRAPGG | (SEQ ID NO: 315) | TFNSYSIN | (SEQ ID NO: 84) |
| 10 | C4 | LEQSGAEVKKPGSSVRVSCRPPGG | (SEQ ID NO: 316) | TFNSYSIN | (SEQ ID NO: 85) |
| 11 | C5 | LEQSGAEVKKPGSSVRVSCRAPGG | (SEQ ID NO: 317) | TLNSYSIN | (SEQ ID NO: 86) |
| 12 | C6 | LEQSGAEVKKPGSSVRVSCRPPGG | (SEQ ID NO: 318) | TFNSYSIN | (SEQ ID NO: 87) |
| 13 | D1 | LE SGGGLVQPGGSLRLSCEASGY | (SEQ ID NO: 319) | YFSSFAMS | (SEQ ID NO: 88) |
| 14 | D2 | LEQSGGGLVQPGGSLRLSCEASGY | (SEQ ID NO: 320) | YFSSFAMS | (SEQ ID NO: 89) |
| 15 | D3 | LE SGGGLVQPGGSLRLSCEASGY | (SEQ ID NO: 321) | YFSSFAMS | (SEQ ID NO: 90) |
| 16 | D4 | LE SGGGLVQPGGSLRLSCEASGY | (SEQ ID NO: 322) | YFSSFAMS | (SEQ ID NO: 91) |
| 17 | E | LEQSGAELKKPGSSVKVSCKPSDG | (SEQ ID NO: 323) | TFRAYTLS | (SEQ ID NO: 92) |
| 18 | F | LEQSGNEVKKPGASVKVSCRAYGY | (SEQ ID NO: 324) | NFGSERLS | (SEQ ID NO: 93) |
| 19 | G | LEQSGAEMKKPGASLKVSCKTSGY | (SEQ ID NO: 325) | TFDDYGVT | (SEQ ID NO: 94) |
| 20 | H1 | LEQSGAEVKKPGSSVKVSCEASGG | (SEQ ID NO: 326) | TFDNYSLN | (SEQ ID NO: 95) |
| 21 | H1* | LEQSGAEVKKPGSSVKVSCEASGG | (SEQ ID NO: 326) | TFDNYSLN | (SEQ ID NO: 95) |
| 22 | H1** | LEQSGAEVKKPGSSVKVSCEASGG | (SEQ ID NO: 326) | TFDNYSLN | (SEQ ID NO: 95) |
| 23 | H2 | LEQSGAEVKKPGSSVKVSCETSGG | (SEQ ID NO: 327) | TFDNYALN | (SEQ ID NO: 96) |
| 24 | H3 | LEQSGAEVKKPGSSVKVSCETSGG | (SEQ ID NO: 328) | TLDNYALN | (SEQ ID NO: 97) |
| 25 | I | LE SGGGLVQPGRSLRLSCKASGF | (SEQ ID NO: 329) | NFAQYTMN | (SEQ ID NO: 98) |
| 26 | J1 | LEQSGPEVKKPGSSVKVSCKGSGD | (SEQ ID NO: 330) | RFNDPVT | (SEQ ID NO: 99) |
| 27 | J2 | LEQSGPEVKKPGSSVKVSCKDSGD | (SEQ ID NO: 331) | TFNEPVT | (SEQ ID NO: 100) |
| 28 | J3 | LEQSGPEVKKPGSSVKVSCKGSGD | (SEQ ID NO: 332) | TFNDPVT | (SEQ ID NO: 101) |
| 29 | J3* | LEQSGPEVKKPGSSVKVSCKGSGD | (SEQ ID NO: 332) | TFNDPVT | (SEQ ID NO: 101) |
| 30 | J4 | LEQSGAEVKKPGSSVRVSCEVSGD | (SEQ ID NO: 333) | TFREPVS | (SEQ ID NO: 102) |
| 31 | K | LEQSGPGLVKPGRPFSLTCAISGD | (SEQ ID NO: 334) | SVSSDSAAWN | (SEQ ID NO: 103) |
| 32 | L1 | LEQSGAEVKKPGSSVKVSCKASGD | (SEQ ID NO: 335) | TFRSYVIT | (SEQ ID NO: 104) |
| 33 | L2 | LEQSGAEVKMPGSSVKVSCKASGD | (SEQ ID NO: 336) | TFRSSVIT | (SEQ ID NO: 105) |
| 34 | L3 | LEQSGAEVKKPGSSVKVSCKASGD | (SEQ ID NO: 337) | TFRSYVIT | (SEQ ID NO: 106) |
| 35 | L4 | LEQSGAEVKKPGSSVKVSCKASGD | (SEQ ID NO: 338) | TFRSYVIT | (SEQ ID NO: 107) |
| 36 | M | LEQSGAEVKKPGASVKVSCKASGY | (SEQ ID NO: 339) | TFTNYAIT | (SEQ ID NO: 108) |

| | | |
|---|---|---|
| Fab | FRAMEWORK 2 | CDR2 |

| | | | | |
|---|---|---|---|---|
| A | WVRQAPGQGLEWVGG | (SEQ ID NO: 340) | INPISGTINYAQRFQG | (SEQ ID NO: 109) |
| B1 | WVRQAPGQGLEWMGG | (SEQ ID NO: 341) | IILMTGKANYAQKFQG | (SEQ ID NO: 110) |
| B2 | WVRQAPGQGLEWMGG | (SEQ ID NO: 342) | IILMTGKANYAQKFQG | (SEQ ID NO: 111) |
| B3 | WVRQAPGQGLEWMGG | (SEQ ID NO: 343) | IILMTGKANYAQKFQG | (SEQ ID NO: 112) |
| C1 | WVRQAPGHGLEWVGT | (SEQ ID NO: 344) | FIMPFGTSKYAQKFQG | (SEQ ID NO: 113) |
| C1* | WVRQAPGHGLEWVGT | (SEQ ID NO: 344) | FIPMFGTSKYAQKFQG | (SEQ ID NO: 113) |
| C2 | WVRQAPGHGLEWVGT | (SEQ ID NO: 345) | LIPMFGTSSYAQKFQG | (SEQ ID NO: 114) |
| C2* | WVRQAPGHGLEWVGT | (SEQ ID NO: 345) | LIPMFGTSSYAQKFQG | (SEQ ID NO: 114) |
| C3 | WVRQAPGHGLEWVGT | (SEQ ID NO: 346) | LIPMFGTSNYAQKFQG | (SEQ ID NO: 115) |
| C4 | WVRQAPGHGLEWVGT | (SEQ ID NO: 347) | LIPMFGTSKYAQKLQG | (SEQ ID NO: 116) |
| C5 | WVRQAPGHGLEWVGT | (SEQ ID NO: 348) | LIPMFGTSNYAQKFQG | (SEQ ID NO: 117) |
| C6 | WVRQAPGHGLEWVGT | (SEQ ID NO: 349) | IIPMFGTSKYAQKLQG | (SEQ ID NO: 118) |
| D1 | WVRQTPGKGLEWVSS | (SEQ ID NO: 350) | IAGGTLGRTSYRDSVKG | (SEQ ID NO: 119) |
| D2 | WVRQTPGKGLEWVSS | (SEQ ID NO: 351) | IAGGTLGRTSYRDSVKG | (SEQ ID NO: 120) |
| D3 | WVRQTPGKGLEWVSS | (SEQ ID NO: 352) | IAGGTLGRTSYRDSVKG | (SEQ ID NO: 121) |
| D4 | WVRQTPGKGLEWVSS | (SEQ ID NO: 353) | IAGGTLGRTSYRDSVKG | (SEQ ID NO: 122) |
| E | WVRQAPGQTLEWMGR | (SEQ ID NO: 354) | IMPTVGITNYAQKFQG | (SEQ ID NO: 123) |
| F | WVRQAPGQGLEWMGW | (SEQ ID NO: 355) | ISAYNGGINYSQKFQG | (SEQ ID NO: 124) |
| G | WVRQAPGQGLEWMGW | (SEQ ID NO: 356) | ISAYSGNTFYARKFQG | (SEQ ID NO: 125) |
| H1 | WVRQAPGQGLEWIGG | (SEQ ID NO: 357) | VVPLFGTTKYAQKFQG | (SEQ ID NO: 126) |
| H1* | WVRQAPGQGLEWIGG | (SEQ ID NO: 357) | VVPLFGTTKYAQKFQG | (SEQ ID NO: 126) |
| H1** | WVRQAPGQGLEWIGG | (SEQ ID NO: 357) | VVPLFGTTKYAQKFQG | (SEQ ID NO: 126) |
| H2 | WVRQAPGQGLEWIGG | (SEQ ID NO: 358) | VVPLFGTTKYAQKFQG | (SEQ ID NO: 127) |
| H3 | WVRQAPGQGLEWIGG | (SEQ ID NO: 359) | VVPLFGTTRNAQKFQG | (SEQ ID NO: 128) |
| I | WVRQAPGKGLEWIGL | (SEQ ID NO: 360) | IRTTAYDAATHYAASVEG | (SEQ ID NO: 129) |
| J1 | WVRQAPGQGLEWIGG | (SEQ ID NO: 361) | IIPAFGATKYAQKFQG | (SEQ ID NO: 130) |
| J2 | WVRQAPGQGLEWIGG | (SEQ ID NO: 362) | IIPAFGVTKYAQKFQG | (SEQ ID NO: 131) |
| J3 | WVRQAPGQGLEWIGG | (SEQ ID NO: 363) | IIPLFGAAKYAQKFQG | (SEQ ID NO: 132) |
| J3* | WVRQAPGQGLEWIGG | (SEQ ID NO: 363) | IIPLFGAAKYAQKFQG | (SEQ ID NO: 132) |
| J4 | WVRQAPGQGFEWIGG | (SEQ ID NO: 364) | IIPMFGATHYAQKLQG | (SEQ ID NO: 133) |
| K | WVRQSPSRGLEWLGR | (SEQ ID NO: 365) | TFYRSKWYYDYTVSVKS | (SEQ ID NO: 134) |
| L1 | WARQAPGQGLEWMGA | (SEQ ID NO: 366) | IIPFFGTTNLAQKFQG | (SEQ ID NO: 135) |
| L2 | WARQAPGQGLEWMGA | (SEQ ID NO: 367) | IIPFFGTTNLAQKFQG | (SEQ ID NO: 136) |
| L3 | WARQAPGQGLEWMGA | (SEQ ID NO: 368) | IIPFFGTTNLAQKFQG | (SEQ ID NO: 137 |
| L4 | WARQAPGQGLEWMGA | (SEQ ID NO: 369) | IIPFFGTTNLAQKFQG | (SEQ ID NO: 138) |
| M | WVRQAPGQGLEWMGW | (SEQ ID NO: 370) | ISGDSTNTYYGQKFQG | (SEQ ID NO: 139) |

| Fab | FRAMEWORK 3 | CDR3 | FRAMEWORK 4 |
|---|---|---|---|
| A | RVTMTADESMTTVYMELSSLRSEDTAMYYCAR | ENKFRYCRGGSCYSGAFDM | WGQGTMVTVSSAS |
| | (SEQ ID NO: 371) | (SEQ ID NO: 140) | (SEQ ID NO: 402) |
| B1 | RVTITADRSTTTAYMEMSSLTSDDTAIYYCAR | DPYVYAGDDVWSLSR | WGQGTLVIVSSAS |
| | (SEQ ID NO: 372) | (SEQ ID NO: 141) | (SEQ ID NO: 403) |
| B2 | RVTITADRATATAYMEMSSLTSDDTAIYYCAR | DPYVYAGDDVRSLSR | WGQGTPVIVSSAS |
| | (SEQ ID NO: 373) | (SEQ ID NO: 142) | (SEQ ID NO: 404) |
| B3 | RVTITADRATATAYMEMSSLTSDDTAIYYCAR | DPYVYAGDDVWSLSR | WGQGTPVIVSSAS |
| | (SEQ ID NO: 374) | (SEQ ID NO: 143) | (SEQ ID NO: 405) |
| C1 | RVTITADGSSGTAYMDLNSLRSDDTAFYYCVR | PETPRYCSGGFCYGEFDN | WGQGTLVTVSSAS |
| | (SEQ ID NO: 375) | (SEQ ID NO: 144) | (SEQ ID NO: 406) |
| C1* | RVTITADGSSGTAYMDLNSLRSDDTAFYYCVR | PETPRYCSGGFCYGEFDN | WGQGTLVTVSSAS |
| | (SEQ ID NO: 375) | (SEQ ID NO: 144) | (SEQ ID NO: 406) |
| C2 | RVTITADGSSGTAYMELNSLRSDDTAVYYCVR | PETPRYCRGGFCYGEFDN | WGQGTLVTVSSAS |
| | (SEQ ID NO: 376) | (SEQ ID NO: 145) | (SEQ ID NO: 407) |
| C2* | RVTITADGSSGTAYMELNSLRSDDTAVYYCVR | PETPRYCRGGFCYGEFDN | WGQGTLVTVSSAS |
| | (SEQ ID NO: 376) | (SEQ ID NO: 145) | (SEQ ID NO: 407) |
| C3 | RVTITADGSSGTAYMELNSLRSDDTAVYYCVR | PETPRYCSGGVCYGEFDN | WGQGTLVTVSSAS |
| | (SEQ ID NO: 377) | (SEQ ID NO: 146) | (SEQ ID NO: 408) |
| C4 | RVTITADGSSGTAYMELNSLRSDDTAVYYCVR | PETPRYCSGGFCYGEFDN | WGQGTLVTVSSAS |
| | (SEQ ID NO: 378) | (SEQ ID NO: 147) | (SEQ ID NO: 409) |
| C5 | RVTITADGSSGTAYMELNSLRSDDTAVYYCVR | PETPRYCSGGFCYGEFDN | WGQGTLVTVSSAS |
| | (SEQ ID NO: 379) | (SEQ ID NO: 148) | (SEQ ID NO: 410) |
| C6 | RVTITADGSSGTAYMELNSLRSDDTAVYYCVR | PETPRYCSGGFCYGEFDN | WGQGTLVTVSSAS |
| | (SEQ ID NO: 380) | (SEQ ID NO: 149) | (SEQ ID NO: 411) |
| D1 | RFTISRDNSKNTVFLHMNNLRPEDTAVYYCAK | DPLLFAGGPNWFDH | WGQGTLVTVSSAS |
| | (SEQ ID NO: 381) | (SEQ ID NO: 150) | (SEQ ID NO: 412) |
| D2 | RFTISRDNSKNTVFLHMNNLRPEDTAVYYCAK | FPLLFAGGPNWFDH | WGQGTLVTVSSAS |
| | (SEQ ID NO: 382) | (SEQ ID NO: 151) | (SEQ ID NO: 413) |
| D3 | RFTISRDNSKNTMFLHMNNLRPEDTAVYYCAK | DPLLFAGGPNWFDH | WGQGTLVTVSSAS |
| | (SEQ ID NO: 383) | (SEQ ID NO: 152) | (SEQ ID NO: 414) |
| D4 | RFTISRDNSKNTVFLHMSNLRPEDTAVYYCAK | DPLLFAGGPNWFDH | WGQGTLVTVSSAS |
| | (SEQ ID NO: 384) | (SEQ ID NO: 153) | (SEQ ID NO: 415) |
| E | RVTISADMSTATAYMELSSLRSDDTAIYYCAK | GPYVGLGEGFSE | WGQGTLVTVSSAS |
| | (SEQ ID NO: 385) | (SEQ ID NO: 154) | (SEQ ID NO: 416) |
| F | RFTMTTDTSTRTGYMELRNLRSDDTAVYYCAR | GGGTE | WGQGTLVIVSSDE |
| | (SEQ ID NO: 386) | (SEQ ID NO: 155) | (SEQ ID NO: 417) |
| G | RVTMTTDPSTRTAYMELRSLRSDDTAVYFCAR | DRGLAINGVVFPYFGLDV | WGQGTTVTVSSAS |
| | (SEQ ID NO: 387) | (SEQ ID NO: 156) | (SEQ ID NO: 418) |
| H1 | RVTISDDKSTGTGHMELRSLRSEDTAVYYCVR | SVTPRHCGGGFCYGEFDY | WGQGTLVTVSSAS |
| | (SEQ ID NO: 388) | (SEQ ID NO: 157) | (SEQ ID NO: 419) |
| H1* | RVTISDDKSTGTGHMELRSLRSEDTAVYYCVR | SVTPRHCGGGGCYGEFDY | WGQGTLVTVSSAS |
| | (SEQ ID NO: 388) | (SEQ ID NO: 157) | (SEQ ID NO: 419) |
| H1** | RVTISDDKSTGTGHMELRSLRSEDTAVYYCVR | SVTPRHCGGGFCYGEFDY | WGQGTLVTVSSAS |
| | (SEQ ID NO: 388) | (SEQ ID NO: 157) | (SEQ ID NO: 419) |
| H2 | RVTISDDKSTGTGHMELRSLRSEDTAVYYCVR | SVTPRHCGGGFCYGEFDY | WGQGTLVTVSSAS |
| | (SEQ ID NO: 389) | (SEQ ID NO: 158) | (SEQ ID NO: 420) |
| H3 | RVTISDDKSTGTGHMELRSLRSEDTAVYYCVR | SVTPRYCGGGFCYGEFDY | WGQGTLVTVSSAS |
| | (SEQ ID NO: 390) | (SEQ ID NO: 159) | (SEQ ID NO: 421) |
| I | RFTISRDDSKSTAYLQINGLKTEDTAVYYCAR | PHGPGLSLGIYSAEYFDE | WGQGTLVTVSSAS |
| | (SEQ ID NO: 391) | (SEQ ID NO: 160) | (SEQ ID NO: 422) |
| J1 | RVVISADASTDTAYMELSSLRSEDTAVYYCAK | VGVRGIILVGGLAMNWLDP | WGQGTLVTVSAAS |
| | (SEQ ID NO: 392) | (SEQ ID NO: 161) | (SEQ ID NO: 423) |
| J2 | RVIISADASTATAYLELSSLRSEDTAVYYCAK | VGLRGIVMVGGLAMNWLDP | WGQGTQVTVSSAS |
| | (SEQ ID NO: 393) | (SEQ ID NO: 162) | (SEQ ID NO: 424) |
| J3 | RVTISADASALTTYMELSSLRPEDTAVYYCAK | VGLRGITLVGGLAMNWLDP | WGQGTLITVSSAS |
| | (SEQ ID NO: 394) | (SEQ ID NO: 163) | (SEQ ID NO: 425) |
| J3* | RVTISADASALTTYMELSSLRPEDTAVYYCAK | VGLRGITLVGGLAMNWLDP | WGQGTLITVSSAS |
| | (SEQ ID NO: 394) | (SEQ ID NO: 163) | (SEQ ID NO: 425) |
| J4 | RITISADQSTNTVYMELSSLRSDDTAVYYCAK | VGLRGINMVGGLAMNWFDP | WGQGTLVTVSSAS |
| | (SEQ ID NO: 395) | (SEQ ID NO: 164) | (SEQ ID NO: 426) |
| K | RITINSKTSKNQFSLHLNSVTPEDTAVYYCVR | DFYIGPTRDVYYGMDV | WGQGTTVTVSSAS |
| | (SEQ ID NO: 396) | (SEQ ID NO: 165) | (SEQ ID NO: 427) |
| L1 | RVTITADESTQTVYMDLSSLRSDDTAVYYCAK | AGDLSVGGLAGGVPHLRHFDP | WGQGTLVTVSSAS |
| | (SEQ ID NO: 397) | (SEQ ID NO: 166) | (SEQ ID NO: 428) |
| L2 | RVTITADESTKTVYMDLSSLRSDDTAVYYCAK | AGDLSVGGVLAGGVPHLRHFDP | WGQGTLVTVSSAS |
| | (SEQ ID NO: 398) | (SEQ ID NO: 167) | (SEQ ID NO: 429) |
| L3 | RVTITADESTKTVYMDLSSLTSDDTAVYYCAK | AGDLSVGGVLAGGVPHLRHFDP | WGQGTLVTVSSAS |
| | (SEQ ID NO: 399) | (SEQ ID NO: 168) | (SEQ ID NO: 430) |
| L4 | RVTITADESTKTVYMDLSSLRSDDTAVYYCAK | AGDLSVGGVLAGGVPHLRHFDP | WGQGTLVTVSSAS |
| | (SEQ ID NO: 400) | (SEQ ID NO: 169) | (SEQ ID NO: 431) |
| M | RVTMTTDTSTSTAYMELTSLTSEDTAVYYCAR | ESLYMIAFGRVIWPPLDY | WGQGTLVTISSAS |
| | (SEQ ID NO: 401) | (SEQ ID NO: 170) | (SEQ ID NO: 432) |

TABLE E-3
Anti-HCV E2 Fabs (IgGκ, Light chain)

| | | |
|---|---|---|
| Fab | FRAMEWORK 1 | CDR1 |

| | | | | |
|---|---|---|---|---|
| A | EL TQSPATLSVSPGESATLSC | (SEQ ID NO: 433) | RASQSVSDN LA | (SEQ ID NO: 171) |
| B1 | ELTLTQSPGTLSLSPGERATLSC | (SEQ ID NO: 434) | RASQSVSNS YLA | (SEQ ID NO: 172) |
| B2 | TLTQSPDSLAVSLGERATINC | (SEQ ID NO: 435) | KSSQSVLYSSNNKNVLA | (SEQ ID NO: 173) |
| B3 | ELVMTQSPGTLSLSPGERATLSC | (SEQ ID NO: 436) | RASQRVGSS YLA | (SEQ ID NO: 174) |
| C1 | ELTLTQSPGTLSLSPGERATLSC | (SEQ ID NO: 437) | RASQSVSGN YLA | (SEQ ID NO: 175) |
| C1* | EL TQSPSTLSLSPGEGATLSC | (SEQ ID NO: 438) | RPSQSVSRN YLA | (SEQ ID NO: 176) |
| C2 | EL TQSPGTLSLSPGERAALSC | (SEQ ID NO: 439) | RASQSISTN YLA | (SEQ ID NO: 177) |
| C2* | EL TQSPGTLSLSPGERATLSC | (SEQ ID NO: 440) | RASQSVS YLA | (SEQ ID NO: 178) |
| C3 | EL TQSPGTLSLSPGERATLSC | (SEQ ID NO: 441) | RASQSVSSS YLA | (SEQ ID NO: 179) |
| C4 | EL TQSPGTLSLSPGERATLSC | (SEQ ID NO: 442) | RASQSVSSN YLA | (SEQ ID NO: 180) |
| C5 | EL TQSPATLYVSPGERATLSC | (SEQ ID NO: 443) | RASQSVPDN HLA | (SEQ ID NO: 181) |
| C6 | EL TQSPATLSVSPGESATLSC | (SEQ ID NO: 444) | RASQSVSSN LA | (SEQ ID NO: 182) |
| D1 | EL TQSPGTLSLSPGERATLSC | (SEQ ID NO: 445) | RASQSVSSS YLA | (SEQ ID NO: 183) |
| D2 | TL TQSPATLSVSPGERATLSC | (SEQ ID NO: 446) | RASQTTSDN LA | (SEQ ID NO: 184) |
| D3 | ELTLTQSPGTLSLSPGREATLSC | (SEQ ID NO: 447) | RASQTVSSS YLA | (SEQ ID NO: 185) |
| D4 | ELVMTQSPGTLSLSPGERATLSC | (SEQ ID NO: 448) | RASQSVSSS YLA | (SEQ ID NO: 186) |
| E | ELVLTQSPLSLPVTLGQPASISC | (SEQ ID NO: 449) | RSTQSLVYSDGNT YLN | (SEQ ID NO: 187) |
| F | ELQMTQSPSFLSASVGDRVTITC | (SEQ ID NO: 450) | RASQGISS YLA | (SEQ ID NO: 188) |
| G | EL TQSPVSLPVTPGEPASISC | (SEQ ID NO: 451) | RSSQSLLHSNGNH YLD | (SEQ ID NO: 189) |
| H1 | ELTLTQSPGTLSLSPGERATLSC | (SEQ ID NO: 452) | RASQSISTN YLA | (SEQ ID NO: 190) |
| H1* | EL TQSPATLSVSPGERATLSC | (SEQ ID NO: 453) | RASRGISSN LA | (SEQ ID NO: 191) |
| H1** | ELTLTQSPGTLSLSPGERATLSC | (SEQ ID NO: 454) | RASQSVSSDS LA | (SEQ ID NO: 192) |
| H2 | ELTLTQSPGTLSLSPGERGTLSC | (SEQ ID NO: 455) | RASQSVSSS YLA | (SEQ ID NO: 193) |
| H3 | EL TQSPATLSVSPGERATLSC | (SEQ ID NO: 456) | RASQSVSSN LA | (SEQ ID NO: 194) |
| I | ELTLTQSPATLSVSPGERATLFC | (SEQ ID NO: 457) | RANQSVGRN LA | (SEQ ID NO: 195) |
| J1 | ELVLTQSPGTLSLSPGERATLSC | (SEQ ID NO: 458) | RASQSVSSS YLA | (SEQ ID NO: 196) |
| J2 | EL TQSPGTLSLSPGERATLSC | (SEQ ID NO: 459) | RASQSVSS YLA | (SEQ ID NO: 197) |
| J3 | EL TQSPGTLSLSPGERATLSC | (SEQ ID NO: 460) | RASQSVSS YLA | (SEQ ID NO: 198) |
| J3* | EL TQSPGTLSLSPGERGTLSC | (SEQ ID NO: 461) | RASQSVSS YLA | (SEQ ID NO: 199) |
| J4 | ELTLTQSPGTLSLSPGERATLSC | (SEQ ID NO: 462) | RASQSVSS YLA | (SEQ ID NO: 200) |
| K | EL TQSPGTLSLSPGERATLSC | (SEQ ID NO: 463) | RASQSVSSNS LA | (SEQ ID NO: 201) |
| L1 | EL TQSPGTLSLSPGERATLSC | (SEQ ID NO: 464) | RASQSITSR YLA | (SEQ ID NO: 202) |
| L2 | EL TQSPGTLSLSPGERATLSC | (SEQ ID NO: 464) | RASQSITSR YLA | (SEQ ID NO: 202) |
| L3 | ELVMTQSPATLSLSPGERATLSC | (SEQ ID NO: 465) | RASQSVGS YLA | (SEQ ID NO: 203) |
| L4 | EL TQSPGTLSLSPGERATLSC | (SEQ ID NO: 466) | RAGQTVASNS LA | (SEQ ID NO: 204) |
| M | ELTLTQSPGTLSLSPGERATLSC | (SEQ ID NO: 467) | RASQSIRSS YLA | (SEQ ID NO: 205) |

| Fab | FRAMEWORK 2 | CDR2 | FRAMEWORK 3 |
|---|---|---|---|
| A | WYQQKPGQAPRLLIY | GASSRAP | AIPGRFSGSGSGTDFTLTISRLEPEDLAVYHC |
| | (SEQ ID NO: 468) | (SEQ ID NO: 206) | (SEQ ID NO: 503) |
| B1 | WYQQKPGQAPRLLIY | GASSRAT | GIPDRFSGSGSGTDFTLTISRLEPEDFAVYYC |
| | (SEQ ID NO: 469) | (SEQ ID NO: 207) | (SEQ ID NO: 504) |
| B2 | WYQQKPGQPPQLLIY | WASTRES | GVPDRFSGSGSGTDGTLTISSLQAEDVAVYFC |
| | (SEQ ID NO: 470) | (SEQ ID NO: 208) | (SEQ ID NO: 505) |
| B3 | WYQQKPGQAPRLLVY | GASSRAT | GIPDRFSGSGSGTDFTLTISRLQPEDFAVYYC |
| | (SEQ ID NO: 471) | (SEQ ID NO: 209) | (SEQ ID NO: 506) |
| C1 | WYQQKPGQAPRLLIY | GASNRAT | GIPHRFSGSGSGTDFTLTISRLEPEDFAVYYC |
| | (SEQ ID NO: 472) | (SEQ ID NO: 210) | (SEQ ID NO: 507) |
| C1* | WYQQKPGQAPRLLIY | GASTRAT | GIPDRFSGSGSGTNFTLTISRLEPEDFAVYFC |
| | (SEQ ID NO: 473) | (SEQ ID NO: 211) | (SEQ ID NO: 508) |
| C2 | WYQQKPGQAPRLLIY | GTSSRAT | SIPDRFSGTGSGTDFSLTISRLEPEDSAVYYC |
| | (SEQ ID NO: 474) | (SEQ ID NO: 212) | (SEQ ID NO: 509) |
| C2* | WYQQKPGQAPRLLIY | GASSRAT | GIPDRFSGSGSGTDFTLTISRLEPEDFAVYYC |
| | (SEQ ID NO: 475) | (SEQ ID NO: 213) | (SEQ ID NO: 510) |
| C3 | WYQQKPGQAPRLLIY | GASSRAT | GIPDRFSGSGSGTDFTLTISGLEPEDFAVYYC |
| | (SEQ ID NO: 476) | (SEQ ID NO: 214) | (SEQ ID NO: 511) |
| C4 | WYQQKPGQAPRLLIY | GASSRAT | GIPDRFSGSGSGTNFTLTISRLEPEDFAVYYC |
| | (SEQ ID NO: 477) | (SEQ ID NO: 215) | (SEQ ID NO: 512) |
| C5 | WYQQKPGQTPRLLIY | GASKRAT | GIPDRFSGSGSGTDGTLTISRLEPEDFAVYYC |
| | (SEQ ID NO: 478) | (SEQ ID NO: 216) | (SEQ ID NO: 513) |
| C6 | WYQQKPGQAPRLLIY | GASTRAT | GIPARFSGSGSGTDFTLTISRLEPEDFAVYYC |
| | (SEQ ID NO: 479) | (SEQ ID NO: 217) | (SEQ ID NO: 514) |
| D1 | WYQQKPGQAPRLLIY | GASSRAT | GIPDRFSGSGSGTDFTLTISRLEPEDFAVYYC |
| | (SEQ ID NO: 480) | (SEQ ID NO: 218) | (SEQ ID NO: 515) |
| D2 | WYQQKPGQAPRLLIY | GASSRAT | GIPDRFSGSGSGTDFTLTISRLEPEDFAVYYC |
| | (SEQ ID NO: 481) | (SEQ ID NO: 219) | (SEQ ID NO: 516) |
| D3 | WYQQKPGQAPRLLIY | GASSRAT | GIPDRFSGSGSGTDFTLTISRLEPEDFAVYYC |
| | (SEQ ID NO: 482) | (SEQ ID NO: 220) | (SEQ ID NO: 517) |
| D4 | WYQQKPGQAPRLLIY | GASSRAT | GIPDRFSGSGSGTDFTLTITTLEPEDFAVYYC |
| | (SEQ ID NO: 483) | (SEQ ID NO: 221) | (SEQ ID NO: 518) |
| E | WFHQRAGQPPRRLIY | KVSNRDS | GVPERFSGSGSGTDFTLKISRVEAEDVGIYYC |
| | (SEQ ID NO: 484) | (SEQ ID NO: 222) | (SEQ ID NO: 519) |
| F | WYQQKPGKAPKLLIS | SVSTLQS | GVSSRFSGSGSGTGFTLTISSLQSEDSATYYC |
| | (SEQ ID NO: 485) | (SEQ ID NO: 223) | (SEQ ID NO: 520) |
| G | WYLQKPGQSPQLLMY | LGSNRAS | GVPDRFSGSGSGTDFTLKISRVEAEDVGVYYC |
| | (SEQ ID NO: 486) | (SEQ ID NO: 224) | (SEQ ID NO: 521) |
| H1 | WYQQKPGQAPRLLIY | GASSRAT | GIPDRFSGSGSGTDFTLTISRLEPEDFAVYYC |
| | (SEQ ID NO: 487) | (SEQ ID NO: 225) | (SEQ ID NO: 522) |
| H1* | WYQQKPGQAPRLLIY | GASSRAT | GIPDRFSGSGSGTDFTLTISRLEPEDFAVYYC |
| | (SEQ ID NO: 488) | (SEQ ID NO: 226) | (SEQ ID NO: 523) |
| H1** | WYQQKPGQAPRLLIY | GASRRAT | GIPDRFSGSGSGTDFTLTISRLEPEDLGVYYC |
| | (SEQ ID NO: 489) | (SEQ ID NO: 227) | (SEQ ID NO: 524) |
| H2 | WYQQKPGQAPRLLIY | GASSRAT | GIPDRFSGSGSGTDFTLTISRLEPEDFAVYYC |
| | (SEQ ID NO: 490) | (SEQ ID NO: 228) | (SEQ ID NO: 525) |
| H3 | WYQQKPGQAPRLLIY | GASTRAT | GIPARFSGSGSGTDFTLTVSRLEPEDSAVYFC |
| | (SEQ ID NO: 491) | (SEQ ID NO: 229) | (SEQ ID NO: 526) |
| I | WYQQKPGQAPRLLIY | GISTRTT | TTPTRFSGSGSGTDFTLTISRLQSEDFAVYYC |
| | (SEQ ID NO: 492) | (SEQ ID NO: 230) | (SEQ ID NO: 527) |
| J1 | WYQQKPGQAPRLLIY | GASSRAT | GIPDRFSGSGSGTDFALTITRLEPEDFAVYYC |
| | (SEQ ID NO: 493) | (SEQ ID NO: 231) | (SEQ ID NO: 528) |
| J2 | WYQQKPGQAPRLLIY | GASSRAT | GIPDRFSGSGSGTDFTLTISRLEPEDFAVYYC |
| | (SEQ ID NO: 494) | (SEQ ID NO: 232) | (SEQ ID NO: 529) |
| J3 | WYQQKPGQAPRLLIY | GASSRAT | GIPDRFSGSGSGTDFTLTISGLEPEDFAVYYC |
| | (SEQ ID NO: 495) | (SEQ ID NO: 233) | (SEQ ID NO: 530) |
| J3* | WYQQKPGQAPRLLIY | GASSRAT | GIPDRFSGSGSGTDFTLTISRLEPEDFAVYY |
| | (SEQ ID NO: 496) | (SEQ ID NO: 234) | (SEQ ID NO: 531) |
| J4 | WYQQKPGQAPRLLIY | GASSRAT | GIPDRFSGSGSGTDFTLTISRLEPEDFAVYYC |
| | (SEQ ID NO: 497) | (SEQ ID NO: 235) | (SEQ ID NO: 532) |
| K | WYQQKPGLAPRLLIY | GASSRAT | GIPDRFSGSGSGTGFTLTISTLEPEDFAIYYC |
| | (SEQ ID NO: 498) | (SEQ ID NO: 236) | (SEQ ID NO: 533) |
| L1 | WYQQKPGQAPRLLIY | GASSRAT | GIPDRFSGSGSGTDFTLTISRLEPEDFAVYYC |
| | (SEQ ID NO: 499) | (SEQ ID NO: 237) | (SEQ ID NO: 534) |
| L2 | WYQQKPGQAPRLLIY | GASSRAT | GIPDRDSGSGSGTDFTLTISRLEPEDFAVYYC |
| | (SEQ ID NO: 499) | (SEQ ID NO: 237) | (SEQ ID NO: 534) |
| L3 | WYQQKPGQAPRLLIY | DASNRAT | GIPARFSGSGSGTDFTLTISSLEPEDFAVYFC |
| | (SEQ ID NO: 500) | (SEQ ID NO: 238) | (SEQ ID NO: 535) |
| L4 | WYQHKPGQAPRLLIY | GASIRAS | GIPDRFSGSGSGTDFTLTISRLEPEDFAVYYC |
| | (SEQ ID NO: 501) | (SEQ ID NO: 239) | (SEQ ID NO: 536) |
| M | WYQQKPGQAPRLLIY | AAAIRAT | GIPDRFSGSGSGTDFTLTISRLEPEDFAVYFC |
| | (SEQ ID NO: 502) | (SEQ ID NO: 240) | (SEQ ID NO: 537) |

| | | |
|---|---|---|
| Fab | CDR3 | FRAMEWORK 4 |

| | | | | |
|---|---|---|---|---|
| A | QQYGAS PWT | (SEQ ID NO: 241) | FGQGTKVEIKRTVAAPSVFIFPPSDEQLKSGTASVV | (SEQ ID NO: 538) |
| B1 | QQYGSS PQT | (SEQ ID NO: 242) | FGQGTKVEIKRTVAAPSVFIFPPSDEQLKSGTASVV | (SEQ ID NO: 539) |
| B2 | QQYYAT PFT | (SEQ ID NO: 243) | FGQGTKVEIKRTVAAPSVFIFPPSDEQLKSGTASVV | (SEQ ID NO: 540) |
| B3 | QQYGTT | (SEQ ID NO: 244) | FGQGTRVDIKRTVAAPSVSIFPPSDEQLKSGTASVV | (SEQ ID NO: 541) |
| C1 | QQYGSS PT | (SEQ ID NO: 245) | FGQGTRVDIKRTVAAPSVFIFPPSDEQLKSGTASV | (SEQ ID NO: 542) |
| C1* | QHYGNS PPYT | (SEQ ID NO: 246) | FGQGTKLEIKRTVAAPSVFIFPP | (SEQ ID NO: 543) |
| C2 | QQYGTS PFT | (SEQ ID NO: 247) | FGPGTKVDIKRTVAAPSVFIFPPS | (SEQ ID NO: 544) |
| C2* | QQYGSS PQT | (SEQ ID NO: 248) | FGQGTKVEIKRTVAAPSVFIFPPSDEQLKSGTASVV | (SEQ ID NO: 545) |
| C3 | QQYGSS PLT | (SEQ ID NO: 249) | FGGGTKVE KRTVAAPSVFIFPPSD | (SEQ ID NO: 546) |
| C4 | QHYGSS SYT | (SEQ ID NO: 250) | FGQGTRVEIKRTVAAPSVFIFPPSDEQLKSGTASVV | (SEQ ID NO: 547) |
| C5 | QQYGSS PQT | (SEQ ID NO: 251) | FGQGTKVEIKRTVAAPSVFIFPPSDEQLKSGTASVV | (SEQ ID NO: 548) |
| C6 | QQYGGSPPYT | (SEQ ID NO: 252) | FGQGTKLEIKRTVAAPSVFIFPPSDEQLKSGTASVV | (SEQ ID NO: 549) |
| D1 | QQYGSS PQT | (SEQ ID NO: 253) | FGQGTKVEIKRTVAAPSVFIFPPSDEQLKSGTASVV | (SEQ ID NO: 550) |
| D2 | QQYGSS PQT | (SEQ ID NO: 254) | FGQGTKVEIKFTVAAPSVFIFPPSDEQLKSGTASVV | (SEQ ID NO: 551) |
| D3 | QQYGSS PQT | (SEQ ID NO: 255) | FGQGTKVEIKRTVAAPSVFIFPPSDEQLKSGTASVV | (SEQ ID NO: 552) |
| D4 | QQYGSS PQT | (SEQ ID NO: 256) | FGQGTKVQIKRTVAAPSVFIFPPSDEQLKSGTASVV | (SEQ ID NO: 553) |
| E | MQGAHW PPT | (SEQ ID NO: 257) | FGGGTKVEINRTVAAPSVFIFPPSDEQLKSGTA | (SEQ ID NO: 554) |
| F | EQLNSF PYT | (SEQ ID NO: 258) | FGQGTKLEIKRTVAAPSVFIFPPSD | (SEQ ID NO: 555) |
| G | MQGLQT PWT | (SEQ ID NO: 259) | FGQGTKVEIKRTVAAPSVFIFPPSD | (SEQ ID NO: 556) |
| H1 | QQYGSS PLT | (SEQ ID NO: 260) | FGGGTKVEIKRTVAAPSVFIFPPSD | (SEQ ID NO: 557) |
| H1* | QQYGSS PQT | (SEQ ID NO: 261) | FGQGTEVEIKRTVAAPSVFIFPPSDEQ | (SEQ ID NO: 558) |
| H1** | QQYGPS PPGYT | (SEQ ID NO: 262) | FGQGTKLEIKRTVAAPSVFIFPPSDEQLKSGTASVV | (SEQ ID NO: 559) |
| H2 | QQYGSS PQT | (SEQ ID NO: 263) | FGQGTKVEIKRTVAAPSVFIFPPSDEQLKSGTASVV | (SEQ ID NO: 560) |
| H3 | QQYRS PLT | (SEQ ID NO: 264) | FGGGTKVEIKRTVAAPSVFIFPPSDEQLKSGTASVV | (SEQ ID NO: 561) |
| I | QQYNKWPPWT | (SEQ ID NO: 265) | FGQGTKLEIKRTVAAPSVFVFPPS | (SEQ ID NO: 562) |
| J1 | QQYGSS PQT | (SEQ ID NO: 266) | FGQGTKVEIKRTVAAPSVFIFPPSDEQLKSGTASVV | (SEQ ID NO: 563) |
| J2 | QQYGSS PQT | (SEQ ID NO: 267) | FGQGTKVEIKRTVAAPSVFIFPPSDEQLKSGTASVV | (SEQ ID NO: 564) |
| J3 | QQYGSS PQT | (SEQ ID NO: 268) | FGQGTKVEIKRTVAAPSVFIFPPSDEQLKSGTASVV | (SEQ ID NO: 565) |
| J3* | QQYGSS PQT | (SEQ ID NO: 269) | FGQGTKVEIKRTVAAPSVFIFPPSDEQLKSGTASVV | (SEQ ID NO: 566) |
| J4 | QQYGSS PQT | (SEQ ID NO: 270) | FGQGTEVEIKRTVAAPSVFIFPPSDEQLKSGTASVV | (SEQ ID NO: 567) |
| K | QQYGGS PPRFT | (SEQ ID NO: 271) | FGPGTKVDIRRTVAAPSVFIFPPSDEQLKSGTASVV | (SEQ ID NO: 568) |
| L1 | QQYGDS VG | (SEQ ID NO: 272) | FGPGTKVEIKRTVAAPSVFIFPPSDEQLKSGTASVV | (SEQ ID NO: 569) |
| L2 | QQYGDS VG | (SEQ ID NO: 272) | FGPGTKVEIKRTVAAPSVFIFPPSDEQLKSGTASVV | (SEQ ID NO: 569) |
| L3 | QQYGSS PLT | (SEQ ID NO: 273) | FGQGTKVEIKRTVAAPSVFIFPPSDEQLKSGTASVV | (SEQ ID NO: 570) |
| L4 | QQYGLS ST | (SEQ ID NO: 274) | FGQGTRLEIKRTVAAPSVFIFPPSDEQLKSGTASVV | (SEQ ID NO: 571) |
| M | HHYGGS PRT | (SEQ ID NO: 275) | FGQGTKVEIKRTVAAPSVFIFPPSDEQLKSGTASVV | (SEQ ID NO: 572) |

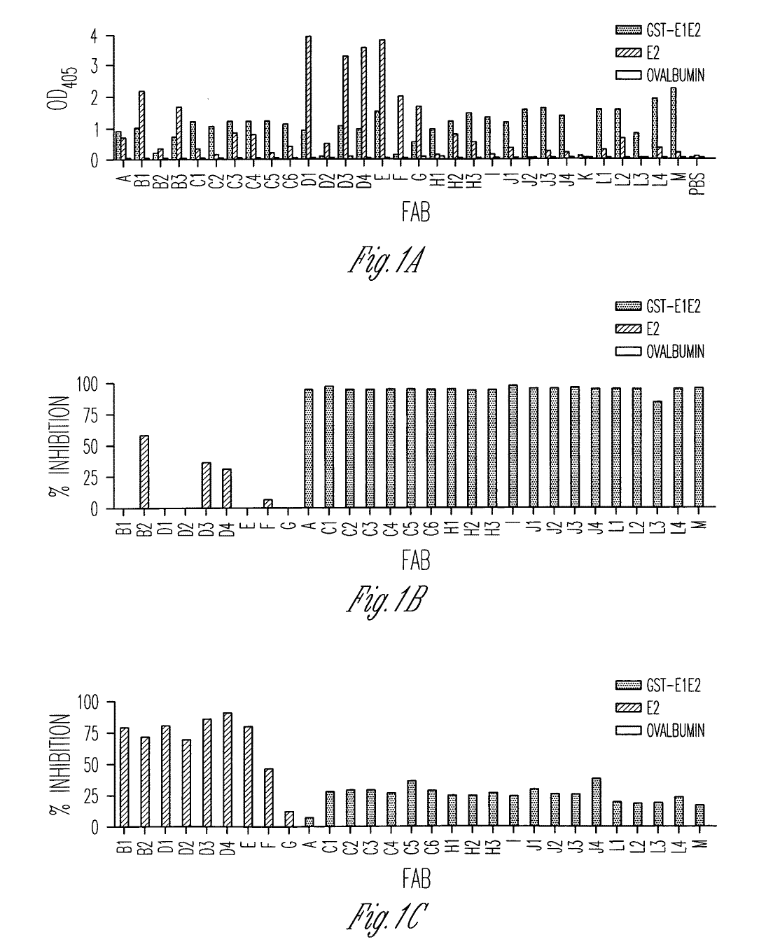

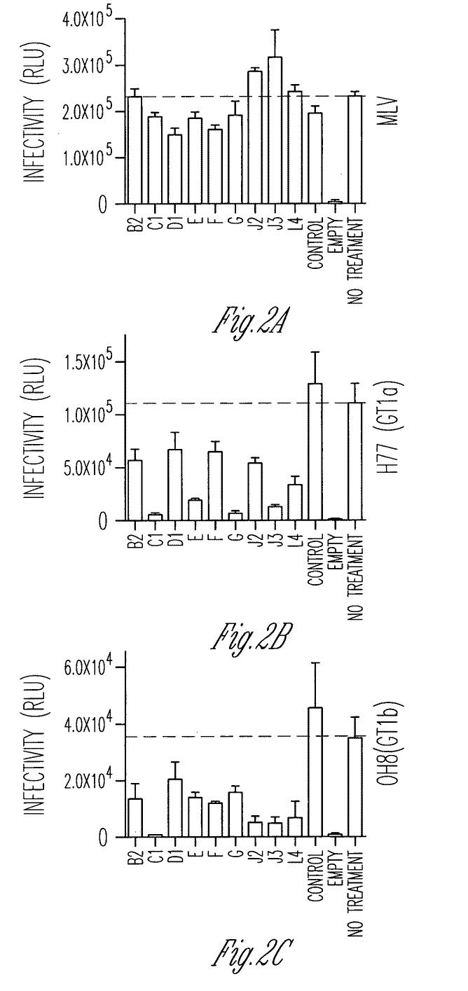

The binding properties of soluble Fabs prepared from the phage-Fab clones were characterized (FIG. 1). This allowed the Fabs to be sorted into three groups recognizing three antigenic regions (AR) of HCV E2 as shown in the table below.

TABLE E-4
Three Distinct Antigenic Regions Defined by the Fab Panel

| | | | Competition | Competition |
|---|---|---|---|---|
| | | Epitope | with MAb | with MAb |
| AR | AR Recognized by Fab | Presentation | AR3A | H53 |
| 1 | B1, B2, B3, D1, D2, D3, | E2 > E1E2 | No/Partial | Strong |
| | D4, E (63%) | | | |
| 2 | F, G (2%) | E2 > E1E2 | No | Partial |
| 3 | A, C1, C2, C3, C4, C5, | E1E2 > E2 | E1E2 > E2 | Partial |
| | C6, H1, H2, H3, I, J1, | | | |
| | J2, J3, J4, L1, L2, | | | |
| | L3, L4, M (35%) | | | |

The numbers in parenthesis denote the percentage of clones recognizing each AR in the phage-display panning. It is important to note that highly isolate-specific antibodies, e.g. those against HVR1, would unlikely be selected in this study due to the use of heterologous antigens in the panning. Fab K was excluded in this table due to its poor signal in FIG. 1.

A total of seven Fabs from different heavy-chain groups recognizing the three different antigenic regions were converted into full-length IgG1s and their binding properties were evaluated (Table E-5). In addition, the neutralizing activities of the mAbs were studied using a panel of HCV pseudotype virus particles (HCVpp) displaying E1 E2 from diverse genotypes (Table E-6). See Wakita et al. Nat. Med. 11: 791-96 (2005); Bartosch et al., J. Exp. Med. 197: 633-42 (2003); Hsu et al., Proc. Natl. Acad. Sci. USA 100: 7271-7276 (2003)).

TABLE E-5
Binding properties of E2-specific IgGs

| | | | | | Apparent | |
|---|---|---|---|---|---|---|
| | | | | | affinity for E1- | |
| | | | | IC50 E1-E2 | E2 (nM)^{d} | |
| | Derived from | | | binding to | (GT1a) | (GT1a) |
| IgG1 | Fab | Specificity | Epitope | CD81-LEL^{a} | HCV-1^{b} | H77^{c} |
| AR1A | B2 | AR1 | Discontinuous | 5.7 | 2.6 | 3.8 |
| AR1B | D1 | AR1 | Discontinuous | — | 0.4 | 0.6 |
| AR2A | G | AR2 | Discontinuous | — | 3.1 | 1.6 |
| AR3A | C1 | AR3 | Discontinuous | 0.5 | 1.3 | 3.7 |
| AR3B | J2 | AR3 | Discontinuous | 1.6 | 2.0 | 6.0 |
| AR3C | H3 | AR3 | Discontinuous | 2.0 | 1.4 | 2.3 |
| AR3D | L4 | AR3 | Discontinuous | 1.0 | 2.4 | 4.0 |

^{a}Antibody concentration (nM) to inhibit 50% of E1-E2 (isolate H77) binding to immobilized recombinant large extracellular loop of CD81 (CD81-LEL).
^{b}Vaccinia-expressed E1-E2.
^{c}E1-E2 produced by transfected 293T cells.
^{d}Apparent affinity is defined as the antibody concentration required to achieve half-maximal binding in an ELISA. Data shown are the means of at least two independent experiments. All mAbs bind natively folded, but not reduced and denatured, E2. GT1a indicates genotype 1a, GT2a indicates genotype 2a and dashes indicate that no significant inhibition or binding was observed with the highest mAb concentration tested.

TABLE E-6
Neutralizing activity (IC50) of E2-specific IgGs

| | HCVpp^{b} | | | | | | | | | |
|---|---|---|---|---|---|---|---|---|---|---|
| | 1a | | 1b | | 2a | | 2b | 4 | 5 | Control |
| IgG^{a} | H77 | H | OH8 | UKN1B12.16 | J6 | UKN2A1.2 | UKN2B1.1 | UKN4.21.16 | UKN5.15.7 | VSV |
| AR1A | — | | — | — | — | — | — | — | — | — |
| AR1B | — | | — | — | — | — | — | — | — | — |
| AR2A | 1 | 5 | — | — | — | 5 | 10 | 1 | 10 | — |
| AR3A | 1 | 1 | 5 | 1 | 10 | 10 | 10 | 1 | 1 | — |
| AR3B | 1 | 1 | 5 | 1 | 10 | 5 | 10 | 1 | 1 | — |
| AR3C | 1 | 1 | 5 | 1 | 10 | 10 | 10 | 1 | 1 | — |
| AR3D | 1 | 1 | 5 | 1 | 50 | 25 | 25 | 1 | 10 | — |

^{a}mAbs at 50, 25, 10, 5 or 1 μg/mL were tested for virus neutralization, and the lowest antibody concentrations that reduced >50% of virus infectivity are shown. Dashes indicate no or <50% virus neutralization with 50 μg/mL mAb. Data shown are the means of at least two experiments.
^{b}Neutralization of HCVpp was determined by the reduction in luciferase activity in Huh-7 cells infected with HCVpp displaying Env from different HCV isolates. The panel of HCVpps shown includes HCV Env proteins that produce a signal at least tenfold higher than the background signal induced by the control pseudotype virus generated without HCV Env cDNA. Many HCV Env proteins, including CH35 (genotype 1b), UKN3A1.28c (genotype 3a), UKN6.5.8 (genotype 6) and 13 different KP Env clones (genotype 1a, see FIG. 7B), did not produce a consistent signal tenfold higher than background and were excluded from this analysis.

The above results indicate that all recombinant mAbs bound the E1-E2 complex from HCV genotype 1a with approximately similar apparent affinities, in the range of 0.4-6 nM, but only antigenic region 3 (AR3)-specific mAbs reacted with genotype 2a HCV, suggesting that epitopes in AR3 are highly conserved. Monoclonal antibodies AR1A and AR3A-D inhibited the binding of E1-E2 to the virus co-receptor CD81 (Pileri et al. Science 282, 938-41 (1998); Cocquerel et al., J. Virol. 77, 10677-83 (2003)) at nanomolar concentrations, suggesting that these antibodies could potentially block HCV interaction with CD81 and thereby inhibit infection.

In addition, these experiments indicate the following.

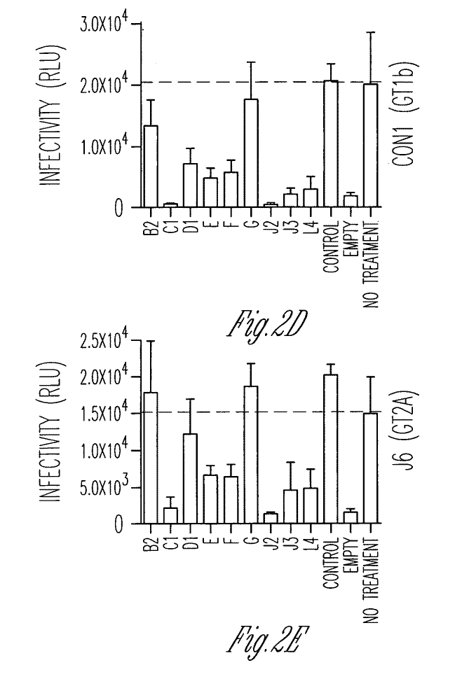

First, antibodies that bind E2 in an ELISA did not necessarily neutralize the corresponding virus. The AR1-specific antibodies bound recombinant E1-E2 from genotype 1a HCV isolate H77 with a similar or higher affinity than AR3-specific antibodies, but they did not neutralize the virus, suggesting that the AR1 epitopes are available on isolated envelope proteins but not on infectious virions. Of note, the Fab fragments of antibodies AR1A and AR1B (that is, B2 and D1, Table E-5) did neutralize HCV pp-H77 (FIG. 2), indicating that steric hindrance, possibly by E1 (FIG. 1A), prevents virus neutralization by whole AR1-specific antibodies.

Second, the ability of the antibodies to inhibit E1-E2 binding to CD81 in the ‘neutralization of binding’ assay (Rosa et al., Proc. Natl. Acad. Sci. USA 93: 1759-63 (1996)) did not fully predict virus neutralization.

Third, and most notably, the AR3-specific antibodies bound E1-E2 from both genotypes 1a and 2a at nanomolar affinities and cross-neutralized many HCVpps tested. These results show that AR3 is a relatively conserved neutralizing site on HCV E2.

The specificity, affinity and neutralizing activities of the E2-specific human monoclonal antibodies were evaluated by mapping the antigenic regions using competition ELISA and alanine-mutagenesis scanning. Results are shown in the following tables.

TABLE E-7
Antibody Competition

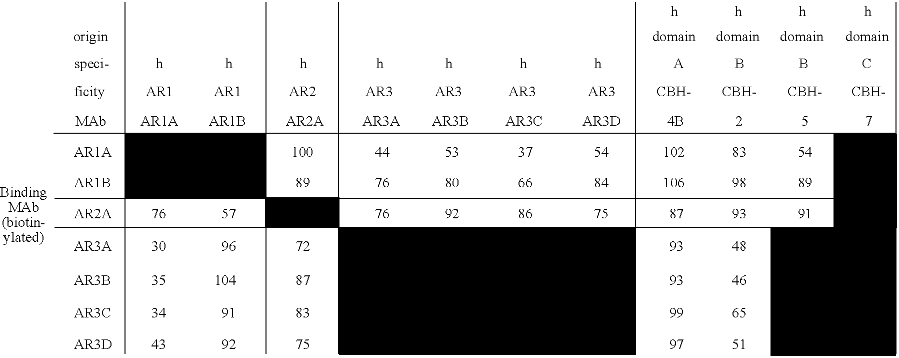

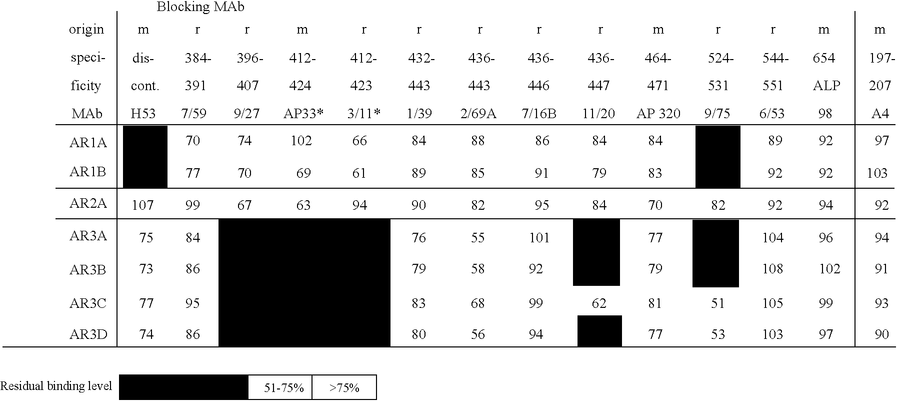

Numbers indicate percentage of residual binding signals of biotinylated human mAbs in the presence of blocking mAbs. Extent of competition is grouped by intensity of shading.
Origin:
h, human;
m, mouse;
r, rat.

TABLE E-8
Alanine-scanning Mutagenesis

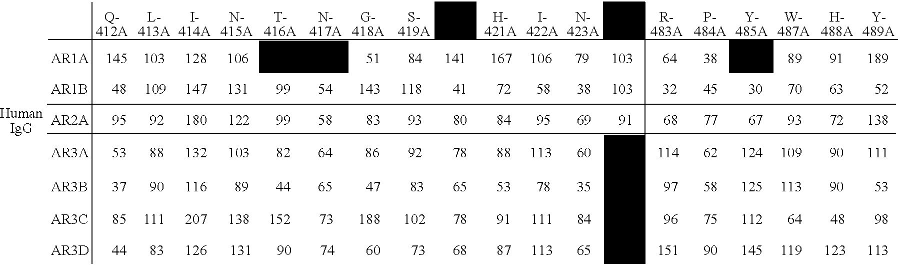

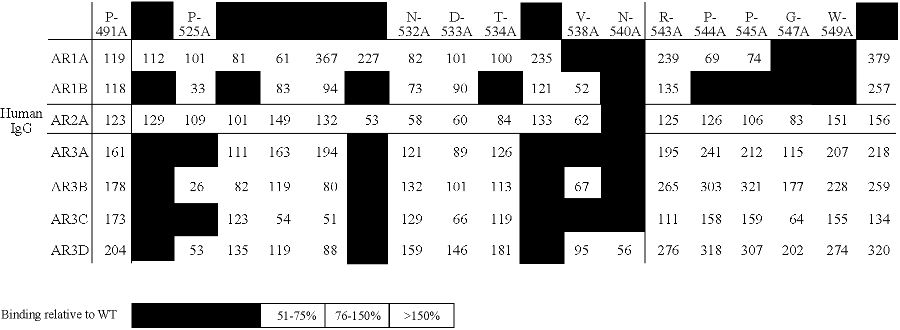

The panel of variants (top row) includes substitutions at conserved residues in the putative CD81-binding regions of E2. Substitutions important for CD81 binding are shaded and include L413A, W420A, H421A, I422A, N423A, S424A, G523A, T526A, Y527A, W529A, G530A, D535A, V538A, N540A and F550A. (Owsianka, A.M. et al. J. Virol. 80, 8695-8704 (2006)).
The enhancement in binding or extend of reduction in binding are indicated by shading.

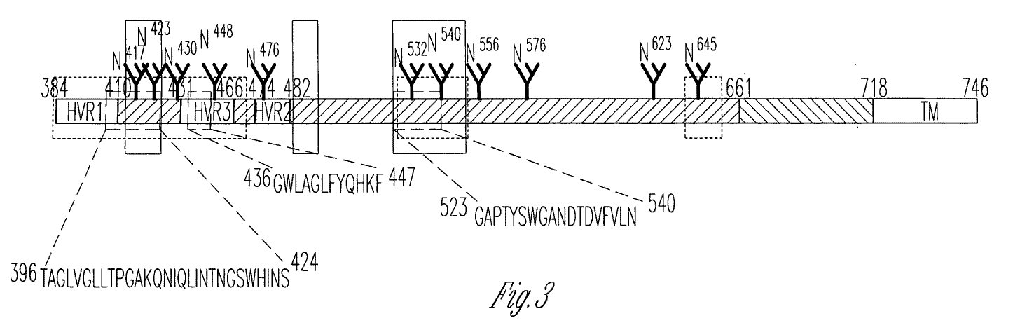

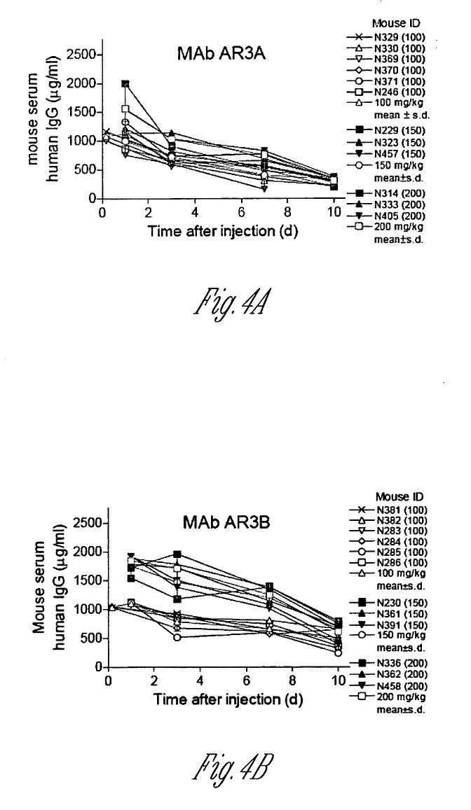

The antibody competition study shows that mAbs AP33 and 3/11 (*) recognize epitopes partially dependent on proper protein folding (Tarr, A. W. et al. Hepatology 43, 592-601 (2006)). The results confirm the broad designation of the antigenic regions and suggest that the discontinuous epitopes in AR3 are formed by at least three segments between amino acids 396-424, 436-447 and 523-540; the first and third segments also contribute to the CD81-binding domain of E2 (Owsianka, A. M. et al. J. Virol. 80, 8695-8704 (2006)), and the conserved residues Ser424, Gly523, Pro525, Gly530, Asp535, Val538 and Asn540 (Owsianka, A. M. et al. J. Virol. 80, 8695-8704 (2006)) are probably involved in the binding of the AR3-specific antibodies (FIG. 3).

A key question is whether broadly neutralizing AR3-specific antibodies can protect against infection by heterologous HCV quasispecies. As a first step to evaluate the mAbs and establish the essential parameters for passive antibody protection, the human liver-chimeric Alb-uPA/SCID mouse model was used (Kneteman, N. M. et al. Hepatology 43, 1346-1353 (2006); Lindenbach, B. D. et al. Proc. Natl. Acad. Sci. USA 103, 3805-3809 (2006)). Although this animal model is not suitable for studying virus pathogenesis, owing to its lack of a functional adaptive immune system, the question of whether antibodies can protect against HCV challenge is appropriate.

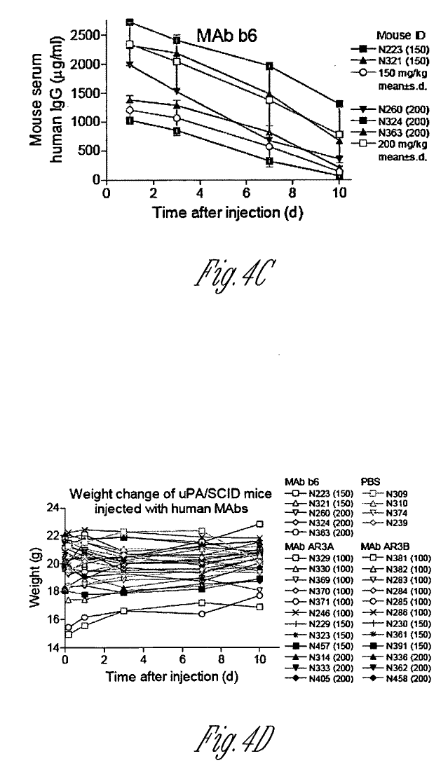

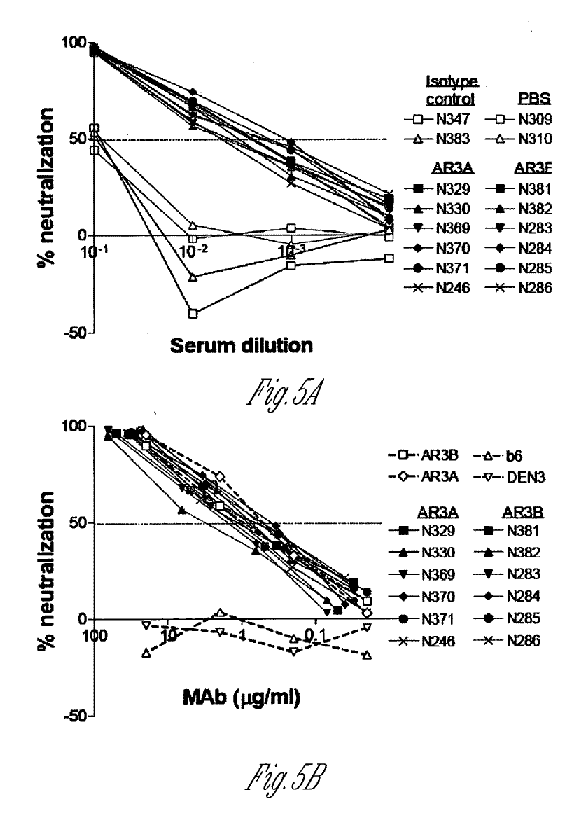

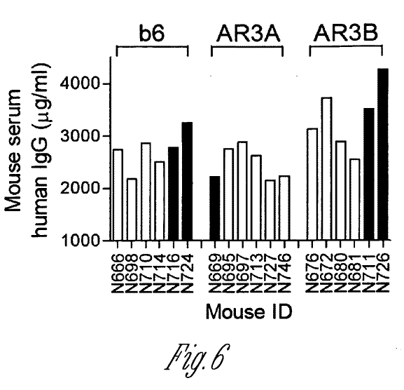

The kinetics and tolerability were first established in the animal model for the antibodies AR3A AR3B and a human isotype control IgG1 to HIV-1, b6. The antibodies did not show adverse effects in control mice, and a dose of 200 mg/kg given through intraperitoneal injection was required to achieve mean serum titers approximately 100× higher than in vitro neutralization titers (FIG. 4). Such titers have previously been found to be necessary to achieve sterilizing immunity in other viral disease models. The observed half-lives of mAbs AR3A, AR3B and b6 were 6.0±2.2 d, 9.0±1.3 d and 7.3±1.8 d (mean±s.d.), respectively, and their specific neutralizing activities (that is, neutralizing activity relative to serum mAb concentration) were stable for at least 10 days in the mice (FIG. 5).

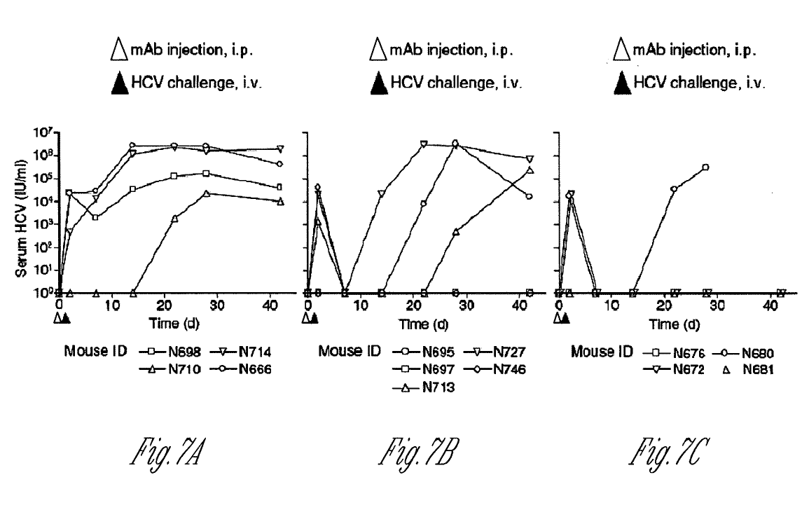

The mAbs were administered intraperitoneally in passive transfer experiments to mice with high levels of human liver chimerism (see Example 1), and the mean serum titers of mAbs AR3A, AR3B and the control mAb b6, at 24 hours after injection were ˜2.5±0.3 mg/mL, 3.1±0.5 mg/mL and 2.6±0.3 mg/mL, respectively (FIG. 6). To simulate a natural human exposure to virus, we inoculated genotype 1a HCV-infected human serum intravenously into the mice. The partial amino acid sequences (residues 384-622) of forty HCVs found in the viral quasispecies population in the HCV genotype 1a-infected human serum are shown below.

TABLE E-9
Cloned Variant Sequences of E2 Amino Acid Residues 384-622

| | |
|---|---|
| Name | Sequence |

| | |
|---|---|
| KP S9 | ETHVTGGATAHGASVLASLLTTGAKQNIQLINTNGSWHINRTALNCNDSLHTGFIAGLFYYNKFNSSGCPERLASCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCVIGGVGNNTLRCPTDCFRKHPDATYSRCGSGPWITPRCLVDYPYRLWHYPCTI |
| | (SEQ ID NO: 701) |
| KP R14 | ETHVTGGATAHGASVLASLLTTGAKQNIQLINTNGSWHINRTALNCNDSLHTGFIAGLFYYNKFNSSGCPERLAGCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCVIGGVGNNTLRCPTDCFRKHPDATYSRCGSGPWITPRCLVDYPYRLWHYPCTI |
| | (SEQ ID NO: 702) |
| KP S6 | ETHVTGGATAHGASVLASLLTTGAKQNIQLINTNGSWHINRTALNCNDSLHTGFIAGLFYYNKFNSSGCPERLASCRRLDD |
| | FAQGWGPISHVNVSGPGERPYCWHYPPRPCGIVPARDVCGPVYCFTPSPVVVGTTDRAGAPSYNWGANETDVFVLNNTRPP |
| | LGNWFGCTWMNSTGFTKVCGAPPCVIGGVGNNTLRCPTDCFRKHPDATYSRCGSGPWITPRCLVDYPYRLWHYPCTI |
| | (SEQ ID NO: 703) |
| KP S18 | ETHVTGGATAHGASVLASLLTTGAKQNIQLINTNGSWHINRTALNCNDSLHTGFIAGLFYYNKFNSSGCPERLASCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMDSTGFTKVCGAPPCVIGGVGNNTLRCPTDCFRKHPDATYSRCGSGPWITPRCLVDYPYRLWHYPCTI |
| | (SEQ ID NO: 704) |
| KP S16 | ETHVTGGATAHGASVLASLLTTGAKQNIQLINTNGSWHINRTALNCNDSLHTGFIAGLFYYNKFNSSGCPERLASCRRLDD |
| | FAQGQGPISHVNVSGPGERPYCWHYPPRPCGIVPARDVCGPVYCFTPSPVVVGTTDRAGATPYNWGANETDVFVLNNTRPP |
| | LGNWFGCTWMNSTGFTKVCGALPCVIGGVGNNTLRCPTDCFRKHPDATYSRCGSGPWITPRCLVDYPYRLWHYPCTI |
| | (SEQ ID NO: 705) |
| KP R8 | ETHVTGGATAHGASVLASLLTTGAKQNIQLINTNGSWHINRTALNCNDSLHTGFIAGLFYYNKFNSSGCPERLASCRRLDD |
| | FAQGQGPISHVNVSGPGERPYCWHYPPRPCGIVPARDVCGPVYCFTPSPVVVGTTDRAGATPYNWGANETDVFVLNNTRPP |
| | LGNWFGCTWMNSTGFTKVCGAPPCVIGGVGSNTLRCPTDCFRKHPDATYSRCGSGPWITPRCLVDYPYRLWHYPCTI |
| | (SEQ ID NO: 706) |
| KP S20 | ETHVTGGATAHGASVLASLLTTGAKQNIQLINTNGSWHVNRTALNCNDSLHTGFIAGLFYYNKFNSSGCPERLASCRRLDD |
| | FAQGWGPISHVNVSGPGERPYCWHYPPRPCGIVPARDVCGPVHCFTPSPVVVGTTDRAGAPTYNWGANETDVFVLNNTRPP |
| | LGNWFGCTWMNSTGFTKVCGAPPCVIGGVGNNTLRCPTDCFRKHPDATYSRCGSGPWITPRCLVDYPYRLWHYPCTI |
| | (SEQ ID NO: 707) |
| KP S4 | ETHVTGGATAHGASVLASLLTTGAKQNIQLINTNGSWHINRTALNCNDSLHTGFIVGLFYYNKFNSSGCPERLASCRRLDD |
| | FAQWGWPISYVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCVIGGVGNNTLRCPTDCFRKHPDATYSRCGSGPWITPRCLVDYPRYLWHYPCTI |
| | (SEQ ID NO: 708) |
| KP R3 | ETHVTGGATAHGASVLASLLTTGAKQNIQLINTNGSWHINTRALNCNDSLHTGFIAGLFYYNKFDSSGCPERLASCRRLDD |
| | FAQGQGPISYVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCVIGGVGNNTLRCPTDVFRKHPDATYSRCGSGPWITPRCLVDYPYRLWHYPCTI |
| | (SEQ ID NO: 709) |
| KP S3 | ETHVTGGATAHGASVLASLLTTGAKQNIQLINTNGSWHINRTALNCNDSLHTGFIAGLFYYNKFDSSGCPERLASCRRLDD |
| | FAQGWGPSIYVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCDIGGVGNNTLRCPTDCFRKHPDATYSRCGSGPWITPRCLVDYPYRLWHYPCTI |
| | (SEQ ID NO: 710) |
| KP S12 | ETHVTGGATAHGASVLASLLTPGAKQHVQLINTNGSWHINRTALNCNDSLHTGFIAGLFYYNKFDSSGCPERLASCRRLDD |
| | FAQGWGPSIYVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCVIGGVGNNTLRCPTDCFRKHPDATYSRCGSGPWITPRCLVDYPRYLWHYPCTI |
| | (SEQ ID NO: 711) |
| KP S15 | ETHVTGGATAHGASVLASLLTPGAKQNIQLINTNGSWHINRTALNCNDSLHTGFVAGLFYYNKFDSSGCPERLASCRRLDD |
| | FAQGQGPISHVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCVIGGVGNNTLRCPTDCFRKHPDATYSRCGSGPWITPRCLVDYPRYLWHYPCTI |
| | (SEQ ID NO: 712) |
| KP S5 | ETHVTGGATAHGASVLASLLTPGAKQNIQLINTNGSWHINRTALNCNDSLHTGFIAGLFYYNKFDSSGCLERLASCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCAIGGVGNNTLRCPTDCFRKHPDATYSRCGSGPWITPRCLVDYPRYLWHYPCTI |
| | (SEQ ID NO: 713) |
| KP R7 | ETHVTGGATAHGASVLASLLTPGAKQNIQLINTNGSWHINRTALNCNDSLHTGFIAGLFYYNKFNSSGCPERLASCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCAIGGVGNNTLRCPTDCFRKHPDATYSRCGSGPWITPRCLVDYPYRLWHYPCTI |
| | (SEQ ID NO: 714) |
| KP R11 | ETHVTGGATAHGASVLASLLTPGAKQNIQLINTNGSWHINRTALNCNDSLHTGFIAGLFYYNKFNSSGCPERLASCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMNTTGFTKVCGAPSCVIGGVGNNTLRCPTDCFRKHPDATYSRCGSGPWITPRCLVDYPYRLWHYPCTI |
| | (SEQ ID NO: 715) |
| KP R1 | RTHVTGGATAHGASVLASLLTPGAKQNIQLINTNGSWHINRTALNCNDSLHTGFIAGLFYYNKFNSSGCPERLASCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMNTTGFTKVCGAPPCVIGGVGNNTLRCPTDCFRKHPDATYSRCGSGPWITPRCLVDYPYRLWHYPCTI |
| | (SEQ ID NO: 716) |
| KP R12 | ETHVTGGATAHGASVLASLLTPGAKQNIQLINTNGSWHINRTALNCNDSLHTGFIAGLFYYNKFNSSGCPERLASCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCVIGGVGNNTLRCPTDCFRKHPDATYSRCGSGPWITPRCLVDYPRYLWHYPCTI |
| | (SEQ ID NO: 717) |
| KP S7 | ETHVTGGATAHGASVLASLLTPGAKQNIQLINTNGSWHINRTALNCNDSLHTGFIAGLFYYNKFNSSGCPERLASCRRLDD |
| | FAQGWGPSIHVNVSGPGERPYCWHYPPRPCGICPARDVCGPVYCFTPSPVVVGTTDRAGAPTYNWGANETDVFVLNNTRPP |
| | LGNWFGCTWMNSTGFTKVCGAPPCVIGGVGNNTLRCPTDCFRKHPDATYSRCGSGPCITPRCLVDYPYRLWHYPCTI |
| | (SEQ ID NO: 718) |
| KP R15 | ETHVTGGATAHGASVLTSLLTTGAKQNIQLINTNGSWHINRTALNCDNSLHTGFIAGLFYYNKFNSSGCPERLASCRRLDD |
| | FAQGWGPISHVNVSGPGERPYCWHYPPRPCGIVPARDVCGPVYCFTPSPVVVGTTDRAGAPTYNWGANETIDFVLNNTRPP |
| | LGNWFGCTWMNSTGFTKVCGAPPCVIGGVGNNTLRCPTDCFRKHPDATYSRCGSGWPWIPRCLVDYPYRLWHYPCTI |
| | (SEQ ID NO: 719) |
| KP R18 | ETYVTGGATAHGASVLASLLTTGAKQNIQLINTNGSWHINRTALNCNDSLHTGFIAGLFYYNKFNSSGCPERLASCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCVIGGVGNNTLRCPTDCFRKHPDATYSRCGSGPWITPRCLVDYPRYLWHYPCIT |
| | (SEQ ID NO: 720) |
| KP S11 | ETHVTGGATAHGASVFASLLTTGAKQNIQLINTNGSWHINRTALNCNDSLHTGFIAGLFYYNKFNSSGCPERLASCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCVIGGVGNNTLRCPRDCFRKHPDATYSRCGSGPWITPRCLVDYPYRLWHYPCTI |
| | (SEQ ID NO: 721) |
| KP R20 | ETHVTGGATAHGASVFASLLTTGAKQNIQLINTNGSWHINRTALNCNDSLHTGFIAGLFYYNKFDSSGCPERLASCRRLDD |
| | FAQGWGPISHVNVSGPGERPYCWHYPPRPCGIVPARDVCGPVYCFTPSPVVVGTTDRAGAPTYNWGANETDVFVLNNTRPP |
| | LGNWFGCTWMNSTGFTKVCGAPPCVIGGVGNNTLRCPTDCFRKHPDATYSRCGSGPWITPRCLVDYPYRLWHYPCTI |
| | (SEQ ID NO: 722) |
| H77 | ETHVTGGSAGRTTAGLVGLLTPGAKQNIQLINTNGSWHINSTALNCNESLNTGWLAGLFYQHKFNSSGCPERLASCRRLTD |
| | FAQGWGPISYANGSGLDERPYCWHYPPRPCGIVPAKSVCGPVYCFTPSPVVVGTTDRSGAPTYSWGANDTDVFVLNNTRPP |
| | LGNWFGCTWMNSTGFTKVCGAPPCVIGGVGNNTLLCPTDCFRKHPEATYSRCGSGPWITPRCMVDYPYRLWHYPCTI |
| | (SEQ ID NO: 723) |
| UKN1b12.16 | RTRTTGGSAAQTTYGLTSLFRSGPSQKIQLVNTNGSWHINRTALNCNDSLNTGFLAALFYVRNFNSSGCPERMASCRPIDT |
| | FDQGWGPITYTEPHSLDQRPYCWHYAPQPCGIVPAAQVCGPVYCFTPSPVVVGTTDRFGAPTYTWGENETDVLILNNTRPP |
| | QGNWFGCTWMNGTGFTKTCGGPPCNIGGAGNNTLICPTDCFRKHPEATYTRCGSGPWLTPRCMVDYPYRLWHYPCTV |
| | (SEQ ID NO: 724) |

The alignment of these sequences are shown in FIG. 7B. Infection was monitored by assessing serum viral load up to 6 weeks after inoculation (FIG. 7A). Protection in this mouse model is defined as the absence of serum HCV RNA as detected by quantitative PCR at or after 6 days post virus challenge. All mice in the control group (n=4) were infected, and serum viral load was maintained at >10,000 RNA copies/mL until the completion of the study. In mice that received mAb AR3A (n=5) or AR3B (n=4), HCV was detected the day after challenge in five of nine mice, but was cleared 6 days after virus challenge. High levels of HCV RNA were detected in four mice between weeks 2 and 4, indicating virus replication concurrent with the decay of antibody in these mice. By week 6, when the mAbs would have decayed to <10% of the initial serum level (FIG. 5), two of five mice receiving mAb AR3A and three of four mice receiving mAb AR3B were still protected. The protection was highly significant compared to the isotype control antibody group (two-tailed log-rank test: AR3A, P=0.0298; AR3B, P=0.0171). The experiments ended at week 6 because two mice became morbid and were killed on day 41 and day 45, respectively, but the remaining mice were monitored to week 8, and a signal below the sensitivity of the quantitative PCR assay (6.0×10² international units/mL) was noted in one additional mouse in each neutralizing antibody-treated group (mice N681 and N697).

In summary, (i) it is possible to use mAbs against AR3 to protect against challenge with a heterologous HCV quasispecies swarm, consistent with the notion that AR3 is the principal conserved neutralizing antibody determinant of HCV; (ii) high concentrations of the mAbs were required for protection, suggesting that more potent antibody preparations will likely be required in immunotherapy, but that the mAbs described will be useful for comparative in vitro studies with newly identified mAbs and combinations of mAbs; and (iii) considering that one-third of the 115 phage-Fab clones isolated in this study are AR3 specific and are diverse in their heavy-chain sequences (FIGS. 1, 2 and Table E-4), and similar mAbs were isolated from different HCV-infected donors elsewhere (Table E-7) (Keck, Z. Y. et al. J. Virol. 78, 9224-9232 (2004)), AR3 seems to be relatively immunogenic in humans and thus a favorable target for vaccine design. So, despite the enormous diversity of HCV, the prospects for developing a vaccine against this virus, that may target both conserved B and T cell epitopes (Elmowalid, G. A. et al. Proc. Natl. Acad. Sci. USA 104, 8427-8432 (2007); Folgori, A. et al. Nat. Med. 12, 190-197 (2006)), seem favorable.

### Example 3

### Generation and Characterization of HCVE2 Mutants

The HCV E2 glycoprotein is a major target for virus neutralizing antibodies and an important component in a HCV vaccine. E2 has encoded several features to evade antibodies. First, E2 encodes regions that are highly mutable. Rapid changes in viral sequence facilitate virus escape. Second, E2 is highly glycosylated and the associated glycans help shield the neutralizing epitopes from antibodies. Despite these escape features, we have identified the antigenic region 3 (AR3) on E2 as a relatively conserved target for antibody neutralization in vitro and antibody protection in vivo. The amino acid residues important for the binding of AR3-specific antibodies is described above. The following show how these residues organize together to form the AR3 conformational epitopes.

To identify a form of E2 that displays AR3 properly while silencing some of the variable sequences that are usually immunogenic but are not targets of broadly neutralizing antibodies, a panel of E2 truncation mutants was constructed. To identify the minimal E2 fragment that displays the CD81-binding sites and the broadly neutralizing epitopes correctly, the binding of these E2 mutants with CDE81-LEL or various mAb were studied.

Construction of Expression DNA Plasmids of E2 Mutants

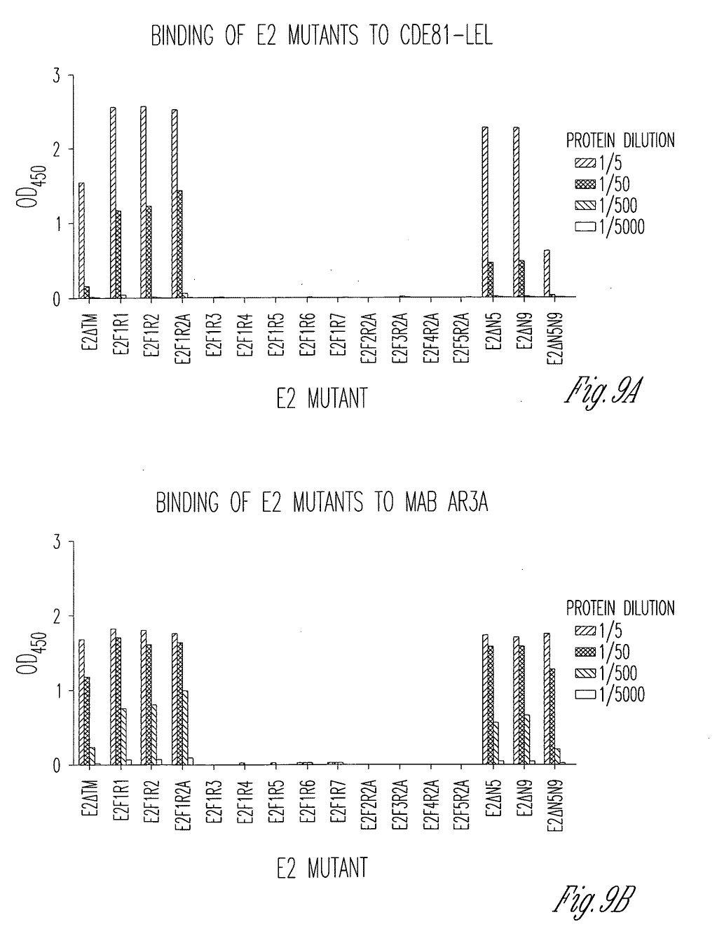

The E2 mutants were constructed by deletion of highly variable regions, specific N-glycosylation signals, or every other cysteine residues from C— or N-terminus of wildtype (WT) E2. The panel of E2 mutants in fusion with the Flag tags at their C-terminii is illustrated in FIG. 8, and their sequences are shown in Table E12.

TABLE E-12
Hepatitis C Virus E2 Glycoprotein Mutants

| E2 & Mutants | | | C-terminal |
|---|---|---|---|
| (also named) | Signal Peptide | E2 Mutant Sequence | Flag Tag |
| E2ΔTM | MDAMKRGLCCVLLLCGAVFVSPSQEI | ETHVTGGNAGRTTAGLVGLLTPGAKQNIGLINTNGSWHINSTALNC | LEDYKDDDDK |
| | HARFRRGAR | NESLNTGWLAGLFYQHKFNSSGCPERLASCRRLTDFAQGWGPISYA | (SEQ ID NO: 726) |
| | (SEQ ID NO: 725) | NGSGLDERPYCWHYPPRPCGIVPAKSVCGPVYCFTPSPVVVGTTDR | |
| | | SGAPTYSWGANDTDVFVLNNTRPPLGNWFGCTWMNSTGFTKVCGAP | |
| | | PCVIGGVGNNTLLCPTDCFRKHPEATYSRCGSGPWITPRCMVDYPY | |
| | | RLWHYPCTINYTIFKVRMYVGGVEHRLEAACNWTRGERCDLEDRDR | |
| | | SELSPLLLSTTQWQVLPCSFTTLPALSTGLIHLHQNIVDVQYLYGV | |
| | | GSSIASWAIKWE | |
| | | (SEQ ID NO: 727) | |
| E2₄₁₂₋₆₆₁ | MDAMKRGLCCVLLLCFAGFVSPSQEI | QLINTNGSWHINSTALNCNESLNTGWLAGLFYQHKFNSSGCPERLA | LEDYKDDDDK |
| (E2f1r1) | HARFRRGAR | SCRRLTDFAQGWGPISYANGSGLDERPYCWHYPPRPCGIVPAKSVC | (SEQ ID NO: 726) |
| | (SEQ ID NO: 725) | GPVYCFTPSPVVVGTTDRSGAPTYSWGANDTDVFVLNNTRPPGWN | |
| | | FGCTWMNSTGFTKVCGAPPCVIGGVGNNTLLCPTDCFRKHPEATYS | |
| | | RCGSGPWITPRCMVDYPYRLWHYPCTINYTIFKVRMYVGGVEHRLE | |
| | | AACNWTRGERCDLEDRDRSE | |
| | | (SEQ ID NO: 728) | |
| E2₄₁₂₋₆₄₇ | MDAMKRGLCCVLLLCGAVFVSPSQEI | QLINTNGSWHINSTALNCNESLNTGWLAGLFYQHKFNSSGCPERLA | LEDYKDDDDK |
| (E2f1r2) | HARFRRGAR | SCRRLTDFAQWGWPISYANGSGLDERPYCWHYPPRPCGIVPAKSVC | (SEQ ID NO: 726) |
| | (SEQ ID NO: 725) | GPVYCFTPSPVVVGTTDRSGAPTYSWGANDTDVFVLNNTRPPLGNW | |
| | | FGCTWMNSTGFTKVCGAPPCVIGGVGNNTLLCPTDCFRKHPEATYS | |
| | | RCGSGPWITPRCMVDYPYRLWHYPCTINYTIFKVRMYVGGVEHRLE | |
| | | AACNWTR | |
| | | (SEQ ID NO: 729) | |
| E2₄₁₂₋₆₄₅ | MDAMKRGLCCVLLLCGAVFVSPSQEI | QLINTNGSWHINSTALNCNESLNTGWLAGLFYQHKFNSSGCPERLA | LEDYKDDDDK |
| (E2f1r2a) | HARFRRGAR | SCRRLTDFAQGWGPISYANGSGLDERPYCWHYPPRPCGIVPAKSVC | (SEQ ID NO: 726) |
| | (SEQ ID NO: 725) | GPVYCFTPSPVVVGTTDRSGAPTYSWGANDTDVFVLNNTRPPLGWN | |
| | | FGCTWMNSTGFTKVCGAPPCVIGGVGNNTLLCPTDCFRKHPEATYS | |
| | | RCGSGPWITPRCMVDYPYRLWHYPCTINYTIFKVRMYVGGVEHRLE | |
| | | AACN | |
| | | (SEQ ID NO: 730) | |
| E2₄₁₂₋₆₁₁ | MDAMKRGLCCVLLLCGAVFVSPSQEI | QLINTNGSWHINSTALNCNESLNTGWLAGLFYQHKFNSSGCPERLA | LEDYKDDDDK |
| (E2f1r3) | HARFRRGAR | SCRRLTDFAQGWGPISYANGSGLDERPYCWHYPPRPCGIVPAKSVC | (SEQ ID NO: 726) |
| | (SEQ ID NO: 725) | GPVYCFTPSPVVVGTTDRSGAPTYSWGANTDTDVFVLNNTRPPLGNW | |
| | | FGCTWMNSTGFTKVCGAPPCVIGGVGNNTLLCPTDCFRKHPEATYS | |
| | | RCGSGPWITPRCMVDY | |
| | | (SEQ ID NO: 731) | |
| E2₄₁₂₋₅₈₉ | MDAMKRGLCCVLLLCGAVFVSPSQEI | QLINTNGSWHINSTALNCNESLNTGWLAGLFYQHKFNSSGCPERLA | LEDYKDDDDK |
| (E2f1r4) | HARFRRGAR | SCRRLTDFAQGWGPISYANGSGLDERPYCWHYPPRPCGIVPAKSVC | (SEQ ID NO: 726) |
| | (SEQ ID NO: 725) | GPVYCFTPSPVVVGTTDRSGAPTYSWGANDTDVFVLNNTRPPLGNW | |
| | | FGCTWMNSTGFTKVCGAPPCVIGGVGNNTLLCPTDCFRKH | |
| | | (SEQ ID NO: 732) | |
| E2₄₁₂₋₅₇₄ | MDAMKRGLCCVLLLCGAVFVSPSQEI | QLINTNGSWHINSTALNCNESLNTWGLAGLFYQHKFNSSGCPERLA | LEDYKDDDDK |
| (E2f1r5) | HARFRRGAR | SCRRLTDFAQGWGPISYANGSGLDERPYCWHYPPRPCGIVPAKSVC | (SEQ ID NO: 726) |
| | (SEQ ID NO: 725) | GPVYCFTPSPVVVGTTDRSGAPTYSWGANDTDVFVLNNTRPPLGNW | |
| | | FGCTWMNSTGFTKVCGAPPCVIGGV | |
| | | (SEQ ID NO: 733) | |
| E2₄₁₂₋₅₅₇ | MDAMKRGLCCVLLLCGAVFVSPSQEI | QLINTNGSWHINSTALNCNESLNTWGLAGLFYQHKFNSSGCPERLA | LEDYKDDDDK |
| (E2f1r6) | HARFRRGAR | SCRLLTDFAQGWGPISYANGSGLDERPYCWHYPPRPCGIVPAKSVC | (SEQ ID NO: 726) |
| | (SEQ ID NO: 725) | GPVYCFTPSPVVVGTTDRSGAPTYSWGANDTDVFVLNNTRPPLGNW | |
| | | FGCTTWMNS | |
| | | (SEQ ID NO: 734) | |
| E2₄₁₂₋₅₀₅ | DMAMKRGLCCVLLLCGAVFVPSPQEI | QLINTNGSWHINSTALNCNESLNTGWLAGLFYQHKFNSSGCPERLA | LEDYKDDDDK |
| (E2f1r7) | HARFRRGAR | SCRRLTDFAQGWGPISYANGSGLDERPYCWHYPPRPCGIVPAKSVC | (SEQ ID NO: 726) |
| | (SEQ ID NO: 725) | GP | |
| | | (SEQ ID NO: 735) | |
| E2₄₅₆₋₆₄₅ | MDAMKRGLCCVLLLCGAVFVSPSQEI | LASCRRLTDFAQGWGPISYANGSGLDERPYCWHYPPRPCGIVPAKS | LEDYKDDDDK |
| (E2f2r2a) | HARFRRGAR | VCGPVYCFTPSPVVVGTTDRSGAPTYSWGANDTDVFVLNNTRPPLG | (SEQ ID NO: 726) |
| | (SEQ ID NO: 725) | NWFGCTWMNSTGFTKVCGAPPCVIGGVGNNTLLCPTDCFRKHPEAT | |
| | | YSRCGSGPWITPRCMVDYPYRLWHYPCTINYTIFKVRMYVGGVEHR | |
| | | LEAACN | |
| | | (SEQ ID NO: 736) | |
| E2₄₉₂₋₆₄₅ | MDAMKRGLCCVLLLCGAVFVSPSQEI | RPCGIVPAKSVCGPVYCFTPSPVVVGTTDRSGAPTYSWGANDTDVF | LEDYKDDDDK |
| (E2f3r2a) | HARFRRGAR | VLNNTRPPLGNWFGCTWMNSTGFTKVCGAPPCVIGGVGNNTLLCPT | (SEQ ID NO: 726) |
| | (SEQ ID NO: 725) | DCFRKHPEATYSRCGSGPWITPRCMVDYPYRLWHYPCTINYTIFKV | |
| | | RMYVGGVEHRLEAACN | |
| | | (SEQ ID NO: 737) | |
| E2₅₀₆₋₆₄₅ | MDAMKRGLCCVLLLCGAVFVSPSQEI | VYCFTPSPVVVGTTDRSGAPTYSWGANTDVFVLNNTRPPLGNWFG | LEDYKDDDDK |
| (E2f4r2a) | HARFRRGAR | CTWMNSTGFTKVCGAPPCVIGGVGNNTLLCPTDCFRKHPEATYSRC | (SEQ ID NO: 726) |
| | (SEQ ID NO: 725) | GSGPWITPRCMVDYPYRLWHYPCTINYTIFKVRMYVGGVEHRLEAA | |
| | | CN | |
| | | (SEQ ID NO: 738) | |
| E2₅₅₈₋₆₄₅ | MDAMKRGLCCVLLLCGAVFVSPSQEI | TGFTKVCGAPPCVIGGVGNNTLLCPTDCFRKHPEATYSRCGSGPWI | LEDYKDDDDK |
| (E2f5r2a) | HARFRRGAR | TPRCMVDYPYRLWHYPCTINYTIFKVRMYVGGVEHRLEAACN | (SEQ ID NO: 726) |
| | (SEQ ID NO: 725) | (SEQ ID NO: 739) | |
| E2ΔN5 | MDAMKRGLCCVLLLCGAVFVSPSQEI | QLINTNGSWHINSTALNCNESLNTGWLAGLFYQHKFNSSGCPERLA | LEDYKDDDDK |
| | HARFRRGAR | SCGSSGCWHYPPRPCGIVPAKSVCGPVYCFTPSPVVVGTTDRSGAP | (SEQ ID NO: 726) |
| | (SEQ ID NO: 725) | TYSWGANDTDVFVLNNTRPPLGNWFGCTWMNSTGFTKVCGAPPCVI | |
| | | GGVGNNTLLCPTDCFRKHPEATYSRCGSGPWITPRCMVDYPYRLWH | |
| | | YPCTINYTIFKVRMYVGGVEHRLEAACN | |
| | | (SEQ ID NO: 740) | |
| E2ΔN9 | MDAMKRGLCCVLLLCGAVFVSPSQEI | QLINTNGSWHINSTALNCNESLNTGWLAGLFYQHKFNSSGCPERLA | LEDYKDDDDK |
| | HARFRRGAR | SCRRLTDFAQGWGPISYANGSGLDERPYCWHYPPRPCGIVPAKSVC | (SEQ ID NO: 726) |
| | (SEQ ID NO: 725) | GPVYCFTPSPVVVGTTDRSGAPTYSWGANDTDVFVLNNTRPPLGNW | |
| | | FGCTWMNSTGFTKVCGAPPCGSSGCPTDCFRKHPEATYSRCGSGPW | |
| | | ITPRCMVDYPYRLWHYPCTINYTIFKVRMYVGGVEHRLEAACN | |
| | | (SEQ ID NO: 741) | |
| E2ΔN5N9 | MDAMKRGLCCVLLLCGAVGVSPSQEI | QLINTNGSWHINSTALNCNESLNTGWLAGLFYQHKFNSSGCPERLA | LEDYKDDDDK |
| | HARFRRGAR | SCGSSGCWHYPPRPCGIVPAKSVCGPVYCFTPSPVVVGTTDRSGAP | (SEQ ID NO: 726) |
| | (SEQ ID NO: 725) | TYSWGANDTDVFVLNNTRPPLGNWFGCTWMNSTGFTKVCGAPPCGS | |
| | | SGCPTDCFRKHPEATYSRCGSGPWITPRCMVDYPYRLWHYPCTINY | |
| | | TIFKVRMYVGGVEHRLEAACN | |
| | | (SEQ ID NO: 742) | |

| | |
|---|---|
| E2₃₈₄₋₇₄₆ | ETHVTGGSAGRTTAGLVGLLTPGAKQNIQLINTNGSWHINSTALNCNESLNTGWLAGLFYQHKFNSSGCPERLASCRRLTDFAQGWGPIS |
| | YANGSGLDERPYCWHYPPRPCGIVPAKSVCGPVYCFTSPVVVGTTDRSGAPTYSWGANDTDVFVLNNTRPPLGNWFGCTWMNSTGFTKVC |
| | GAPPCVIGGVGNNTLLCPTDCFRKHPEATYSRCGSGPWITPRCMVDYPYRLWHYPCTINYTIFKVRMYVGGVEHLEAACNWTRGERCDLE |
| | DRDRSELSPLLLSTTQWQVLPCSFTTLPALSTGLIHLHQNIVDVQYLYGVGSSIASWAIKWEYVVLLFLLLADARVCSCLWMMLLISQAE |
| | A |
| | (SEQ ID NO: 743) |

The cDNA encoding these mutants were generated by polymerase chain reaction (PCR) or by splicing by overlap extension polymerase chain reaction (SOE-PCR) as described in Horton et al., Biotechniques 8:528-535 (1990). In the reaction, the plasmid pCV-H77c (Genbank accession# AF011751) encoding wildtype E2 gene of the isolate H77 was used as a template. The primers used in the reactions are enlisted below.

TABLE E-13
Primers for cloning E2 mutants

| | SEQ | |
|---|---|---|
| Primer | ID NO: | Sequence (from 5′-to-3′) |
| E2wtF | 744 | AATAACGCGTGAAACCCACGTCACCGG |
| E2f1F | 745 | AATAACGCGTCAACTGATCAACACCAACG |
| E2f2F | 746 | AATAACGCGTTTGGCCAGCTGCCGACGC |
| E2f3F | 747 | AATAACGCGTAGACCTTGTGGCATTGTGC |
| E2f4F | 748 | AATAACGCGTGTATATTGCTTCACTCCCAG |
| E2f5F | 749 | AATAACGCGTACTGGATTCACCAAAGTGTG |
| E2wtR | 750 | TATTCTCGAGCTCCCACTTAATGGCCCAG |
| E2r1 | 751 | TATTCTCGAGCTCGGACCTGTCCCTGTC |
| E2r2 | 752 | TATTCTCGAGCCGCGTCCAGTTGCAGGC |
| E2r2a | 753 | TATTCTCGAGGTTGCAGGCCGCTTCCAGC |
| E2r3 | 754 | TATTCTCGAGGTAGTCGACCATGCACCTG |
| E2r4 | 755 | TATTCTCGAGATGTTTGCGGAAGCAATCAG |
| E2r5 | 756 | TATTCTCGAGCACCCCTCCGATGACACAAG |
| E2r6 | 757 | TATTCTCGAGTGAGTTCATCCAGGTACAAC |
| E2r7 | 758 | TATTCTCGAGCGGGCCACACACGCTCTTTG |
| delHVR2F | 759 | TGCGGCTCTAGCGGATGCTGGCACTACCCTCCAAG |
| delHVR2R | 760 | CAGCATCCGCTAGAGCCGCAGCTGGCCAACCTCTC |
| delHVR3F | 761 | TGTGGAAGCTCTGGCTGCCCCACTGATTGCTTCC |
| delHVR3R | 762 | GCAGCCAGAGCTTCCACAAGGGGGCGCTCCGCAC |

The experimental conditions for generating the E2 mutant genes are shown below.

TABLE E-14
Generation of E2 mutants by PCR and SOE-PCR

| PCR | Template | Forward primer | Reverse primer | Product |
|---|---|---|---|---|
| 1 | pCV-H77c | E2wtF | E2wtR | E2ΔTM |
| 2 | pCV-H77c | E2f1F | E2r1 | E2(412-661) |
| 3 | pCV-H77c | E2f1F | E2r2 | E2(412-647) |
| 4 | pCV-H77c | E2f1F | E2r2a | E2(412-645) |
| 5 | pCV-H77c | E2f1F | E2r3 | E2(412-611) |
| 6 | pCV-H77c | E2f1F | E2r4 | E2(412-589) |
| 7 | pCV-H77c | E2f1F | E2r5 | E2(412-574) |
| 8 | pCV-H77c | E2f1F | E2r6 | E2(412-557) |
| 9 | pCV-H77c | E2f1F | E2r7 | E2(412-505) |
| 10 | pCV-H77c | E2f2F | E2r2a | E2(456-645) |
| 11 | pCV-H77c | E2f3F | E2r2a | E2(492-645) |
| 12 | pCV-H77c | E2f4F | E2r2a | E2(506-645) |
| 13 | pCV-H77c | E2f5F | E2r2a | E2(558-645) |
| 14 | pCV-H77c | E2f1F | delHVR2R | product#14 |
| 15 | pCV-H77c | delHVR2F | E2r2a | product#15 |
| 16 | product#14 and | E2f1F | E2r2a | E2ΔN5 |
| | product#15 | | | |
| 17 | pCV-H77c | E2f1F | delHVR3R | product#17 |
| 18 | pCV-H77c | delHVR3F | E2r2a | product#18 |
| 19 | product#17 and | E2f1F | E2r2a | E2ΔN9 |
| | product#18 | | | |
| 20 | E2ΔN5 | E2f1F | delHVR3R | product#20 |
| 21 | product#17 and | E2f1F | E2r2a | E2ΔN5N9 |
| | product#20 | | | |

PCR conditions: 94° C., 3 min; 25 cycles of (94° C., 30 s; 55° C., 30 s; 70° C., 90 s); & 70° C., 10 min
PCR system: Platinum Pfx DNA polymerase (Invitrogen)
PCR instrument: GeneAmp PCR System 9700 (Applied Biosystems)

The PCR products generated in Table E-14 were resolved by agarose gel electroporesis and the DNA bands of correct size were excised and purified. The products were either used as templates in a second PCR, or were digested with Mlu I and Xho I restriction enzymes. The digested products were gel-purified and inserted between the BssH II and Xho I sites of the plasmid pCMV-Tag4A-tpaJR-FLgp120 (Pantophlet et al., J Virol 77:642-658 (2003); Law et al., J Virol 81:4272-4285 (2007). The inserted products replaced the HIV genes in the plasmid and are in frame with a 5′-signal peptide and a 3′-FLAG tag to facilitate protein secretion and for detection. The nucleotide sequences of the E2 mutants were verified by DNA sequencing.

Expression of E2 Mutants

The E2 mutants were expressed by transient transfection of 293T cells. Cell monolayers were co-transfected with the expression plasmids encoding the different E2 mutants and pAdVAntage plasmid (Promega) at 1:1 ratio by polyethylenimine (Boussif et al., Proc Natl Acad Sci USA 92:7297-7301 (1995). Cell supernatants were collected 3 days post-transfection and were clarified by centrifugation.

To identify E2 mutants correctly presenting the different conformation-dependent epitopes, a panel of monoclonal antibodies (MAbs) or the HCV co-receptor CD81 was used to capture the mutants in a capture ELISA. MAb AR1A, AR1B, AR2A, AR3A, AR3B, AR3C, AR3D or maltose binding protein fused-large extracellular loop of CD81 (CD81-LEL) Chan-Fook et al., Virology 273:60-66 (2000) at 5 μg/mL were coated onto ELISA microwells overnight at 4° C. After the microwells were blocked with 4% non-fat milk (Bio-Rad) and 0.05% Tween 20 in PBS, serially diluted transfected cell supernatants from above were added to the microwells for 1 hour at room temperature.

Mutants with correctly folded antibody epitopes or CD81-binding sites were captured by the corresponding reagents and the captured mutants were detected with a mouse anti-FLAG tag MAb (Sigma), followed by a secondary antibody (Peroxidase-conjugated AffiniPure Goat Anti-mouse IgG from Jackson ImmunoResearch Laboratories) and the colorimetic peroxidase substrate TMB (Pierce). The peroxidase reaction was stopped by adding sulfuric acid.

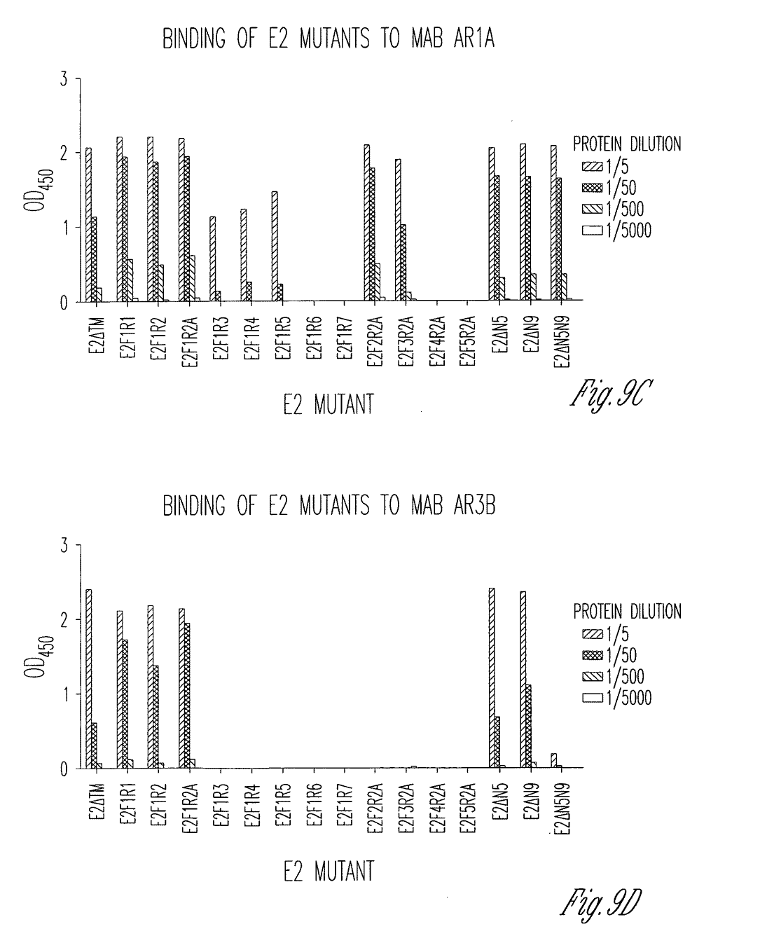

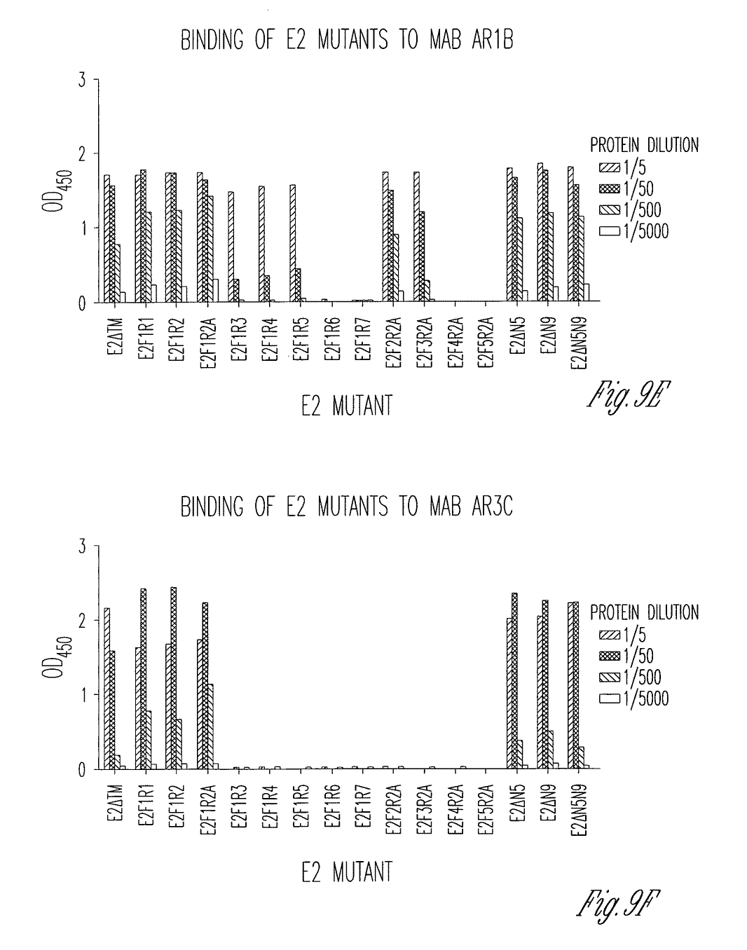

Specific binding of the E2 mutants to the capturing reagents were detected by measuring the absorbances of the samples at 450 nm using a microplate reader (Molecular Devices). The results are summarized in FIG. 9. The CD81-binding sites and AR3 are presented well on the E2 mutants E2ΔTM, E2f1r1, E2f1r2, E2f1r2a, E2ΔN5 and E2ΔN9. The mutant E2ΔN5N9 was captured by MAbs AR3A or AR3C at a comparable level to the above mutants but at a much reduced level by CD81-LEL, MAbs AR3B or AR3D. In contrast, the mutants E2f1r3, E2f1r4, E2f1r5, E2f2r2a and E2f3captured by the non-neutralizing MAbs AR1A and AR1B but not CD81-LEL or AR3-specific MAbs, suggesting that the CD81-binding sites and the broadly neutralizing epitopes in AR3 are not present or folded correctly in these mutants.

The fact that fragments E2f1r1 and E2f1r2a bind to the conformation-dependent, broadly neutralizing MAb AR3A and CD81-LEL indicates that the E2 residues 412-645 and cysteines 1-16 are important for correct folding of AR3 (within this region, residues 460-485 and 570-580 are not required). Of note, E2ΔTM binds all Abs recognizing AR1, 2 and 3, but weakly to CD81-LEL.

### Example 4

### Purification of E2 Mutants

Generally, the HCV envelope E1 and E2 glycoproteins are technically challenging to produce as E1 does not fold properly in the absence of E2 (Michalak et al., J Gen Virol 78:2299-2306 (1997) and Patel et al., Virology 279:58-68 (2001)) and efficient production of E2 is influenced by E1 (Cocquerel et al., J Virol 77:10677-10683 (2003), Brazzoli et al., Virology 332:438-453 (2005)). A truncated version of E2 (known as E2661) can be expressed independently and retained its function in binding to the co-receptor CD81 (Michalak et al., J Gen Virol 78:2299-2306 (1997); Flint et al., J Virol 73:6235-6244 (1999); Flint et al., J Virol 74:702-709 (2000)). However, this truncated E2 has not been shown to be produced in a highly purified form suitable for biochemical analysis and crystallization attempts (Flint et al., J Virol 74:702-709 (2000)).

To purify E2 displaying corrected folded AR3 epitopes, a protein production and purification method was developed. The plasmids encoding the E2 mutants pE2ΔTM and pE2f1r2a were co-transfected with pAdVAntage plasmid (Promega) at 1:1 ratio into FreeStyle 293 cells (Invitrogen) using 293fectin Transfection Reagent (Invitrogen). Cell supernatants were collected twice at 3-day and 5-day post-transfection. If necessary, kifunensine (at 7.5 μM, Cayman Chemical) (Elbein et al., J Biol Chem 265:15599-15605 (1990); Chang et al., Structure 15:267-273 (2007)) was added to cell culture media to improve glycan homogeneity on E2. The E2 mutants were purified by antibody affinity chromatography. To purify correctly folded E2 mutants, the MAb AR3A, which can distinguish folded from misfolded protein, was used. The MAb AR3A recognizes a conformation-dependent epitope on E2, neutralizes HCV in vitro and offers protection against HCV infection in vivo as shown above. It also binds natively folded E2 at high affinity but not denatured and reduced E2.

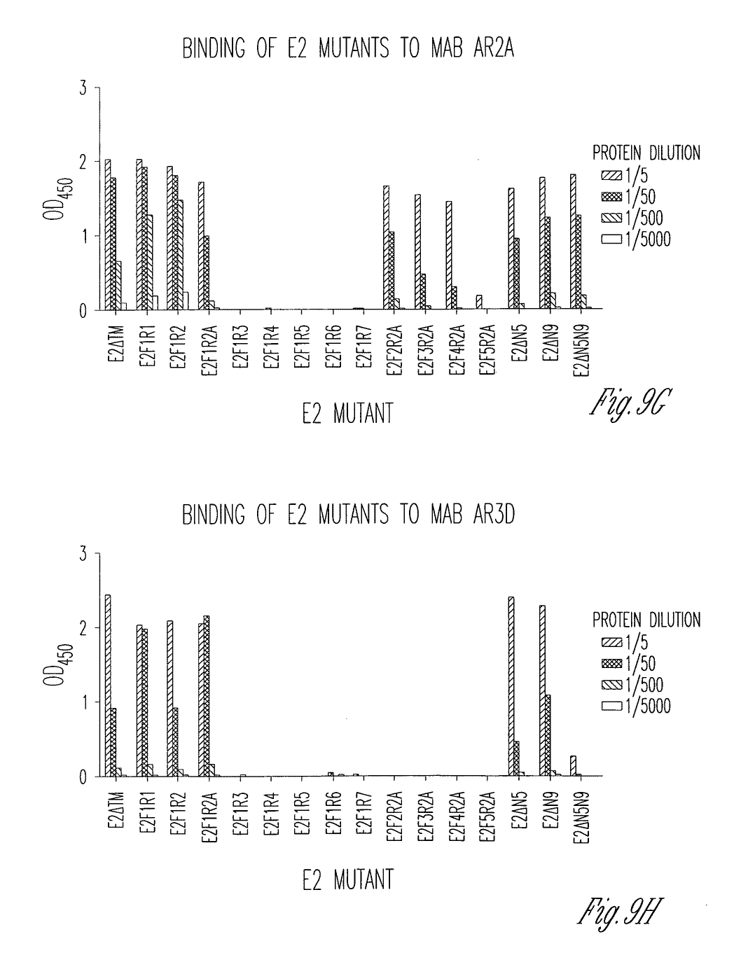

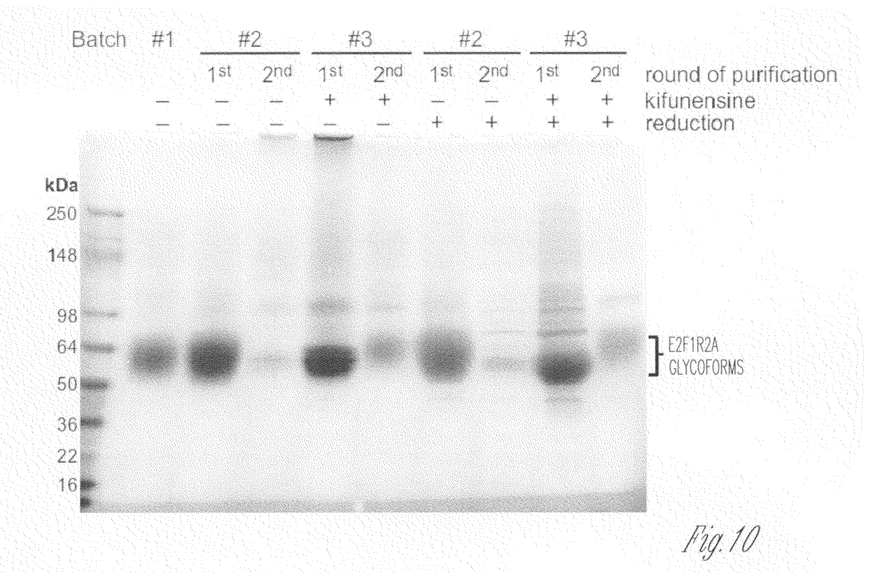

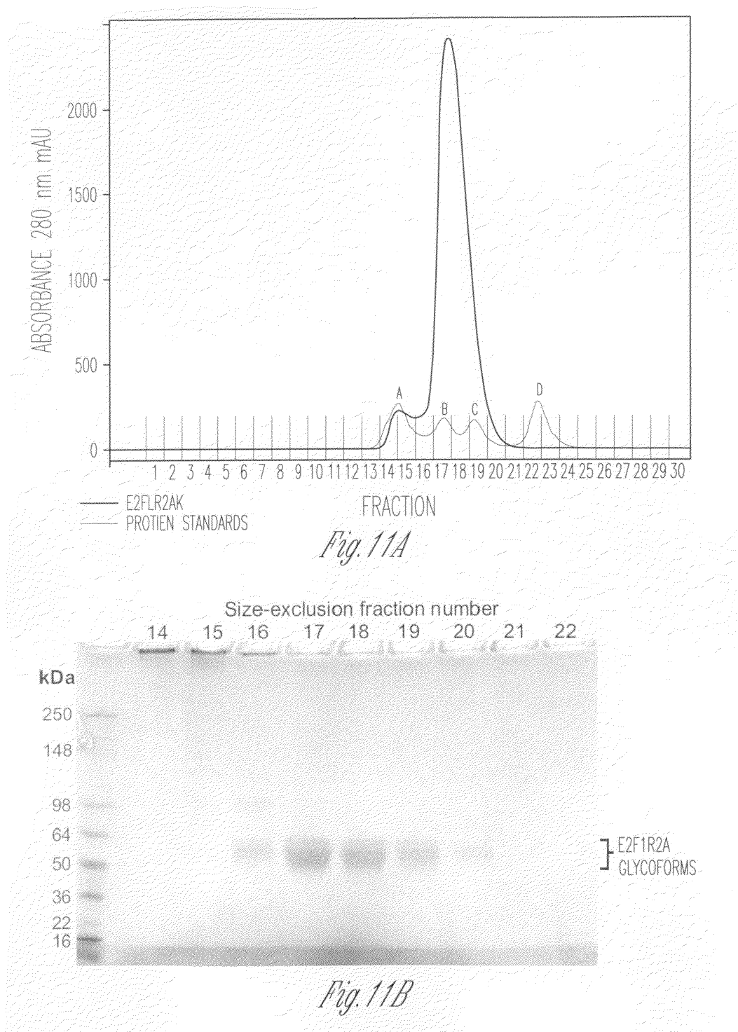

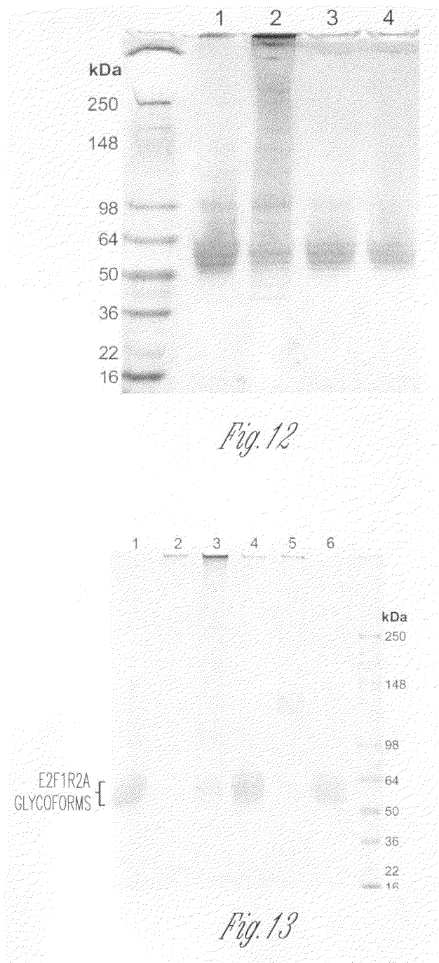

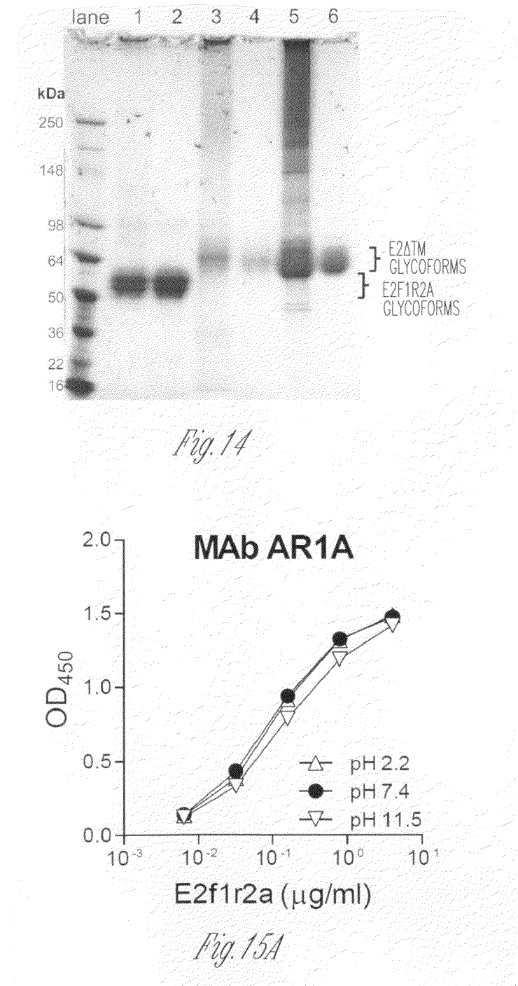

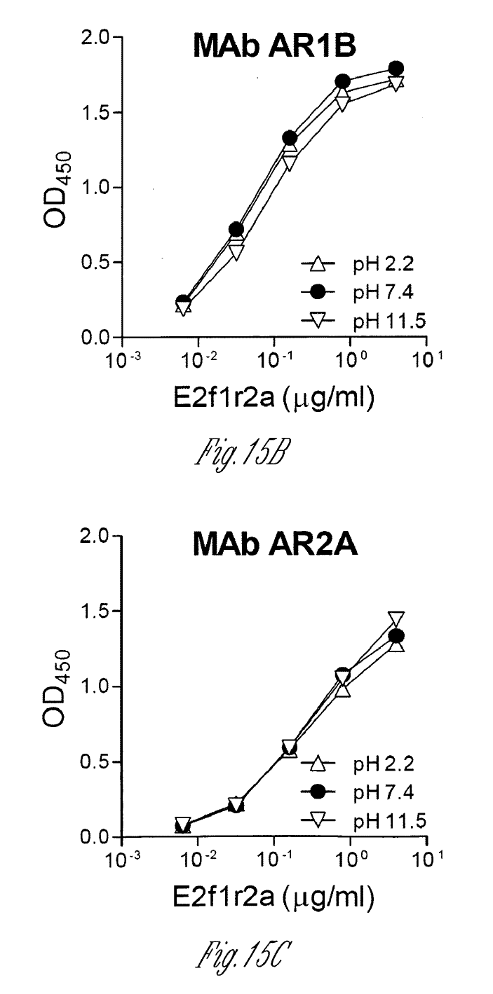

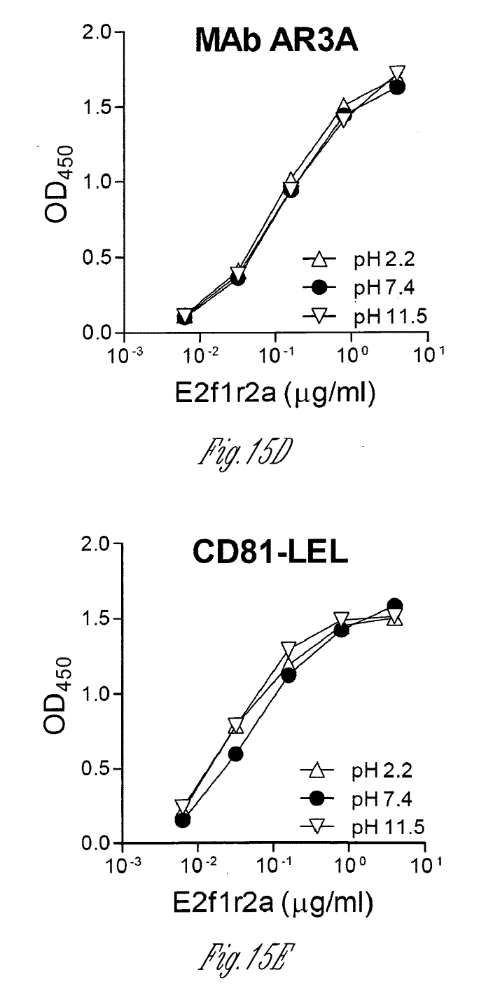

To prepare conjugated MAb AR3A-affinity matrix, MAb AR3A was first captured by Protein A-Sepharose (GE Healthcare) at a ratio of 10 mg MAb per mL Sepharose beads. After overnight incubation, the beads were washed 3 times with 0.2 M sodium borate buffer (pH 9). MAb AR3A was then crosslinked chemically to the Protein A-beads using dimethyl pimelimidate (Thermo Scientific). The reaction was stopped after 1 hour incubation at room temperature by pelleting the beads and washing the beads 3 times with 0.2 M ethanolamine (pH 8). The MAb-conjugated beads were packed into an Econo-Column (Bio-Rad) and the beads were rinsed once with 0.2 M glycine (pH 2.2) followed by PBS to equilibrate the column for affinity purification of the E2 mutants. Cell supernatants containing the E2 mutants were clarified by low-speed centrifugation and filtration through a 0.22-μm filter before loading onto the affinity columns by gravity flow. The flow-throughs were collected and the columns were washed with PBS. Bound proteins were released from the affinity columns using different elution conditions and the antigenicity of the eluted proteins were investigated (see below). The eluants were concentrated and monomers of the E2 mutants were purified by size-exclusion chromatography using a Superdex 75 column (Amersham Biosciences). The purified proteins were evaluated by SDS-PAGE (FIGS. 10-14) and quantified by the Bradford method (Bradford et al., Anal Biochem 72:248-254 (1976)) (Quick Start Bradford Dye Reagent, BioRad) or optical density reading at 280 nm based on calculated extinction coefficients listed in Table E-15.

TABLE E-15
Biochemical properties of E2 mutants

| | | | | | | 1 absorb- |
|---|---|---|---|---|---|---|
| | | | | | | ance |
| | | | | | | (280 nm) |
| | | | Molecular | | Molar | corrected |
| | | Length | weight | | extinction | to |
| | E2 mutants | (residues) | (Da) | pI | coefficient | (mg/mL) |
| 1 | E2ΔTM | 344 | 38020 | 6.79 | 95330 | 0.4 |
| 2 | E2(412-661) | 260 | 29124 | 6.63 | 75580 | 0.39 |
| 3 | E2(412-647) | 247 | 27563 | 7.91 | 75460 | 0.37 |
| 4 | E2(412-645) | 244 | 27119 | 7.56 | 69770 | 0.39 |
| 5 | E2(412-611) | 210 | 23036 | 6.44 | 58720 | 0.39 |
| 6 | E2(412-589) | 188 | 20565 | 6.44 | 50230 | 0.41 |
| 7 | E2(412-574) | 173 | 18864 | 5.85 | 49990 | 0.38 |
| 8 | E2(412-557) | 156 | 17276 | 5.47 | 49750 | 0.35 |
| 9 | E2(412-505) | 104 | 11594 | 5.83 | 29880 | 0.39 |
| 10 | E2(456-645) | 200 | 22213 | 7.57 | 56870 | 0.39 |
| 11 | E2(492-645) | 164 | 18146 | 7.6 | 41410 | 0.44 |
| 12 | E2(506-645) | 151 | 16891 | 6.3 | 41170 | 0.41 |
| 13 | E2(558-645) | 99 | 11210 | 6.96 | 21300 | 0.53 |
| 14 | E2ΔN5 | 222 | 24499 | 7.56 | 61520 | 0.4 |
| 15 | E2ΔN9 | 237 | 26369 | 7.56 | 69770 | 0.38 |
| 16 | E2ΔN5N9 | 215 | 23749 | 7.56 | 61520 | 0.39 |

Note:
The properties were calculated using VectorNTI software (version 10, Invitrogen). Signal peptides and post-translational modifications of the mutants were excluded in the calculation

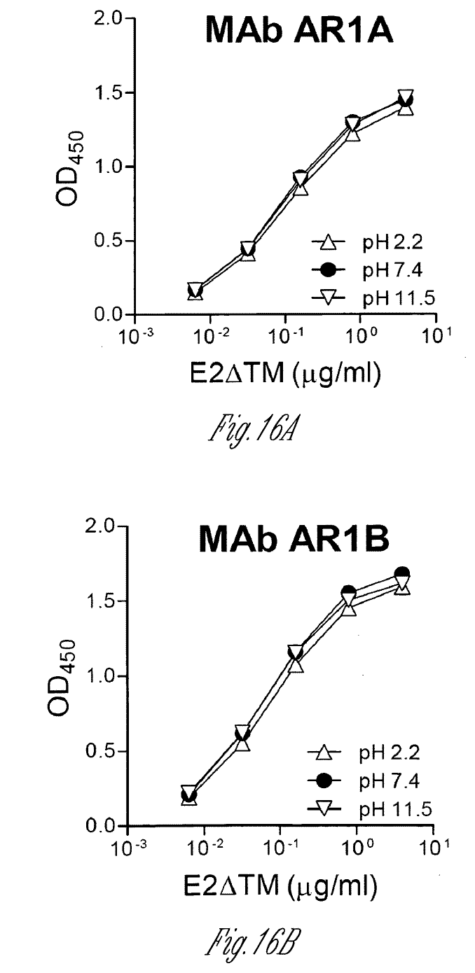

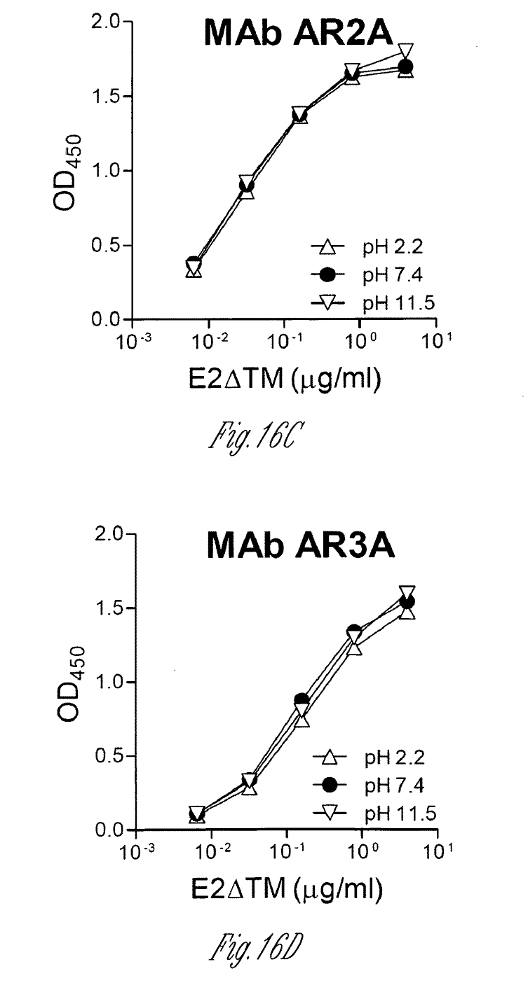

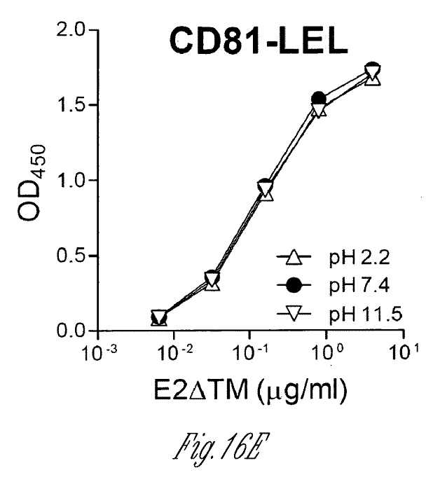

Three protein elution conditions, 0.2M glycine pH 2.2, 2M sodium thiocyanate (pH adjusted to pH 7.4 with 50 mM Tris-HCl) and 0.2M glycine pH 11.5, were examined, and the purified proteins were found to be essentially the same under the different conditions (FIGS. 15-16).

The recombinant E2 fragment E2f1r2a can be purified to greater than 90% by a single affinity chromatography step. In addition, the purification method is applicable to E2f1r2a produced in the presence of the plant alkaloid kifunensine, a potent inhibitor of the glycoprotein processing Δ-mannosidase I. N-glycans on recombinant proteins produced in the presence of kifunensine are almost exclusively high-mannose type oligosaccharides, which can be readily trimmed by endoglycosidase H digestion to improve protein homogeneity.

Overall, the results show that the E2 mutants E2ΔTM and E2f1r2a can be purified as monomers. The recombinant E2 fragments purified by the above method adopt a native fold as found on viral surface. The purified E2 mutants will be extremely useful in research and discovery of anti-viral drugs and HCV vaccines.

### Cited Documents

1. Barbas III, C. F., Burton, D. R., Scott, J. K. & Silverman, G. J. Phage Display: A Laboratory Manual, (Cold Spring Harbor Laboratory Press, New York, 2001).
2. Bartosch, B., Dubuisson, J. & Cosset, F. L. J. Exp. Med. 197, 633-642 (2003).
3. Boom, R. et al. J. Clin. Microbiol. 28, 495-503 (1990).
4. Boussif O, et al. Proc Natl Acad Sci USA, 92, 7297-7301 (1995).
5. Bradford M M. Anal Biochem, 72, 248-254 (1976).
6. Brazzoli M, et al., Virology, 332, 438-453 (2005).
7. Burton, D. R. et al. Science 266, 1024-1027 (1994).
8. Chan-Fook C, et al. Virology, 273, 60-66 (2000).
9. Clayton, R. F. et al. J. Virol. 76, 7672-7682 (2002).
10. Cocquerel et al. J Virol, 77, 10677-10683 (2003).
11. Cocquerel, L., Meunier, J. C., Pillez, A., Wychowski, C. & Dubuisson, J. J. Virol. 72, 2183-2191 (1998).
12. Connor, R. I., Chen, B. K., Choe, S. & Landau, N. R. Virology 206, 935-944 (1995).
13. Cooper, S. et al Immunity 10, 439-449 (1999).
14. Deleersnyder, V. et al. J. Virol. 71, 697-704 (1997).
15. Dubuisson, J. et al. J. Virol. 68, 6147-6160 (1994).
16. Elbein A D, et al. Structure, 15, 267-273 (2007).
17. Elmowalid, G. A. et al. Proc. Natl. Acad. Sci. USA 104, 8427-8432 (2007).
18. Flint M, et al. J Virol, 2000, 74, 702-709.
19. Flint M, et al. J Virol, 73, 6235-6244. (1999).
20. Folgori, A. et al. Nat. Med. 12, 190-197 (2006).
21. He, J. et al. J. Virol. 69, 6705-6711 (1995).
22. Horton R M, et al. Biotechniques, 8, 528-535 (1990).
23. Hsu, M. et al. Proc. Natl. Acad. Sci. USA 100, 7271-7276 (2003).
24. Keck Z Y, et al. J. Virol, 81, 1043-1047 (2007).
25. Keck Z Y, et al., J Virol, 78, 9224-9232 (2004).
26. Keck Z Y, et al., J Virol, 79, 13199-13208 (2005).
27. Kneteman, N. M. et al. Hepatology 43, 1346-1353 (2006).
28. Lavillette, D. et al. Hepatology 41, 265-274 (2005).
29. Law M, et al. J Virol, 81, 4272-4285 (2007).
30. Law M, et al. Nat Med, 14, 25-27 (2008).
31. Lesniewski, R. et al. J. Med. Virol. 45, 415-422 (1995).
32. Lindenbach, B. D. et al. Proc. Natl. Acad. Sci. USA 103, 3805-3809 (2006).
33. Maruyama, T. et al. Am. J. Pathol. 165, 53-61 (2004).
34. Maruyama, T. et al. J. Infect. Dis. 179 1 Suppl 1, S235-239 (1999).
35. McKeating, J. A. et al. J. Virol. 78, 8496-8505 (2004).
36. Mercer, D. F. et al. Nat. Med. 7, 927-933 (2001).
37. Michalak J P, et al. J Gen Virol, 78, 2299-2306 (1997).
38. Motulsky, H. Survival curves. in GraphPad Prism4 Statistics Guide: Statistical analyses for laboratory and clinical researchers 107-117 (GraphPad Software, San Diego, 2005).
39. Mukhopadhyay S, et al. Nat Rev Microbiol, 3, 13-22 (2005).
40. Owsianka, A. et al. J. Virol. 79, 11095-11104 (2005).
41. Owsianka, A. M. et al. J. Virol. 80, 8695-8704 (2006).
42. Pantophlet R, et al. J Virol, 77, 642-658 (2003).
43. Patel J, et al. Virology, 279, 58-68 (2001).
44. Pileri, P. et al. Science 282, 938-941 (1998).
45. Rosa, D. et al. Proc. Natl. Acad. Sci. USA 93, 1759-1763 (1996).
46. Selby, M. et al. J. Immunol. 162, 669-676 (1999).
47. Tarr, A. W. et al. Hepatology 43, 592-601 (2006).
48. Troesch, M. et al. Virology 352, 357-367 (2006).
49. Voisset C, et al. Biol Cell, 96, 413-420 (2004).
50. Wakita, T. et al. Nat. Med. 11, 791-796 (2005).
51. Yagnik A T, et al. Proteins, 40, 355-366 (2000).
52. Zhong, J. et al. Proc. Natl. Acad. Sci. U.S.A. 102, 9294-9299 (2005).

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the statements. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the statements. As used herein and in the appended statements, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as statemented. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended statements.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

What is claimed is:

1. A modified hepatitis C viral E2 polypeptide, the discontinuous epitopes of which comprise, from the amino to the carboxy termini: (1) an amino acid segment, the sequence of which corresponds to amino acid residues 396 to 424 of a select hepatitis C virus, (2) an amino acid segment, the sequence of which corresponds to amino acid residues 436 to 447 of the select hepatitis C virus, and (3) an amino acid segment, the sequence of which corresponds to amino acid 523 to 540 of the select hepatitis C virus; wherein the polypeptide comprises two or more amino acid substitutions at positions 416, 417, 483, 484, 485, 538, 540, 544, 545, 547, 549 or any combinations thereof, and a deletion of amino acid residues 384 to 395 relative to the full-length E2 polypeptide of the select hepatitis C virus.

2. The polypeptide of claim 1, wherein the first amino acid segment has the sequence of any one of SEQ ID NOs: 791-815; the second amino acid segment has the sequence of any one of SEQ ID NOs: 815-840 and the third amino acid segment has the sequence of any one of SEQ ID NOs: 841-865.

3. The polypeptide of claim 1, wherein the first amino acid segment is TAGLVGLLTPGAKQNIQLINTNGSWHINS (SEQ ID NO: 694), the second amino acid segment is GWLAGLFYQHKF (SEQ ID NO: 695) and the third amino acid segment is GAPTYSWGANDTDVFVLN (SEQ ID NO: 696).

4. The polypeptide of claim 1, wherein the first and second segments are separated by about 10 amino acid residues.

5. The polypeptide of claim 1 wherein the second and third segments are separated by about 50 amino acid residues.

6. The polypeptide of claim 1, wherein the first and second segments are separated by about 10 amino acid residues and the second and third segments are separated by about 50 amino acid residues.

7. The polypeptide of claim 1 that comprises the sequence of SEQ ID NO: 866, 867, 868, 869 or 870.

8. The polypeptide of claim 1, the sequence of which consists of SEQ ID NO: 866, 867, 868, 869 or 870.

9. The polypeptide of claim 1, the sequence of which comprises a segment defined by (a) amino acids 396 to 746 of a hepatitis C virus; (b) amino acids 396 to 717 of a hepatitis C virus; (c) amino acids 396 to 661 of a hepatitis C virus; (d) amino acids 396 to 647 of a hepatitis C virus or (e) amino acids 396 to 645 of a hepatitis C virus.

10. The polypeptide of statement 1, further comprising an amino or carboxy terminal tag.

11. The polypeptide of claim 10, wherein the tag is an N-terminal ubiquitin signal, a poly-histidine sequence, a FLAG sequence, an HA sequence, a myc sequence, a V5 sequence, a chitin binding protein sequence, a maltose binding protein sequence or a glutathione-S-transferase sequence.

12. An isolated nucleic acid that encodes the polypeptide of claim 1.

13. The isolated nucleic acid of claim 12 that comprises a sequence encoding a polypeptide of SEQ ID NO: 866, 867, 868, 869 or 870.

14. The isolated nucleic acid of claim 12, the sequence of which comprises SEQ ID NO: 874, 875, 876, 877, 878, 879, 880 or 881.

15. The isolated nucleic acid of claim 12 operably linked to an expression control sequence.

16. The isolated nucleic acid of claim 12, wherein the expression control sequence is a viral, phage, bacterial, or mammalian promoter.

17. An expression vector that comprises the nucleic acid of claim 12.

18. The expression vector of claim 17, wherein the nucleic acid encoding the polypeptide is operably linked to an expression control sequence.

19. The expression vector of claim 18, wherein the expression control sequence is a promoter.

20. The expression vector of claim 19, wherein the promoter is a viral promoter.

21. The expression vector of claim 19, wherein the promoter is a bacterial promoter.

22. The expression vector of claim 19, wherein the promoter is a mammalian promoter.

23. A cell comprising the expression vector of claim 17.

24. The cell of claim 23 that is a bacterial cell.

25. The cell of claim 23 that is a mammalian cell.

26. The cell of claim 23 that is a Chinese hamster ovary cell.

27. A method of eliciting an immune response in a mammal comprising administering to the mammal the polypeptide of claim 1.

28. The method of claim 27, wherein the polypeptide is in a pharmaceutical composition that comprises a pharmaceutically acceptable carrier.

29. The method of claim 27, wherein the mammal is a mouse, sheep, goat, horse, rabbit, hamster, rat or human.

30. The method of claim 27, further comprising obtaining a blood sample from the mammal.

31. The method of claim 27, further comprising isolating an antibody or antibody-producing cell from the mammal.

32. The method of claim 31, wherein the antibody is a cross-neutralizing antibody.

33. The method of claim 27, wherein the polypeptide is in an amount effective to prevent or treat hepatitis C viral infection in the mammal.

34. The method of claim 27, further comprising administering to the mammal a second dose of the polypeptide at a selected time after the first administration.

35. The method of claim 27, wherein the mammal has been exposed to a hepatitis C virus.

36. The method of claim 27, wherein the mammal is a human.

37. The antibody of claim 31.

38. The antibody of claim 37, which is a Fab or F(ab′)2.

39. The antibody of claim 37, which is Fab C1, J2, H3 or L4.

40. The antibody of claim 37, which is a monoclonal antibody.

41. The antibody of claim 37, which is an IgG antibody.

42. The antibody of claim 37, which is IgG AR3A, AR3B, AR3C or AR3D.

43. The antibody of claim 37, which is a murine antibody.

44. A method of eliciting an immune response in a mammal comprising administering to the mammal the nucleic acid of claim 12.

45. The method of claim 44, wherein the nucleic acid comprises a sequence encoding a polypeptide of SEQ ID NO: 866, 867, 868, 869 or 870.

46. The method of claim 44, wherein the nucleic acid has a sequence that comprises SEQ ID NO: 874, 875, 876, 877, 878, 879, 880 or 881.

47. The method of claim 44, wherein the nucleic acid is operably linked to an expression control sequence.

48. The method of claim 47, wherein the expression control sequence is a viral, phage, bacterial, or mammalian promoter.

49. The method of claim 48, wherein the promoter is a SV40 promoter, a Rous Sarcoma Virus promoter, or a cytomegalovirus immediate early promoter.

50. A method of eliciting an immune response in a mammal comprising administering to the mammal the expression vector of claim 17.

51. The method of claim 50, wherein the nucleic acid encoding the polypeptide comprises a sequence encoding a polypeptide of SEQ ID NO: 866, 867, 868, 869 or 870.

52. The method of claim 50, wherein the nucleic acid encoding the polypeptide has a sequence that comprises SEQ ID NO: 874, 875, 876, 877, 878, 879, 880 or 881.

53. The method of claim 50, wherein the nucleic acid encoding the polypeptide is operably linked to an expression control sequence.

54. The method of claim 53, wherein the expression control sequence is a viral, phage, bacterial, or mammalian promoter.

55. The method of claim 54, wherein the promoter is a SV40 promoter, a Rous Sarcoma Virus promoter, or a cytomegalovirus immediate early promoter.

56. A pharmaceutical composition comprising the polypeptide of claim 1 and a pharmaceutically acceptable carrier.

57. A pharmaceutical composition comprising the isolated nucleic acid of claim 12 and a pharmaceutically acceptable carrier.

58. A pharmaceutical composition comprising the expression vector of claim 17 and a pharmaceutically acceptable carrier.

59. A pharmaceutical composition comprising the antibody of claim 37 and a pharmaceutically acceptable carrier.

60. A purified preparation of the polypeptide of claim 1, wherein at least 80% of the polypeptides of claim 1 are in a conformation capable of binding to a conformation-dependent cross-neutralizing antibody.

61. A purified preparation of the antibody of claim 31, wherein the antibody is at least 5% of the antibodies in the preparation.

62. A method for determining whether a mammal has been infected with a hepatitis C virus comprising contacting a blood sample from the mammal with the polypeptide of claim 1 and determining whether the polypeptide of claim 1 binds specifically with an antibody from the blood of the mammal to form a polypeptide-antibody complex, wherein the presence of the complex indicates that the mammal has been infected with a hepatitis C virus and the absence of the complex indicates that the mammal has not been infected with the virus.


SEQUENCE LISTING (881 sequences)

SEQ ID NO: 1 - protein, 141 aa, Homo sapiens
LEQSGAEVKKPGSSVKVSCKASGGTFSSFVINWVRQAPGQGLEWVGGIFQAPGPEREWLRDINPISGTINYAQRFQGRVTMTADESMTTVYMELSSLRSEDTAMYYCARENKFRYCRGGSCYSGAFDMWGQGTMVTVSSAS

SEQ ID NO: 2 - protein, 123 aa, Homo sapiens
LEQSGAEVKKPGSSVKVSCRASGSPFSSYTITWVRQAPGQGLEWMGGIILMTGKANYAQKFQGRVTITADRSTTTAYMEMSSLTSDDTAIYYCARDPYVYAGDDVWSLSRWGQGTLVIVSSAS

SEQ ID NO: 3 - protein, 123 aa, Homo sapiens
LEQSGAEVKKPGSSVKVSCRASGSPYSSYTITWVRQAPGQGLEWMGGIILMTGKANYAQKFQGRVTITADRATATAYMEMSSLTSDDTAIYYCARDPYVYAGDDVRSLSRWGQGTPVIVSSAS

SEQ ID NO: 4 - protein, 123 aa, Homo sapiens
LEQSGAEVKKPGSSVKVSCRASGSPYSSYTITWVRQAPGQGLEWMGGIILMTGKANYAQKFQGRVTITADRATATAYMEMSSLTSDDTAIYYCARDPYVYAGDDVWSLSRWGQGTPVIVSSAS

SEQ ID NO: 5 - protein, 126 aa, Homo sapiens
LEQSGAEVKTPGSSVRVSCRPPGGNFNSYSINWVRQAPGHGLEWVGTFIPMFGTSKYAQKFQGRVTITADGSSGTAYMDLNSLRSDDTAFYYCVRPETPRYCSGGFCYGEFDNWGQGTLVTVSSAS

SEQ ID NO: 6 - protein, 126 aa, Homo sapiens
LEQSGAEVKKPGSSVRVSCRAPGGTFNSYSVNWVRQAPGHGLEWVGTLIPMFGTSSYAQKFQGRVTITADGSSGTAYMELNSLRSDDTAVYYCVRPETPRYCRGGFCYGEFDNWGQGTLVTVSSAS

SEQ ID NO: 7 - protein, 126 aa, Homo sapiens
LEQSGAEVKEPGSSVRVSCRAPGGTFNSYSINWVRQAPGHGLEWVGTLIPMFGTSNYAQKFQGRVTITADGSSGTAYMELNSLRSDDTAVYYCVRPETPRYCSGGVCYGEFDNWGQGTLVTVSSAS

SEQ ID NO: 8 - protein, 125 aa, Homo sapiens
LESGAEVKKPGSSVRVSCRPPGGTFNSYSINWVRQAPGHGLEWVGTIIPMFGTSKYAQKLQGRVTITADGSSGTAYMELNSLRSDDTAVYYCVRPETPRYCSGGFCYGEFDNWGQGTLVTVSSAS

SEQ ID NO: 9 - protein, 126 aa, Homo sapiens
LEQSGAEVKKPGSSVRVSCRAPGGTLNSYSINWVRQAPGHGLEWVGTLIPMFGTSNYAQKFQGRVTITADGSSGTAYMELNSLRSDDTAVYYCVRPETPRYCSGGFCYGEFDNWGQGTLVTVSSAS

SEQ ID NO: 10 - protein, 126 aa, Homo sapiens
LEQSGAEVKKPGSSVRVSCRPPGGTFNSYSINWVRQAPGHGLEWVGTIIPMFGTSKYAQKLQGRVTITADGSSGTAYMELNSLRSDDTAVYYCVRPETPRYCSGGFCYGEFDNWGQGTLVTVSSAS

SEQ ID NO: 11 - protein, 122 aa, Homo sapiens
LESGGGLVQPGGSLRLSCEASGYYFSSFAMSWVRQTPGKGLEWVSSIAGGTLGRTSYRDSVKGRFTISRDNSKNTVFLHMNNLRPEDTAVYYCAKDPLLFAGGPNWFDHWGQGTLVTVSSAS

SEQ ID NO: 12 - protein, 123 aa, Homo sapiens
LEQSGGGLVQPGGSLRLSCEASGYYFSSFAMSWVRQTPGKGLEWVSSIAGGTLGRTSYRDSVKGRFTISRDNSKNTVFLHMNNLRPEDTAVYYCAKDPLLFAGGPNWFDHWGQGTLVTVSSAS

SEQ ID NO: 13 - protein, 122 aa, Homo sapiens
LESGGGLVQPGGSLRLSCEASGYYFSSFAMSWVRQTPGKGLEWVSSIAGGTLGRTSYRDSVKGRFTISRDNSKNTMFLHMNNLRPEDTAVYYCAKDPLLFAGGPNWFDHWGQGTLVTVSSAS

SEQ ID NO: 14 - protein, 122 aa, Homo sapiens
LESGGGLVQPGGSLRLSCEASGYYFSSFAMSWVRQTPGKGLEWVSSIAGGTLGRTSYRDSVKGRFTISRDNSKNTVFLHMSNLRPEDTAVYYCAKDPLLFAGGPNWFDHWGQGTLVTVSSAS

SEQ ID NO: 15 - protein, 120 aa, Homo sapiens
LEQSGAELKKPGSSVKVSCKPSDGTFRAYTLSWVRQAPGQTLEWMGRIMPTVGITNYAQKFQGRVTISADMSTATAYMELSSLRSDDTAIYYCAKGPYVGLGEGFSEWGQGTLVTVSSAS

SEQ ID NO: 16 - protein, 113 aa, Homo sapiens
LEQSGNEVKKPGASVKVSCRAYGYNFGSERLSWVRQAPGQGLEWMGWISAYNGGINYSQKFQGRFTMTTDTSTRTGYMELRNLRSDDTAVYYCARGGGTEWGQGTLVIVSSDE

SEQ ID NO: 17 - protein, 126 aa, Homo sapiens
LEQSGAEMKKPGASLKVSCKTSGYTFDDYGVTWVRQAPGQGLEWMGWISAYSGNTFYARKFQGRVTMTTDPSTRTAYMELRSLRSDDTAVYFCARDRGLAINGVVFPYFGLDVWGQGTTVTVSSAS

SEQ ID NO: 18 - protein, 126 aa, Homo sapiens
LEQSGAEVKKPGSSVKVSCEASGGTFDNYSLNWVRQAPGQGLEWIGGVVPLFGTTKYAQKFQGRVTISDDKSTGTGHMELRSLRSEDTAVYYCVRSVTPRHCGGGFCYGEFDYWGQGTLVTVSSAS

SEQ ID NO: 19 - protein, 126 aa, Homo sapiens
LEQSGAEVKKPGSSVKVSCETSGGTFDNYALNWVRQAPGQGLEWIGGVVPLFGTTKYAQKFQGRVTISDDKSTGTGHMELRSLRSEDTAVYYCVRSVTPRHCGGGFCYGEFDYWGQGTLVTVSSAS

SEQ ID NO: 20 - protein, 126 aa, Homo sapiens
LEQSGAEVKKPGSSVKVSCETSGGTLDNYALNWVRQAPGQGLEWIGGVVPLFGTTRNAQKFQGRVTISDDKSTGTGHMELRSLRSEDTAVYYCVRSVTPRYCGGGFCYGEFDYWGQGTLVTVSSAS

SEQ ID NO: 21 - protein, 127 aa, Homo sapiens
LESGGGLVQPGRSLRLSCKASGFNFAQYTMNWVRQAPGKGLEWIGLIRTTAYDAATHYAASVEGRFTISRDDSKSTAYLQINGLKTEDTAVYYCARPHGPGLSLGIYSAEYFDEWGQGTLVTVSSAS

SEQ ID NO: 22 - protein, 126 aa, Homo sapiens
LEQSGPEVKKPGSSVKVSCKGSGDRFNDPVTWVRQAPGQGLEWIGGIIPAFGATKYAQKFQGRVVISADASTDTAYMELSSLRSEDTAVYYCAKVGVRGIILVGGLAMNWLDPWGQGTLVTVSAAS

SEQ ID NO: 23 - protein, 126 aa, Homo sapiens
LEQSGPEVKKPGSSVKVSCKDSGDTFNEPVTWVRQAPGQGLEWIGGIIPAFGVTKYAQKFQGRVIISADASTATAYLELSSLRSEDTAVYYCAKVGLRGIVMVGGLAMNWLDPWGQGTQVTVSSAS

SEQ ID NO: 24 - protein, 126 aa, Homo sapiens
LEQSGPEVKKPGSSVKVSCKGSGDTFNDPVTWVRQAPGQGLEWIGGIIPLFGAAKYAQKFQGRVTISADASALTTYMEMSSLRPEDTAVYYCAKVGLRGITLVGGLAMNWLDPWGQGTLITVSSAS

SEQ ID NO: 25 - protein, 126 aa, Homo sapiens
LEQSGAEVKKPGSSVRVSCEVSGDTFREPVSWVRQAPGQGFEWIGGIIPMFGATHYAQKLQGRITISADQSTNTVYMELRSLRSDDTAVYYCAKVGLRGINMVGGLAMNWFDPWGQGTLVTVSSAS

SEQ ID NO: 26 - protein, 127 aa, Homo sapiens
LEQSGPGLVKPGRPFSLTCAISGDSVSSDSAAWNWVRQSPSRGLEWLGRTFYRSKWYYDYTVSVKSRITINSDTSKNQFSLHLNSVTPEDTAVYYCVRDFYIGPTRDVYYGMDVWGQGTTVTVSSAS

SEQ ID NO: 27 - protein, 130 aa, Homo sapiens
LEQSGAEVKKPGSSVKVSCKASGDTFRSYVITWARQAPGQGLEWMGAIIPFFGTTNLAQKFQGRVTITADESTQTVYMDLSSLRSDDTAVYYCAKAGDLSVGGVLAGGVPHLRHFDPWGQGTLVTVSSAS

SEQ ID NO: 28 - protein, 130 aa, Homo sapiens
LEQSGAEVKMPGSSVKVSCKASGDTFRSSVITWARQAPGQGLEWMGAIIPFFGTTNLAQKFQGRVTITADESTKTVYMDLSSLRSDDTAVYYCAKAGDLSVGGVLAGGVPHLRHFDPWGQGTLVTVSSAS

SEQ ID NO: 29 - protein, 130 aa, Homo sapiens
LEQSGAEVKKPGSSVKVSCKASGDTFRSYVITWARQAPGQGLEWMGAIIPFFGTTNLAQKFQGRVTITADESTKTVYMDLSSLTSDDTAVYYCAKAGDLSVGGVLAGGVPHLRHFDPWGQGTLVTVSSAS

SEQ ID NO: 30 - protein, 130 aa, Homo sapiens
LEQSGAEVKKPGSSVKVSCKASGDTFRSYVITWARQAPGQGLEWMGAIIPFFGTTNLAQKFQGRVTITADESTKTVYMDLSSLRSDDTAVYYCAKAGDLSVGGVLAGGVPHLRHFDPWGQGTLVTVSSAS

SEQ ID NO: 31 - protein, 126 aa, Homo sapiens
LEQSGAEVKKPGASVKVSCKASGYTFTNYAITWVRQAPGQGLEWMGWISGDSTNTYYGQKFQGRVTMTTDTSTSTAYMELTSLTSEDTAVYYCARESLYMIAFGRVIWPPLDYWGQGTLVTISSAS

SEQ ID NO: 32 - protein, 131 aa, Homo sapiens
ELTQSPATLSVSPGESATLSCRASQSVSDNLAWYQQKPGQAPRLLIYGASSRAPAIPGRFSGSGSGTDFTLTISRLEPEDLAVYHCQQYGASPWTFGQGTKVEIKRTVAAPSVFIFPPSDEQLKSGTASVV

SEQ ID NO: 33 - protein, 134 aa, Homo sapiens
ELTLTQSPGTLSLSPGERATLSCRASQSVSNSYLAWYQQKPGQAPRLLIYGASSRATGIPDRFSGSGSGTDFTLTISRLEPEDFAVYYCQQYGSSPQTFGQGTKVEIKRTVAAPSVFIFPPSDEQLKSGTASVV

SEQ ID NO: 34 - protein, 137 aa, Homo sapiens
TLTQSPDSLAVSLGERATINCKSSQSVLYSSNNKNVLAWYQQKPGQPPQLLIYWASTRESGVPDRFSGSGSGTDFTLTISSLQAEDVAVYFCQQYYATPFTFGQGTKVEIKRTVAAPSVFIFPPSDEQLKSGTASVV

SEQ ID NO: 35 - protein, 131 aa, Homo sapiens
ELVMTQSPGTLSLSPGERATLSCRASQRVGSSYLAWYQQKPGQAPRLLVYGASSRATGIPDRFSGSGSGTDFTLTISRLQPEDFAVYYCQQYGTTFGQGTRVDIKRTVAAPSVSIFPPSDEQLKSGTASVV

SEQ ID NO: 36 - protein, 132 aa, Homo sapiens
ELTLTQSPGTLSLSPGERATLSCRASQSVSGNYLAWYQQKPGQAPRLLIYGASNRATGIPHRFSGSGSGTDFTLTISRLEPEDFAVYYCQQYGSSPTFGQGTRVDIKRTVAAPSVFIFPPSDEQLKSGTASV

SEQ ID NO: 37 - protein, 120 aa, Homo sapiens
ELTQSPSTLSLSPGEGATLSCRPSQSVSRNYLAWYQQKPGQAPRLLIYGASTRATGIPDRFSGSGSGTNFTLTISRLEPEDFAVYFCQHYGNSPPYTFGQGTKLEIKRTVAAPSVFIFPP

SEQ ID NO: 38 - protein, 120 aa, Homo sapiens
ELTQSPGTLSLSPGERAALSCRASQSISTNYLAWYQQKPGQAPRLLIYGTSSRATGIPDRFSGTGSGTDFSLTISRLEPEDSAVYYCQQYGTSPFTFGPGTKVDIKRTVAAPSVFIFPPS

SEQ ID NO: 39 - protein, 130 aa, Homo sapiens
ELTQSPGTLSLSPGERATLSCRASQSVSYLAWYQQKPGQAPRLLIYGASSRATGIPDRFSGSGSGTDFTLTISRLEPEDFAVYYCQQYGSSPQTFGQGTKVEIKRTVAAPSVFIFPPSDEQLKSGTASVV

SEQ ID NO: 40 - protein, 121 aa, Homo sapiens
ELTQSPGTLSLSPGERATLSCRASQSVSSSYLAWYQQKPGQAPRLLIYGASSRATGIPDRFSGSGSGTDFTLTISGLEPEDFAVYYCQQYGSSPLTFGGGTKVEIKRTVAAPSVFIFPPSD

SEQ ID NO: 41 - protein, 132 aa, Homo sapiens
ELTQSPGTLSLSPGERATLSCRASQSVSSNYLAWYQQKPGQAPRLLIYGASSRATGIPDRFSGSGSGTNFTLTISRLEPEDFAVYYCQHYGSSSYTFGQGTRVEIKRTVAAPSVFIFPPSDEQLKSGTASVV

SEQ ID NO: 42 - protein, 132 aa, Homo sapiens
ELTQSPATLYVSPGERATLSCRASQSVPDNHLAWYQQKPGQTPRLLIYGASKRATGIPDRFSGSGSGTDFTLTISRLEPEDFAVYYCQQYGSSPQTFGQGTKVEIKRTVAAPSVFIFPPSDEQLKSGTASVV

SEQ ID NO: 43 - protein, 132 aa, Homo sapiens
ELTQSPGTLSVSPGEAATLSCRASQSVSSNLAWYQQKPGQAPRLLIYGASTRATGIPARFSGSGSGTDFTLTISRLEPEDFAVYYCQQYGGSPPYTFGQGTKLEIKRTVAAPSVFIFPPSDEQLKSGTASVV

SEQ ID NO: 44 - protein, 131 aa, Homo sapiens
ELTQSPGTLSLSPGERATLSCRASQSVSSSYLAWYQQKPGQAPRLLIYGASSRATGIPDRFSGSGSGTDFTLTISRLEPEDFAVYYCQQYGSSPQTFGQGTKVEIKRTVAAPSVFIFPPSDEQLKSGTASV

SEQ ID NO: 45 - protein, 120 aa, Homo sapiens
TLTQSPATLSVSPGERATLSCRASQTTSDNLAWYQQKPGQAPRLLIYGASSRATGIPDRFSGSGSGTDFTLTISRLEPEDFAVYYCQQYGSSPQTFGQGTKVEIKRTVAAPSVFIFPPSD

SEQ ID NO: 46 - protein, 131 aa, Homo sapiens
ELTLTQSPGTLSLSPGERATLSCRASQTVSSSYLAWYQQKPGQAPRLLIYGASSRATGIPDRFSGSGSGTDFTLTISRLEPEDFAVYYCQQYGSSPQTFGQGTKVEIKRTVAAPSVFIFPPSDEQLKSGTA

SEQ ID NO: 47 - protein, 131 aa, Homo sapiens
ELVMTQSPGTLSLSPGERATLSCRASQSVSSSYLAWYQQKPGQAPRLLIYGASSRATGIPDRFSGSGSGTDFTLTITRLEPEDFAVYYCQQYGSSPQTFGQGTKVQIKRTVAAPSVFIFPPSDEQLKSGTA

SEQ ID NO: 48 - protein, 136 aa, Homo sapiens
ELVLTQSPLSLPVTLGQPASISCRSTQSLVYSDGNTYLNWFHQRAGQPPRRLIYKVSNRDSGVPERFSGSGSGTDFTLKISRVEAEDVGIYYCMQGAHWPPTFGGGTKVEINRTVAAPSVFIFPPSDEQLKSGTAS

SEQ ID NO: 49 - protein, 122 aa, Homo sapiens
ELQMTQSPSFLSASVGDRVTITCRASQGISSYLAWYQQKPGKAPKLLISSVSTLQSGVSSRFSGSGSGTGFTLTISSLQSEDSATYYCEQLNSFPYTFGQGTKLEIKRTVAAPSVFIFPPSD

SEQ ID NO: 50 - protein, 125 aa, Homo sapiens
ELTQSPVSLPVTPGEPASISCRSSQSLLHSNGNHYLDWYLQKPGQSPQLLMYLGSNRASGVPDRFSGSGSGTDFTLKISRVEAEDVGVYYCMQGLQTPWTFGQGTKVEIKRTVAAPSVFIFPPSD

SEQ ID NO: 51 - protein, 123 aa, Homo sapiens
ELTLTQSPGTLSLSPGERATLSCRASQSISTNYLAWYQQKPGQAPRLLIYGASRRATGIPDRFSGSGSGTDFTLTISRLEPEDFAVYYCQQYGSSPLTFGGGTKVEIKRTVAAPSVFIFPPSD

SEQ ID NO: 52 - protein, 122 aa, Homo sapiens
ELTQSPATLSVSPGERATLSCRASRGISSNLAWYQQKPGQAPRLLIYGASSRATGIPDRFSGSGSGTDFTLTISRLEPEDFAVYYCQQYGSSPQTFGQGTEVEIKRTVAAPSVFIFPPSDEQ

SEQ ID NO: 53 - protein, 136 aa, Homo sapiens
ELTLTQSPGTLSLSPGERATLSCRASQSVSSDSLAWYQQKPGQAPRLLIYGASRRATGIPDRFSGSGSGTDFTLTISRLEPEDLGVYYCQQYGPSPPGYTFGQGTKLEIKRTVAAPSVFIFPPSDEQLKSGTASVV

SEQ ID NO: 54 - protein, 134 aa, Homo sapiens
ELTLTQSPGTLSLSPGERGTLSCRASQSVSSSYLAWYQQKPGQAPRLLIYGASSRATGIPDRFSGSGSGTDFTLTISRLEPEDFAVYYCQQYGSSPQTFGQGTKVEIKRTVAAPSVFIFPPSDEQLKSGTASVV

SEQ ID NO: 55 - protein, 130 aa, Homo sapiens
ELTQSPATLSVSPGERATLSCRASQSVSSNLAWYQQKPGQAPRLLIYGASTRATGIPARFSGSGSGTDFTLTVSRLEPEDSAVYFCQQYRSPLTFGGGTKVEIKRTVAAPSVFIFPPSDEQLKSGTASVV

SEQ ID NO: 56 - protein, 122 aa, Homo sapiens
ELTLTQSPATLSVSPGERATLFCRANQSVGRNLAWYQQKPGQAPRLLIYGISTRTTTTPTRFSGSGSGTDFTLTISRLQSEDFAVYYCQQYNKWPPWTFGQGTKLEIKRTVAAPSVFVFPPS

SEQ ID NO: 57 - protein, 134 aa, Homo sapiens
ELVLTQSPGTLSLSPGERATLSCRASQSVSSSYLAWYQQKPGQAPRLLIYGASSRATGIPDRFSGSGSGTDFALTITRLEPEDFAVYYCQQYGSSPQTFGQGTKVEIKRTVAAPSVFIFPPSDEQLKSGTASVV

SEQ ID NO: 58 - protein, 131 aa, Homo sapiens
ELTQSPGTLSLSPGERATLSCRASQSVSSYLAWYQQKPGQAPRLLIYGASSRATGIPDRFSGSGSGTDFTLTISRLEPEDFAVYYCQQYGSSPQTFGQGTKVEIKRTVAAPSVFIFPPSDEQLKSGTASVV

SEQ ID NO: 59 - protein, 131 aa, Homo sapiens
ELTQSPGTLSLSPGERATLSCRASQSVSSYLAWYQQKPGQAPRLLIYGASSRATGIPDRFSGSGSGTDFTLTISGLEPEDFAVYYCQQYGSSPQTFGQGTKVEIKRTVAAPSVFIFPPSDEQLKSGTASVV

SEQ ID NO: 60 - protein, 133 aa, Homo sapiens
EFTLTQSPGTLSLSPGERGTLSCRASQSVSSYLAWYQQKPGQAPRLLIYGASSRATGIPDRFSGSGSGTDFTLTISGLEPEDFAVYYCQQYGSSPQTFGQGTKVEIKRTVAAPSVFIFPPSDEQLKSGTASVV

SEQ ID NO: 61 - protein, 133 aa, Homo sapiens
ELTLTQSPGTLSLSPGERATLSCRASQSVSSYLAWYQQKPGQAPRLLIYGASSRATGIPDRFSGSGSGTDFTLTISRLEPEDFAVYYCQQYGSSPQTFGQGTEVEIKRTVAAPSVFIFPPSDEQLKSGTASVV

SEQ ID NO: 62 - protein, 134 aa, Homo sapiens
ELTQSPGTLSLSPGERATLSCRASQSVSSNSLAWYQQKPGLAPRLLIYGASSRATGIPDRFSGSGSGTGFTLTISTLEPEDFAIYYCQQYGGSPPRFTFGPGTKVDIRRTVAAPSVFIFPPSDEQLKSGTASVV

SEQ ID NO: 63 - protein, 132 aa, Homo sapiens
ELTQSPGTLSLSPGERATLSCRASQSITSRYLAWYQQKPGQAPRLLIYGASSRATGIPDRFSGSGSGTDFTLTISRLEPEDFAVYYCQQYGDSPVGFGPGTKVEIKRTVAAPSVFIFPPSDEQLKSGTASVV

SEQ ID NO: 64 - protein, 133 aa, Homo sapiens
ELVMTQSPATLSLSPGERATLSCRASQSVGSYLAWYQQKPGQAPRLLIYDASNRATGIPARFSGSGSGTDFTLTISSLEPEDFAVYFCQQYGSSPLTFGQGTKVEIKRTVAAPSVFIFPPSDEQLKSGTASVV

SEQ ID NO: 65 - protein, 132 aa, Homo sapiens
ELTQSPGTLSLSPGERATLSCRAGQTVASNSLAWYQHKPGQAPRLLIYGASIRASGIPDRFSGSGSGTDFTLTISRLEPEDFAVYYCQQYGLSPSTFGQGTRLEIKRTVAAPSVFIFPPSDEQLKSGTASVV

SEQ ID NO: 66 - protein, 130 aa, Homo sapiens
ELTLTQSPGTLSLSPGERATLSCRASQSIRSSYLAWYQQKPGQAPRLLIYAAASRATGIPDRFSGSGSGTDFTLTISRLEPEDFAVYFCHHYGGSPRTFGQGTKVEIKRTVAAPSVFIFPPSDEQLKSGT

SEQ ID NO: 67 - protein, 126 aa, Homo sapiens
LLESGPGLVKPSQTLSLTCDISGDSVSSHSAAWNWIRQSPRGLEWLGRTYYRSNWYNDYSVSVRSRIIISADTSKNQFSLQLNSVTPEDTAVYYCARDRFYGPGGDLYYGLDVWGQGTTVTVSSAS

SEQ ID NO: 68 - protein, 126 aa, Homo sapiens
LLESGPGLVKPSQTLSLTCDISGDSVSSHSAAWNWIRQSPRGLEWLGRTYYRSNWYNDYSVSVRSRIIISADTSKNQLSLQLNSVTPEDTAVYYCARDRFYGPGGDLYYGLDVWGQGTTVTVSSAS

SEQ ID NO: 69 - protein, 126 aa, Homo sapiens
LLESGPGLVKPSQTLSLTCDISGDSVSSNSAAWNWIRQSPRGLEWLGRTFYRSNWYNDYAVSVKSRITINPDTSKNQFSLQLNSVTPEDTAVYYCARDLYIGPTTDFYYGMDVWGQGTTVTVSSAS

SEQ ID NO: 70 - protein, 126 aa, Homo sapiens
LLESGPGLVRPSQTLSLTCDISGDSVSSNSAAWNWIRQSPRGLEWLGRTFYRSNWYNDYAVSVKSRITINPDTSKNQFSLQLNSVTPEDTAVYYCARDLYIGPTTDFYYGMDVWGQGTTVTVSSAS

SEQ ID NO: 71 - protein, 126 aa, Homo sapiens
LLESGPGLVKPSQTLSLTCAISGDSVSSNSAAWNWIRQSPRGLEWLGRTFYRSKWYYDYAVSVKSRITINPDTSKNQFSLHLNSVTPEDTAVYYCVRDFYIGPTRDFYYGMDVWGQGTTVTVSSAS

SEQ ID NO: 72 - protein, 123 aa, Homo sapiens
ELTQSPGTLSLSPGERATLSCRASQSVSSSYKAWYQQKPGQAPRLLIYGASSRATGIPDRFSGSGSGTDFTLTISRLEPEDFAVYYCQQYGSSPQTFGQGTKVEIKRTVAAPSVFIFPPSDEQ

SEQ ID NO: 73 - protein, 123 aa, Homo sapiens
ELTQSPGTLSLSPGERGTLSCRASQSVSSSYKAWYQQKPGQAPRLLIYGASSRATGIPDRFSGSGSGTDFTLTISRLEPEDFAVYYCQQYGSSPQTFGQGTKVEIKRTVAAPSVFIFPPSDEQ

SEQ ID NO: 74 - protein, 123 aa, Homo sapiens; identical to SEQ ID NO: 72

SEQ ID NO: 75 - protein, 125 aa, Homo sapiens
ELTLTQSPGTLSLSPGERATLSCRASQSVSSSYKAWYQQKPGQAPRLLIYGASSRATGIPDRFSGSGSGTDFTLTISRLEPEDFAVYYCQQYGSSPQTFGQGTKVEIKRTVAAPSVFIFPPSDEQ

SEQ ID NO: 76 - protein, 123 aa, Homo sapiens; identical to SEQ ID NO: 72

SEQ ID NO: 77 - protein, 123 aa, Homo sapiens; identical to SEQ ID NO: 72

SEQ ID NO: 78 - protein, 8 aa, Homo sapiens
TFSSFVIN

SEQ ID NO: 79 - protein, 8 aa, Homo sapiens
PFSSYTIT

SEQ ID NO: 80 - protein, 8 aa, Homo sapiens
PYSSYTIT

SEQ ID NO: 81 - protein, 8 aa, Homo sapiens
PYSSYTIT

SEQ ID NO: 82 - protein, 8 aa, Homo sapiens
NFNSYSIN

SEQ ID NO: 83 - protein, 8 aa, Homo sapiens
TFNSYSVN

SEQ ID NO: 84 - protein, 8 aa, Homo sapiens
TFNSYSIN

SEQ ID NO: 85 - protein, 8 aa, Homo sapiens
TFNSYSIN

SEQ ID NO: 86 - protein, 8 aa, Homo sapiens
TLNSYSIN

SEQ ID NO: 87 - protein, 8 aa, Homo sapiens
TFNSYSIN

SEQ ID NO: 88 - protein, 8 aa, Homo sapiens
YFSSFAMS

SEQ ID NO: 89 - protein, 8 aa, Homo sapiens
YFSSFAMS

SEQ ID NO: 90 - protein, 8 aa, Homo sapiens
YFSSFAMS

SEQ ID NO: 91 - protein, 8 aa, Homo sapiens
YFSSFAMS

SEQ ID NO: 92 - protein, 8 aa, Homo sapiens
TFRAYTLS

SEQ ID NO: 93 - protein, 8 aa, Homo sapiens
NFGSERLS

SEQ ID NO: 94 - protein, 8 aa, Homo sapiens
TFDDYGVT

SEQ ID NO: 95 - protein, 8 aa, Homo sapiens
TFDNYSLN

SEQ ID NO: 96 - protein, 8 aa, Homo sapiens
TFDNYALN

SEQ ID NO: 97 - protein, 8 aa, Homo sapiens
TLDNYALN

SEQ ID NO: 98 - protein, 8 aa, Homo sapiens
NFAQYTMN

SEQ ID NO: 99 - protein, 7 aa, Homo sapiens
RFNDPVT

SEQ ID NO: 100 - protein, 7 aa, Homo sapiens
TFNEPVT

SEQ ID NO: 101 - protein, 7 aa, Homo sapiens
TFNDPVT

SEQ ID NO: 102 - protein, 7 aa, Homo sapiens
TFREPVS

SEQ ID NO: 103 - protein, 10 aa, Homo sapiens
SVSSDSAAWN

SEQ ID NO: 104 - protein, 8 aa, Homo sapiens
TFRSYVIT

SEQ ID NO: 105 - protein, 8 aa, Homo sapiens
TFRSSVIT

SEQ ID NO: 106 - protein, 8 aa, Homo sapiens
TFRSYVIT

SEQ ID NO: 107 - protein, 8 aa, Homo sapiens
TFRSYVIT

SEQ ID NO: 108 - protein, 8 aa, Homo sapiens
TFTNYAIT

SEQ ID NO: 109 - protein, 16 aa, Homo sapiens
INPISGTINYAQRFQG

SEQ ID NO: 110 - protein, 16 aa, Homo sapiens
IILMTGKANYAQKFQG

SEQ ID NO: 111 - protein, 16 aa, Homo sapiens
IILMTGKANYAQKFQG

SEQ ID NO: 112 - protein, 16 aa, Homo sapiens
IILMTGKANYAQKFQG

SEQ ID NO: 113 - protein, 16 aa, Homo sapiens
FIPMFGTSKYAQKFQG

SEQ ID NO: 114 - protein, 16 aa, Homo sapiens
LIPMFGTSSYAQKFQG

SEQ ID NO: 115 - protein, 16 aa, Homo sapiens
LIPMFGTSNYAQKFQG

SEQ ID NO: 116 - protein, 16 aa, Homo sapiens
IIPMFGTSKYAQKLQG

SEQ ID NO: 117 - protein, 16 aa, Homo sapiens
LIPMFGTSNYAQKFQG

SEQ ID NO: 118 - protein, 16 aa, Homo sapiens
IIPMFGTSKYAQKLQG

SEQ ID NO: 119 - protein, 17 aa, Homo sapiens
IAGGTLGRTSYRDSVKG

SEQ ID NO: 120 - protein, 17 aa, Homo sapiens
IAGGTLGRTSYRDSVKG

SEQ ID NO: 121 - protein, 17 aa, Homo sapiens
IAGGTLGRTSYRDSVKG

SEQ ID NO: 122 - protein, 17 aa, Homo sapiens
IAGGTLGRTSYRDSVKG

SEQ ID NO: 123 - protein, 16 aa, Homo sapiens
IMPTVGITNYAQKFQG

SEQ ID NO: 124 - protein, 16 aa, Homo sapiens
ISAYNGGINYSQKFQG

SEQ ID NO: 125 - protein, 16 aa, Homo sapiens
ISAYSGNTFYARKFQG

SEQ ID NO: 126 - protein, 16 aa, Homo sapiens
VVPLFGTTKYAQKFQG

SEQ ID NO: 127 - protein, 16 aa, Homo sapiens
VVPLFGTTKYAQKFQG

SEQ ID NO: 128 - protein, 16 aa, Homo sapiens
VVPLFGTTRNAQKFQG

SEQ ID NO: 129 - protein, 18 aa, Homo sapiens
IRTTAYDAATHYAASVEG

SEQ ID NO: 130 - protein, 16 aa, Homo sapiens
IIPAFGATKYAQKFQG

SEQ ID NO: 131 - protein, 16 aa, Homo sapiens
IIPAFGVTKYAQKFQG

SEQ ID NO: 132 - protein, 16 aa, Homo sapiens
IIPLFGAAKYAQKFQG

SEQ ID NO: 133 - protein, 16 aa, Homo sapiens
IIPMFGATHYAQKLQG

SEQ ID NO: 134 - protein, 17 aa, Homo sapiens
TFYRSKWYYDYTVSVKS

SEQ ID NO: 135 - protein, 16 aa, Homo sapiens
IIPFFGTTNLAQKFQG

SEQ ID NO: 136 - protein, 16 aa, Homo sapiens
IIPFFGTTNLAQKFQG

SEQ ID NO: 137 - protein, 16 aa, Homo sapiens
IIPFFGTTNLAQKFQG

SEQ ID NO: 138 - protein, 16 aa, Homo sapiens
IIPFFGTTNLAQKFQG

SEQ ID NO: 139 - protein, 16 aa, Homo sapiens
ISGDSTNTYYGQKFQG

SEQ ID NO: 140 - protein, 19 aa, Homo sapiens
ENKFRYCRGGSCYSGAFDM

SEQ ID NO: 141 - protein, 15 aa, Homo sapiens
DPYVYAGDDVWSLSR

SEQ ID NO: 142 - protein, 15 aa, Homo sapiens
DPYVYAGDDVRSLSR

SEQ ID NO: 143 - protein, 15 aa, Homo sapiens
DPYVYAGDDVWSLSR

SEQ ID NO: 144 - protein, 18 aa, Homo sapiens
PETPRYCSGGFCYGEFDN

SEQ ID NO: 145 - protein, 18 aa, Homo sapiens
PETPRYCRGGFCYGEFDN

SEQ ID NO: 146 - protein, 18 aa, Homo sapiens
PETPRYCSGGVCYGEFDN

SEQ ID NO: 147 - protein, 18 aa, Homo sapiens
PETPRYCSGGFCYGEFDN

SEQ ID NO: 148 - protein, 18 aa, Homo sapiens
PETPRYCSGGFCYGEFDN

SEQ ID NO: 149 - protein, 18 aa, Homo sapiens
PETPRYCSGGFCYGEFDN

SEQ ID NO: 150 - protein, 14 aa, Homo sapiens
DPLLFAGGPNWFDH

SEQ ID NO: 151 - protein, 14 aa, Homo sapiens
DPLLFAGGPNWFDH

SEQ ID NO: 152 - protein, 14 aa, Homo sapiens
DPLLFAGGPNWFDH

SEQ ID NO: 153 - protein, 14 aa, Homo sapiens
DPLLFAGGPNWFDH

SEQ ID NO: 154 - protein, 12 aa, Homo sapiens
GPYVGLGEGFSE

SEQ ID NO: 155 - protein, 5 aa, Homo sapiens
GGGTE

SEQ ID NO: 156 - protein, 18 aa, Homo sapiens
DRGLAINGVVFPYFGLDV

SEQ ID NO: 157 - protein, 18 aa, Homo sapiens
SVTPRHCGGGFCYGEFDY

SEQ ID NO: 158 - protein, 18 aa, Homo sapiens
SVTPRHCGGGFCYGEFDY

SEQ ID NO: 159 - protein, 18 aa, Homo sapiens
SVTPRYCGGGFCYGEFDY

SEQ ID NO: 160 - protein, 18 aa, Homo sapiens
PHGPGLSLGIYSAEYFDE

SEQ ID NO: 161 - protein, 19 aa, Homo sapiens
VGVRGIILVGGLAMNWLDP

SEQ ID NO: 162 - protein, 19 aa, Homo sapiens
VGLRGIVMVGGLAMNWLDP

SEQ ID NO: 163 - protein, 19 aa, Homo sapiens
VGLRGITLVGGLAMNWLDP

SEQ ID NO: 164 - protein, 19 aa, Homo sapiens
VGLRGINMVGGLAMNWFDP

SEQ ID NO: 165 - protein, 16 aa, Homo sapiens
DFYIGPTRDVYYGMDV

SEQ ID NO: 166 - protein, 22 aa, Homo sapiens
AGDLSVGGVLAGGVPHLRHFDP

SEQ ID NO: 167 - protein, 22 aa, Homo sapiens
AGDLSVGGVLAGGVPHLRHFDP

SEQ ID NO: 168 - protein, 22 aa, Homo sapiens
AGDLSVGGVLAGGVPHLRHFDP

SEQ ID NO: 169 - protein, 22 aa, Homo sapiens
AGDLSVGGVLAGGVPHLRHFDP

SEQ ID NO: 170 - protein, 18 aa, Homo sapiens
ESLYMIAFGRVIWPPLDY

SEQ ID NO: 171 - protein, 9 aa, Homo sapiens
RASQSVSDN

SEQ ID NO: 172 - protein, 9 aa, Homo sapiens
RASQSVSNS

SEQ ID NO: 173 - protein, 14 aa, Homo sapiens
KSSQSVLYSSNNKN

SEQ ID NO: 174 - protein, 9 aa, Homo sapiens
RASQRVGSS

SEQ ID NO: 175 - protein, 9 aa, Homo sapiens
RASQSVSGN

SEQ ID NO: 176 - protein, 9 aa, Homo sapiens
RPSQSVSRN

SEQ ID NO: 177 - protein, 9 aa, Homo sapiens
RASQSISTN

SEQ ID NO: 178 - protein, 9 aa, Homo sapiens
RASQSVSDN

SEQ ID NO: 179 - protein, 9 aa, Homo sapiens
RASQSVSSS

SEQ ID NO: 180 - protein, 9 aa, Homo sapiens
RASQSVSSN

SEQ ID NO: 181 - protein, 9 aa, Homo sapiens
RASQSVPDN

SEQ ID NO: 182 - protein, 9 aa, Homo sapiens
RASQSVSSN

SEQ ID NO: 183 - protein, 9 aa, Homo sapiens
RASQSVSSS

SEQ ID NO: 184 - protein, 9 aa, Homo sapiens
RASQTTSDN

SEQ ID NO: 185 - protein, 9 aa, Homo sapiens
RASQTVSSS

SEQ ID NO: 186 - protein, 9 aa, Homo sapiens
RASQSVSSS

SEQ ID NO: 187 - protein, 13 aa, Homo sapiens
RSTQSLVYSDGNT

SEQ ID NO: 188 - protein, 8 aa, Homo sapiens
RASQGISS

SEQ ID NO: 189 - protein, 13 aa, Homo sapiens
RSSQSLLHSNGNH

SEQ ID NO: 190 - protein, 9 aa, Homo sapiens
RASQSISTN

SEQ ID NO: 191 - protein, 9 aa, Homo sapiens
RASRGISSN

SEQ ID NO: 192 - protein, 10 aa, Homo sapiens
RASQSVSSDS

SEQ ID NO: 193 - protein, 9 aa, Homo sapiens
RASQSVSSS

SEQ ID NO: 194 - protein, 9 aa, Homo sapiens
RASQSVSSN

SEQ ID NO: 195 - protein, 9 aa, Homo sapiens
RANQSVGRN

SEQ ID NO: 196 - protein, 9 aa, Homo sapiens
RASQSVSSS

SEQ ID NO: 197 - protein, 8 aa, Homo sapiens
RASQSVSS

SEQ ID NO: 198 - protein, 8 aa, Homo sapiens
RASQSVSS

SEQ ID NO: 199 - protein, 8 aa, Homo sapiens
RASQSVSS

SEQ ID NO: 200 - protein, 8 aa, Homo sapiens
RASQSVSS

SEQ ID NO: 201 - protein, 10 aa, Homo sapiens
RASQSVSSNS

SEQ ID NO: 202 - protein, 9 aa, Homo sapiens
RASQSITSR

SEQ ID NO: 203 - protein, 8 aa, Homo sapiens
RASQSVGS

SEQ ID NO: 204 - protein, 10 aa, Homo sapiens
RAGQTVASNS

SEQ ID NO: 205 - protein, 9 aa, Homo sapiens
RASQSIRSS

SEQ ID NO: 206 - protein, 7 aa, Homo sapiens
GASSRAP

SEQ ID NO: 207 - protein, 7 aa, Homo sapiens
GASSRAT

SEQ ID NO: 208 - protein, 7 aa, Homo sapiens
WASTRES

SEQ ID NO: 209 - protein, 7 aa, Homo sapiens
GASSRAT

SEQ ID NO: 210 - protein, 7 aa, Homo sapiens
GASNRAT

SEQ ID NO: 211 - protein, 7 aa, Homo sapiens
GASTRAT

SEQ ID NO: 212 - protein, 7 aa, Homo sapiens
GTSSRAT

SEQ ID NO: 213 - protein, 7 aa, Homo sapiens
GASSRAT

SEQ ID NO: 214 - protein, 7 aa, Homo sapiens
GASSRAT

SEQ ID NO: 215 - protein, 7 aa, Homo sapiens
GASSRAT

SEQ ID NO: 216 - protein, 7 aa, Homo sapiens
GASKRAT

SEQ ID NO: 217 - protein, 7 aa, Homo sapiens
GASTRAT

SEQ ID NO: 218 - protein, 7 aa, Homo sapiens
GASSRAT

SEQ ID NO: 219 - protein, 7 aa, Homo sapiens
GASSRAT

SEQ ID NO: 220 - protein, 7 aa, Homo sapiens
GASSRAT

SEQ ID NO: 221 - protein, 7 aa, Homo sapiens
GASSRAT

SEQ ID NO: 222 - protein, 7 aa, Homo sapiens
KVSNRDS

SEQ ID NO: 223 - protein, 7 aa, Homo sapiens
SVSTLQS

SEQ ID NO: 224 - protein, 7 aa, Homo sapiens
LGSNRAS

SEQ ID NO: 225 - protein, 7 aa, Homo sapiens
GASRRAT

SEQ ID NO: 226 - protein, 7 aa, Homo sapiens
GASSRAT

SEQ ID NO: 227 - protein, 7 aa, Homo sapiens
GASRRAT

SEQ ID NO: 228 - protein, 7 aa, Homo sapiens
GASSRAT

SEQ ID NO: 229 - protein, 7 aa, Homo sapiens
GASTRAT

SEQ ID NO: 230 - protein, 7 aa, Homo sapiens
GISTRTT

SEQ ID NO: 231 - protein, 7 aa, Homo sapiens
GASSRAT

SEQ ID NO: 232 - protein, 7 aa, Homo sapiens
GASSRAT

SEQ ID NO: 233 - protein, 7 aa, Homo sapiens
GASSRAT

SEQ ID NO: 234 - protein, 7 aa, Homo sapiens
GASSRAT

SEQ ID NO: 235 - protein, 7 aa, Homo sapiens
GASSRAT

SEQ ID NO: 236 - protein, 7 aa, Homo sapiens
GASSRAT

SEQ ID NO: 237 - protein, 7 aa, Homo sapiens
GASSRAT

SEQ ID NO: 238 - protein, 7 aa, Homo sapiens
DASNRAT

SEQ ID NO: 239 - protein, 7 aa, Homo sapiens
GASIRAS

SEQ ID NO: 240 - protein, 7 aa, Homo sapiens
AAASRAT

SEQ ID NO: 241 - protein, 9 aa, Homo sapiens
QQYGASPWT

SEQ ID NO: 242 - protein, 9 aa, Homo sapiens
QQYGSSPQT

SEQ ID NO: 243 - protein, 9 aa, Homo sapiens
QQYYATPFT

SEQ ID NO: 244 - protein, 6 aa, Homo sapiens
QQYGTT

SEQ ID NO: 245 - protein, 8 aa, Homo sapiens
QQYGSSPT

SEQ ID NO: 246 - protein, 10 aa, Homo sapiens
QHYGNSPPYT

SEQ ID NO: 247 - protein, 9 aa, Homo sapiens
QQYGTSPFT

SEQ ID NO: 248 - protein, 9 aa, Homo sapiens
QQYGSSPQT

SEQ ID NO: 249 - protein, 9 aa, Homo sapiens
QQYGSSPLT

SEQ ID NO: 250 - protein, 9 aa, Homo sapiens
QHYGSSSYT

SEQ ID NO: 251 - protein, 9 aa, Homo sapiens
QQYGSSPQT

SEQ ID NO: 252 - protein, 10 aa, Homo sapiens
QQYGGSPPYT

SEQ ID NO: 253 - protein, 9 aa, Homo sapiens
QQYGSSPQT

SEQ ID NO: 254 - protein, 9 aa, Homo sapiens
QQYGSSPQT

SEQ ID NO: 255 - protein, 9 aa, Homo sapiens
QQYGSSPQT

SEQ ID NO: 256 - protein, 9 aa, Homo sapiens
QQYGSSPQT

SEQ ID NO: 257 - protein, 9 aa, Homo sapiens
MQGAHWPPT

SEQ ID NO: 258 - protein, 9 aa, Homo sapiens
EQLNSFPYT

SEQ ID NO: 259 - protein, 9 aa, Homo sapiens
MQGLQTPWT

SEQ ID NO: 260 - protein, 9 aa, Homo sapiens
QQYGSSPLT

SEQ ID NO: 261 - protein, 9 aa, Homo sapiens
QQYGSSPQT

SEQ ID NO: 262 - protein, 11 aa, Homo sapiens
QQYGPSPPGYT

SEQ ID NO: 263 - protein, 9 aa, Homo sapiens
QQYGSSPQT

SEQ ID NO: 264 - protein, 8 aa, Homo sapiens
QQYRSPLT

SEQ ID NO: 265 - protein, 10 aa, Homo sapiens
QQYNKWPPWT

SEQ ID NO: 266 - protein, 9 aa, Homo sapiens
QQYGSSPQT

SEQ ID NO: 267 - protein, 9 aa, Homo sapiens
QQYGSSPQT

SEQ ID NO: 268 - protein, 9 aa, Homo sapiens
QQYGSSPQT

SEQ ID NO: 269 - protein, 9 aa, Homo sapiens
QQYGSSPQT

SEQ ID NO: 270 - protein, 9 aa, Homo sapiens
QQYGSSPQT

SEQ ID NO: 271 - protein, 11 aa, Homo sapiens
QQYGGSPPRFT

SEQ ID NO: 272 - protein, 9 aa, Homo sapiens
QQYGDSPVG

SEQ ID NO: 273 - protein, 9 aa, Homo sapiens
QQYGSSPLT

SEQ ID NO: 274 - protein, 9 aa, Homo sapiens
QQYGLSPST

SEQ ID NO: 275 - protein, 9 aa, Homo sapiens
HHYGGSPRT

SEQ ID NO: 276 - protein, 8 aa, Homo sapiens
SSHSAAWN

SEQ ID NO: 277 - protein, 8 aa, Homo sapiens
SSHSAAWN

SEQ ID NO: 278 - protein, 8 aa, Homo sapiens
SSNSAAWN

SEQ ID NO: 279 - protein, 8 aa, Homo sapiens
SSNSAAWN

SEQ ID NO: 280 - protein, 8 aa, Homo sapiens
SSNSAAWN

SEQ ID NO: 281 - protein, 17 aa, Homo sapiens
TYYRSNWYNDYSVSVRS

SEQ ID NO: 282 - protein, 17 aa, Homo sapiens
TYYRSNWYNDYSVSVRS

SEQ ID NO: 283 - protein, 17 aa, Homo sapiens
TFYRSNWYNDYAVSVKS

SEQ ID NO: 284 - protein, 17 aa, Homo sapiens
TFYRSNWYNDYAVSVKS

SEQ ID NO: 285 - protein, 17 aa, Homo sapiens
TFYRSKWYYDYAVSVKS

SEQ ID NO: 286 - protein, 16 aa, Homo sapiens
DRFYGPGGDLYYGLDV

SEQ ID NO: 287 - protein, 16 aa, Homo sapiens
DRFYGPGGDLYYGLDV

SEQ ID NO: 288 - protein, 16 aa, Homo sapiens
DLYIGPTTDFYYGMDV

SEQ ID NO: 289 - protein, 16 aa, Homo sapiens
DLYIGPTTDFYYGMDV

SEQ ID NO: 290 - protein, 16 aa, Homo sapiens
DFYIGPTRDFYYGMDV

SEQ ID NO: 291 - protein, 12 aa, Homo sapiens
RASQSVSSSYKA

SEQ ID NO: 292 - protein, 12 aa, Homo sapiens
RASQSVSSSYKA

SEQ ID NO: 293 - protein, 12 aa, Homo sapiens
RASQSVSSSYKA

SEQ ID NO: 294 - protein, 12 aa, Homo sapiens
RASQSVSSSYKA

SEQ ID NO: 295 - protein, 12 aa, Homo sapiens
RASQSVSSSYKA

SEQ ID NO: 296 - protein, 12 aa, Homo sapiens
RASQSVSSSYKA

SEQ ID NO: 297 - protein, 7 aa, Homo sapiens
GASSRAT

SEQ ID NO: 298 - protein, 7 aa, Homo sapiens
GASSRAT

SEQ ID NO: 299 - protein, 7 aa, Homo sapiens
GASSRAT

SEQ ID NO: 300 - protein, 7 aa, Homo sapiens
GASSRAT

SEQ ID NO: 301 - protein, 7 aa, Homo sapiens
GASSRAT

SEQ ID NO: 302 - protein, 7 aa, Homo sapiens
GASSRAT

SEQ ID NO: 303 - protein, 9 aa, Homo sapiens
QQYGSSPQT

SEQ ID NO: 304 - protein, 9 aa, Homo sapiens
QQYGSSPQT

SEQ ID NO: 305 - protein, 9 aa, Homo sapiens
QQYGSSPQT

SEQ ID NO: 306 - protein, 9 aa, Homo sapiens
QQYGSSPQT

SEQ ID NO: 307 - protein, 9 aa, Homo sapiens
QQYGSSPQT

SEQ ID NO: 308 - protein, 9 aa, Homo sapiens
QQYGSSPQT

SEQ ID NO: 309 - protein, 24 aa, Homo sapiens
LEQSGAEVKKPGSSVKVSCKASGG

SEQ ID NO: 310 - protein, 24 aa, Homo sapiens
LEQSGAEVKKPGSSVKVSCRASGS

SEQ ID NO: 311 - protein, 24 aa, Homo sapiens
LEQSGAEVKKPGSSVKVSCRASGS

SEQ ID NO: 312 - protein, 24 aa, Homo sapiens
LEQSGAEVKKPGSSVKVSCRASGS

SEQ ID NO: 313 - protein, 24 aa, Homo sapiens
LEQSGAEVKTPGSSVRVSCRPPGG

SEQ ID NO: 314 - protein, 24 aa, Homo sapiens
LEQSGAEVKKPGSSVRVSCRAPGG

SEQ ID NO: 315 - protein, 24 aa, Homo sapiens
LEQSGAEVKEPGSSVRVSCRAPGG

SEQ ID NO: 316 - protein, 23 aa, Homo sapiens
LESGAEVKKPGSSVRVSCRPPGG

SEQ ID NO: 317 - protein, 24 aa, Homo sapiens
LEQSGAEVKKPGSSVRVSCRAPGG

SEQ ID NO: 318 - protein, 24 aa, Homo sapiens
LEQSGAEVKKPGSSVRVSCRPPGG

SEQ ID NO: 319 - protein, 23 aa, Homo sapiens
LESGGGLVQPGGSLRLSCEASGY

SEQ ID NO: 320 - protein, 24 aa, Homo sapiens
LEQSGGGLVQPGGSLRLSCEASGY

SEQ ID NO: 321 - protein, 23 aa, Homo sapiens
LESGGGLVQPGGSLRLSCEASGY

SEQ ID NO: 322 - protein, 23 aa, Homo sapiens
LESGGGLVQPGGSLRLSCEASGY

SEQ ID NO: 323 - protein, 24 aa, Homo sapiens
LEQSGAELKKPGSSVKVSCKPSDG

SEQ ID NO: 324 - protein, 24 aa, Homo sapiens
LEQSGNEVKKPGASVKVSCRAYGY

SEQ ID NO: 325 - protein, 24 aa, Homo sapiens
LEQSGAEMKKPGASLKVSCKTSGY

SEQ ID NO: 326 - protein, 24 aa, Homo sapiens
LEQSGAEVKKPGSSVKVSCEASGG

SEQ ID NO: 327 - protein, 24 aa, Homo sapiens
LEQSGAEVKKPGSSVKVSCETSGG

SEQ ID NO: 328 - protein, 24 aa, Homo sapiens
LEQSGAEVKKPGSSVKVSCETSGG

SEQ ID NO: 329 - protein, 23 aa, Homo sapiens
LESGGGLVQPGRSLRLSCKASGF

SEQ ID NO: 330 - protein, 24 aa, Homo sapiens
LEQSGPEVKKPGSSVKVSCKGSGD

SEQ ID NO: 331 - protein, 24 aa, Homo sapiens
LEQSGPEVKKPGSSVKVSCKDSGD

SEQ ID NO: 332 - protein, 24 aa, Homo sapiens
LEQSGPEVKKPGSSVKVSCKGSGD

SEQ ID NO: 333 - protein, 24 aa, Homo sapiens
LEQSGAEVKKPGSSVRVSCEVSGD

SEQ ID NO: 334 - protein, 24 aa, Homo sapiens
LEQSGPGLVKPGRPFSLTCAISGD

SEQ ID NO: 335 - protein, 24 aa, Homo sapiens
LEQSGAEVKKPGSSVKVSCKASGD

SEQ ID NO: 336 - protein, 24 aa, Homo sapiens
LEQSGAEVKMPGSSVKVSCKASGD

SEQ ID NO: 337 - protein, 24 aa, Homo sapiens
LEQSGAEVKKPGSSVKVSCKASGD

SEQ ID NO: 338 - protein, 24 aa, Homo sapiens
LEQSGAEVKKPGSSVKVSCKASGD

SEQ ID NO: 339 - protein, 24 aa, Homo sapiens
LEQSGAEVKKPGASVKVSCKASGY

SEQ ID NO: 340 - protein, 15 aa, Homo sapiens
WVRQAPGQGLEWVGG

SEQ ID NO: 341 - protein, 15 aa, Homo sapiens
WVRQAPGQGLEWMGG

SEQ ID NO: 342 - protein, 15 aa, Homo sapiens
WVRQAPGQGLEWMGG

SEQ ID NO: 343 - protein, 15 aa, Homo sapiens
WVRQAPGQGLEWMGG

SEQ ID NO: 344 - protein, 15 aa, Homo sapiens
WVRQAPGHGLEWVGT

SEQ ID NO: 345 - protein, 15 aa, Homo sapiens
WVRQAPGHGLEWVGT

SEQ ID NO: 346 - protein, 15 aa, Homo sapiens
WVRQAPGHGLEWVGT

SEQ ID NO: 347 - protein, 15 aa, Homo sapiens
WVRQAPGHGLEWVGT

SEQ ID NO: 348 - protein, 15 aa, Homo sapiens
WVRQAPGHGLEWVGT

SEQ ID NO: 349 - protein, 15 aa, Homo sapiens
WVRQAPGHGLEWVGT

SEQ ID NO: 350 - protein, 15 aa, Homo sapiens
WVRQTPGKGLEWVSS

SEQ ID NO: 351 - protein, 15 aa, Homo sapiens
WVRQTPGKGLEWVSS

SEQ ID NO: 352 - protein, 15 aa, Homo sapiens
WVRQTPGKGLEWVSS

SEQ ID NO: 353 - protein, 15 aa, Homo sapiens
WVRQTPGKGLEWVSS

SEQ ID NO: 354 - protein, 15 aa, Homo sapiens
WVRQAPGQTLEWMGR

SEQ ID NO: 355 - protein, 15 aa, Homo sapiens
WVRQAPGQGLEWMGW

SEQ ID NO: 356 - protein, 15 aa, Homo sapiens
WVRQAPGQGLEWMGW

SEQ ID NO: 357 - protein, 15 aa, Homo sapiens
WVRQAPGQGLEWIGG

SEQ ID NO: 358 - protein, 15 aa, Homo sapiens
WVRQAPGQGLEWIGG

SEQ ID NO: 359 - protein, 15 aa, Homo sapiens
WVRQAPGQGLEWIGG

SEQ ID NO: 360 - protein, 15 aa, Homo sapiens
WVRQAPGKGLEWIGL

SEQ ID NO: 361 - protein, 15 aa, Homo sapiens
WVRQAPGQGLEWIGG

SEQ ID NO: 362 - protein, 15 aa, Homo sapiens
WVRQAPGQGLEWIGG

SEQ ID NO: 363 - protein, 15 aa, Homo sapiens
WVRQAPGQGLEWIGG

SEQ ID NO: 364 - protein, 15 aa, Homo sapiens
WVRQAPGQGFEWIGG

SEQ ID NO: 365 - protein, 15 aa, Homo sapiens
WVRQSPSRGLEWLGR

SEQ ID NO: 366 - protein, 15 aa, Homo sapiens
WARQAPGQGLEWMGA

SEQ ID NO: 367 - protein, 15 aa, Homo sapiens
WARQAPGQGLEWMGA

SEQ ID NO: 368 - protein, 15 aa, Homo sapiens
WARQAPGQGLEWMGA

SEQ ID NO: 369 - protein, 15 aa, Homo sapiens
WARQAPGQGLEWMGA

SEQ ID NO: 370 - protein, 15 aa, Homo sapiens
WVRQAPGQGLEWMGW

SEQ ID NO: 371 - protein, 32 aa, Homo sapiens
RVTMTADESMTTVYMELSSLRSEDTAMYYCAR

SEQ ID NO: 372 - protein, 32 aa, Homo sapiens
RVTITADRSTTTAYMEMSSLTSDDTAIYYCAR

SEQ ID NO: 373 - protein, 32 aa, Homo sapiens
RVTITADRATATAYMEMSSLTSDDTAIYYCAR

SEQ ID NO: 374 - protein, 32 aa, Homo sapiens; identical to SEQ ID NO: 373

SEQ ID NO: 375 - protein, 32 aa, Homo sapiens
RVTITADGSSGTAYMDLNSLRSDDTAFYYCVR

SEQ ID NO: 376 - protein, 32 aa, Homo sapiens
RVTITADGSSGTAYMELNSLRSDDTAVYYCVR

SEQ ID NO: 377 - protein, 32 aa, Homo sapiens; identical to SEQ ID NO: 376

SEQ ID NO: 378 - protein, 32 aa, Homo sapiens; identical to SEQ ID NO: 376

SEQ ID NO: 379 - protein, 32 aa, Homo sapiens; identical to SEQ ID NO: 376

SEQ ID NO: 380 - protein, 32 aa, Homo sapiens; identical to SEQ ID NO: 376

SEQ ID NO: 381 - protein, 32 aa, Homo sapiens
RFTISRDNSKNTVFLHMNNLRPEDTAVYYCAK

SEQ ID NO: 382 - protein, 32 aa, Homo sapiens; identical to SEQ ID NO: 381

SEQ ID NO: 383 - protein, 32 aa, Homo sapiens
RFTISRDNSKNTMFLHMNNLRPEDTAVYYCAK

SEQ ID NO: 384 - protein, 32 aa, Homo sapiens
RFTISRDNSKNTVFLHMSNLRPEDTAVYYCAK

SEQ ID NO: 385 - protein, 32 aa, Homo sapiens
RVTISADMSTATAYMELSSLRSDDTAIYYCAK

SEQ ID NO: 386 - protein, 32 aa, Homo sapiens
RFTMTTDTSTRTGYMELRNLRSDDTAVYYCAR

SEQ ID NO: 387 - protein, 32 aa, Homo sapiens
RVTMTTDPSTRTAYMELRSLRSDDTAVYFCAR

SEQ ID NO: 388 - protein, 32 aa, Homo sapiens
RVTISDDKSTGTGHMELRSLRSEDTAVYYCVR

SEQ ID NO: 389 - protein, 32 aa, Homo sapiens; identical to SEQ ID NO: 388

SEQ ID NO: 390 - protein, 32 aa, Homo sapiens; identical to SEQ ID NO: 388

SEQ ID NO: 391 - protein, 32 aa, Homo sapiens
RFTISRDDSKSTAYLQINGLKTEDTAVYYCAR

SEQ ID NO: 392 - protein, 32 aa, Homo sapiens
RVVISADASTDTAYMELSSLRSEDTAVYYCAK

SEQ ID NO: 393 - protein, 32 aa, Homo sapiens
RVIISADASTATAYLELSSLRSEDTAVYYCAK

SEQ ID NO: 394 - protein, 32 aa, Homo sapiens
RVTISADASALTTYMEMSSLRPEDTAVYYCAK

SEQ ID NO: 395 - protein, 32 aa, Homo sapiens
RITISADQSTNTVYMELRSLRSDDTAVYYCAK

SEQ ID NO: 396 - protein, 32 aa, Homo sapiens
RITINSDTSKNQFSLHLNSVTPEDTAVYYCVR

SEQ ID NO: 397 - protein, 32 aa, Homo sapiens
RVTITADESTQTVYMDLSSLRSDDTAVYYCAK

SEQ ID NO: 398 - protein, 32 aa, Homo sapiens
RVTITADESTKTVYMDLSSLRSDDTAVYYCAK

SEQ ID NO: 399 - protein, 32 aa, Homo sapiens
RVTITADESTKTVYMDLSSLTSDDTAVYYCAK

SEQ ID NO: 400 - protein, 32 aa, Homo sapiens; identical to SEQ ID NO: 398

SEQ ID NO: 401 - protein, 32 aa, Homo sapiens
RVTMTTDTSTSTAYMELTSLTSEDTAVYYCAR

SEQ ID NO: 402 - protein, 13 aa, Homo sapiens
WGQGTMVTVSSAS

SEQ ID NO: 403 - protein, 13 aa, Homo sapiens
WGQGTLVIVSSAS

SEQ ID NO: 404 - protein, 13 aa, Homo sapiens
WGQGTPVIVSSAS

SEQ ID NO: 405 - protein, 13 aa, Homo sapiens
WGQGTPVIVSSAS

SEQ ID NO: 406 - protein, 13 aa, Homo sapiens
WGQGTLVTVSSAS

SEQ ID NO: 407 - protein, 13 aa, Homo sapiens
WGQGTLVTVSSAS

SEQ ID NO: 408 - protein, 13 aa, Homo sapiens
WGQGTLVTVSSAS

SEQ ID NO: 409 - protein, 13 aa, Homo sapiens
WGQGTLVTVSSAS

SEQ ID NO: 410 - protein, 13 aa, Homo sapiens
WGQGTLVTVSSAS

SEQ ID NO: 411 - protein, 13 aa, Homo sapiens
WGQGTLVTVSSAS

SEQ ID NO: 412 - protein, 13 aa, Homo sapiens
WGQGTLVTVSSAS

SEQ ID NO: 413 - protein, 13 aa, Homo sapiens
WGQGTLVTVSSAS

SEQ ID NO: 414 - protein, 13 aa, Homo sapiens
WGQGTLVTVSSAS

SEQ ID NO: 415 - protein, 13 aa, Homo sapiens
WGQGTLVTVSSAS

SEQ ID NO: 416 - protein, 13 aa, Homo sapiens
WGQGTLVTVSSAS

SEQ ID NO: 417 - protein, 13 aa, Homo sapiens
WGQGTLVIVSSDE

SEQ ID NO: 418 - protein, 13 aa, Homo sapiens
WGQGTTVTVSSAS

SEQ ID NO: 419 - protein, 13 aa, Homo sapiens
WGQGTLVTVSSAS

SEQ ID NO: 420 - protein, 13 aa, Homo sapiens
WGQGTLVTVSSAS

SEQ ID NO: 421 - protein, 13 aa, Homo sapiens
WGQGTLVTVSSAS

SEQ ID NO: 422 - protein, 13 aa, Homo sapiens
WGQGTLVTVSSAS

SEQ ID NO: 423 - protein, 13 aa, Homo sapiens
WGQGTLVTVSAAS

SEQ ID NO: 424 - protein, 13 aa, Homo sapiens
WGQGTQVTVSSAS

SEQ ID NO: 425 - protein, 13 aa, Homo sapiens
WGQGTLITVSSAS

SEQ ID NO: 426 - protein, 13 aa, Homo sapiens
WGQGTLVTVSSAS

SEQ ID NO: 427 - protein, 13 aa, Homo sapiens
WGQGTTVTVSSAS

SEQ ID NO: 428 - protein, 13 aa, Homo sapiens
WGQGTLVTVSSAS

SEQ ID NO: 429 - protein, 13 aa, Homo sapiens
WGQGTLVTVSSAS

SEQ ID NO: 430 - protein, 13 aa, Homo sapiens
WGQGTLVTVSSAS

SEQ ID NO: 431 - protein, 13 aa, Homo sapiens
WGQGTLVTVSSAS

SEQ ID NO: 432 - protein, 13 aa, Homo sapiens
WGQGTLVTISSAS

SEQ ID NO: 433 - protein, 21 aa, Homo sapiens
ELTQSPATLSVSPGESATLSC

SEQ ID NO: 434 - protein, 23 aa, Homo sapiens
ELTLTQSPGTLSLSPGERATLSC

SEQ ID NO: 435 - protein, 21 aa, Homo sapiens
TLTQSPDSLAVSLGERATINC

SEQ ID NO: 436 - protein, 23 aa, Homo sapiens
ELVMTQSPGTLSLSPGERATLSC

SEQ ID NO: 437 - protein, 23 aa, Homo sapiens
ELTLTQSPGTLSLSPGERATLSC

SEQ ID NO: 438 - protein, 21 aa, Homo sapiens
ELTQSPSTLSLSPGEGATLSC

SEQ ID NO: 439 - protein, 21 aa, Homo sapiens
ELTQSPGTLSLSPGERAALSC

SEQ ID NO: 440 - protein, 21 aa, Homo sapiens
ELTQSPGTLSLSPGERATLSC

SEQ ID NO: 441 - protein, 21 aa, Homo sapiens
ELTQSPGTLSLSPGERATLSC

SEQ ID NO: 442 - protein, 21 aa, Homo sapiens
ELTQSPGTLSLSPGERATLSC

SEQ ID NO: 443 - protein, 21 aa, Homo sapiens
ELTQSPATLYVSPGERATLSC

SEQ ID NO: 444 - protein, 21 aa, Homo sapiens
ELTQSPGTLSVSPGEAATLSC

SEQ ID NO: 445 - protein, 21 aa, Homo sapiens
ELTQSPGTLSLSPGERATLSC

SEQ ID NO: 446 - protein, 21 aa, Homo sapiens
TLTQSPATLSVSPGERATLSC

SEQ ID NO: 447 - protein, 23 aa, Homo sapiens
ELTLTQSPGTLSLSPGERATLSC

SEQ ID NO: 448 - protein, 23 aa, Homo sapiens
ELVMTQSPGTLSLSPGERATLSC

SEQ ID NO: 449 - protein, 23 aa, Homo sapiens
ELVLTQSPLSLPVTLGQPASISC

SEQ ID NO: 450 - protein, 23 aa, Homo sapiens
ELQMTQSPSFLSASVGDRVTITC

SEQ ID NO: 451 - protein, 21 aa, Homo sapiens
ELTQSPVSLPVTPGEPASISC

SEQ ID NO: 452 - protein, 23 aa, Homo sapiens
ELTLTQSPGTLSLSPGERATLSC

SEQ ID NO: 453 - protein, 21 aa, Homo sapiens
ELTQSPATLSVSPGERATLSC

SEQ ID NO: 454 - protein, 23 aa, Homo sapiens
ELTLTQSPGTLSLSPGERATLSC

SEQ ID NO: 455 - protein, 23 aa, Homo sapiens
ELTLTQSPGTLSLSPGERGTLSC

SEQ ID NO: 456 - protein, 21 aa, Homo sapiens
ELTQSPATLSVSPGERATLSC

SEQ ID NO: 457 - protein, 23 aa, Homo sapiens
ELTLTQSPATLSVSPGERATLFC

SEQ ID NO: 458 - protein, 23 aa, Homo sapiens
ELVLTQSPGTLSLSPGERATLSC

SEQ ID NO: 459 - protein, 21 aa, Homo sapiens
ELTQSPGTLSLSPGERATLSC

SEQ ID NO: 460 - protein, 21 aa, Homo sapiens
ELTQSPGTLSLSPGERATLSC

SEQ ID NO: 461 - protein, 23 aa, Homo sapiens
EFTLTQSPGTLSLSPGERGTLSC

SEQ ID NO: 462 - protein, 23 aa, Homo sapiens
ELTLTQSPGTLSLSPGERATLSC

SEQ ID NO: 463 - protein, 21 aa, Homo sapiens
ELTQSPGTLSLSPGERATLSC

SEQ ID NO: 464 - protein, 21 aa, Homo sapiens
ELTQSPGTLSLSPGERATLSC

SEQ ID NO: 465 - protein, 23 aa, Homo sapiens
ELVMTQSPATLSLSPGERATLSC

SEQ ID NO: 466 - protein, 21 aa, Homo sapiens
ELTQSPGTLSLSPGERATLSC

SEQ ID NO: 467 - protein, 23 aa, Homo sapiens
ELTLTQSPGTLSLSPGERATLSC

SEQ ID NO: 468 - protein, 15 aa, Homo sapiens
WYQQKPGQAPRLLIY

SEQ ID NO: 469 - protein, 15 aa, Homo sapiens
WYQQKPGQAPRLLIY

SEQ ID NO: 470 - protein, 15 aa, Homo sapiens
WYQQKPGQPPQLLIY

SEQ ID NO: 471 - protein, 15 aa, Homo sapiens
WYQQKPGQAPRLLVY

SEQ ID NO: 472 - protein, 15 aa, Homo sapiens
WYQQKPGQAPRLLIY

SEQ ID NO: 473 - protein, 15 aa, Homo sapiens
WYQQKPGQAPRLLIY

SEQ ID NO: 474 - protein, 15 aa, Homo sapiens
WYQQKPGQAPRLLIY

SEQ ID NO: 475 - protein, 15 aa, Homo sapiens
WYQQKPGQAPRLLIY

SEQ ID NO: 476 - protein, 15 aa, Homo sapiens
WYQQKPGQAPRLLIY

SEQ ID NO: 477 - protein, 15 aa, Homo sapiens
WYQQKPGQAPRLLIY

SEQ ID NO: 478 - protein, 15 aa, Homo sapiens
WYQQKPGQTPRLLIY

SEQ ID NO: 479 - protein, 15 aa, Homo sapiens
WYQQKPGQAPRLLIY

SEQ ID NO: 480 - protein, 15 aa, Homo sapiens
WYQQKPGQAPRLLIY

SEQ ID NO: 481 - protein, 15 aa, Homo sapiens
WYQQKPGQAPRLLIY

SEQ ID NO: 482 - protein, 15 aa, Homo sapiens
WYQQKPGQAPRLLIY

SEQ ID NO: 483 - protein, 15 aa, Homo sapiens
WYQQKPGQAPRLLIY

SEQ ID NO: 484 - protein, 15 aa, Homo sapiens
WFHQRAGQPPRRLIY

SEQ ID NO: 485 - protein, 15 aa, Homo sapiens
WYQQKPGKAPKLLIS

SEQ ID NO: 486 - protein, 15 aa, Homo sapiens
WYLQKPGQSPQLLMY

SEQ ID NO: 487 - protein, 15 aa, Homo sapiens
WYQQKPGQAPRLLIY

SEQ ID NO: 488 - protein, 15 aa, Homo sapiens
WYQQKPGQAPRLLIY

SEQ ID NO: 489 - protein, 15 aa, Homo sapiens
WYQQKPGQAPRLLIY

SEQ ID NO: 490 - protein, 15 aa, Homo sapiens
WYQQKPGQAPRLLIY

SEQ ID NO: 491 - protein, 15 aa, Homo sapiens
WYQQKPGQAPRLLIY

SEQ ID NO: 492 - protein, 15 aa, Homo sapiens
WYQQKPGQAPRLLIY

SEQ ID NO: 493 - protein, 15 aa, Homo sapiens
WYQQKPGQAPRLLIY

SEQ ID NO: 494 - protein, 15 aa, Homo sapiens
WYQQKPGQAPRLLIY

SEQ ID NO: 495 - protein, 15 aa, Homo sapiens
WYQQKPGQAPRLLIY

SEQ ID NO: 496 - protein, 15 aa, Homo sapiens
WYQQKPGQAPRLLIY

SEQ ID NO: 497 - protein, 15 aa, Homo sapiens
WYQQKPGQAPRLLIY

SEQ ID NO: 498 - protein, 15 aa, Homo sapiens
WYQQKPGLAPRLLIY

SEQ ID NO: 499 - protein, 15 aa, Homo sapiens
WYQQKPGQAPRLLIY

SEQ ID NO: 500 - protein, 15 aa, Homo sapiens
WYQQKPGQAPRLLIY

SEQ ID NO: 501 - protein, 15 aa, Homo sapiens
WYQHKPGQAPRLLIY

SEQ ID NO: 502 - protein, 15 aa, Homo sapiens
WYQQKPGQAPRLLIY

SEQ ID NO: 503 - protein, 32 aa, Homo sapiens
AIPGRFSGSGSGTDFTLTISRLEPEDLAVYHC

SEQ ID NO: 504 - protein, 32 aa, Homo sapiens
GIPDRFSGSGSGTDFTLTISRLEPEDFAVYYC

SEQ ID NO: 505 - protein, 32 aa, Homo sapiens
GVPDRFSGSGSGTDFTLTISSLQAEDVAVYFC

SEQ ID NO: 506 - protein, 32 aa, Homo sapiens
GIPDRFSGSGSGTDFTLTISRLQPEDFAVYYC

SEQ ID NO: 507 - protein, 32 aa, Homo sapiens
GIPHRFSGSGSGTDFTLTISRLEPEDFAVYYC

SEQ ID NO: 508 - protein, 32 aa, Homo sapiens
GIPDRFSGSGSGTNFTLTISRLEPEDFAVYFC

SEQ ID NO: 509 - protein, 32 aa, Homo sapiens
GIPDRFSGTGSGTDFSLTISRLEPEDSAVYYC

SEQ ID NO: 510 - protein, 32 aa, Homo sapiens; identical to SEQ ID NO: 504

SEQ ID NO: 511 - protein, 32 aa, Homo sapiens
GIPDRFSGSGSGTDFTLTISGLEPEDFAVYYC

SEQ ID NO: 512 - protein, 32 aa, Homo sapiens
GIPDRFSGSGSGTNFTLTISRLEPEDFAVYYC

SEQ ID NO: 513 - protein, 32 aa, Homo sapiens; identical to SEQ ID NO: 504

SEQ ID NO: 514 - protein, 32 aa, Homo sapiens
GIPARFSGSGSGTDFTLTISRLEPEDFAVYYC

SEQ ID NO: 515 - protein, 32 aa, Homo sapiens; identical to SEQ ID NO: 504

SEQ ID NO: 516 - protein, 32 aa, Homo sapiens; identical to SEQ ID NO: 504

SEQ ID NO: 517 - protein, 32 aa, Homo sapiens; identical to SEQ ID NO: 504

SEQ ID NO: 518 - protein, 32 aa, Homo sapiens
GIPDRFSGSGSGTDFTLTITRLEPEDFAVYYC

SEQ ID NO: 519 - protein, 32 aa, Homo sapiens
GVPERFSGSGSGTDFTLKISRVEAEDVGIYYC

SEQ ID NO: 520 - protein, 32 aa, Homo sapiens
GVSSRFSGSGSGTGFTLTISSLQSEDSATYYC

SEQ ID NO: 521 - protein, 32 aa, Homo sapiens
GVPDRFSGSGSGTDFTLKISRVEAEDVGVYYC

SEQ ID NO: 522 - protein, 32 aa, Homo sapiens; identical to SEQ ID NO: 504

SEQ ID NO: 523 - protein, 32 aa, Homo sapiens; identical to SEQ ID NO: 504

SEQ ID NO: 524 - protein, 32 aa, Homo sapiens
GIPDRFSGSGSGTDFTLTISRLEPEDLGVYYC

SEQ ID NO: 525 - protein, 32 aa, Homo sapiens; identical to SEQ ID NO: 504

SEQ ID NO: 526 - protein, 32 aa, Homo sapiens
GIPARFSGSGSGTDFTLTVSRLEPEDSAVYFC

SEQ ID NO: 527 - protein, 32 aa, Homo sapiens
TTPTRFSGSGSGTDFTLTISRLQSEDFAVYYC

SEQ ID NO: 528 - protein, 32 aa, Homo sapiens
GIPDRFSGSGSGTDFALTITRLEPEDFAVYYC

SEQ ID NO: 529 - protein, 32 aa, Homo sapiens; identical to SEQ ID NO: 504

SEQ ID NO: 530 - protein, 32 aa, Homo sapiens; identical to SEQ ID NO: 511

SEQ ID NO: 531 - protein, 32 aa, Homo sapiens; identical to SEQ ID NO: 504

SEQ ID NO: 532 - protein, 32 aa, Homo sapiens; identical to SEQ ID NO: 504

SEQ ID NO: 533 - protein, 32 aa, Homo sapiens
GIPDRFSGSGSGTGFTLTISTLEPEDFAIYYC

SEQ ID NO: 534 - protein, 32 aa, Homo sapiens; identical to SEQ ID NO: 504

SEQ ID NO: 535 - protein, 32 aa, Homo sapiens
GIPARFSGSGSGTDFTLTISSLEPEDFAVYFC

SEQ ID NO: 536 - protein, 32 aa, Homo sapiens; identical to SEQ ID NO: 504

SEQ ID NO: 537 - protein, 32 aa, Homo sapiens
GIPDRFSGSGSGTDFTLTISRLEPEDFAVYFC

SEQ ID NO: 538 - protein, 36 aa, Homo sapiens
FGQGTKVEIKRTVAAPSVFIFPPSDEQLKSGTASVV

SEQ ID NO: 539 - protein, 36 aa, Homo sapiens; identical to SEQ ID NO: 538

SEQ ID NO: 540 - protein, 36 aa, Homo sapiens; identical to SEQ ID NO: 538

SEQ ID NO: 541 - protein, 36 aa, Homo sapiens
FGQGTRVDIKRTVAAPSVSIFPPSDEQLKSGTASVV

SEQ ID NO: 542 - protein, 35 aa, Homo sapiens
FGQGTRVDIKRTVAAPSVFIFPPSDEQLKSGTASV

SEQ ID NO: 543 - protein, 23 aa, Homo sapiens
FGQGTKLEIKRTVAAPSVFIFPP

SEQ ID NO: 544 - protein, 24 aa, Homo sapiens
FGPGTKVDIKRTVAAPSVFIFPPS

SEQ ID NO: 545 - protein, 36 aa, Homo sapiens; identical to SEQ ID NO: 538

SEQ ID NO: 546 - protein, 25 aa, Homo sapiens
FGGGTKVEIKRTVAAPSVFIFPPSD

SEQ ID NO: 547 - protein, 36 aa, Homo sapiens
FGQGTRVEIKRTVAAPSVFIFPPSDEQLKSGTASVV

SEQ ID NO: 548 - protein, 36 aa, Homo sapiens; identical to SEQ ID NO: 538

SEQ ID NO: 549 - protein, 36 aa, Homo sapiens
FGQGTKLEIKRTVAAPSVFIFPPSDEQLKSGTASVV

SEQ ID NO: 550 - protein, 35 aa, Homo sapiens
FGQGTKVEIKRTVAAPSVFIFPPSDEQLKSGTASV

SEQ ID NO: 551 - protein, 25 aa, Homo sapiens
FGQGTKVEIKRTVAAPSVFIFPPSD

SEQ ID NO: 552 - protein, 33 aa, Homo sapiens
FGQGTKVEIKRTVAAPSVFIFPPSDEQLKSGTA

SEQ ID NO: 553 - protein, 33 aa, Homo sapiens
FGQGTKVQIKRTVAAPSVFIFPPSDEQLKSGTA

SEQ ID NO: 554 - protein, 34 aa, Homo sapiens
FGGGTKVEINRTVAAPSVFIFPPSDEQLKSGTAS

SEQ ID NO: 555 - protein, 25 aa, Homo sapiens
FGQGTKLEIKRTVAAPSVFIFPPSD

SEQ ID NO: 556 - protein, 25 aa, Homo sapiens
FGQGTKVEIKRTVAAPSVFIFPPSD

SEQ ID NO: 557 - protein, 25 aa, Homo sapiens
FGGGTKVEIKRTVAAPSVFIFPPSD

SEQ ID NO: 558 - protein, 27 aa, Homo sapiens
FGQGTEVEIKRTVAAPSVFIFPPSDEQ

SEQ ID NO: 559 - protein, 36 aa, Homo sapiens; identical to SEQ ID NO: 549

SEQ ID NO: 560 - protein, 36 aa, Homo sapiens; identical to SEQ ID NO: 538

SEQ ID NO: 561 - protein, 36 aa, Homo sapiens
FGGGTKVEIKRTVAAPSVFIFPPSDEQLKSGTASVV

SEQ ID NO: 562 - protein, 24 aa, Homo sapiens
FGQGTKLEIKRTVAAPSVFVFPPS

SEQ ID NO: 563 - protein, 36 aa, Homo sapiens; identical to SEQ ID NO: 538

SEQ ID NO: 564 - protein, 36 aa, Homo sapiens; identical to SEQ ID NO: 538

SEQ ID NO: 565 - protein, 36 aa, Homo sapiens; identical to SEQ ID NO: 538

SEQ ID NO: 566 - protein, 35 aa, Homo sapiens; identical to SEQ ID NO: 550

SEQ ID NO: 567 - protein, 36 aa, Homo sapiens
FGQGTEVEIKRTVAAPSVFIFPPSDEQLKSGTASVV

SEQ ID NO: 568 - protein, 36 aa, Homo sapiens
FGPGTKVDIRRTVAAPSVFIFPPSDEQLKSGTASVV

SEQ ID NO: 569 - protein, 36 aa, Homo sapiens
FGPGTKVEIKRTVAAPSVFIFPPSDEQLKSGTASVV

SEQ ID NO: 570 - protein, 36 aa, Homo sapiens; identical to SEQ ID NO: 538

SEQ ID NO: 571 - protein, 36 aa, Homo sapiens
FGQGTRLEIKRTVAAPSVFIFPPSDEQLKSGTASVV

SEQ ID NO: 572 - protein, 32 aa, Homo sapiens
FGQGTKVEIKRTVAAPSVFIFPPSDEQLKSGT

SEQ ID NO: 573 - protein, 26 aa, Homo sapiens
LLESGPGLVKPSQTLSLTCDISGDSV

SEQ ID NO: 574 - protein, 26 aa, Homo sapiens
LLESGPGLVKPSQTLSLTCDISGDSV

SEQ ID NO: 575 - protein, 26 aa, Homo sapiens
LLESGPGLVKPSQTLSLTCDISGDSV

SEQ ID NO: 576 - protein, 26 aa, Homo sapiens
LLESGPGLVRPSQTLSLTCDISGDSV

SEQ ID NO: 577 - protein, 26 aa, Homo sapiens
LLESGPGLVKPSQTLSLTCAISGDSV

SEQ ID NO: 578 - protein, 14 aa, Homo sapiens
WIRQSPRGLEWLGR

SEQ ID NO: 579 - protein, 14 aa, Homo sapiens
WIRQSPRGLEWLGR

SEQ ID NO: 580 - protein, 14 aa, Homo sapiens
WIRQSPRGLEWLGR

SEQ ID NO: 581 - protein, 14 aa, Homo sapiens
WIRQSPRGLEWLGR

SEQ ID NO: 582 - protein, 14 aa, Homo sapiens
WIRQSPRGLEWLGR

SEQ ID NO: 583 - protein, 27 aa, Homo sapiens
RIIISADTSKNQFSLQLNSVTPEDTAV

SEQ ID NO: 584 - protein, 27 aa, Homo sapiens
RIIISADTSKNQLSLQLNSVTPEDTAV

SEQ ID NO: 585 - protein, 27 aa, Homo sapiens
RITINPDTSKNQFSLQLNSVTPEDTAV

SEQ ID NO: 586 - protein, 27 aa, Homo sapiens
RITINPDTSKNQFSLQLNSVTPEDTAV

SEQ ID NO: 587 - protein, 27 aa, Homo sapiens
RITINPDTSKNQFSLHLNSVTPEDTAV

SEQ ID NO: 588 - protein, 13 aa, Homo sapiens
WGQGTTVTVSSAS

SEQ ID NO: 589 - protein, 13 aa, Homo sapiens
WGQGTTVTVSSAS

SEQ ID NO: 590 - protein, 13 aa, Homo sapiens
WGQGTTVTVSSAS

SEQ ID NO: 591 - protein, 13 aa, Homo sapiens
WGQGTTVTVSSAS

SEQ ID NO: 592 - protein, 13 aa, Homo sapiens
WGQGTTVTVSSAS

SEQ ID NO: 593 - protein, 21 aa, Homo sapiens
ELTQSPGTLSLSPGERATLSC

SEQ ID NO: 594 - protein, 21 aa, Homo sapiens
ELTQSPGTLSLSPGERGTLSC

SEQ ID NO: 595 - protein, 21 aa, Homo sapiens
ELTQSPGTLSLSPGERATLSC

SEQ ID NO: 596 - protein, 23 aa, Homo sapiens
ELTLTQSPGTLSLSPGERATLSC

SEQ ID NO: 597 - protein, 21 aa, Homo sapiens
ELTQSPGTLSLSPGERATLSC

SEQ ID NO: 598 - protein, 21 aa, Homo sapiens
ELTQSPGTLSLSPGERATLSC

SEQ ID NO: 599 - protein, 15 aa, Homo sapiens
WYQQKPGQAPRLLIY

SEQ ID NO: 600 - protein, 15 aa, Homo sapiens
WYQQKPGQAPRLLIY

SEQ ID NO: 601 - protein, 15 aa, Homo sapiens
WYQQKPGQAPRLLIY

SEQ ID NO: 602 - protein, 15 aa, Homo sapiens
WYQQKPGQAPRLLIY

SEQ ID NO: 603 - protein, 15 aa, Homo sapiens
WYQQKPGQAPRLLIY

SEQ ID NO: 604 - protein, 15 aa, Homo sapiens
WYQQKPGQAPRLLIY

SEQ ID NO: 605 - protein, 29 aa, Homo sapiens
GIPDRFSGSGSGTDFTLTISRLEPEDFAV

SEQ ID NO: 606 - protein, 27 aa, Homo sapiens
FGQGTKVEIKRTVAAPSVFIFPPSDEQ

SEQ ID NO: 607-610 - skipped (4 entries)

SEQ ID NO: 611 - DNA, 423 bp, Homo sapiens
ctcgagcagtctggggctgaggtgaagaagcctgggtcctcggtgaaagtctcctgcaaggcgtctgggggcaccttcagcagctttgttatcaactgggtgcgacaggcccctggacaagggctagagtgggtgggagggatcttccaggcccccggaccagagcgtgagtggctgcgggacattaacccaatctctgggacgataaactacgcacagaggttccagggcagagtcacgatgaccgcggacgaatccatgaccacagtctacatggagctgagcagtctgagatctgaagacacggccatgtattactgtgcgagagaaaacaaattcagatactgtcgtggtggtagttgctactctggtgcttttgatatgtggggccaggggacaatggtcaccgtctcttcagcctcc

SEQ ID NO: 612 - DNA, 369 bp, Homo sapiens
ctcgagcagtctggggcagaggtgaagaagccggggtcctcagtgaaagtctcctgcagggcctctggaagcccctttggtagttacacgatcacttgggtgcgacaggcccctggacaaggacttgagtggatgggaggaatcatcctgatgactggtaaagcgaactacgcacagaagttccagggcagagtcacgatcactgcggacagatcaacgaccacagcctacatggaaatgagcagcctgacatctgacgacacggccatttattactgtgcgagagatccctatgtatatgcaggggatgacgtgtggtctttgtctcggtggggccagggaaccctggttatcgtctcctcagcctcc

SEQ ID NO: 613 - DNA, 369 bp, Homo sapiens
ctcgagcagtctggggcagaggtgaagaagccggggtcctcagtgaaagtgtcctgcagggcctctggaagcccctatagtagttacacgatcacttgggtgcgacaggcccctggacaaggacttgagtggatgggaggaatcatcctgatgactggtaaagcgaactacgcacagaagttccagggcagagtcaccatcactgcggacagagcaacggccacagcctacatggaaatgagcagcctgacatctgacgacacggccatatattactgtgcgagagatccctatgtttatgcaggggatgacgtgcggtctttgtctcggtggggccagggaaccccggtcatcgtctcctcagcctcc

SEQ ID NO: 614 - DNA, 369 bp, Homo sapiens
ctcgagcagtctggggcagaggtgaagaagccggggtcctcagtgaaagtgtcctgcagggcctctggaagcccctatagtagttacacgatcacttgggtgcgacaggcccctggacaaggacttgagtggatgggaggaatcatcctgatgactggtaaagcgaactacgcacagaagttccagggcagagtcaccatcactgcggacagagcaacggccacagcctacatggaaatgagcagcctgacatctgacgacacggccatatattactgtgcgagagatccctatgtttatgcaggggatgacgtgtggtctttgtctcggtggggccagggaaccccggtcatcgtctcctcagcctcc

SEQ ID NO: 615 - DNA, 378 bp, Homo sapiens
ctcgagcagtctggggctgaggtgaagacgcctgggtcctcggtgagggtctcctgcaggcctcctggaggcaacttcaacagttatagtataaactgggtccgacaggcccctggacacggccttgagtgggtggggactttcatccctatgtttggaacctcaaagtacgcgcagaagtttcaggggagagtcacgattaccgcggacgggtcctcgggcaccgcttacatggacctgaacagcctgagatctgacgacacggccttttactactgtgtgcgtcctgaaacgcccagatattgtagtggcggtttctgctatggtgagtttgacaactggggccagggaaccctggtcaccgtctcctcagcctcc

SEQ ID NO: 616 - DNA, 378 bp, Homo sapiens
ctcgagcagtctggggctgaagtgaagaagcctgggtcttcggtgagagtctcctgcagggctcctggtggcaccttcaacagctatagtgtgaactgggtccgacaggcccctgggcacggccttgagtgggtggggacgctcatccctatgtttggtacctcaagttacgcgcagaagttccaggggagagtcaccattaccgcggacgggtcctcgggcaccgcctacatggaactgaacagcctgagatctgacgacacggccgtctactactgtgtgcgacctgaaacgcccagatattgtcgtggcggtttctgctatggtgaatttgacaactggggccagggaaccctggtcaccgtctcctcggcctcc

SEQ ID NO: 617 - DNA, 378 bp, Homo sapiens
ctcgagcagtctggggctgaggtgaaggagcctgggtcctcggtgagggtctcctgcagggctcctggaggcaccttcaacagctatagtataaattgggtccgacaggcccctggacacggccttgagtgggtggggacgctcatccctatgtttggtacctcaaactacgcgcagaagttccaggggagagtcaccattaccgcggacgggtcctcgggcaccgcctacatggagctgaacagcctgagatctgacgacacggccgtatactactgtgtgcgacctgaaacgcccagatattgtagtggcggtgtctgctatggtgaatttgacaactggggccagggaaccctggtcaccgtctcctcagcctcc

SEQ ID NO: 618 - DNA, 375 bp, Homo sapiens
ctcgagtctggggctgaggtgaagaagcctgggtcctcggtgagggtctcctgcaggcctcctggaggcaccttcaatagctatagtataaactgggtccgacaggcccctggacacggccttgagtgggtggggacgatcatccctatgtttggaacctcaaagtacgcgcagaagttgcaggggagagtcacgattaccgcggacgggtcctcgggcaccgcttacatggagctgaacagcctgagatctgacgacacggccgtatattactgtgtgcgacctgaaacgcccagatattgtagtggcggtttctgctatggtgaatttgacaactggggccagggaaccctggtcaccgtctcctcagcctcc

SEQ ID NO: 619 - DNA, 378 bp, Homo sapiens
ctcgagcagtctggggctgaggtgaagaagcctgggtcctcggtgagggtctcctgcagggctcctggaggcaccctcaacagctatagtataaactgggtccgacaggcccctggacacggccttgagtgggtggggacgctcatccctatgtttggtacctcaaactacgcgcagaagttccaggggagagtcaccattaccgcggacgggtcctcgggcaccgcctacatggagctgaacagcctgagatctgacgacacggccgtatactactgtgtgcgacctgaaacgcccagatattgtagtggcggtttctgctatggtgaatttgacaactggggccagggaaccctggtcaccgtctcctcagcctcc

SEQ ID NO: 620 - DNA, 378 bp, Homo sapiens
ctcgagcagtctggggctgaggtgaagaagcctgggtcctcggtgagggtctcctgcaggcctccgggaggcaccttcaacagttatagtataaactgggtccgacaggcccctggacacggccttgagtgggtggggacgatcatccctatgtttggaacctcaaaatacgcgcagaagttgcaggggagagtcacgattaccgcggacgggtcctcgggcaccgcttacatggagctgaacagcctgagatctgacgacacggccgtatactactgtgtgcgacctgaaacgcccagatattgtagtggcggtttctgctatggtgaatttgacaattggggccagggaaccctggtcaccgtctcctcagcctcc

SEQ ID NO: 621 - DNA, 366 bp, Homo sapiens
ctcgagtctgggggaggcttggtccagccgggggggtccctgagactctcctgtgaagcctctggatattatttcagtagctttgccatgagttgggtccgccagactccagggaagggactggagtgggtctccagtattgctggtggtactcttggaagaacatcctatagagactccgtgaagggccgcttcaccatctccagagacaactccaagaatacggtgtttctccacatgaacaacctgagacccgaggacacggccgtctattattgtgcgaaagatccacttctcttcgcaggaggacctaattggttcgaccactggggccagggaaccctggtcaccgtctcctcagcctcc

SEQ ID NO: 622 - DNA, 369 bp, Homo sapiens
ctcgagcagtctgggggaggcttggtccagccgggggggtccctgagactctcctgtgaagcctctggatattatttcagtagctttgccatgagttgggtccgccagactccagggaagggactggagtgggtctccagtattgctggtggtactcttggaagaacatcctatagagactccgtgaagggccgcttcaccatctccagagacaactccaagaatacggtgtttctccacatgaacaacctgagacccgaggacacggccgtctattattgtgcgaaagatccacttctcttcgcaggaggacctaattggttcgaccactggggccagggaaccctggtcaccgtctcctcagcctcc

SEQ ID NO: 623 - DNA, 366 bp, Homo sapiens
ctcgagtctgggggaggcttggtccagccgggggggtccctgagactctcctgtgaagcctctggatattatttcagtagctttgccatgagttgggtccgccagactccagggaagggactggagtgggtctccagtattgctggtggtactcttggaagaacatcctatagagactccgtgaagggccgcttcaccatctccagagacaactccaagaatacaatgtttctccacatgaacaacctgagacccgaggacacggccgtctattattgtgcgaaagatccacttctcttcgcaggaggacctaattggttcgaccactggggccagggaaccctggtcaccgtctcctcagcctcc

SEQ ID NO: 624 - DNA, 366 bp, Homo sapiens
ctcgagtctgggggaggcttggtccagccgggggggtccctgagactctcctgtgaagcctctggatattatttcagtagctttgccatgagttgggtccgccagactccagggaagggactggagtgggtctccagtattgctggtggtactcttggaagaacatcctatagagactccgtgaagggccgcttcaccatctccagagacaactccaagaatacggtgtttctccacatgagcaacctgagacccgaggacacggccgtctattattgtgcgaaagatccacttctcttcgcaggaggacctaattggttcgaccactggggccagggaaccctggtcaccgtctcctcagcctcc

SEQ ID NO: 625 - DNA, 360 bp, Homo sapiens
ctcgagcagtctggggctgagctgaagaagcctgggtcttcggtgaaggtctcctgcaagccctctgatggcaccttcagggcctatactctcagctgggtgcgacaggcccctggacaaacgcttgagtggatgggcaggatcatgcctactgttggcataacaaactacgcacagaaattccagggcagagtcaccatttccgcggacatgtccacggccacagcctacatggaactgagcagcctgcgatctgacgacacggccatatattactgtgcgaaaggcccgtacgttggccttggggaagggttctcggagtggggccagggaactctggtcaccgtctcctcagcctcc

SEQ ID NO: 626 - DNA, 354 bp, Homo sapiens
ctcgagcagtcaggaaatgaggtgaagaagccaggggcctcagtgaaagtctcctgccgggcttatggctacaattttggcagcgaacgtctcagctgggtgcgacaggcccctggacaaggccttgagtggatgggatggatcagcgcttacaatggtggcataaactattcacagaagttccagggcagattcaccatgaccacagacacgtccacgcgcacaggctacatggaattgaggaacctcagatctgacgacacggccgtctattactgtgcgagagggggggggactgagtggggccagggaaccctggtcatcgtctcctcagatgagtcctcctcagcctcc

SEQ ID NO: 627 - DNA, 378 bp, Homo sapiens
ctcgagcagtcaggagctgagatgaagaagcctggggcctcattgaaggtctcctgcaagacttctggttacacgtttgacgactatggtgtcacctgggtgcgacaggcccctggacaaggacttgagtggatgggctggatcagcgcttacagtggtaacacattctatgcacggaagttccagggcagagtcaccatgaccacagacccatccacgcgcactgcctacatggagctgaggagcctgagatctgacgacacggccgtgtatttctgtgcgagagatcctggtcttgcgattaatggagtggttttcccctacttcggtttggacgtctggggccaagggaccacggtcaccgtctcatcagcctcc

SEQ ID NO: 628 - DNA, 378 bp, Homo sapiens
ctcgagcagtctggggctgaggtgaagaagcctggctcctcggtgaaagtctcctgcgaggcttctggaggcaccttcgacaactattctctcaattgggtgcgacaggcccctggacaaggacttgagtggataggaggggtcgtccctttgttcggtacaacaaaatacgcacagaagttccagggcagagtcacgataagcgacgacaaatcgacgggcacaggacacatggagttgagaagcctgagatcggaagacacggccgtctattattgtgtgagatcagtcacgcccagacattgtggtggtgggttttgctacggtgaatttgactactggggccagggaaccctggtcaccgtctcctcagcctcc

SEQ ID NO: 629 - DNA, 378 bp, Homo sapiens
ctcgagcagtctggggctgaggtgaagaagcctggctcctcggtgaaagtctcctgcgagacttctggagggactttcgacaactatgctctcaattgggtgcgacaggcccctggacaaggacttgagtggataggaggggtcgtccctttgttcggtacaacaaaatacgcacagaagttccagggcagagtcacgataagcgacgacaaatcgacgggcacaggacacatggagttgagaagcctgagatcggaagacacggccgtctattattgtgtgagatcagtcacgcccagacattgtggtggtgggttttgctacggtgaatttgactactggggccagggaaccctggtcaccgtctcctcagcctcc

SEQ ID NO: 630 - DNA, 378 bp, Homo sapiens
ctcgagcagtctggggctgaggtgaagaagcctggctcctcggtgaaagtctcctgcgagacttctggagggaccttggacaactatgctctcaattgggtgcgacaggcccctggacaaggacttgagtggatcggaggggtcgtccctttgtttggtacaacaagaaacgcacagaagttccagggcagagtcacgataagcgacgacaaatcgacgggcacaggacacatggagttgagaagcctgcgatcggaagacacggccgtttattattgtgtgagatcagtcacgcccagatattgtggtggtgggttttgctacggtgaatttgactactggggccagggaaccctggtcaccgtctcctcagcctcc

SEQ ID NO: 631 - DNA, 381 bp, Homo sapiens
ctcgagtctgggggaggcttggtacagccagggcggtccctgagactctcctgcaaagcgtctggcttcaattttgctcagtatactatgaactgggtccgccaggctccagggaagggactggagtggatcgggctcattagaaccacagcttatgatgcggctacacattatgctgcgtctgtggagggcagattcaccatttccagagatgattccaaaagtaccgcctatctgcagataaacggcctgaaaaccgaggacacagccgtctattactgtgctagaccccatggacccgggttaagtcttggcatttacagcgctgaatacttcgatgagtggggccagggcaccctggtcaccgtctcctcagcctcc

SEQ ID NO: 632 - DNA, 378 bp, Homo sapiens
ctcgagcagtctgggcctgaggtgaagaagcctgggtcctcggtgaaggtctcctgcaagggttctggagacaggttcaacgatcctgtcacctgggtgcgacaggcccctggacaaggccttgagtggatcggaggaatcatccctgcgtttggtgcaacaaagtatgcacagaagttccagggcagagtcgtcatttccgcggacgcatccacggacacggcctacatggaactgagcagcctgagatctgaagacacggccgtctattattgtgcgaaagtaggcgtgcggggcattattttggttgggggcctggcgatgaactggctcgacccctggggccagggaaccctagtcaccgtctccgcagcctcc

SEQ ID NO: 633 - DNA, 378 bp, Homo sapiens
ctcgagcagtctgggcctgaggtgaagaagcctgggtcgtcggtgaaggtctcctgcaaggattctggagacaccttcaacgaacctgtcacctgggtgcgacaggcccctggacaaggccttgagtggatcggaggaatcatccctgcgtttggtgtgacaaagtacgcacagaaattccagggccgagtcatcatttccgcggacgcatctacggccacggcctatttggagctgagcagtctgagatctgaagacacggccgtctattactgtgcgaaagttggcctgcggggcattgtaatggttgggggcctggcgatgaactggctcgacccctggggccagggaacccaagtcaccgtctcctcagcctcc

SEQ ID NO: 634 - DNA, 378 bp, Homo sapiens
ctcgagcagtctgggcctgaggtgaagaagcctgggtcctcggtgaaagtctcctgcaagggttctggagacaccttcaacgatcctgtcacctgggtgcgacaggcccctggacaaggccttgagtggatcggaggaatcatccctctgtttggtgcagcaaaatacgcacagaagttccagggcagagtcacgatttccgcggacgcatcagcgttaacgacctacatggagatgagcagcctgagaccggaagacacggccgtctattattgtgcgaaagtgggtctgcggggcattactttggttgggggcctggcgatgaactggctcgacccctggggccagggaaccctaatcacggtctcctcagcctcc

SEQ ID NO: 635 - DNA, 378 bp, Homo sapiens
ctcgagcagtctggggctgaggtgaagaagcctgggtcctcggtgagagtctcctgcgaggtttctggagacaccttcagggagcctgtcagttgggtgcgacaggcccctggacaaggatttgagtggatcggaggaatcatccctatgtttggcgcaacacattacgcacagaagttacagggcagaatcactatttctgcggaccaatcgacgaacacagtctacatggaactgaggagcctgagatctgacgacacggcagtttattattgtgcgaaagttggactgcggggcattaatatggttgggggcctggcgatgaactggttcgacccctggggccagggaaccctagtcaccgtctcctcagcctcc

SEQ ID NO: 636 - DNA, 381 bp, Homo sapiens
ctcgagcagtcaggtccaggactggtgaagcctggcagacccttttcactcacctgtgccatctccggggacagtgtctctagcgacagtgctgcttggaactgggtcaggcagtccccgtcgagaggccttgagtggctgggaaggacattctacaggtccaagtggtattatgattatacagtatctgtgaaaagtcgaatcaccatcaactcagacacatccaagaaccagttttccctgcacctcaactctgtgactcccgaggacacggctgtgtattattgtgtaagagatttttatattggcccaaccagagacgtctactacggtatggacgtctggggccaagggaccacggtcaccgtctcctcagcctcc

SEQ ID NO: 637 - DNA, 390 bp, Homo sapiens
ctcgagcagtctggggctgaggtgaagaagcctgggtcgtcggtgaaggtctcctgcaaggcttctggagacaccttcagaagttatgtcatcacatgggcgcgacaggcccctggacaagggcttgagtggatgggagcgatcatccctttctttggaacaacaaacctcgcacagaaattccagggcagagtcacgattaccgcggacgaatcaacgcagacagtctacatggacttgagcagcctgagatctgacgacacggccgtttattattgtgcgaaagccggagatctttcagttgggggagttctcgccggcggggttccgcacttgcgacattttgacccctggggccagggaaccctggtcaccgtctcctcggcctcc

SEQ ID NO: 638 - DNA, 390 bp, Homo sapiens
ctcgagcagtctggggctgaggtgaagatgcccgggtcgtcggtgaaggtctcctgcaaggcttctggagacacgttcagaagttctgttatcacatgggcgcgacaggcccctggacaggggcttgagtggatgggagcgatcatccctttctttggaacaacaaacctcgcacagaagttccagggcagagtcacgattaccgcggacgaatcaacgaagacagtctacatggacttgagcagcctgagatctgatgacacggccgtttattattgcgcgaaagccggagatctttcagttgggggagttctcgccggcggggttccgcacttgcgacatttcgacccctggggccagggaaccctggtcaccgtctcctcggcctcc

SEQ ID NO: 639 - DNA, 390 bp, Homo sapiens
ctcgagcagtctggggctgaggtgaagaagcctgggtcgtcggtgaaggtctcctgcaaggcttctggagacaccttcagaagttatgttatcacatgggcgcgacaggcccctggacaagggcttgagtggatgggagcgatcatccctttctttggaacaacaaacctcgcacagaagttccagggcagagtcacgattaccgcggacgaatcaacgaagacagtctacatggacttgagcagcctgacatctgatgacacggccgtttattattgtgcgaaagccggagatctttcagttgggggagttctcgccggcggggttccgcacttgcgacatttcgacccctggggccagggaaccctggtcaccgtctcctcggcctcc

SEQ ID NO: 640 - DNA, 390 bp, Homo sapiens
ctcgagcagtctggggctgaggtgaagaagcctgggtcgtcggtgaaggtctcctgcaaggcttctggagacaccttcagaagttatgttatcacatgggcgcgacaggcccctggacaagggcttgagtggatgggagcgatcatccctttctttggaacaacaaacctcgcacagaagttccagggcagagtcacgattaccgcggacgaatcaacgaagacagtctacatggacttgagcagcctgagatctgatgacacggccgtttattattgtgcgaaagccggagatctttcagttgggggagttctcgccggcggggttccgcacttgcgacatttcgacccctggggccagggaaccctggtcaccgtctcctcggcctcc

SEQ ID NO: 641 - DNA, 378 bp, Homo sapiens
ctggagcagtcaggagctgaggtgaagaagcctggggcctcagtgaaggtctcctgcaaggcctctggttacacttttactaactatgcaattacctgggtgcgacaggcccctggacaaggtcttgagtggatgggatggatcagcggtgacagcactaacacatactatggtcagaagttccagggaagagtcaccatgacgacagacacatccacgagcacagcctacatggagttgacgagcctgacatctgaggacacggccgtgtattactgtgcgagagaatcgctctatatgattgcgtttgggagagttatatggccaccacttgactactggggccagggaactctggtcaccatctcctctgcctcc

SEQ ID NO: 642 - DNA, 393 bp, Homo sapiens
gagctcacacagtctccagccaccctgtctgtgtctcctggggaaagcgccaccctctcctgcagggccagtcagagtgttagcgacaacttagcctggtaccagcagaaacctggccaggctcccaggctcctcatctatggtgcatccagcagggcccctgccatcccaggcaggttcagtggcagtgggtctgggacagacttcactctcaccatcagcagactggagcctgaagatcttgcagtgtatcactgtcagcagtatggtgcgtcaccttggacgttcggccaagggaccaaggtggaaatcaaacgaactgtggctgcaccatctgtcttcatcttcccgccatctgatgagcagttgaaatctggaactgcctctgttgtg

SEQ ID NO: 643 - DNA, 402 bp, Homo sapiens
gagctcacactcacgcagtctccaggcaccctgtctttgtctccaggggaaagagccaccctctcctgtagggccagtcagagtgttagcaacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcaacagtatggtagctcacctcagacgttcggccaagggaccaaggtggaaatcaaacgaactgtggctgcaccatctgtcttcatcttcccgccatctgatgagcagttgaaatctggaactgcctctgttgtg

SEQ ID NO: 644 - DNA, 411 bp, Homo sapiens
gagctcacccagtctccagactccctggctgtgtctctgggcgagagggccaccatcaactgcaagtccagccagagtgttttatacagctccaacaataagaactacttagcttggtaccagcagaaaccagggcaacctcctcagctgctcatttactgggcatctacccgggaatccggggtccctgaccgattcagtggcagcgggtctgggacagatttcactctcaccatcagcagcctgcaggctgaagatgtggcagtttatttctgtcagcaatattatagtactcccttcactttcggccctgggaccaaagtggaaatcaaacgaactgtggctgcaccatctgtcttcatcttcccgccatctgatgagcagttgaaatctggaactgcctctgttgtg

SEQ ID NO: 645 - DNA, 393 bp, Homo sapiens
gagctcgtgatgacacagtctccaggcaccctgtctttgtctccaggggaaagagccaccctctcctgcagggccagtcagcgtgttggcagcagctacttagcctggtaccagcagaaacctggccaggctcccaggctcctcgtctatggtgcatccagcagggccactggcatcccagacaggttcagtggcagtgggtctgggacagacttcactctcaccatcagcaggctgcagcctgaagattttgcagtatattactgtcagcagtatggtacgacgttcggccaagggaccagggtggacatcaaacgaactgtggctgcaccatctgtctccatcttcccgccatctgatgagcagttgaaatctggaactgcctctgttgtg

SEQ ID NO: 646 - DNA, 399 bp, Homo sapiens
gagctcacactcacgcagtctccaggcaccctgtctttgtctccagggaaaagagccaccctctcctgcagggccagtcagagtgttagcggcagctacttagcctggtaccagcagaaacctggccaggctcccaggctcctcatctatggtgcatccaacagggccactggcatcccacacaggttcagtggcagtgggtctgggacagacttcactctcaccatcagcagactggagcctgaggattttgcagtgtattactgtcagcagtatggttcctcaccgacgttcggccaggggaccagggtggacatcaaacgaactgtggctgcaccatctgtcttcatcttcccgccatctgatgagcagttgaaatctggaactgcctctgttgtg

SEQ ID NO: 647 - DNA, 360 bp, Homo sapiens
gagctcacgcagtctccaagcaccctgtctttgtctccaggggaaggagccaccctctcctgcaggcccagtcagagtgttagtagaaactacctagcctggtatcagcagaaacctggccaggctcccagactcctcatctatggtgcgtccaccagggccaccggcatcccagacaggttcagtggcagtgggtctgggacaaacttcactctcaccatcagcagactggagcctgaagattttgcagtgtatttctgtcagcactatggtaactcacctccatacacttttggccaggggaccaagctggagatcaaacgaactgtggctgcaccatctgtcttcatcttcccgcca

SEQ ID NO: 648 - DNA, 360 bp, Homo sapiens
gagctcacgcagtctccaggcaccctgtctttgtctccaggggaaagagccgccctctcttgcagggccagtcagagtattagcaccaactacttagcctggtaccagcagaaaccaggccaggctcccaggctcctcatctatggtacgtccaacagggccactggcatcccagacaggttcagtggcactgggtctgggacagacttcagtctcaccatcagcagactggagcctgaagattctgcagtgtattactgtcagcagtatggtacctcaccattcactttcggccctgggaccaaagtggatatcaaacgaactgtggctgcaccatctgtcttcatcttcccgccatct

SEQ ID NO: 649 - DNA, 396 bp, Homo sapiens
gagctcacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatctagcagggccactggcatcccagacaggttcagtggcagtgggtctgggacagacttcactctcaccatcagcagactggagcctgaagattttgcagtgtattactgccaacagtatggtagctcacctcagacgttcggccaagggaccaaggtggaaatcaaacgaactgtggctgcaccatctgtcttcatcttcccgccatctgatgagcagttgaaatctggaactgcctctgttgtg

SEQ ID NO: 650 - DNA, 360 bp, Homo sapiens
gagctcacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcggactggagcctgaagattttgcagtgtattactgtcagcagtatggaagctcacctctcactttcggcggagggaccaaggtggagatcaaacgaactgtggctgcaccatctgtcttcatcttcccgccatct

SEQ ID NO: 651 - DNA, 396 bp, Homo sapiens
gagctcacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcactatggaagttcatcgtacactttcggccaggggaccagggtggagatcaaacgaactgtggctgcaccatctgtcttcatcttcccgccatctgatgagcagttgaaatctggaactgcctctgttgtg

SEQ ID NO: 652 - DNA, 396 bp, Homo sapiens
gagctcacgcagtctccaggcaccttgtatgtgtctcctggggaaagagccaccctctcctgcagggccagtcagagtgttcccgacaaccacttagcctggtaccagcagaaacctggccagactcccaggctcctcatctatggtgcatccaagagggccacgggcatcccagacaggttcagtggcagtgggtctgggacagacttcactctcaccatcagcagactggagcctgaagattttgcagtgtattactgtcagcagtatggtagctcacctcagacgttcggccaagggaccaaggtggaaatcaaacgaactgtggctgcaccatctgtcttcatcttcccgccatctgatgagcagttgaaatctggaactgcctctgttgtg

SEQ ID NO: 653 - DNA, 396 bp, Homo sapiens
gagctcacacagtctccagccaccctgtctgtgtctccaggggaaagcgccaccctctcctgcagggccagtcagagtgttagcagcaacttagcctggtaccagcagaaacctggccaggctcccaggctcctcatctatggtgcatcgaccagggccactggtatcccagccaggttcagtggcagtgggtctgggacagacttcactctcaccatcagcagactggagcctgaagattttgcagtgtattactgtcagcagtatggtggctcacctccgtacacttttggccaggggaccaagctggagatcaaacgaactgtggctgcaccatctgtcttcatcttcccgccatctgatgagcagttgaaatctggaactgcctctgttgtg

SEQ ID NO: 654 - DNA, 378 bp, Homo sapiens
gagctcacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcaacagtatggtagctcacctcagacgttcggccaagggaccaaggtggaaatcaaacgaactgtggctgcaccatctgtcttcatcttcccgccatctgatgagcagttgaaatcg

SEQ ID NO: 655 - DNA, 360 bp, Homo sapiens
gagctcacgcagtctccagccaccctgtctgtgtctccaggggaaagagccaccctctcctgcagggccagtcagactattagcgacaacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtatattactgtcaacagtatggtagctcacctcagacgttcggccaagggaccaaggtggaaatcaaacgaactgtggctgcaccatctgtcttcatcttcccgccatctgat

SEQ ID NO: 656 - DNA, 393 bp, Homo sapiens
gagctcacactcacgcagtctccaggcaccctgtctttgtctccaggggaaagagccaccctctcctgcagggccagtcagactgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcaacagtatggtagctcacctcagacgttcggccaagggaccaaggtggaaatcaaacgaactgtggctgcaccatctgtcttcatcttcccgccatctgatgagcagttgaaatctggaactgct

SEQ ID NO: 657 - DNA, 393 bp, Homo sapiens
gagctcgtgatgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacagattcagtggcagtgggtctgggacagacttcactctcaccatcaccagactggagcctgaagattttgcagtgtattactgtcaacagtatggtagctcacctcagacgttcggccaagggaccaaggtgcaaatcaaacgaactgtggctgcaccatctgtcttcatcttcccgccatctgatgagcagttgaaatctggaactgct

SEQ ID NO: 658 - DNA, 405 bp, Homo sapiens
gagctcgtgttgacgcagtctccactctccctgcccgtcacccttggacagccggcctccatctcctgcaggtctactcagagcctcgtttacagtgatggaaacacctacttgaattggtttcaccagagggcaggccaacctccaaggcgcctaatttataaggtctctaaccgggactctggggtcccagagagattcagcggcagtgggtcaggcactgatttcacactgaaaatcagcagggtggaggctgaggatgttggcatttattactgcatgcaaggagcacactggcctcccactttcggcggagggaccaaggtggagatcaatcgaactgtggctgcaccatctgtcttcatcttcccgccatctgatgagcagttgaaatctggaactgct

SEQ ID NO: 659 - DNA, 363 bp, Homo sapiens
gagctccagatgacccagtctccatccttcctgtccgcttctgtgggagacagagtcaccatcacttgccgggccagtcagggcattagcagttatttagcctggtatcagcaaaaaccagggaaagcccctaagctcctgatctcttctgtatccactttgcaaagtggggtctcatcaaggttcagcggcagtggatctgggacaggattcactctcacaatcagcagcctgcagtctgaagattctgcaacttattactgtgaacaactgaatagtttcccgtacacttttggccaggggaccaagctggagatcaaacgaactgtggctgcaccatctgtcttcatcttcccgccatct

SEQ ID NO: 660 - DNA, 375 bp, Homo sapiens
gagctcactcagtctccagtctccctgcccgtcacccctggagagccggcctccatctcctgcaggtctagtcagagcctcctgcatagtaatggaaaccactatttggattggtacctgcagaagccagggcagtctccacagctcctgatgtatttgggttctaatcgggcctccggggtccctgacaggttcagtggcagtggatcaggcacagattttacactgaaaatcagcagagtggaggctgaggatgttggggtttattactgcatgcaaggtctacagaccccgtggacgttcggccaagggaccaaggtggaaatcaaacgaactgtggctgcaccatctgtcttcatcttcccgccatctgat

SEQ ID NO: 661 - DNA, 369 bp, Homo sapiens
gagctcacactcacgcagtctccaggcaccctgtctttgtctccaggggaaagagccaccctctcctgcagggccagtcagagtattagcagcagctacttagcctggtaccagcagaaacctggccaggctcccaggctcctcatctatggtgcatccagaagggccactggcatcccagacaggttcagtggcagcgggtctgggacagacttcactctcaccatcagcagactggagcctgaagattttgcagtgtattactgtcagcagtatggtagctcaccgctcactttcggcggagggaccaaggtggagatcaaacgaactgtggctgcaccatctgtcttcatcttcccgccatctgat

SEQ ID NO: 662 - DNA, 366 bp, Homo sapiens
gagctcacacagtctccagccaccctgtccgtgtctccaggggaaagagccaccctctcctgcagggccagtcggggtattagcagcaacttagcctggtaccagcagaagcctggccaggctcccaggctcctcatctatggtgcatccagcagggccactggcatcccagacaggttcagtggcagtggatctgggacagacttcactctcaccatcagcagactggagcctgaagattttgcagtgtattactgtcaacagtatggtagctcacctcagacgttcggccaagggaccgaggtggaaatcaaacgaactgtggctgcaccatctgtcttcatcttcccgccatctgatgagcag

SEQ ID NO: 663 - DNA, 408 bp, Homo sapiens
gagctcacactcacgcagtctccaggcaccctgtctttgtctccaggggagagagccaccctctcctgcagggccagtcagagtgttagcagcgactccttagcctggtaccagcagaaacctggccaggctcccaggctcctcatctatggtgcctctagcagggccactggcatcccagacaggttcagtggcagtgggtctgggacagacttcactctcaccatcagcagactggagcctgaagatcttggagtgtattactgtcagcagtatggtccctcacctccggggtacacttttggccaggggaccaagctggagatcaaacgaactgtggctgcaccatctgtcttcatcttcccgccatctgatgagcagttgaaatctggaactgcctctgttgtg

SEQ ID NO: 664 - DNA, 402 bp, Homo sapiens
gagctcacactcacgcagtctccgggcaccctgtctttgtctccaggggaaagaggcaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcagacgttcggccaagggaccaaggtggaaatcaaacgaactgtggctgcaccatctgtcttcatcttcccgccatctgatgagcagttgaaatctggaactgcctctgttgtg

SEQ ID NO: 665 - DNA, 393 bp, Homo sapiens
gagctcacacagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccgtcagcagactggagcctgaagattctgcagtgtatttctgtcagcagtattataggtccccactcactttcggcggagggaccaaggtggagatcaaacgaactgtggctgcaccatctgtcttcatcttcccgccatctgatgagcagttgaaatctggaactgcctctgttgtg

SEQ ID NO: 666 - DNA, 366 bp, Homo sapiens
gagctcacactcacgcagtctcccgccaccctgtctgtttctccaggggaaagagccacccttttttgtagggccaatcagagtgttggccgcaacttagcctggtaccagcagaaacctggccaggctcccaggctcctcatttatggtatatccaccaggaccactactaccccaaccaggttcagtggcagtgggtctgggacagacttcactctcaccatcagccgcctgcagtctgaagattttgcagtttattactgtcagcagtataacaagtggcctccgtggacgttcggccaagggaccaagttggaaatcaaacgaactgtggctgcaccatctgtcttcgtcttcccgccatct

SEQ ID NO: 667 - DNA, 402 bp, Homo sapiens
gagctcgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcgctctcaccatcaccagactggagcctgaagattttgcagtgtattactgtcagcagtatggtagctcacctcagacgttcggccaagggaccaaggtggaaatcaaacgaactgtggctgcaccatctgtcttcatcttcccgccatctgatgagcagttgaaatctggaactgtctctgttgtg

SEQ ID NO: 668 - DNA, 402 bp, Homo sapiens
gcggccgagctcactcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcagacgttcggccaagggaccaaggtggaaatcaaacgaactgtggctgcaccatctgtcttcatcttcccgccatctgatgagcagttgaaatctggaactgcctctgttgtg

SEQ ID NO: 669 - DNA, 393 bp, Homo sapiens
gagctcacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcggactggagcctgaagattttgcagtgtattactgtcaacaatatggtagctcacctcagacgttcggccaagggaccaaggtggaaatcaaacgaactgtggctgcaccatctgtcttcatcttcccgccatctgatgagcagttgaaatctggaactgcctctgtt

SEQ ID NO: 670 - DNA, 402 bp, Homo sapiens
gagttcacactcacgcagtctccaggcaccctgtctttgtctccaggggaaagaggcaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcagacgttcggccaagggaccaaggtggaaatcaaacgaactgtggctgcaccatctgtcttcatcttcccgccatctgatgagcagttgaaatctggaactgcctctgttgtg

SEQ ID NO: 671 - DNA, 402 bp, Homo sapiens
gagctcacactcacgcagtctccaggcaccctgtctttgtctccaggggaaagagccaccctctcctgcagggccagtcagagtgttagcagcagccacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcaacagtatggtagctcacctcagacgttcggccaagggaccgaggtggaaatcaaacgaactgtggctgcaccgtctgtcttcatcttcccgccatctgatgagcagttgaaatctggaactgcctctgttgtg

SEQ ID NO: 672 - DNA, 402 bp, Homo sapiens
gagctcacgcagtctccaggcaccctgtctttgtctccaggggaaagagccaccctttcctgcagggccagtcagagtgttagcagcaactccttagcctggtaccaacagaaacctggcctggctcccaggctcctcatctatggtgcatccagcagggccactggcatcccagagaggttcagtggcagtggggctgggacaggcttcactctcaccatcagcacactggagcctgaagattttgcaatttattactgtcaacaatatggtggctcgcctccaagattcactttcggccctgggaccaaagtggatatcagacgaactgtggctgcaccatctgtcttcatcttcccgccatctgatgagcagttgaaatctggaactgcctctgttgtg

SEQ ID NO: 673 - DNA, 393 bp, Homo sapiens
gagctcacgcagtctccaggcaccctgtcgttgtctccaggggaaagagccactctctcctgcagggccagtcagagtattaccagcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtgactccgtcggtttcggccctgggaccaaagtggaaatcaaacgaactgtggctgcaccatctgtcttcatcttcccgccatctgatgagcagttgaaatctggaactgcctctgttgtg

SEQ ID NO: 674 - DNA, 393 bp, Homo sapiens; identical to SEQ ID NO: 673

SEQ ID NO: 675 - DNA, 399 bp, Homo sapiens
gagctcgtgatgacgcagtctccagccaccctgtctttgtctccaggggaaagagccaccctctcctgcagggccagtcagagtgttggcagctacttagcctggtaccaacagaaacctggccaggctcccaggctcctcatttatgatgcatccaacagggccactggcatcccagccaggttcagtggcagtgggtctgggacagacttcactctcaccatcagcagcctagagcctgaagattttgctgtgtatttttgtcagcagtatggtagctcacccttgacgttcggccaagggaccaaggtggaaatcaaacgaactgtggctgcaccatctgtcttcatcttcccgccatctgatgagcagttgaaatctggaactgcctctgttgtg

SEQ ID NO: 676 - DNA, 393 bp, Homo sapiens
gagctcacgcagtctccaggcaccctgtctttgtctccaggggaaagagccaccctctcttgtagggccggtcagactgttgcgagcaattccttagcctggtatcagcacaaacctggccaggctcccaggctcctcatctatggtgcctccatcagggccagtggcatcccagacaggttcagtggcagtgggtctgggacagacttcactctcaccatcagcagactggagcctgaagattttgcagtgtattactgtcagcagtatggtctttcttccaccttcggccaagggacacgactggagattaaacgaactgtggctgcaccatctgtcttcatcttcccgccatctgatgagcagttgaaatctggaactgcctctgttgtg

SEQ ID NO: 677 - DNA, 390 bp, Homo sapiens
gagctcacactcacgcagtctccaggcaccctgtctttgtctccaggggaaagagccaccctctcctgcagggccagtcagagtattaggagcagttacttagcctggtaccagcagaaacctggccaggctcccaggctcctcatctatgctgcagccagcagggccactggcatcccagacaggttcagtggcagtgggtctgggacagacttcactctcaccatcagcagactggagcctgaagattttgcagtgtatttctgtcatcactatggtggctcacctcggacgttcggccaagggaccaaggtggaaatcaaacgaacggtggctgcaccatctgtcttcatcttcccgccatctgatgagcagttgaaatctggaact

SEQ ID NO: 678 - DNA, 381 bp, Homo sapiens
ctgctcgagtcaggtccaggactggtgaagccctcgcagaccctctcactcacctgtgacatctccggggacagtgtctctagccatagtgctgcttggaactggatcaggcagtccccatcgagaggccttgagtggctgggaaggacatactacaggtccaactggtataatgattatagtgtttctgtgagaagtcgaataatcatcagcgcagacacatccaagaaccagttctccctgcagctgaactctgtgactcccgaggacacggctgtgtattattgtgcaagagatcgcttctacggtcccgggggtgacttgtactacggtctggacgtctggggccaagggaccacggtcaccgtctcctcagcctcc

SEQ ID NO: 679 - DNA, 381 bp, Homo sapiens
ctgctcgagtcaggtccaggactggtgaagccctcgcagaccctctcactcacctgtgacatctcgggggacagtgtctctagccatagtgctgcttggaactggatcaggcagtccccatcgagaggccttgagtggctgggaaggacatactacaggtccaactggtataatgattatagtgtttctgtgagaagtcgaataatcatcagcgcagacacatccaagaaccagctctccctgcagctgaactctgtgactcccgaggacacggctgtgtattattgtgcaagagatcgcttctacggtcccgggggtgacttgtactacggtctggacgtctggggccaagggaccacggtcaccgtctcctcagcctcc

SEQ ID NO: 680 - DNA, 381 bp, Homo sapiens
ctgctcgagtcaggtccaggactggtgaagccctcgcagaccctctcactcacctgtgacatctccggggacagtgtctctagcaacagtgctgcttggaactggatcaggcagtccccatcgagaggccttgagtggctgggaaggacattctacaggtccaactggtataatgattatgcagtatctgtgaaaagtcgaataaccatcaacccagacacatccaagaaccagttctccctgcagctgaactctgtgactcccgaggacacggctgtgtattactgtgcaagagatttatatattggcccaaccacagacttctactacggtatggacgtctggggccaagggaccacggtcaccgtctcctcagcctcc

SEQ ID NO: 681 - DNA, 382 bp, Homo sapiens
gctgctcgagtcaggtccaggactggtgaggccctcgcagaccctctcactcacctgtgacatctccggggacagtgtctctagcaacagtgctgcttggaactggatcaggcagtccccatcgagaggccttgagtggctgggaaggacattctacaggtccaactggtataatgattatgcagtatctgtgaaaagtcgaataaccatcaacccagacacatccaagaaccagttctccctgcagctgaactctgtgactcccgaggacacggctgtgtattactgtgcaagagatttatatattggcccaaccacagacttctactacggtatggacgtctggggccaagggaccacggtcaccgtctcctcagcctcc

SEQ ID NO: 682 - DNA, 382 bp, Homo sapiens
gctgctcgagtcaggtccaggactggtaaagccctcgcagaccctctcactcacctgtgccatctccggggacagtgtctctagcaacagtgctgcttggaactggatcaggcagtccccgtcgagaggccttgagtggctgggaaggacattctacaggtccaagtggtattatgattatgcagtatctgtgaaaagtcgaatcaccatcaacccagacacatccaagaaccagttctccctgcacctgaactctgtgactcccgaggacacggctgtgtattattgtgtaagagatttttatattggcccaaccagagacttctactacggtatggacgtctggggccaagggaccacggtcaccgtctcctcagcctcc

SEQ ID NO: 683 - DNA, 396 bp, Homo sapiens
gagctcacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtggatctgggacagacttcactctcaccatcagcagactggagcctgaagattttgcagtgtattactgtcaacagtatggtagctcacctcagacgttcggccaagggaccaaggtggaaatcaaacgaactgtggctgcaccatctgtcttcatcttcccgccatctgatgagcagttgaaatctggaactgcctctgttgtg

SEQ ID NO: 684 - DNA, 372 bp, Homo sapiens
gagctcacgcagtctccaggcaccctgtctttgtctccaggggaaagaggcaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcagacgttcggccaagggaccaaggtggaaatcaaacgaactgtggctgcaccatctgtcttcatcttcccgccatctgatgagcagtta

SEQ ID NO: 685 - skipped

SEQ ID NO: 686 - DNA, 402 bp, Homo sapiens
gagctcacactcacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcagacgttcggccaagggaccaaggtggaaatcaagcgaactgtggctgcaccatctgtcttcatcttcccgccatctgatgagcagttgaaatctggaactgcttctgttgtg

SEQ ID NO: 687-688 - skipped (2 entries)

SEQ ID NO: 689 - DNA, 381 bp, Homo sapiens; identical to SEQ ID NO: 678

SEQ ID NO: 690 - DNA, 381 bp, Homo sapiens; identical to SEQ ID NO: 679

SEQ ID NO: 691 - DNA, 381 bp, Homo sapiens; identical to SEQ ID NO: 680

SEQ ID NO: 692 - DNA, 382 bp, Homo sapiens; identical to SEQ ID NO: 681

SEQ ID NO: 693 - DNA, 382 bp, Homo sapiens; identical to SEQ ID NO: 682

SEQ ID NO: 694 - protein, 29 aa, Hepatitis C virus
TAGLVGLLTPGAKQNIQLINTNGSWHINS

SEQ ID NO: 695 - protein, 12 aa, Hepatitis C Virus
GWLAGLFYQHKF

SEQ ID NO: 696 - protein, 18 aa, Hepatitis C Virus
GAPTYSWGANDTDVFVLN

SEQ ID NO: 697 - DNA, 18 bp, Hepatitis C Virus
gggatgctgcattgagta

SEQ ID NO: 698 - DNA, 19 bp, Hepatitis C Virus
tgcggaaccggtgagtaca

SEQ ID NO: 699 - DNA, 21 bp, Hepatitis C Virus
aggtttaggattcgtgctcat

SEQ ID NO: 700 - DNA, 27 bp, Hepatitis C Virus
caccctatcaggcagtaccacaaggcc

SEQ ID NO: 701 - protein, 239 aa, Hepatitis C Virus
ETHVTGGATAHGASVLASLLTTGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 702 - protein, 239 aa, Hepatitis C Virus
ETHVTGGATAHGASVLASLLTTGAKQNIQLINTNGSWHINRTALNCNDSLHTGFIAGLFYYNKFNSSGCPERLAGCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 703 - protein, 239 aa, Hepatitis C Virus
ETHVTGGATAHGASVLASLLTTGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPSYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 704 - protein, 239 aa, Hepatitis C Virus
ETHVTGGATAHGASVLASLLTTGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMDSTGFTKVCGAPPCVIGGVGNNTLRCPTDCFRKHPDATYSRCGSGPWITPRCLVDYPYRLWHYPCTI

SEQ ID NO: 705 - protein, 239 aa, Hepatitis C Virus
ETHVTGGATAHGASVLASLLTTGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGALPCVIGGVGNNTLRCPTDCFRKHPDATYSRCGSGPWITPRCLVDYPYRLWHYPCTI

SEQ ID NO: 706 - protein, 239 aa, Hepatitis C Virus
ETHVTGGATAHGASVLASLLTTGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGSNTLRCPTDCFRKHPDATYSRCGSGPWITPRCLVDYPYRLWHYPCTI

SEQ ID NO: 707 - protein, 239 aa, Hepatitis C Virus
ETHVTGGATAHGASVLASLLTTGAKQNIQLINTNGSWHVNRTALNCNDSLHTGFIAGLFYYNKFNSSGCPERLASCRRLDDFAQGWGPISHVNVSGPGERPYCWHYPPRPCGIVPARDVCGPVHCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 708 - protein, 239 aa, Hepatitis C Virus
ETHVTGGATAHGASVLASLLTTGAKQNIQLINTNGSWHINRTALNCNDSLHTGFIVGLFYYNKFNSSGCPERLASCRRLDDFAQGWGPISYVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 709 - protein, 239 aa, Hepatitis C Virus
ETHVTGGATAHGASVLASLLTTGAKQNIQLINTNGSWHINRTALNCNDSLHTGFIAGLFYYNKFDSSGCPERLASCRRLDDFAQGWGPISYVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 710 - protein, 239 aa, Hepatitis C Virus
ETHVTGGATAHGASVLASLLTTGAKQNIQLINTNGSWHINRTALNCNDSLHTGFIAGLFYYNKFDSSGCPERLASCRRLDDFAQGWGPISYVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCDIGGVGNNTLRCPTDCFRKHPDATYSRCGSGPWITPRCLVDYPYRLWHYPCTI

SEQ ID NO: 711 - protein, 239 aa, Hepatitis C Virus
ETHVTGGATAHGASVLASLLTPGAKQHVQLINTNGSWHINRTALNCNDSLHTGFIAGLFYYNKFDSSGCPERLASCRRLDDFAQGWGPISYVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 712 - protein, 239 aa, Hepatitis C Virus
ETHVTGGATAHGASVLASLLTPGAKQNIQLINTNGSWHINRTALNCNDSLHTGFVAGLFYYNKFDSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 713 - protein, 239 aa, Hepatitis C Virus
ETHVTGGATAHGASVLASLLTPGAKQNIQLINTNGSWHINRTALNCNDSLHTGFIAGLFYYNKFDSSGCLERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCAIGGVGNNTLRCPTDCFRKHPDATYSRCGSGPWITPRCLVDYPYRLWHYPCTI

SEQ ID NO: 714 - protein, 239 aa, Hepatitis C Virus
ETHVTGGATAHGASVLASLLTPGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCAIGGVGNNTLRCPTDCFRKHPDATYSRCGSGPWITPRCLVDYPYRLWHYPCTI

SEQ ID NO: 715 - protein, 239 aa, Hepatitis C Virus
ETHVTGGATAHGASVLASLLTPGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNTTGFTKVCGAPSCVIGGVGNNTLRCPTDCFRKHPDATYSRCGSGPWITPRCLVDYPYRLWHYPCTI

SEQ ID NO: 716 - protein, 239 aa, Hepatitis C Virus
ETHVTGGATAHGASVLASLLTPGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNTTGFTKVCGAPPCVIGGVGNNTLRCPTDCFRKHPDATYSRCGSGPWITPRCLVDYPYRLWHYPCTI

SEQ ID NO: 717 - protein, 239 aa, Hepatitis C Virus
ETHVTGGATAHGASVLASLLTPGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 718 - protein, 239 aa, Hepatitis C Virus
ETHVTGGATAHGASVLASLLTPGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPCITPRCLVDYPYRLWHYPCTI

SEQ ID NO: 719 - protein, 239 aa, Hepatitis C Virus
ETHVTGGATAHGASVLTSLLTTGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDIFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 720 - protein, 239 aa, Hepatitis C Virus
ETYVTGGATAHGASVLASLLTTGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 721 - protein, 239 aa, Hepatitis C Virus
ETHVTGGATAHGASVFASLLTTGAKQNIQLINTNGSWHINRTALNCNDSLHTGFIAGLFYYNKFNSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 722 - protein, 239 aa, Hepatitis C Virus
ETHVTGGATAHGASVFASLLTTGAKQNIQLINTNGSWHINRTALNCNDSLHTGFIAGLFYYNKFDSSGCPERLASCRRLDDFAQGWGPISHVNVSGPGERPYCWHYPPRPCGIVPARDVCGPVYCFTPSPVVVGTTDRAGAPTYNWGANETDVFVLNNTRPPLGNWFGCTWMNSTGFTKVCGAPPCVIGGVGNNTLRCPTDCFRKHPDATYSRCGSGPWITPRCLVDYPYRLWHYPCTI

SEQ ID NO: 723 - protein, 239 aa, Hepatitis C Virus
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTI

SEQ ID NO: 724 - protein, 239 aa, Hepatitis C Virus
RTRTTGGSAAQTTYGLTSLFRSGPSQKIQLVNTNGSWHINRTALNCNDSLNTGFLAALFYVRNFNSSGCPERMASCRPIDTFDQGWGPITYTEPHSLDQRPYCWHYAPQPCGIVPAAQVCGPVYCFTPSPVVVGTTDRFGAPTYTWGENETDVLILNNTRPPQGNWFGCTWMNGTGFTKTCGGPPCNIGGAGNNTLICPTDCFRKHPEATYTRCGSGPWLTPRCMVDYPYRLWHYPCTV

SEQ ID NO: 725 - protein, 35 aa, Homo sapiens
MDAMKRGLCCVLLLCGAVFVSPSQEIHARFRRGAR

SEQ ID NO: 726 - protein, 10 aa, Artificial Sequence
  a synthetic peptide
LEDYKDDDDK

SEQ ID NO: 727 - protein, 334 aa, Hepatitis C virus
ETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWE

SEQ ID NO: 728 - protein, 250 aa, Hepatitis C virus
QLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSE

SEQ ID NO: 729 - protein, 237 aa, Hepatitis C virus
QLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTR

SEQ ID NO: 730 - protein, 234 aa, Hepatitis C virus
QLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACN

SEQ ID NO: 731 - protein, 200 aa, Hepatitis C virus
QLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDY

SEQ ID NO: 732 - protein, 178 aa, Hepatitis C virus
QLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKH

SEQ ID NO: 733 - protein, 163 aa, Hepatitis C virus
QLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGV

SEQ ID NO: 734 - protein, 146 aa, Hepatitis C virus
QLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNS

SEQ ID NO: 735 - protein, 94 aa, Hepatitis C virus
QLINTNGSWHINSTALNCNESLNTGWLAGLFYQHKFNSSGCPERLASCRRLTDFAQGWGPISYANGSGLDERPYCWHYPPRPCGIVPAKSVCGP

SEQ ID NO: 736 - protein, 190 aa, Hepatitis C virus
LASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACN

SEQ ID NO: 737 - protein, 154 aa, Hepatitis C virus
RPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACN

SEQ ID NO: 738 - protein, 140 aa, Hepatitis C virus
VYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACN

SEQ ID NO: 739 - protein, 88 aa, Hepatitis C virus
TGFTKVCGAPPCVIGGVGNNTLLCPTDCFRKHPEATYSRCGSGPWITPRCMVDYPYRLWHYPCTINYTIFKVRMYVGGVEHRLEAACN

SEQ ID NO: 740 - protein, 212 aa, Artificial Sequence
  a synthetic peptide
QLINTNGSWHINSTALNCNESLNTGWLAGLFYQHKFNSSGCPERLASCGSSGCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACN

SEQ ID NO: 741 - protein, 227 aa, Artificial Sequence
  a synthetic peptide
QLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCGSSGCPTDCFRKHPEATYSRCGSGPWITPRCMVDYPYRLWHYPCTINYTIFKVRMYVGGVEHRLEAACN

SEQ ID NO: 742 - protein, 205 aa, Artificial Sequence
  a synthetic peptide
QLINTNGSWHINSTALNCNESLNTGWLAGLFYQHKFNSSGCPERLASCGSSGCWHYPPRPCGIVPAKSVCGPVYCFTPSPVVVGTTDRSGAPTYSWGANDTDVFVLNNTRPPLGNWFGCTWMNSTGFTKVCGAPPCGSSGCPTDCFRKHPEATYSRCGSGPWITPRCMVDYPYRLWHYPCTINYTIFKVRMYVGGVEHRLEAACN

SEQ ID NO: 743 - protein, 361 aa, Hepatitis C virus
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 744 - DNA, 27 bp, Hepatitis C virus
aataacgcgtgaaacccacgtcaccgg

SEQ ID NO: 745 - DNA, 29 bp, Hepatitis C virus
aataacgcgtcaactgatcaacaccaacg

SEQ ID NO: 746 - DNA, 28 bp, Hepatitis C virus
aataacgcgtttggccagctgccgacgc

SEQ ID NO: 747 - DNA, 29 bp, Hepatitis C virus
aataacgcgtagaccttgtggcattgtgc

SEQ ID NO: 748 - DNA, 30 bp, Hepatitis C virus
aataacgcgtgtatattgcttcactcccag

SEQ ID NO: 749 - DNA, 30 bp, Hepatitis C virus
aataacgcgtactggattcaccaaagtgtg

SEQ ID NO: 750 - DNA, 29 bp, Hepatitis C virus
tattctcgagctcccacttaatggcccag

SEQ ID NO: 751 - DNA, 28 bp, Hepatitis C virus
tattctcgagctcggacctgtccctgtc

SEQ ID NO: 752 - DNA, 28 bp, Hepatitis C virus
tattctcgagccgcgtccagttgcaggc

SEQ ID NO: 753 - DNA, 29 bp, Hepatitis C virus
tattctcgaggttgcaggccgcttccagc

SEQ ID NO: 754 - DNA, 29 bp, Hepatitis C virus
tattctcgaggtagtcgaccatgcacctg

SEQ ID NO: 755 - DNA, 30 bp, Hepatitis C virus
tattctcgagatgtttgcggaagcaatcag

SEQ ID NO: 756 - DNA, 30 bp, Hepatitis C virus
tattctcgagcacccctccgatgacacaag

SEQ ID NO: 757 - DNA, 30 bp, Hepatitis C virus
tattctcgagtgagttcatccaggtacaac

SEQ ID NO: 758 - DNA, 30 bp, Hepatitis C virus
tattctcgagcgggccacacacgctctttg

SEQ ID NO: 759 - DNA, 35 bp, Heptitis C virus
tgcggctctagcggatgctggcactaccctccaag

SEQ ID NO: 760 - DNA, 35 bp, Hepatitis C virus
cagcatccgctagagccgcagctggccaacctctc

SEQ ID NO: 761 - DNA, 34 bp, Hepatitis C virus
tgtggaagctctggctgccccactgattgcttcc

SEQ ID NO: 762 - DNA, 34 bp, Hepatitis C virus
gcagccagagcttccacaagggggcgctccgcac

SEQ ID NO: 763 - protein, 3010 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYAFYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRQAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGATEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 764 - protein, 3010 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYANEGLGWAGWLLSPRGSRPSWGPTDPRRRSRNLGKVIDTPTCGFADLMGYIPLVGAPLGGVARALAHGVRVLEDSVNYATGNLPGCSFSIFLLALLSCLTVPASAYEVRNVSGIYHVTNDCSNSSIVYEAADLIMHTPGCVPCVREANSSRCWVALTPTLAARDSSIPTATIRRHVDLLVGAAAFCSAMYVGDLCGSVFLVSQLFTFSPRLHQTVQDCNCSIYPGHLTGHRMAWDMMMNWSPTAALVVSQLLRIPQAIVDMVAGAHWGVLAGLAYYPMVGNWAKVLIVMLLFAGVDGTTVTMGGTVARTTYGFTGLFRPGASQKIQLINTNGSWHINRTALNCNDSLNTGFLAALFYTHRFNASGCPERMASCQSIDKFVQGWGPITYAENGSSDQRPYCWHYAPRRCGIVPASQVCGPVYCFTPSPVVVGTTDRSGAPTYSWGENETDVLLLNNTRPPQGNWFGCTWMSSTGFTKTCGGPPCNIGGAGNNTLTCPTDCFRKHPEATYTKCGSGPWLTPRCLVDYPYRLWHYPCTVNFTTFKVRMYVGGVEHRLIAACNWTRGERCNLEDRDRSELSPLLLSTTEWQILPCSYTTLPALSTGLIHLHQNIVDVQYLYGIGSAVVSFVIKWEYVLLFFLLLADARVCACLWMILLIAQAEAALENLVVLNAASVAGAHGILSFLVFFCAAWYIKGRLVPGAAYASYGVWPLLLLLLALPPRAYAMDQGMAASSGGTVLVGLMLLTLSPYYKVVLARLIWWLQYFITRAEAHLQVWVPPLNVRGGRDAVILLTCAVYPELVFDITKLLLAIFGPLMVLQAGIIKMPYFVRAQGLIRACMLVRKVAGGHYVQMAFMKLAALTGTYVYDHLTPLRDWAHTGLRDLAVAVEPVVFSDMETKIITWGADTAECGDIILGYRSSARRGREILLGPADSLEGQGWRLLAPITAYAQQTRGLLGCIITSLTGRDKNQVEGEVQVVSTATQSFLATCVNGVCWTVFHGAGSKTLAGPKGPITQMYTNVDQDLVGWQAAPGMRSLTPCTCGSSDLYLVTRHADVIPVRRRGDGRGSLLSPRPVSYLKGSSGGPLLWPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGTDPNIRTGARTITTGAPITYSTYGKFFADGGCSGGAYDIIICDECHSTDSTTILGIGTVLDRAETAGARLVVLATATPPGSTTVPHPNIEEVALPNTGEIPFYGRAIPIEFIKGGRHLIFCPSKKKCDELAAKLSALGINAVAYYRGLDVSVIPTSGDVVVVATDALMTGYTGDFDSVIDCNTCVTQTVDFSLDPTFTIETTTVPQDAVSRTQRRGRTGRGRGGIYRFVTPGERPSGMFDSSVLCECYDAGCAWYELTPAETTVRLRAYLNTPGLPVCQDHLEFWESVFTGLNHIDAHFLSQTKQAGDNFPYLVAYQATVCARAQAPPPSWDQMWKCLIWLKPVLHGPTPLLYRLGAVQNEITLTHPITKLIMASMSADLEVVTSTWVLVGGVLAALAAYCLTTGSVVIVGRIILSGRPAVIPDREVLYREFDEMEECASHLPYIEQGVQLAEQFKQKALGLLQTATKQAEAAAPVVESKWRALETFWAKHMWNFISGIQYLAALSTLPGNPAIASLMAFTASITSPLTTQNTLLFNILGGWVAAQLAPASAASAFVGAGSAGAAIGTIGLGKVLVDILAGYGAGVAGALVAFKVMSGEMPSTEDLVNLLPAILSPGALVVGVVCAAILRRHVGPGEGAVQWMNRLIAFASRGNHDSPTHYVPESDAAARVTQILSSLTITQLLKRLHQWINEDCSTPCSGSWLRDVWDWICTVLTDFKTWLQSKLLPRLPGVPFFSCQRGYKGVWRGDGIMQTTCPCGAQITGHVKNGSMRIVGPKTCSNTWHGTFPINAYTTGPCTPAPTPNYSRALWRVAAEEYVEVTRVGDFHYVTGMTTDNVKCPCQVPAPEFFTEVDGVRLHRYAPACKTLLREEVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESESKVVILDSFDPLRAEEGEGEVSVAAEILRKSKKFPPALPEWARPDYNPPLLESWKDPDYVPPVVHGCPLPPAKAPPIPPPRRKRTVVLTESTVSSALAELAVKTFGSSESSAVDSGTATAPPDQVSDNGDKGSDAESYSSMPPLEGEPGDPDLSDGSWSTVSEEASEDVVCCSMSYSWTGALITPCAAEESKLPINALSNSLLRHHNMVYATTSRSAGLRQKKVTFDRLQVLDDHYRDVLKEMKAKASTVKAKLLSVEEACKLTPPHSAKSKFGYGAKDVRNLSSRAVNHIRSVWKDLLEDTETPIDTTIMAKSEVFCVQPEKGGRKPARLIVFPDLGVRVCEKMALYDVVSTLPQAVMGPSYGFQYSPGQRVEFLVNAWKSKKCPMGFSYDTRCFDSTVTESDIRTEESIYQCCDLAPEAKQAIKSLTERLYIGGPLTNSKGQNCGYRRCRASVVLTTSCGNTLTCYLKASAACRAAKLQDCTMLVNGDDLVVICESAGTQEDAANLRAFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALECQIYGACYSIEPLDLPQIIERLHGLSAFSLHSYSPGEINRVASCLRKLGVPPLRVWRHRARRVRAKLLSQGGRAATCGKYLFNWAVRTKLKLTPIPAASRLDLSSWFVAGYSGGDIYHSVSHARPRWFMLCLLLLSVGVGIYLLPNR

SEQ ID NO: 765 - protein, 3011 aa, Hepatitis C virus
MSTNPKPQRKTKRNTNRRPQDVKFPGGGQIVGGVYLLPRRGPRVGVRATRKTSERSQPRGRRQPIPKARRPEGRSWAQPGYPWPLYGNEGCGWAGWLLSPRGSRPSWGPSDPRRRSRNLGKVIDTLTCGFADLMGYIPLVGAPLGGAARALAHGVRVLEDGVNYATGNLPGCSFSIFLLALLSCLTVPASAVGVRNSSGVYHVTNDCPNASVVYETENLIMHLPGCVPYVREGNASRCWVSLSPTVAARDSRVPVSEVRRRVDSIVGAAAFCSAMYVGDLCGSIFLVGQIFTFSPRHHWTTQDCNCSIYPGHVTGHRMAWDMMMNWSPTGALVVAQLLRIPQAIVDMIAGAHWGVLAGLAYYSMVGNWAKVVVVLLLFAGVDAETRVTGGAAGHTAFGFASFLAPGAKQKIQLINTNGSWHINRTALNCNESLDTGWLAGLLYYHKFNSSGCPERMASCQPLTAFDQGWGPITHEGNASDDQRPYCWHYALRPCGIVPAKKVCGPVYCFTPSPVVVGTTDRAGVPTYRWGANETDVLLLNNSRPPMGNWFGCTWMNSSGFTKTCGAPACNIGGSGNNTLLCPTDCFRKHPDATYSRCGSGPWLTPRCLVDYPYRLWHYPCTVNYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEAALENLIVLNAASLVGTHGIVPFFIFFCAAWYLKGKWAPGLAYSVYGMWPLLLLLLALPQRAYALDQELAASCGATVFICLAVLTLSPYYKQYMARGIWWLQYMLTRAEALLQVWVPPLNARGGRDGVVLLTCVLHPHLLFEITKIMLAILGPLWILQASLLKVPYFVRAHGLIRLCMLVRKTAGGQYVQMALLKLGAFAGTYIYNHLSPLQDWAHSGLRDLAVATEPVIFSRMEIKTITWGADTAACGDIINGLPVSARRGREVLLGPADALTDKGWRLLAPITAYAQQTRGLLGCIITSLTGRDKNQVEGEVQIVSTATQTFLATCVNGVCWTVYHGAGSRTIASASGPVIQMYTNVDQDLVGWPAPQGARSLTPCTCGASDLYLVTRHADVIPVRRRGDNRGSLLSPRPISYLKGSSGGPLLCPMGHAVGIFRAAVCTRGVAKAVDFVPVESLETTMRSPVFTDNSSPPTVPQSYQVAHLHAPTGSGKSTKVPAAYAAQGYKVLVLNPSVAATLGFGAYMSKAHGIDPNVRTGVRTITTGSPITHSTYGKFLADGGCSGGAYDIIICDECHSVDATSILGIGTVLDQAETAGVRLTILATATPPGSVTVPHSNIEEVALSTEGEIPFYGKAIPLNYIKGGRHLIFCHSKKKCDELAAKLVGLGVNAVAFYRGLDVSVIPTTGDVVVVATDALMTGYTGDFDSVIDCNTCVVQTVDFSLDPTFSIETSTVPQDAVSRSQRRGRTGRGKHGIYRYVSPGERPSGMFDSVVLCECYDAGCAWYELTPAETTVRLRAYLNTPGLPVCQDHLEFWESVFTGLTHIDAHFLSQTKQSGENFPYLVAYQATVCARAKAPPPSWDQMWKCLIRLKPTLTGATPLLYRLGGVQNEITLTHPITKYIMACMSADLEVVTSTWVLVGGVLAALAAYCLSTGSVVIVGRIILSGKPAVIPDREVLYREFDEMEECAAHIPYLEQGMHLAEQFKQKALGLLQTASKQAETITPAVHTNWQKLESFWAKHMWNFVSGIQYLAGLSTLPGNPAIASLMSFTAAVTSPLTTQQTLLFNILGGWVAAQLAAPAAATAFVGAGITGAVIGSVGLGKVLVDILAGYGAGVAGALVAFKIMSGEAPTAEDLVNLLPAILSPGALVVGVVCAAILRRHVGPGEGAVQWMNRLIAFASRGNHVSPTHYVPESDASVRVTHILTSLTVTQLLKRLHVWISSDCTAPCAGSWLKDVWDWICEVLSDFKSWLKAKLMPQLPGIPFVSCQRGYRGVWRGEGIMHARCPCGADITGHVKNGSMRIVGPKTCSNTWRGSFPINAHTTGPCTPSPAPNYTFALWRVSAEEYVEVRRLGDFHYITGVTTDKIKCPCQVPSPEFFTEVDGVRLHRYAPPCKPLLRDEVTFSIGLNEYLVGSQLPCEPEPDVAVLTSMLTDPSHITAETAARRLNRGSPPSLASSSASQLSAPSLKATCTTHHDSPDADLITANLLWRQEMGGNITRVESENKIVILDSFDPLVAEEDDREISVPAEILLKSKKFPPAMPIWARPDYNPPLVEPWKRPDYEPPLVHGCPLPPPKPTPVPPPRRKRTVVLDESTVSSALAELATKTFGSSTTSGVTSGEAAESSPAPSCDGELDSEAESYSSMPPLEGEPGDPDLSDGSWSTVSSDGGTEDVVCCSMSYSWTGALITPCAAEETKLPINALSNSLLRHHNLVYSTTSRSAGQRQKKVTFDRLQVLDDHYRDVLKEAKAKASTVKAKLLSVEEACSLTPPHSARSKFGYGAKDVRSHSSKAIRHINSVWQDLLEDNTTPIDTTIMAKNEVFCVKPEKGGRKPARLIVYPDLGVRVCEKRALYDVVKQLPIAVMGTSYGFQYSPAQRVDFLLNAWKSKKNPMGFSYDTRCFDSTVTEADIRTEEDLYQSCDLVPEARAAIRSLTERLYIGGPLTNSKGQNCGYRRCRASGVLTTSCGNTITCYLKASAACRAAKLRDCTMLVCGDDLVVICESAGVQEDAANLRAFTEAMTRYSAPPGDPPQPEYDLELITSCSSNVSVAHDGAGKRVYYLTRDPETPLARAAWETARHTPVNSWLGNIIMFAPTLWVRMVLMTHFFSILIAQEHLEKALDCEIYGAVHSVQPLDLPEIIQRLHGLSAFSLHSYSPGEINRVAACLRKLGVPPLRAWRHRARSVRATLLSQGGRAAICGKYLFNWAVKTKLKLTPLPSASQLDLSNWFTGGYSGGDIYHSVSHVRPRWFFWCLLLLSVGVGIYLLPNR

SEQ ID NO: 766 - protein, 363 aa, Hepatitis C virus
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 767 - protein, 363 aa, Hepatitis C virus
ETIVSGGQAARAMSGLVSLFTPGAKQNIQLINTNGSWHINSTALNCNESLNTGWLAGLIYQHKFNSSGCPERLASCRRLTDFDQGWGPISHANGSGPDQRPYCWHYPPKPCGIVPAKSVCGPVYCFTPSPVVVGTTDRSGAPTYNWGANDTDVFVLNNTRPPLGNWFGCTWMNSTGFTKVCGAPPCVIGGGGNNTLHCPTDCFRKHPEATYSRCGSGPWITPRCLVDYPYRLWHYPCTINYTIFKVRMYVGGVEHRLDAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 768 - protein, 363 aa, Hepatits C virus
TTVTMGGTVARTTYGFTGLFRPGASQKIQLINTNGSWHINRTALNCNDSLNTGFLAALFYTHRFNASGCPERMASCQSIDKFVQGWGPITYAENGSSDQRPYCWHYAPRRCGIVPASQVCGPVYCFTPSPVVVGTTDRSGAPTYSWGENETDVLLLNNTRPPQGNWFGCTWMSSTGFTKTCGGPPCNIGGAGNNTLTCPTDCFRKHPEATYTKCGSGPWLTPRCLVDYPYRLWHYPCTVNFTTFKVRMYVGGVEHRLIAACNWTRGERCNLEDRDRSELSPLLLSTTEWQILPCSYTTLPALSTGLIHLHQNIVDVQYLYGIGSAVVSFVIKWEYVLLFFLLLADARVCACLWMILLIAQAEA

SEQ ID NO: 769 - protein, 363 aa, Hepatitis C virus
TTQVTGGTAGRNAYRLASLFSTGPSQNIQLINSNGSWHINRTALNCNDSLHTGWVAALFYSHKFNSSGRPERMASCRPLTAFDQGWGPITYGGKASNDQRPYCWHYAPRPCGIVPAKEVCGPVYCFTPSPVVVGTTDKYGVPTYTWGENETDVLLLNNSRPPIGNWFGCTWMNSTGFTKTCGAPACNVGGSETNTLSCPTDCFRRHPDATYAKCGSGPWLNPRCMVDYPYRLWHYPCTVNYTIFKIRMFVGGIEHRLTAACNWTRGERCDLDDRDRAELSPLLLSTTQWQVLPCSFTTLPALSTGLIHLHQNIVDVQYLYGLSSVVTSWAIRWEYVVLLFLLLADARICACLWMMLLISQVEA

SEQ ID NO: 770 - protein, 367 aa, Hepatitis C virus
YTHTVGGAAASTANSIAGLLSRGPRQNLQLINSNGSWHINRTALNCHDSLQTGFITALFYARHFNSSGCPERLAACRNIEAFRVGWGALQYEDNVTNPEDMRPYCWHYPPKQCGIVPARSVCGPVYCFTPSPVVVGTTDKLGVPTYTWGENETDVFLLNSTRPPQGPWFGCTWMNSTGFTKTCGAPPCRTRADFNASTDLLCPTDCFRKHPDATYNKCGSGPWLTPRCLIDYPYRLWHYPCTVNYTTFKIRMYVGGVEHRLMAACNFTRGDSCDLSQRDRGQLSPLLHSTTEWAILPCSFSDLPALSTGLLHLHQNIVDVQYMYGLSPALTKYIVRWEWVVLLFLLLADARVCACIWMLILLGQAEA

SEQ ID NO: 771 - protein, 367 aa, Hepatitis C virus
RHHTTGLQVGKTLARVTSLFSIGPKQNIQLINTNGSWHINRTALNCNDSLQTGFIASLFYVNNINSSGCPERMSSCRELDDFRIGWGTLEYETNVTNDEDMRPYCWHYPPKPCGIVPARTVCGPVYCFTPSPIVVGTTDKQGVPTYSWGENETDVFLLNSTRPPRGSWFGCTWMNGTGFTKTCGAPPCRIRRDYNSTLDLLCPTDCFRKHPDTTYLKCGSGPWLTPKCLVEYPYRLWHYPCTVNFTIFKVRMYVGGVEHRFSAACNFTRGDRCRLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 772 - protein, 367 aa, Hepatitis C virus
STYTTGAVVGRSTHLFTSMFSLGSQQRVQLIHTNGSWHINRTALNCNDSLETGFLAALFYTSSFNSSGCPERLAACRSIESFRIGWGSLEYEESVTNDADMRPYCWHYPPRPCGIVPARTVCGPVYCFTPSPVVVGTTDRAGAPTYNWGENETDVFLLNSTRPPKGAWFGCTWMNGTGFTKTCGAPPCRIRKDFNASEDLLCPTDCFRKHPGATYIKCGAGPWLTPRCLVDYPYRLWHYPCTVNYTIYKVRMFVGGIEHRLQAACNFTRGDRCNLEDRDRSQLSPLLHSTTEWAILPCSYTDLPALSTGLLHLHQNIVDVQYLYGLSPAITKYVVKWEWVVLLFLLLADARVCACLWMLLLLGQAEA

SEQ ID NO: 773 - protein, 367 aa, Hepatitis C virus
STYSTGAQAGRAASGFAGLFTRGARQNIQLINTNGSWHINRTALNCNDSLQTGFIASLFYANSFNSSGCPERMAHCRSIEHFRIGWGALEYEENVINEEDMRPYCWHYPPKPCGVVPAKSVCGPVYCFTPSPVVVGTTDKRGVPTYNWGDNETDVFLLNSTRPPKGAWFGCTWMNGTGFTKTCGAPPCRIRRDFNASEDLLCPTDCFRKHPEATYSKCGAGPWLTPRCLIDYPYRLWHYPCTFNYTIFKIRMFVGGIEHRLQAACNFTRGDRCNLDDRDRSQLSPLLHSTTEWAILPCSFTDLPALSTGLIHLHQNIVDVQYLYGLTPAITKYVVKWEWVVLLFLLLADARVCACLWMLILLGQAEA

SEQ ID NO: 774 - protein, 367 aa, Hepatitis C virus
QTHTISGHAARTTHGLVSLFTPGSQQNIQLVNTNGSWHINRTALNCNDSLKTGFIAALFYSHKFNSSGCPQRMSSCRSIEEFRIGWGNLEYEENVTNDDNMRPYCWHYPPRPCGIVPAQTVCGPVYCFTPSPVVVGTTDRRGVPTYTWGENDTDVFLLNSTRPPRGAWFGCTWMNSTGFTKTCGAPPCRIRPDFNSSEDLLCPTDCFRKHSEATYTRCGAGPWLTPKCLFHYPYRLWHYPCTINFTIHKIRMFIGGVEHRLEAACNFTRGDRCNLEDRDRSQLSPLLHSTTEWAILPCTFSDMPALSTGLLHLHQNIVDVQYLYGLSPAITKYIVKWEWVVLLFLLLADARVCACLWMLLLLGQAEA

SEQ ID NO: 775 - protein, 369 aa, Hepatitis C virus
TTYTTGGNAARGASGIVSLFTPGAKQNLQLVNTNGSWHINRTALNCNDSINTGFIAGLIYYHKFNSTGCPQRLSSCKPITFFRQGWGSLTDANITGPSDDKPYCWHYPPRPCDTIRASSVCGPVYCFTPSPVVVGTTDAKGAPTYNWGANETDMFLLQSLRPPSGRWFGCTWMNSTGFTKTCGAPPCNIYGGGGNLNNESDLFCPTDCFRKHPEATYSRCGAGPWLTPRCLVDYPYRLWHYPCTVNFTLFRMRTFVGGFEHRFTAACNWTRGERCNIEDRDRSEQHPLLHSTTELAILPCSFTPMPALSTGLIHLHQNIVDVQYLYGIGSGVVGWALKWEFVILVFLLLADARVCVALWLMLMISQAEA

SEQ ID NO: 776 - protein, 370 aa, Hepatitis C virus
TTHTTGGSAAQATAGFTSFFTRGPSQNLQLVNSNGSWHINSTALNCNDSLNTGFIAGLFYYHKFNSSGCPERMSSCKPITYFNQGWGPLTDANINGPSEDRPYCWHYPPRPCNITKPLNVCGPVYCFTPSPVVVGTTDIKGLPTYRFGVNESDVFLLTSLRPPQGRWFGCVWMNSTGFVKTCGAPPCNIYGGMKDIEANQTHLKCPTDCFRKHHDATFTRCGSGPWLTPRCLVDYPYRLWHYPCTVNFSIFKVRMFVGGHEHRFSAACNWTRGERCDLEDRDRSEQQPLLHSTTDSLILPCSFTPMRRLSTGLIHLHQNIVDVQYLYGVGSAVVGWALKWEFVVLVFLLLADARVCVALWMMLLISQAEA

SEQ ID NO: 777 - protein, 368 aa, Hepatitis C virus
STTITGGVAASGAFTITSLFSTGAKQPLHLVNTNGSWHINRTALNCNDSLNTGFIAGLLYYHKFNSSGCVERMSACSPLDRFAQGWGPLGPANISGPSSEKPYCWHYAPRPCDTVPAQSVCGPVYCFTPSPVVVGATDKRGAPTYTWGENESDVFLLESARPPTEPWFGCTWMNGSGYVKTCGAPPCHIYGGREGKSNNSLVCPTDCFRKHPDATYNRCGAGPWLTPRCLVDYPYRLWHYPCTVNYTIFKVRMFVGGLEHRFNAACNWTRGERCNLEDRDRSEMYPLLHSTTEQAILPCSFVPIPALSTGLIHLHQNIVDVQYLYGISSGLVGWAIKWEFVILIFLLLADARVCVVLWMMMLISQAEA

SEQ ID NO: 778 - protein, 363 aa, Hepatitis C virus
ETHVSGAAVGRSTAGLANLFSSGSKQNLQLINSNGSWHINRTALNCNDSLNTGFLASLFYTHKFNSSGCSERLACCKSLDSYGQGWGPLGVANISGSSDDRPYCWHYAPRPCGIVPASSVCGPVYCFTPSPVVVGTTDHVGVPTYTWGENETDVFLLNSTRPPHGAWFGCVWMNSTGFTKTCGAPPCEVNTNNGTWHCPTDCFRKHPETTYAKCGSGPWITPRCLIDYPYRLWHFPCTANFSVFNIRTFVGGIEHRMQAACNWTRGEVCGLEHRDRVELSPLLLTTTAWQILPCSFTTLPALSTGLIHLHQNIVDVQYLYGVGSAVVSWALKWEYVVLAFLLLADARVSAYLWMMFMVSQVEA

SEQ ID NO: 779 - protein, 362 aa, Hepatitis C virus
QTHITGGKAGRDALTFAGLFTMGGQQHIQLINTNGSWHINRTALNCNDSLNTGFLASLFYYRRFNSSGCPERLASCSSLDSLPQGWGPLGIYQPNVPDTRPYCWNYTPRPCGTVSALTVCGPVYCFTPSPVVVGTTDRRGAPTYTWGENETDVFLLNTTRPPRGAWFGCTWMNSTGFTKSCGGPPCSITANGSTWGCPTDCFRKHPEATYTKCGSGPWLTPRCLVDYPYRLWHYPCTVNYTVFKVRMYIGGIEHRLDAACNWTRGEPCDLEHRDRTEISPLLLSTTQWQVLPCSFTTLPALSTGLIHLHQNIVDVQYLYGVGSAVVSWALKWEYVVLAFLLLAGARICACLWMMLLVAQVEA

SEQ ID NO: 780 - protein, 365 aa, Hepatitis C virus
VTYTTGSSAGSTIHGIANLFTPGSKQNLQLINTNGSWHINRTALNCNDSLQTGFIAGLIYRNKFNSSGCPERLSRCKRLDDLAQGWGKLGAANITGSSDDRPYCWHYAPRPCGVVPASEVCGPVYCFTPSPVAVGTTDRLGVPTYSWGANETDVFILNSTRPPRGAWFGCTWMNGTGFTKTCGAPPCQVQASVANQSWSCPTDCFRKHPETTYTKCGSGPWLTPRCLIDYPYRLWHYPCTVNFSIFKVRMFVAGVEHRLEAACNWTRGEPCGLEHRDRAELSPLLLSTTQWQVLPCSFTPLPALSTGLIHLHQNIVDVQYLYGIGSVVVSWALKWEYVVLAFLLLADARVCACLWMMLLVSQVEA

SEQ ID NO: 781 - protein, 364 aa, Hepatitis C virus
NTRTVGGSAAQGARGLASLFTPGPQQNLQLINTNGSWHINRTALNCNDSLQTGFVAGLLYYHKFNSTGCPQRMASCRPLAAFDQGWGTISYAAVSGPSDDKPYCWHYPPRPCGIVPARGVCGPVYCFTPSPVVVGTTDRKGNPTYSWGENETDIFLLNNTRPPTGNWFGCTWMNSTGFVKTCGAPPCNLGPTGNNSLKCPTDCFRKHPDATYTKCGSGPWLTPRCLVHYPYRLWHYPCTLNYTIFKVRMYIGGLEHRLEVACNWTRGERCDLEDRDRAELSPLLHTTTQWAILPCSFTPTPALSTGLIHLHQNIVDTQYLYGLSSSIVSWAVKWEYIVLAFLLLADARICTCLWIMLLVCQAEA

SEQ ID NO: 782 - protein, 368 aa, Hepatitis C virus
TTTVGHGVARTTAGITGLFSPGASQNLQLIKNGSSWHINRTALNCNDSLQTGFLASLFYVRKFNSSGCPERMAVCKSLADFRQGWGQITYKVNISGPSDDRPYCWHYAPRPCDVVPASTVCGPVYCFTPSPVVIGTTDRRGNPTYTWGENETDVFMLESLRPPTGGWFGCTWMNSTGFTKTCGAPPCQIIPGDYNSSANELLCPTDCFRKHPEATYQRCGSGPWVTPRCLVDYPYRLWHYPCTVNFTVHKVRMFVGGIEHRFDAACNWTRGERCELHDRDRIEMSPLLFSTTQLAILPCSFSTMPALSTGLIHLHQNIVDVQYLYGVSSSVTSWVVKWEYIVLMFLVLADARICTCLWLMLLISNVEA

SEQ ID NO: 783 - protein, 368 aa, Hepatitis C virus
TTTVGRAAGRSAYLFTSIFSSGPNQKIQLINTNGSWHINRTALNCIDSLQTGFLSALFYRSNFNSTGCSERLGACKPLEHFQQGWGPITHKSNITGPSEDRPYCWHYAPRECSVVPASSVCGPVYCFTPSPVVVGTTDRLGNPTYNWGENETDVFMLESLRPPQGGWFGCTWMNSTGFTKTCGAPPCQLIPGDYNSSSNQLLCPTDCFRKHPEATYQKCGSGPWLTPRCLVDYPYRLWHYPCTVNYTIHKVRMFIGGVEHRFDAACNWTRGDRCDLYDRDRIEMSPLLFSTTQLAILPCSFTTMPALSTGLIHLHQNIVDVQYLYGVSSSIVSWAVKWEYVVLMFLVLADARICTCLWLMLLVGKVEA

SEQ ID NO: 784 - protein, 367 aa, Hepatitis C virus
ETYVTGSVTGQTITGFSGLFSSGSQQKLQLVNTNGSWHINRTALNCNDSLQTGFIAALFYTYRFNASGCPARVSSCKPLTYFDQGWGPISYANVSGSSEDKPYCWHYPPRPCGVVPASQVCGPVYCFTPSPVVVGTTDRKGLPTYSWGENESDVFLLESLRPPKGGWYGCTWMNSTGFVKTCGAPPCNIRPDSTGANTTLICPTDCFRKHPEATYAKCGSGPWLTPRCVVDYPYRLWHYPCTQNYTLHKVRMFIGGLEHRFQAACNWTRGDPCNLEDRDRVEMSPLLFSTTELAILPCSFTTMPALSTGLIHLHQNIVDVQYLYGISPSVTSWVIKWEYVVLAFLVLADARICACLWLMLLIGQAEA

SEQ ID NO: 785 - protein, 367 aa, Hepatitis C virus
HTHVTGAVAGRTVGNIASLFSPGSRQNLQLINSNGSWHINRTALNCNDSLQTGFIASLFYFNKFNASGCPDRMSSCKPLTYFDQGWGPISYANVSGSSEDKPYCWHYPPRPCGVVPASQVCGPVYCFTPSPVVVGTTDKKGLPTYTWGENESDVFLLESLRPPKGGWYGCTWMNSTGYVKTCGAPPCNIKPDASSSNTTLTCPTDCFRKHPEATYTRCGSGPWLTPRCLVDYPYRLWHYPCTQNYTIHKVRMFVGGLEHRFQAACNWTRGAPCNLDDRDRVEMSPLLFSTTELAILPCSFTTMPALSTGLIHLHQNIVDVQYLYGISPSITSWVIKWEYIVLAFLLLADARICACLWLMLLIGQAEA

SEQ ID NO: 786 - protein, 363 aa, Hepatitis C virus
TTDVAHSAARTTHGIASLFSPGAHQRLQLINSNGSWHINRTALNCNDSLHTGFLANLFYVHKINDSGCPDRMSSCKPLTSFNKGWGPITYATIEGPSSDRPYCWHYAPRPCGVEPAKNVCGPVYCFTPSPVVVGTTDRVGLPTYTWGENETDVFILESVRPPQGGWFGCTWMNSTGFVKTCGAPPCKLGPGTNNSLVCPTDCFRKHPGATYAKCGSGPWLTPRCLVDYPYRLWHYPCTVNFTLHKIRMYVGGVEHRLTAACNWTRGDPCSLGDRDRAELSPLLFSTTELAILPCTFTPMPALSTGLIHLHQNIVDVQYLYGLTPSVVSWSIKWEYLVLAFLVLADARICACLWLMLMIAQVEA

SEQ ID NO: 787 - protein, 367 aa, Hepatitis C virus
STYVASSVSQATSGLVSLFSAGARQNLQLINTNGSWHINRTALNCNDSLQTGFIASLFYRNKFNATGCPERLSACKTLDSFDQGWGPITYANISGPAVEKPYCWHYPPRPCEVVSALNVCGPVYCFTPSPVVLGTTDRRGNPTYTWGANETDVFMMSSLRPPAGGWYGCTWMNTSGFVKTCGAPPCNIRPNPEENRTETLRCPTDCFRKHPGATYAKCGSGPWLTPRCLVDYPYRLWHYPCTVNYTLHKVRMYIAGSEHRFTAACNWTRGERCDLADRDRIEMSPLLFSTTELAILPCSFTTMPALSTGLIHLHQNVVDVQYLYGLSTSIVNWAIKWEYVVLLFLVLADSRICLALWLMLLIGQAEA

SEQ ID NO: 788 - protein, 365 aa, Hepatitis C virus
TTHIGSSASATTNRLTSFFSPGSKQNVQLIKTNGSWHINRTALNCNDSLHTGFIAGLLYAHRFNSSGCPERLSSCRPLHAFEQGWGPLTYANISGPSNDKPYCWHYPPRPCDIVPARSVCGPVYCFTPSPVVVGTTDRKGLPTYTWGANESDVFLLRSTRPPRGSWFGCTWMNSTGFVKTCGAPPCNTRPVGSGNDTLVCPTDCFRKHPEATYARCGSGPWLTPRCLVNYPYRLWHYPCTVNYTIHKVRMFVGGIEHRFEAACNWTRGERCELDDRDRVEMSPLLFSTTQLSILPCSFTTMPALSTGLIHLHQNIVDVQYLYGVSSAVVSWAVKWEYIVLAFLVLAVARVCACLWLMFLVGQAEA

SEQ ID NO: 789 - protein, 365 aa, Hepatitis C virus
TTGIGYAVSRATSGLTGLFTPGARQNIQLINTNGSWHINRTALNCNDSLQTGFIAGLIYAHKFNSTGCPDRLSWCRSLRSFDQGWGPITYANVSGSSDDRPYCWHYAPRPCTVVPASSVCGPVYCFTPSPVVIGTTDKKGFPTYSWGGNETDVFLLQSARPPRGAWFGCTWMNSTGFVKTCGAPPCNISPPSSSNNSLKCPTDCFRKHPGATYAKCGSGPWLTPRCLVDYPYRLWHYPCTVNYTIHKVRLYLWGIEHRFNAACNWTRGERCELDDRDRIEMSPLLFSTTELSILPCSFTTTPALSTGLIHLHQNVVDVQYLYGLSTAVVSWAVKWEYVVLAFLVLADARICACLWLMFLVGQAEA

SEQ ID NO: 790 - protein, 364 aa, Hepatitis C virus
TTYTTGGTAAHSVYGLSTLFTRGSQQNIQLVNSNGSWHVNRTALNCNDSLQTGFIAGLFYYHKFNSSGCLERMSSCKPITLFDQGWGPITYANTSGPSEDRPYCWHYPPRPCGIVPAREVCGPVYCFTPSPVVIGTTDKKGLPTYNWGENMSDVFLLQSARPPRGAWFGCTWMNSTGYVKTCGAPPCNIGPNTNTSLKCPTDCFRKHPDATYSRCGSGPWLTPRCLVDYPYRLWHYPCTINFTIHKVRMFLGGVEHRFSAACNWTRGERCELDDRDRVEMSPLLFSTTELAILPCSFTTMPALSTGLIHLHQNIVDIQYLYGVSTVLVSWAIKWEYVVLAFLVLADARICACMWLMFLVGQAEA

SEQ ID NO: 791 - protein, 29 aa, Hepatitis C virus
TAGLVGLLTPGAKQNIQLINTNGSWHINS

SEQ ID NO: 792 - protein, 29 aa, Hepatitis C virus
MSGLVSLFTPGAKQNIQLINTNGSWHINS

SEQ ID NO: 793 - protein, 29 aa, Hepatitis C virus
TYGFTGLFRPGASQKIQLINTNGSWHINR

SEQ ID NO: 794 - protein, 29 aa, Hepatitis C virus
AYRLASLFSTGPSQNIQLINSNGSWHINR

SEQ ID NO: 795 - protein, 29 aa, Hepatitis C virus
ANSIAGLLSRGPRQNLQLINSNGSWHINR

SEQ ID NO: 796 - protein, 29 aa, Hepatitis C virus
LARVTSLFSIGPKQNIQLINTNGSWHINR

SEQ ID NO: 797 - protein, 29 aa, Hepatitis C virus
THLFTSMFSLGSQQRVQLIHTNGSWHINR

SEQ ID NO: 798 - protein, 29 aa, Hepatitis C virus
ASGFAGLFTRGARQNIQLINTNGSWHINR

SEQ ID NO: 799 - protein, 29 aa, Hepatitis C virus
THGLVSLFTPGSQQNIQLVNTNGSWHINR

SEQ ID NO: 800 - protein, 29 aa, Hepatitis C virus
ASGIVSLFTPGAKQNLQLVNTNGSWHINR

SEQ ID NO: 801 - protein, 29 aa, Hepatitis C virus
TAGFTSFFTRGPSQNLQLVNSNGSWHINS

SEQ ID NO: 802 - protein, 29 aa, Hepatitis C virus
AFTITSLFSTGAKQPLHLVNTNGSWHINR

SEQ ID NO: 803 - protein, 29 aa, Hepatitis C virus
TAGLANLFSSGSKQNLQLINSNGSWHINR

SEQ ID NO: 804 - protein, 29 aa, Hepatitis C virus
ALTFAGLFTMGGQQHIQLINTNGSWHINR

SEQ ID NO: 805 - protein, 29 aa, Hepatitis C virus
IHGIANLFTPGSKQNLQLINTNGSWHINR

SEQ ID NO: 806 - protein, 29 aa, Hepatitis C virus
ARGLASLFTPGPQQNLQLINTNGSWHINR

SEQ ID NO: 807 - protein, 29 aa, Hepatitis C virus
TAGITGLFSPGASQNLQLIKNGSSWHINR

SEQ ID NO: 808 - protein, 29 aa, Hepatitis C virus
AYLFTSIFSSGPNQKIQLINTNGSWHINR

SEQ ID NO: 809 - protein, 29 aa, Hepatitis C virus
ITGFSGLFSSGSQQKLQLVNTNGSWHINR

SEQ ID NO: 810 - protein, 29 aa, Hepatitis C virus
VGNIASLFSPGSRQNLQLINSNGSWHINR

SEQ ID NO: 811 - protein, 29 aa, Hepatitis C virus
THGIASLFSPGAHQRLQLINSNGSWHINR

SEQ ID NO: 812 - protein, 29 aa, Hepatitis C virus
TSGLVSLFSAGARQNLQLINTNGSWHINR

SEQ ID NO: 813 - protein, 29 aa, Hepatitis C virus
TNRLTSFFSPGSKQNVQLIKTNGSWHINR

SEQ ID NO: 814 - protein, 29 aa, Hepatitis C virus
TSGLTGLFTPGARQNIQLINTNGSWHINR

SEQ ID NO: 815 - protein, 29 aa, Hepatitis C virus
VYGLSTLFTRGSQQNIQLVNSNGSWHVNR

SEQ ID NO: 816 - protein, 12 aa, Hepatitis C virus
GWLAGLFYQHKF

SEQ ID NO: 817 - protein, 12 aa, Hepatitis C virus
GWLAGLIYQHKF

SEQ ID NO: 818 - protein, 12 aa, Hepatitis C virus
GFLAALFYTHRF

SEQ ID NO: 819 - protein, 12 aa, Hepatitis C virus
GWVAALFYSHKF

SEQ ID NO: 820 - protein, 12 aa, Hepatitis C virus
GFITALFYARHF

SEQ ID NO: 821 - protein, 12 aa, Hepatitis C virus
GFIASLFYVNNI

SEQ ID NO: 822 - protein, 12 aa, Hepatitis C virus
GFLAALFYTSSF

SEQ ID NO: 823 - protein, 12 aa, Hepatitis C virus
GFIASLFYANSF

SEQ ID NO: 824 - protein, 12 aa, Hepatitis C virus
GFIAALFYSHKF

SEQ ID NO: 825 - protein, 12 aa, Hepatitis C virus
GFIAGLIYYHKF

SEQ ID NO: 826 - protein, 12 aa, Hepatitis C virus
GFIAGLFYYHKF

SEQ ID NO: 827 - protein, 12 aa, Hepatitis C virus
GFIAGLLYYHKF

SEQ ID NO: 828 - protein, 12 aa, Hepatitis C virus
GFLASLFYTHKF

SEQ ID NO: 829 - protein, 12 aa, Hepatitis C virus
GFLASLFYYRRF

SEQ ID NO: 830 - protein, 12 aa, Hepatitis C virus
GFIAGLIYRNKF

SEQ ID NO: 831 - protein, 12 aa, Hepatitis C virus
GFVAGLLYYHKF

SEQ ID NO: 832 - protein, 12 aa, Hepatitis C virus
GFLASLFYVRKF

SEQ ID NO: 833 - protein, 12 aa, Hepatitis C virus
GFLSALFYRSNF

SEQ ID NO: 834 - protein, 12 aa, Hepatitis C virus
GFIAALFYTYRF

SEQ ID NO: 835 - protein, 12 aa, Hepatitis C virus
GFIASLFYFNKF

SEQ ID NO: 836 - protein, 12 aa, Hepatitis C virus
GFLANLFYVHKI

SEQ ID NO: 837 - protein, 12 aa, Hepatitis C virus
GFIASLFYRNKF

SEQ ID NO: 838 - protein, 12 aa, Hepatitis C virus
GFIAGLLYAHRF

SEQ ID NO: 839 - protein, 12 aa, Hepatitis C virus
GFIAGLIYAHKF

SEQ ID NO: 840 - protein, 12 aa, Hepatitis C virus
GFIAGLFYYHKF

SEQ ID NO: 841 - protein, 18 aa, Hepatitis C virus
GAPTYSWGANDTDVFVLN

SEQ ID NO: 842 - protein, 18 aa, Hepatitis C virus
GAPTYNWGANDTDVFVLN

SEQ ID NO: 843 - protein, 18 aa, Hepatitis C virus
GAPTYSWGENETDVLLLN

SEQ ID NO: 844 - protein, 18 aa, Hepatitis C virus
GVPTYTWGENETDVLLLN

SEQ ID NO: 845 - protein, 18 aa, Hepatitis C virus
GVPTYTWGENETDVFLLN

SEQ ID NO: 846 - protein, 18 aa, Hepatitis C virus
GVPTYSWGENETDVFLLN

SEQ ID NO: 847 - protein, 18 aa, Hepatitis C virus
GAPTYNWGENETDVFLLN

SEQ ID NO: 848 - protein, 18 aa, Hepatitis C virus
GVPTYNWGDNETDVFLLN

SEQ ID NO: 849 - protein, 18 aa, Hepatitis C virus
GVPTYTWGENDTDVFLLN

SEQ ID NO: 850 - protein, 18 aa, Hepatitis C virus
GAPTYNWGANETDMFLLQ

SEQ ID NO: 851 - protein, 18 aa, Hepatitis C virus
GLPTYRFGVNESDVFLLT

SEQ ID NO: 852 - protein, 18 aa, Hepatitis C virus
GAPTYTWGENESDVFLLE

SEQ ID NO: 853 - protein, 18 aa, Hepatitis C virus
GVPTYTWGENETDVFLLN

SEQ ID NO: 854 - protein, 18 aa, Hepatitis C virus
GAPTYTWGENETDVFLLN

SEQ ID NO: 855 - protein, 18 aa, Hepatitis C virus
GVPTYSWGANETDVFILN

SEQ ID NO: 856 - protein, 18 aa, Hepatitis C virus
GNPTYSWGENETDIFLLN

SEQ ID NO: 857 - protein, 18 aa, Hepatitis C virus
GNPTYTWGENETDVFMLE

SEQ ID NO: 858 - protein, 18 aa, Hepatitis C virus
GNPTYNWGENETDVFMLE

SEQ ID NO: 859 - protein, 18 aa, Hepatitis C virus
GLPTYSWGENESDVFLLE

SEQ ID NO: 860 - protein, 18 aa, Hepatitis C virus
GLPTYTWGENESDVFLLE

SEQ ID NO: 861 - protein, 18 aa, Hepatitis C virus
GLPTYTWGENETDVFILE

SEQ ID NO: 862 - protein, 18 aa, Hepatitis C virus
GNPTYTWGANETDVFMMS

SEQ ID NO: 863 - protein, 18 aa, Hepatitis C virus
GLPTYTWGANESDVFLLR

SEQ ID NO: 864 - protein, 18 aa, Hepatitis C virus
GFPTYSWGGNETDVFLLQ

SEQ ID NO: 865 - protein, 18 aa, Hepatitis C virus
GLPTYNWGENMSDVFLLQ

SEQ ID NO: 866 - protein, 351 aa, Hepatitis C virus
TAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 867 - protein, 322 aa, Hepatitis C virus
TAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWE

SEQ ID NO: 868 - protein, 266 aa, Hepatitis C virus
TAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSE

SEQ ID NO: 869 - protein, 252 aa, Hepatitis C virus
TAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWT

SEQ ID NO: 870 - protein, 250 aa, Hepatitis C virus
TAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACN

SEQ ID NO: 871 - protein, 228 aa, Artificial Sequence
  a synthetic peptide
TAGLVGLLTPGAKQNIQLINTNGSWHINSTALNCNESLNTGWLAGLFYQHKFNSSGCPERLASCGSSGCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACN

SEQ ID NO: 872 - protein, 243 aa, Artificial Sequence
  a synthetic peptide
TAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCGSSGCPTDCFRKHPEATYSRCGSGPWITPRCMVDYPYRLWHYPCTINYTIFKVRMYVGGVEHRLEAACN

SEQ ID NO: 873 - protein, 221 aa, Artificial Sequence
  a synthetic peptide
TAGLVGLLTPGAKQNIQLINTNGSWHINSTALNCNESLNTGWLAGLFYQHKFNSSGCPERLASCGSSGCWHYPPRPCGIVPAKSVCGPVYCFTPSPVVVGTTDRSGAPTYSWGANDTDVFVLNNTRPPLGNWFGCTWMNSTGFTKVCGAPPCGSSGCPTDCFRKHPEATYSRCGSGPWITPRCMVDYPYRLWHYPCTINYTIFKVRMYVGGVEHRLEAACN

SEQ ID NO: 874 - DNA, 1053 bp, Hepatitis C virus
acggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttcctcctgcttgcagacgcgcgcgtctgctcctgcttgtggatgatgttactcatatcccaagcggaggcg

SEQ ID NO: 875 - DNA, 966 bp, Hepatitis C virus
acggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggag

SEQ ID NO: 876 - DNA, 798 bp, Hepatitis C virus
acggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgag

SEQ ID NO: 877 - DNA, 756 bp, Hepatitis C virus
acggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacg

SEQ ID NO: 878 - DNA, 750 bp, Hepatitis C virus
acggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaac

SEQ ID NO: 879 - DNA, 684 bp, Artificial Sequence
  a synthetic oligonucleotide
acggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgcggctctagcggatgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaac

SEQ ID NO: 880 - DNA, 729 bp, Artificial Sequence
  a synthetic oligonucleotide
acggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtggaagctctggctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaac

SEQ ID NO: 881 - DNA, 663 bp, Artificial Sequence
  a synthetic oligonucleotide
acggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgcggctctagcggatgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtggaagctctggctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaac